(19) United States
(12) Patent Application Publication
(10) Pub. No.: US 2004/0181038 A1
(43) Pub. Date: Sep. 16, 2004

(54) Novel fabh enzyme, compositions capable of binding to said enzyme and methods of use thereof

(75) Inventors: Cheryl Ann Janson, Bryn Mawr, PA (US); Xiayang Qiu, Audubon, PA (US)

Correspondence Address: GLAXOSMITHKLINE, Corporate Intellectual Property - UW2220, P.O. Box 1539, King of Prussia, PA, 19406-0939, US

(73) Assignee: SmithKline Beecham Corporation

(21) Appl. No.: 10/802,696

(22) Filed: Mar. 17, 2004

Related U.S. Application Data
(63) Continuation of application No. 09/980,945, filed on Dec. 5, 2001.
Application No. 09/980,945 is a 371 of international application No. PCT/US00/15659, filed on Jun. 7, 2000.
(60) Provisional application No. 60/138,124, filed on Jun. 7, 1999.

(51) Int. Cl. C07K 14/245

(52) U.S. Cl. 530/350; 702/19

(57) ABSTRACT

A novel E. coli FabH crystalline structure is identified. Also disclosed are methods of identifying inhibitors of these enzymes and/or active sites, and inhibitors identified by these methods.

### TECHNICAL FIELD OF THE INVENTION

The invention relates to the identification of a novel enzyme active site and methods enabling the design and selection of inhibitors of that active site.

### BACKGROUND OF THE INVENTION

The pathway for the biosynthesis of saturated fatty acids is very similar in prokaryotes and eukaryotes. However, the organization of the biosynthetic apparatus is very different. Vertebrates possess a type I fatty acid synthase (FAS) in which all of the enzymatic activities are encoded on one multifunctional polypeptide, the mature protein being a homodimer. The acyl carrier protein (ACP) is an integral part of the complex. In contrast, in most bacterial and plant FASs (type II) each of the reactions are catalyzed by distinct monofunctional enzymes and the ACP is a discrete protein. Mycobacteria are unique in that they possess both type I and II FASs. There therefore appears to be considerable potential for selective inhibition of the bacterial systems by broad-spectrum antibacterial agents (Rock, C. & Cronan, J. 1996. Biochimica et Biophysica Acta 1302, 1-16; Jackowski, S. 1992. In Emerging Targets in Antibacterial and Antifungal Chemotherapy. Ed. J. Sutcliffe & N. Georgopapadakou. Chapman & Hall, New York; Jackowski, S. et al. (1989). J. Biol. Chem. 264, 7624-7629.)

The first step in the biosynthetic cycle is the condensation of malonyl-ACP with acetyl-CoA by FabH. Prior to this, malonyl-ACP is synthesized from ACP and malonyl-CoA by FabD, malonyl CoA:ACP transacylase. In subsequent rounds malonyl-ACP is condensed with the growing-chain acyl-ACP (FabB and FabF, synthases I and II respectively). The second step in the elongation cycle is ketoester reduction by NADPH-dependent β-ketoacyl-ACP reductase (FabG). Subsequent dehydration by β-hydroxyacyl-ACP dehydrase (either FabA or FabZ) leads to trans-2-enoyl-ACP which is in turn converted to acyl-ACP by enoyl-ACP reductase (FabI). Further rounds of this cycle, adding two carbon atoms per cycle, eventually lead to palmitoyl-ACP whereupon the cycle is stopped largely due to feedback inhibition of FabH and I by palmitoyl-ACP (Heath, et al, (1996), J.Biol.Chem. 271, 1833-1836).

Cerulenin and thiolactomycin are potent and selective inhibitors of bacterial fatty acid biosynthesis. Extensive work with these inhibitors has proved that this biosynthetic pathway is essential for bacterial viability. No marketed antibiotics are targeted against fatty acid biosynthesis, therefore it is unlikely that novel antibiotics would be rendered inactive by known antibiotic resistance mechanisms. There is an unmet need for developing new classes of antibiotic compounds, such as those that target FabH.

FabH enzymes are of interest as potential targets for antibacterial agents.

There is a need in the art for novel FabH enzyme active sites and catalytic sequences to enable identification and structure-based design of inhibitors, which are useful in the treatment or prophylaxis of diseases, particularly diseases caused by bacteria which may share catalytic domains with those of the invention.

### SUMMARY OF THE INVENTION

In one aspect, the present invention provides a novel FabH enzyme active site crystalline form.

In another aspect, the present invention provides a novel FabH composition characterized by the catalytic residues Cys112, His244 and Asn274.

In still another aspect, the present invention provides a novel FabH composition characterized by the active site of 33 amino acid residues (including the catalytic residues).

In yet another aspect, the invention provides a method for identifying inhibitors of the compositions described above which methods involve the steps of: providing the coordinates of the structure of the invention to a computerized modeling system; identifying compounds which will bind to the structure; and screening the compounds identified for FabH inhibitory bioactivity.

In a further aspect, the present invention provides an inhibitor of the catalytic activity of any composition bearing the catalytic domain described above.

Another aspect of this invention includes machine readable media encoded with data representing the coordinates of the three-dimensional structure of the FabH crystal.

Other aspects and advantages of the present invention are described further in the following detailed description of the preferred embodiments thereof.

### BRIEF DESCRIPTION OF THE DRAWINGS

FIG. 1 provides the atomic coordinates of the E. coli FabH dimer.

FIG. 2 provides the atomic coordinates of the E. coli FabH monomer in complex with acetyl-CoA.

FIG. 3 provides a projection of the ribbon diagram of the E. coli FabH dimer. The two monomers are drawn with a light or dark gray shading. The catalytic Cys112 is shown in dark ball-and-stick model.

FIG. 4 provides the ribbon diagram of the E. coli FabH monomer with the catalytic residue Cys112 is shown in dark ball-and-stick model. The N- and C-termini are labeled.

FIG. 5 provides the stereoview of the α-carbon superposition between the structures of FabH and FabF. FabH is drawn in a thin black line and FabF in a thick gray line.

FIG. 6 provides the ribbon diagram of the E. coli FabH monomer with acetylated Cys112 and the CoA molecule in black ball-and-stick model. The orientation of the view is the same as that of FIG. 4.

FIG. 7 provides the superposition of the E. coli FabH catalytic residues in comparison to those of FabF. FabH is drawn in thick gray lines and FabF in thin black lines. FabH residues are label Cys112, His244 and Asn274, which corresponds to Cys163, His303 and His340, respectively.

### DETAILED DESCRIPTION OF THE INVENTION

The present invention provides a novel E. coli FabH crystalline structure, a novel FabH active site, and methods of use of the crystalline form and active site to identify FabH inhibitor compounds (peptide, peptidomimetic or synthetic compositions) characterized by the ability to competitively inhibit binding to the active site of a FabH enzyme. Also provided herein is a novel FabH crystalline structure in complex with the substrate acetyl-CoA, and the identification of acetyl-CoA interacting residues in FabH.

I. The Novel FabH Crystalline Three-Dimensional Structure

The present invention provides a novel FabH crystalline structure based on the E. coli FabH. The amino acid sequences of the FabH are provided in Table 1 as SEQ ID NO:1.

TABLE 1

Met Tyr Thr Lys Ile Ile Gly Thr Gly Ser Tyr Leu Pro Glu Gln
1 5 10 15
Val Arg Thr Asn Ala Asp Leu Glu Lys Met Val Asp Thr Ser Asp
16 20 25 30
Glu Trp Ile Val Thr Arg Thr Gly Ile Arg Glu Arg His Ile Ala
31 35 40 45
Ala Pro Asn Glu Thr Val Ser Thr Met Gly Phe Glu Ala Ala Thr
46 50 55 60
Arg Ala Ile Glu Met Ala Gly Ile Glu Lys Asp Gln Ile Gly Leu
61 65 70 75
Ile Val Val Ala Thr Thr Ser Ala Thr His Ala Phe Pro Ser Ala
76 80 85 90
Ala Cys Gln Ile Gln Ser Met Leu Gly Ile Lys Gly Cys Pro Ala
91 95 100 105
Phe Asp Val Ala Ala Ala Cys Ala Gly Phe Thr Tyr Ala Leu Ser
106 110 115 120
Val Ala Asp Gln Tyr Val Lys Ser Gly Ala Val Lys Tyr Ala Leu
121 125 130 135
Val Val Gly Ser Asp Val Leu Ala Arg Thr Cys Asp Pro Thr Asp
136 140 145 150
Arg Gly Thr Ile Ile Ile Phe Gly Asp Gly Ala Gly Ala Ala Val
151 155 160 165
Leu Ala Ala Ser Glu Glu Pro Gly Ile Ile Ser Thr His Leu His
166 170 175 180
Ala Asp Gly Ser Tyr Gly Glu Leu Leu Thr Leu Pro Asn Ala Asp
181 185 190 195
Arg Val Asn Pro Glu Asn Ser Ile His Leu Thr Met Ala Gly Asn
196 200 205 210
Glu Val Phe Lys Val Ala Val Thr Glu Leu Ala His Ile Val Asp
211 215 220 225
Glu Thr Leu Ala Ala Asn Asn Len Asp Arg Ser Gln Leu Asp Trp
226 230 235 240
Leu Val Pro His Gln Ala Asn Leu Arg Ile Ile Ser Ala Thr Ala
241 245 250 255
Lys Lys Leu Gly Met Ser Met Asp Asn Val Val Val Thr Leu Asp
256 260 265 270
Arg His Gly Asn Thr Ser Ala Ala Ser Val Pro Cys Ala Leu Asp
271 275 280 285
Glu Ala Val Arg Asp Gly Arg Ile Lys Pro Gly Gln Leu Val Leu
286 290 295 300
Leu Glu Ala Phe Gly Gly Gly Phe Thr Trp Gly Ser Ala Leu Val Arg Phe
301 305 310 317

As illustrated herein, the crystal structure is a tightly associated FabH dimer. Each monomer has two structural domains: the N-terminal domain (residues 1-170 of SEQ ID NO:1) and the C-terminal domain (residues 171-317 of SEQ ID NO:1). The two domains are similar in their overall fold: each contains a 5-stranded β-sheet sandwiched between α-helices and covered by other β-strands, α-helices and loops. The structural similarity between the two halves of the protein indicates that FabH is probably evolved from two genes of similar origin. The active site of FabH is at the center of the FabH monomer, formed at the junction of the N- and C-terminal domains. While the core architecture of the E. coli FabH bears some similarity to that of the FabF (Huang, et al, (1998), EMBO J. 17, 1183-1191), large differences exit in the atomic positions of the core β-strands, and the structures outside of the core β-strand are completely different. With amino acid sequence identity between FabH and FabF being below 20%, the large differences are well expected. Therefore, the crystalline structure of E. coli FabH is novel.

As described above, the E. coli FabH is a dimer, each monomer contains an active site. The dimer formation is essential for the FabH activity because the active site of a monomer is comprised of at least Phe87 of the other monomer in the dimer. The present invention provides both a crystalline monomer and dimer structure of E. coli FabH. Inhibitors that perturb or interact with this dimer interface are another target for the design and selection of anti-bacterial agents.

According to the present invention, the crystal structure of E. coli FabH has been resolved at 2.0 Å (crystal form 1), and its selenomethionine mutant protein in complex with acetyl-CoA has been determined at 1.9 Å (crystal form 2). The structure was determined using the methods of MAD phasing and molecular replacement, and refined to R-factors of 18.9% and 27%, respectively.

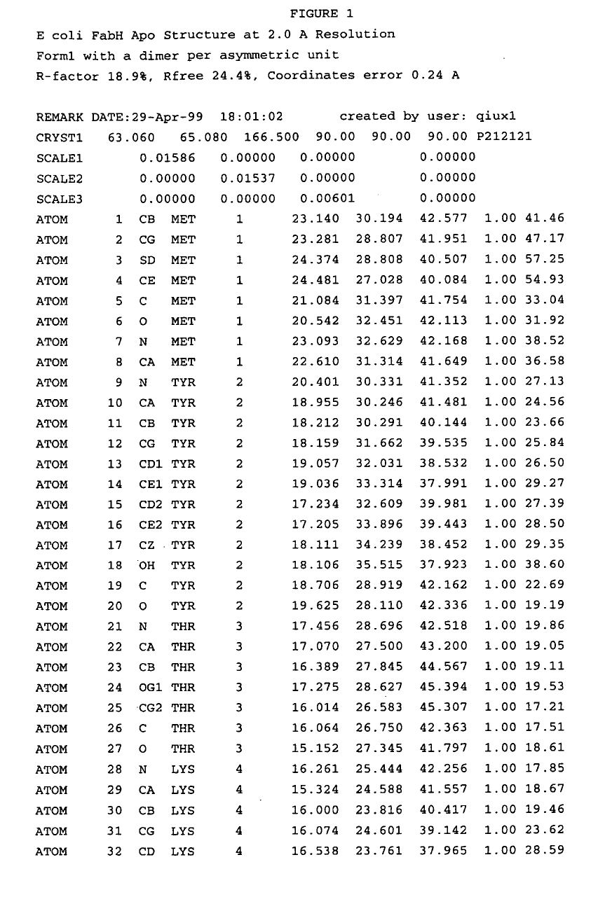

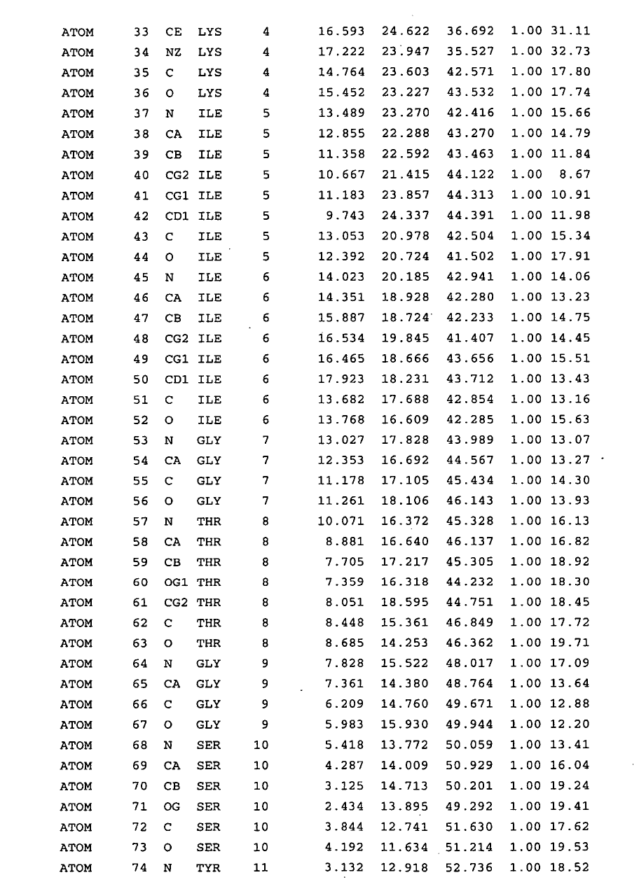

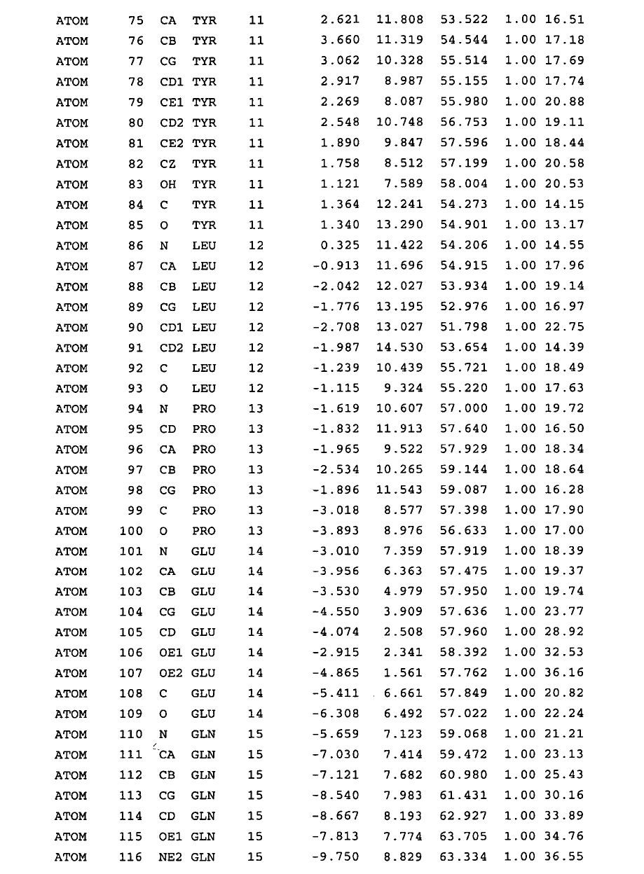

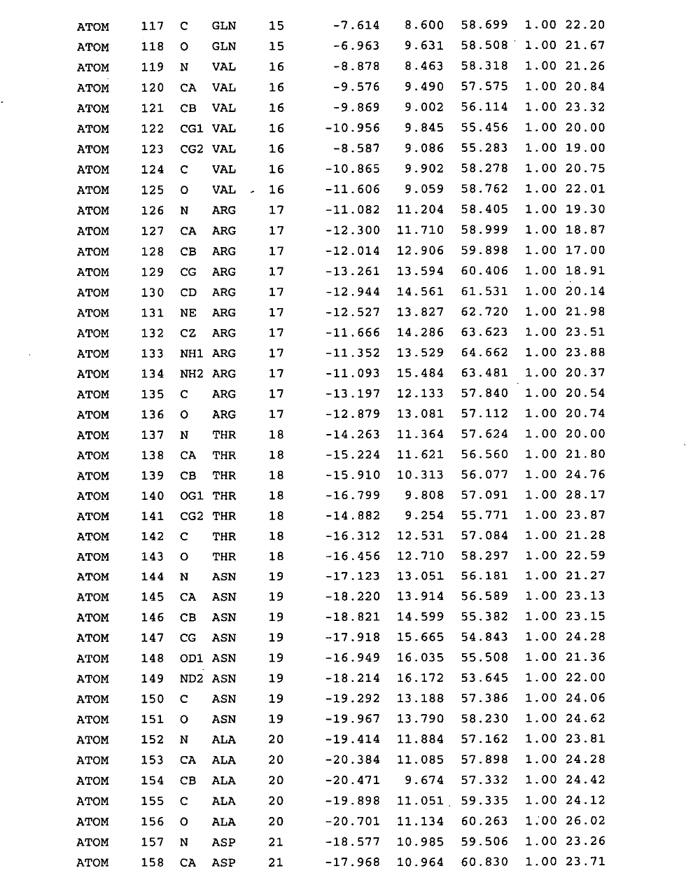

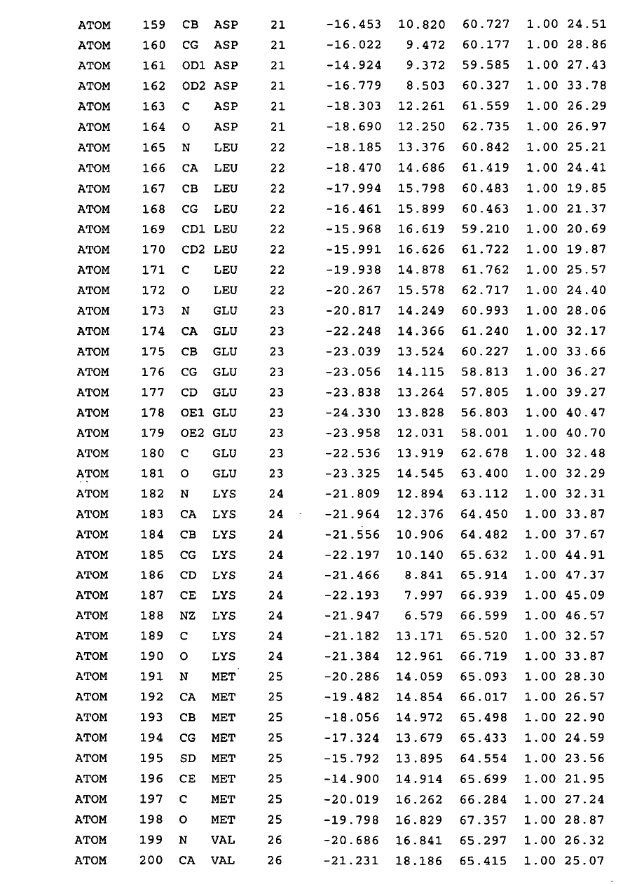

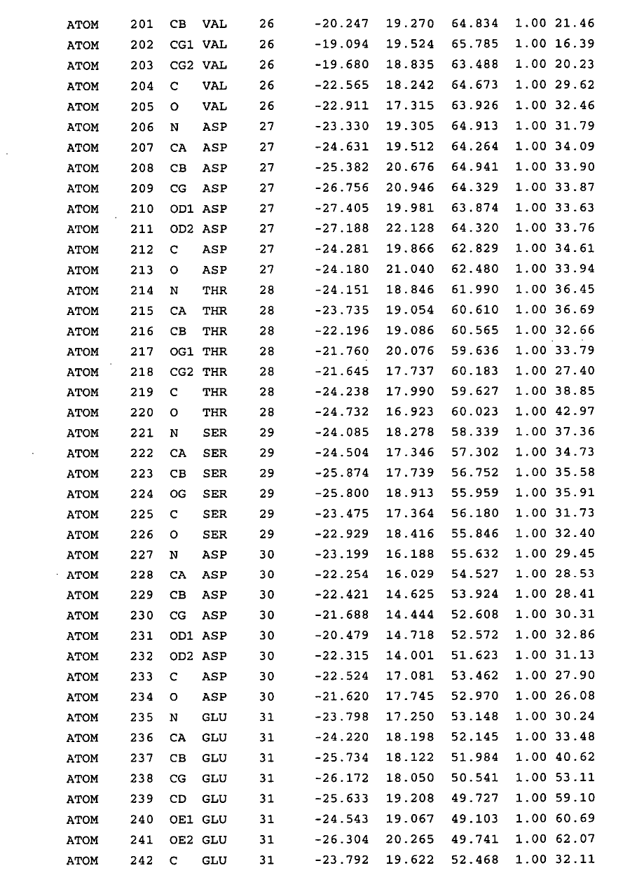

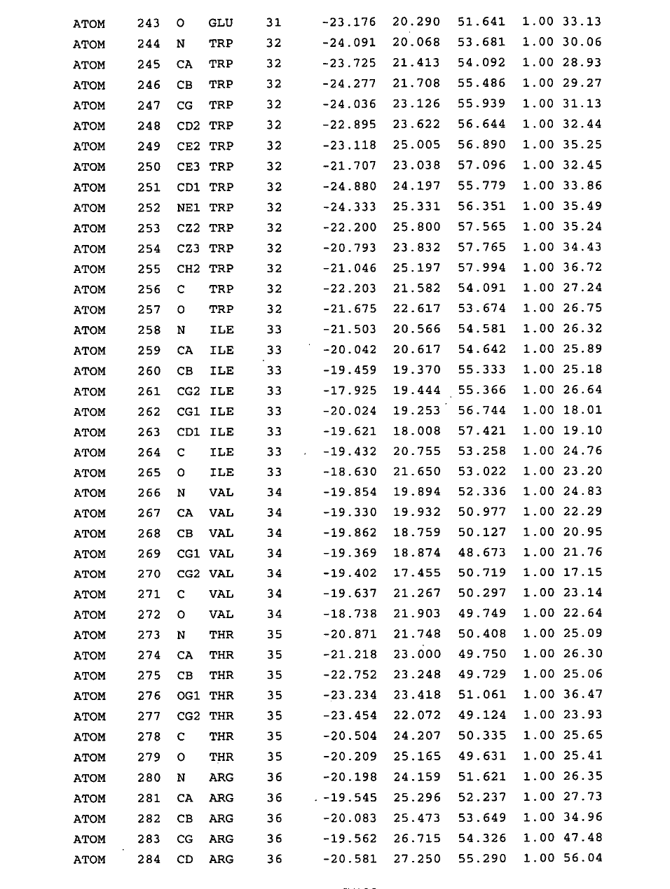

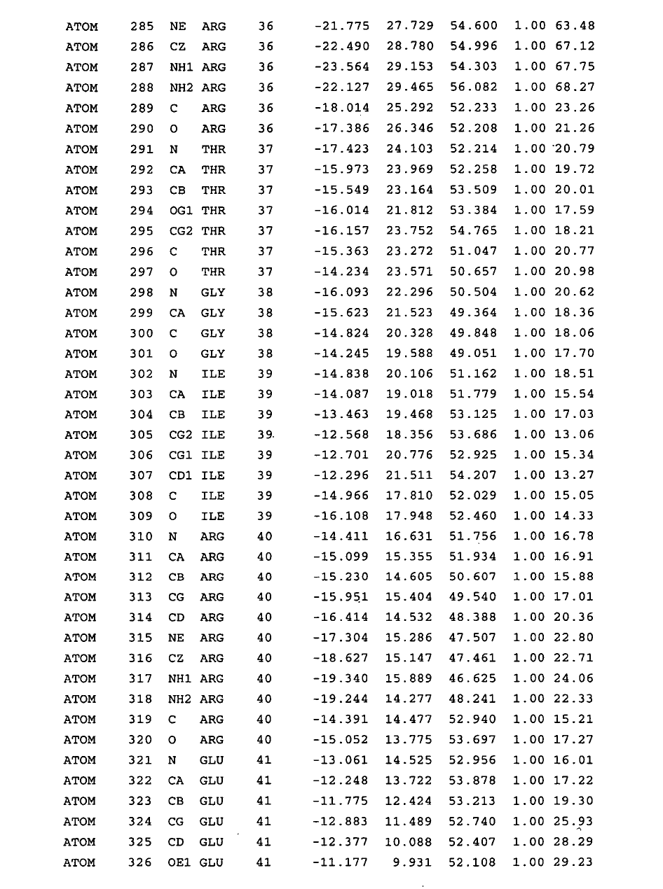

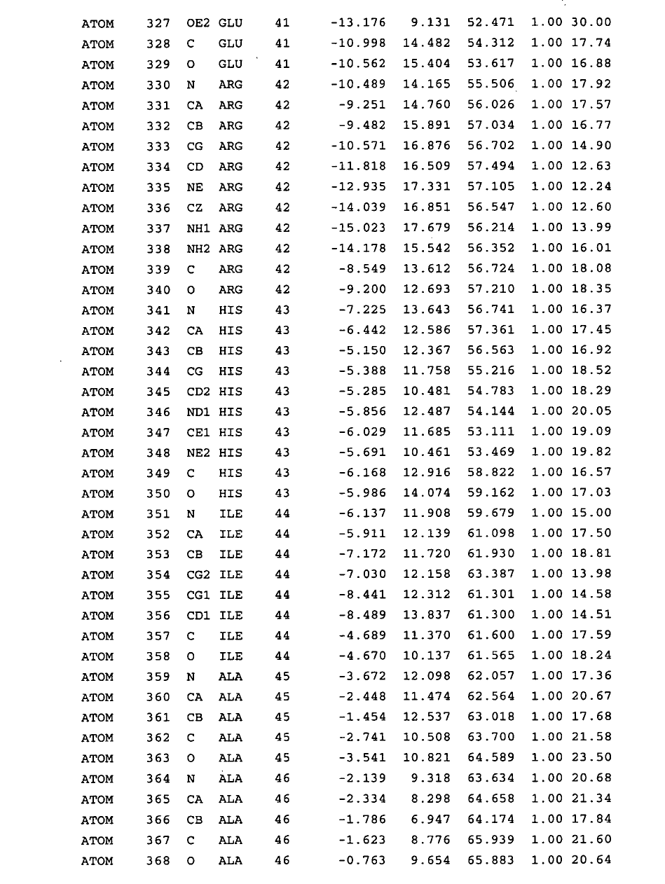

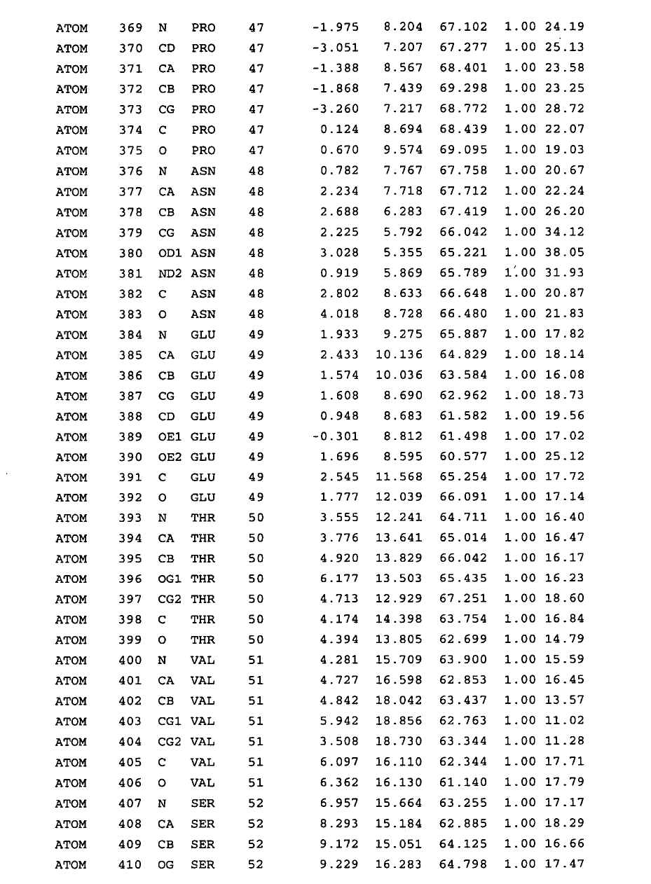

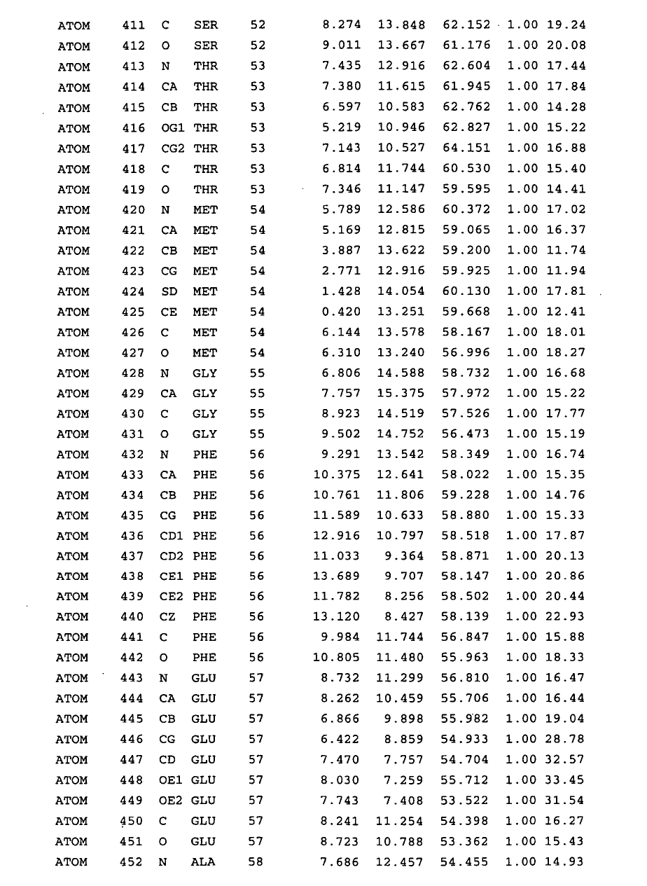

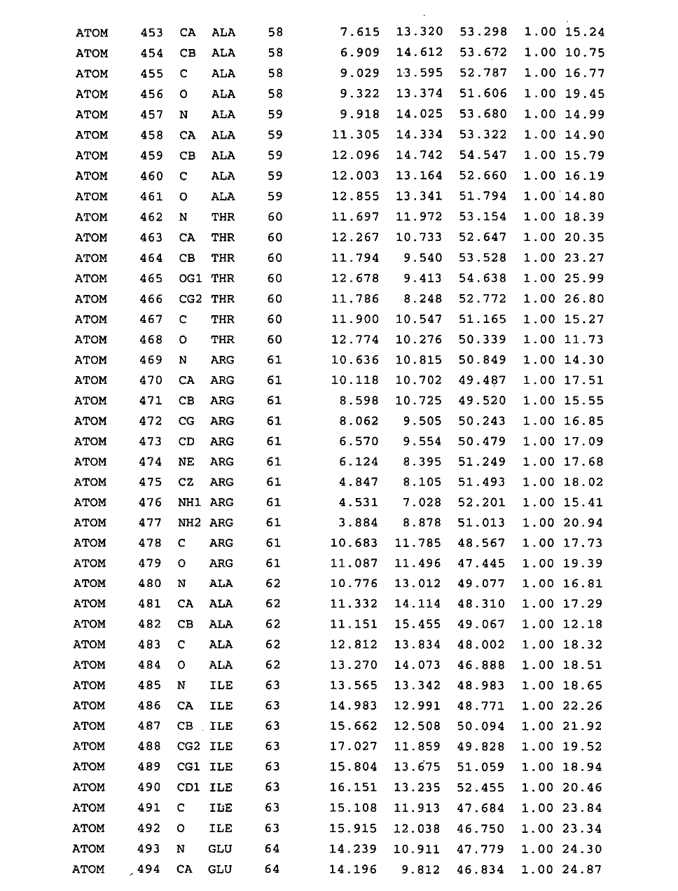

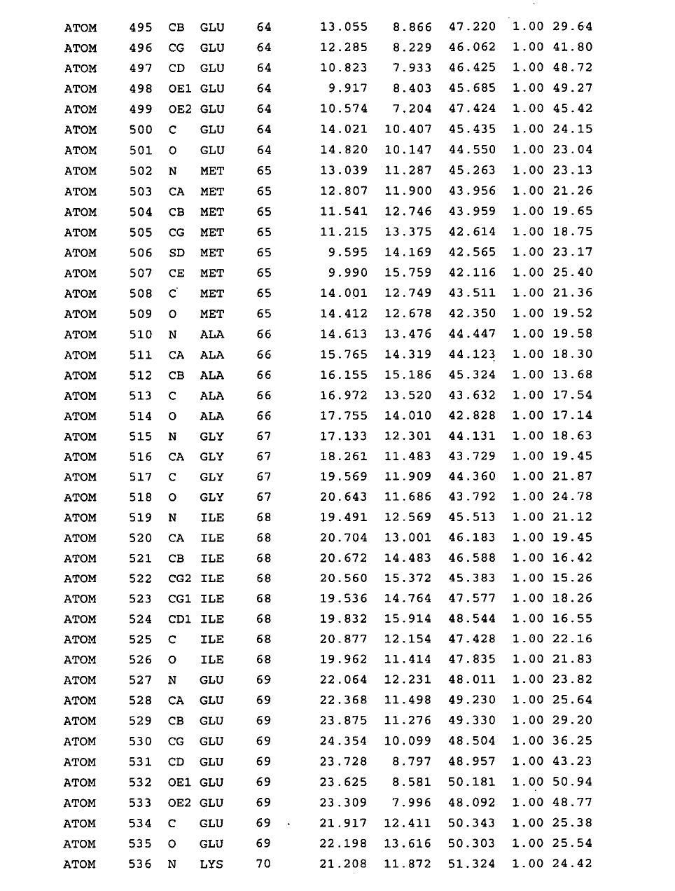

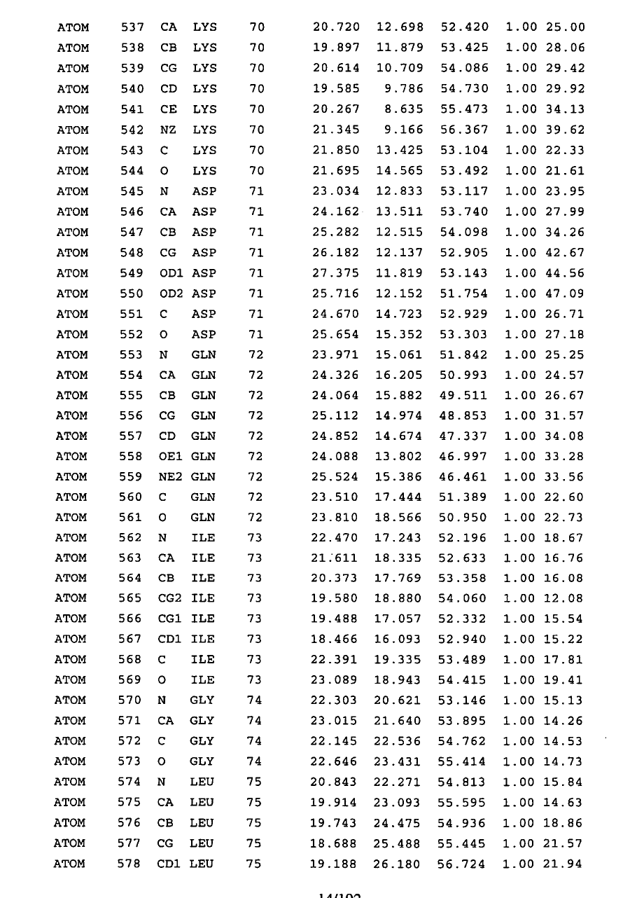

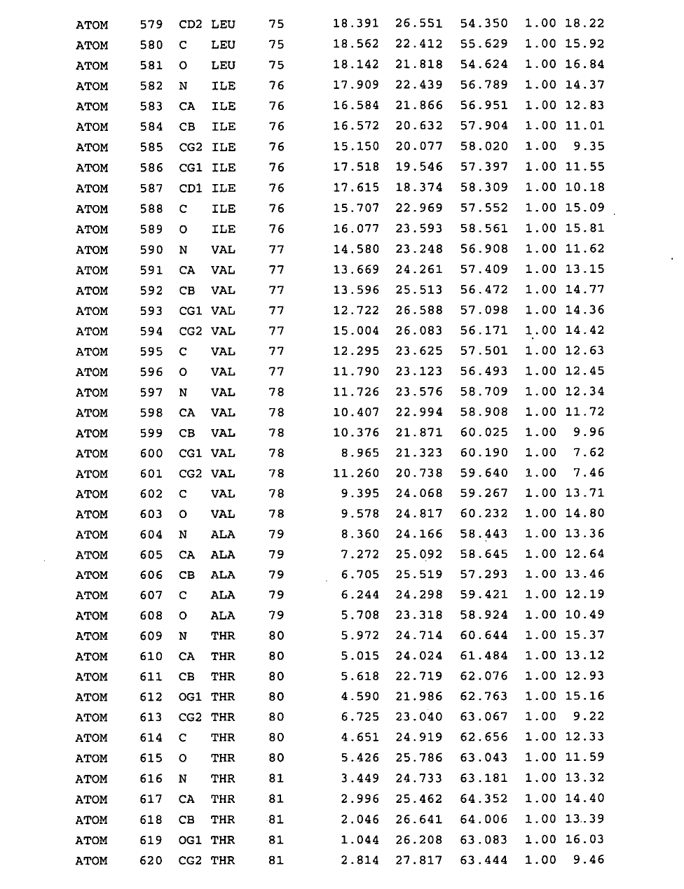

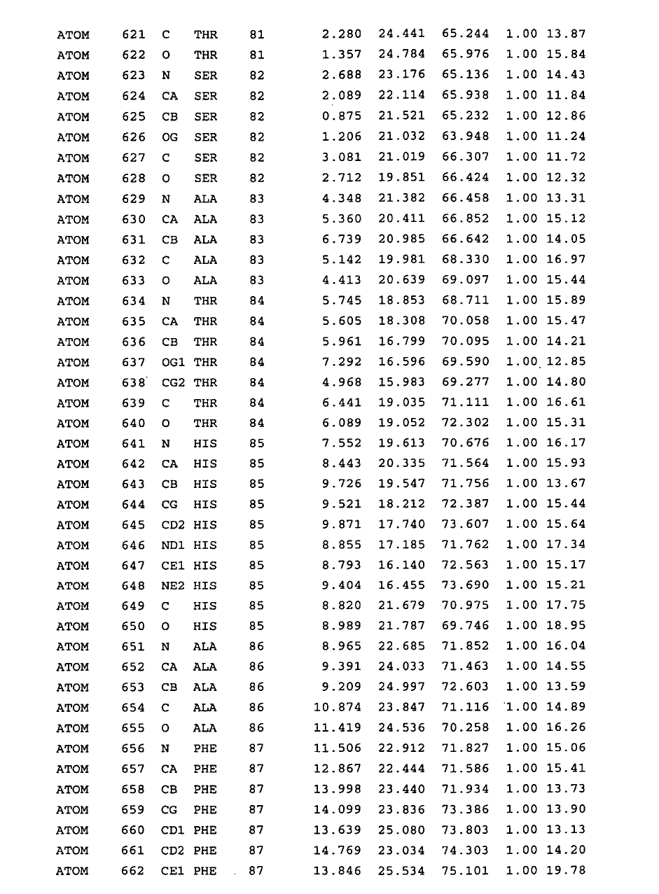

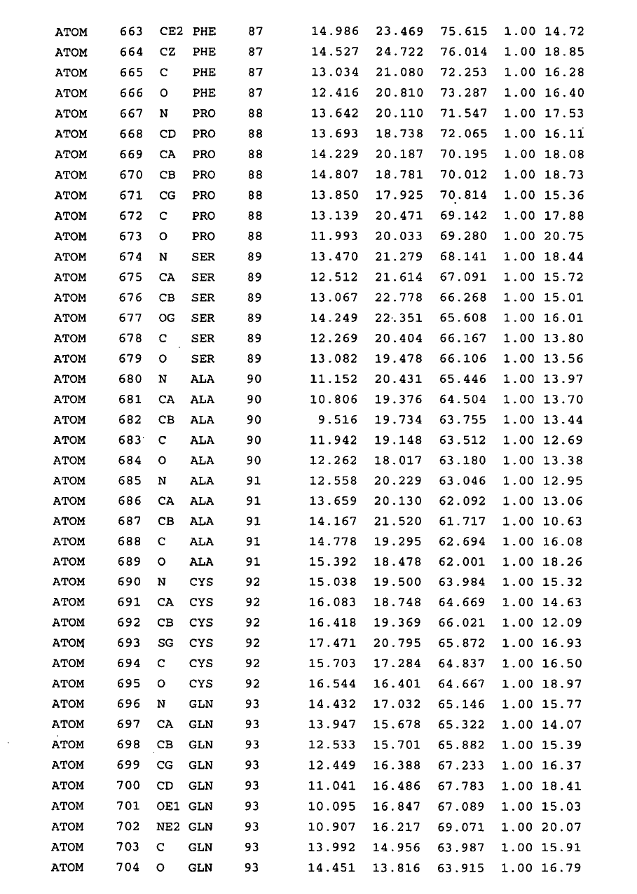

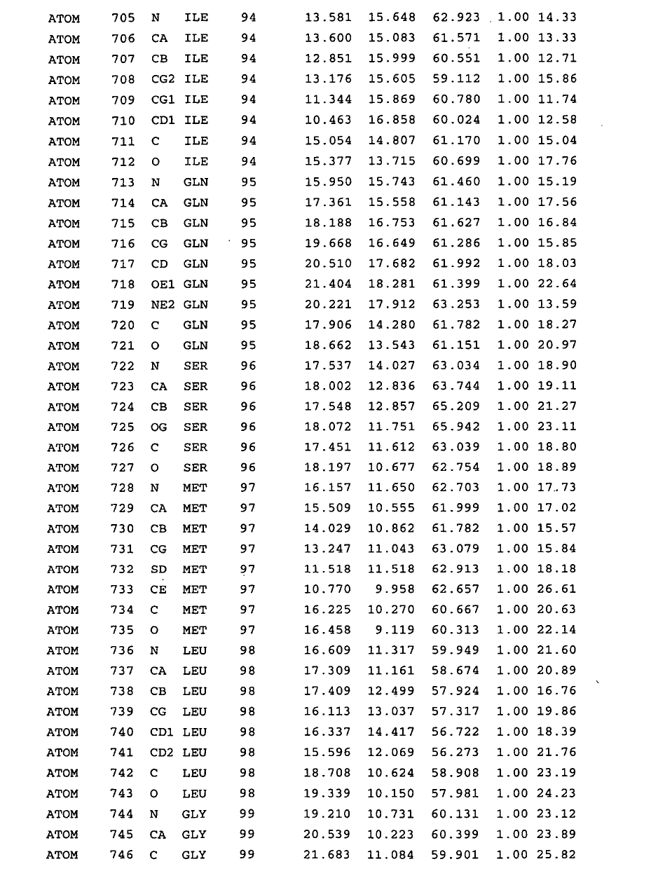

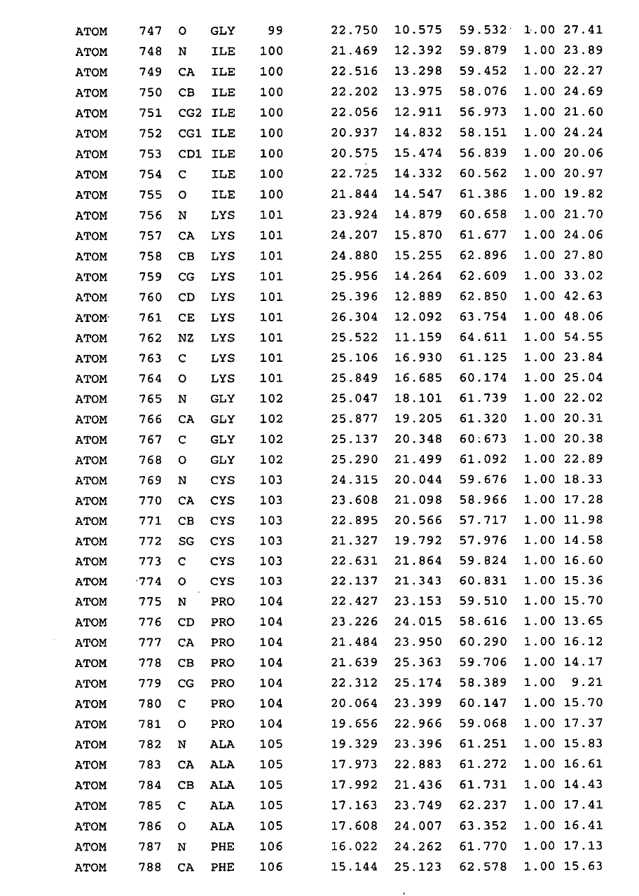

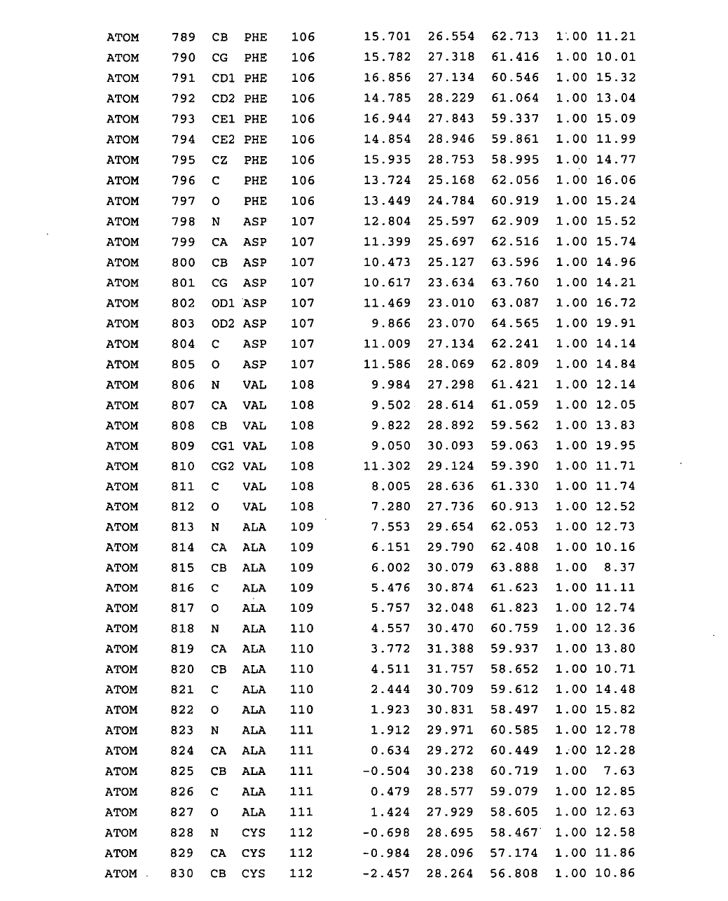

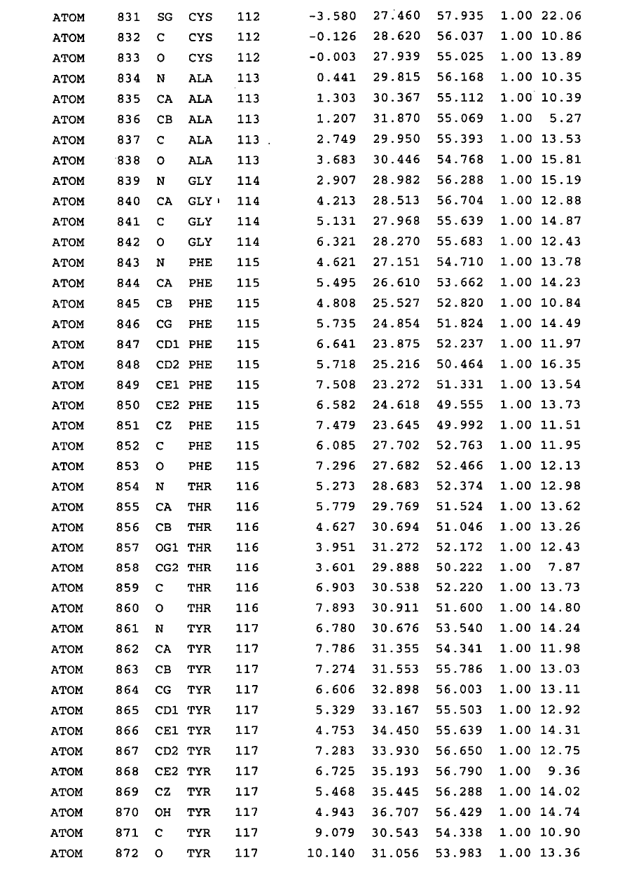

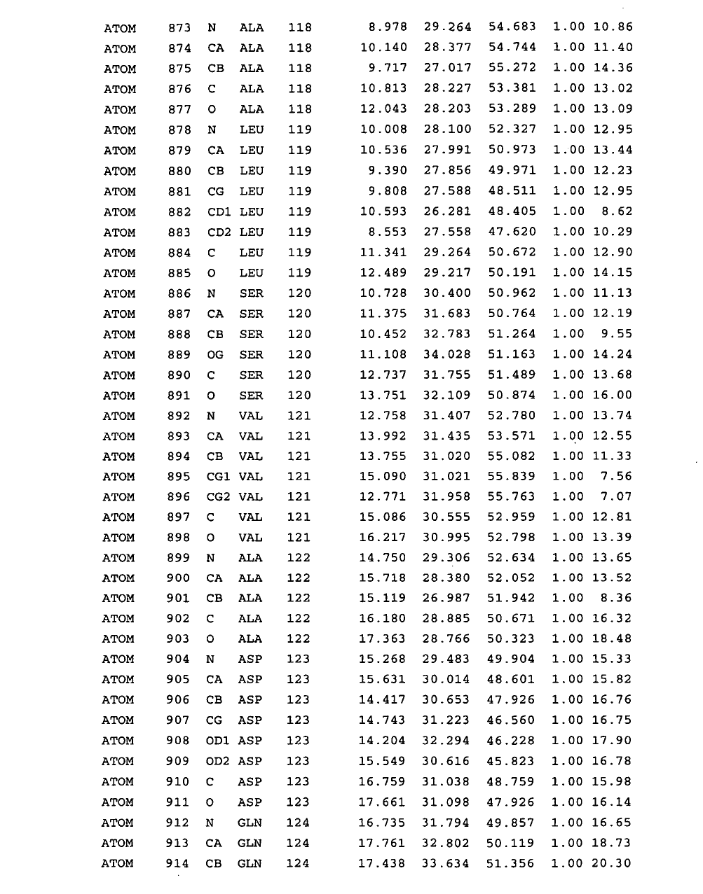

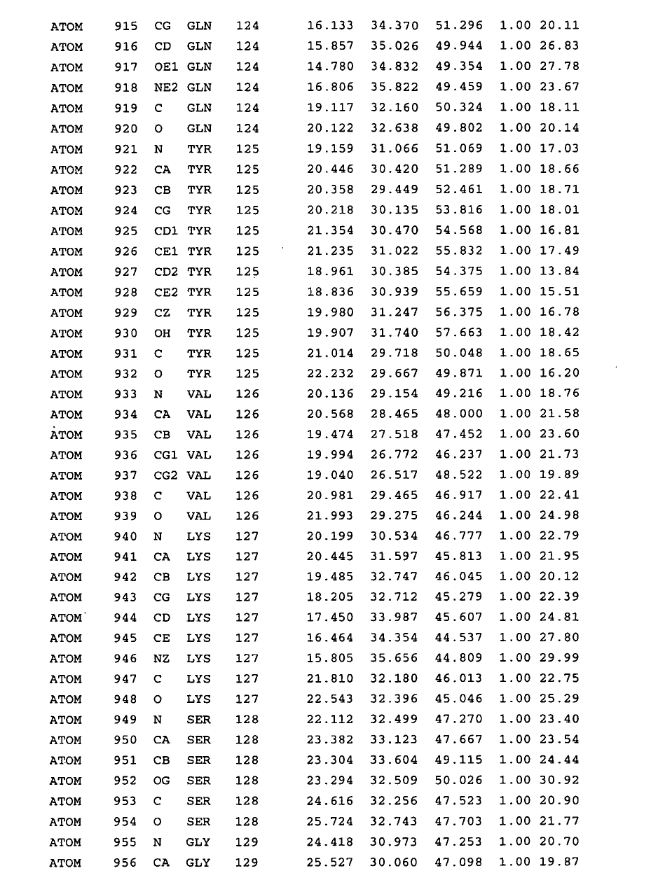

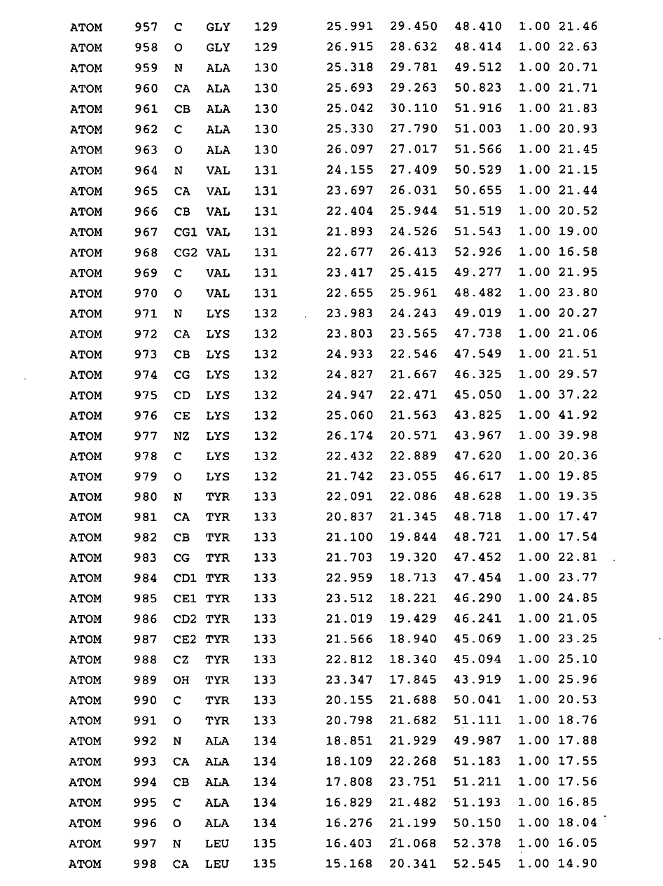

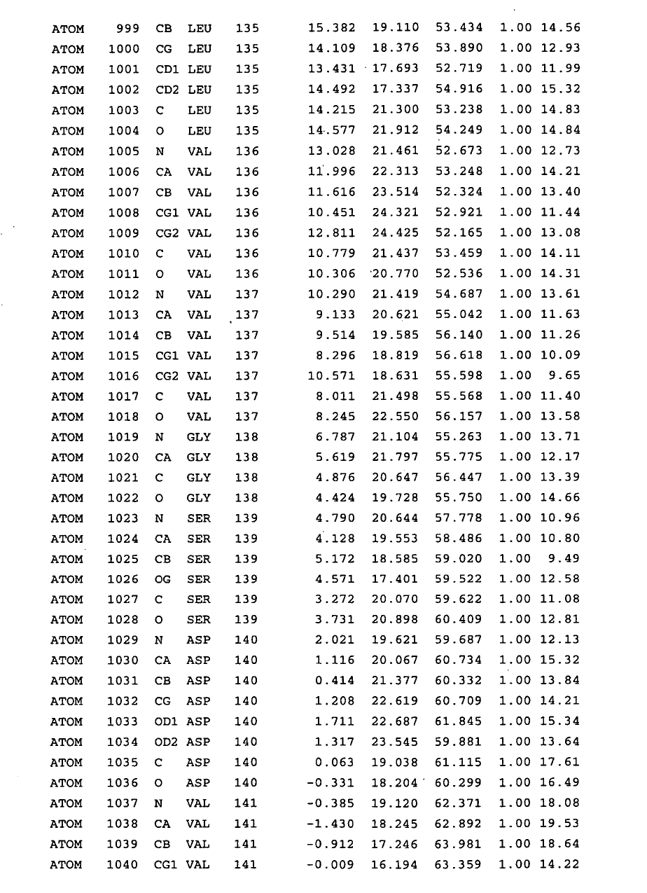

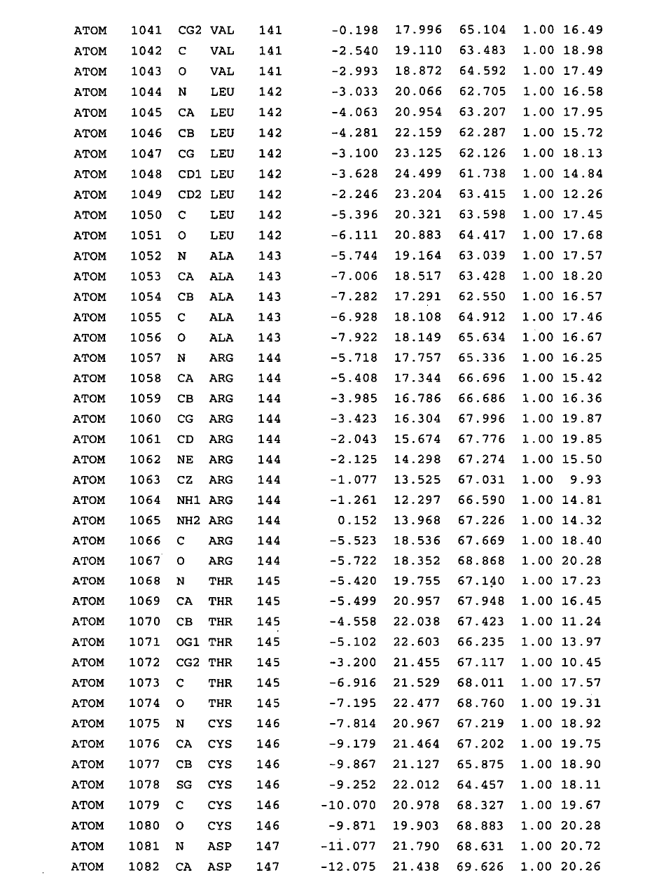

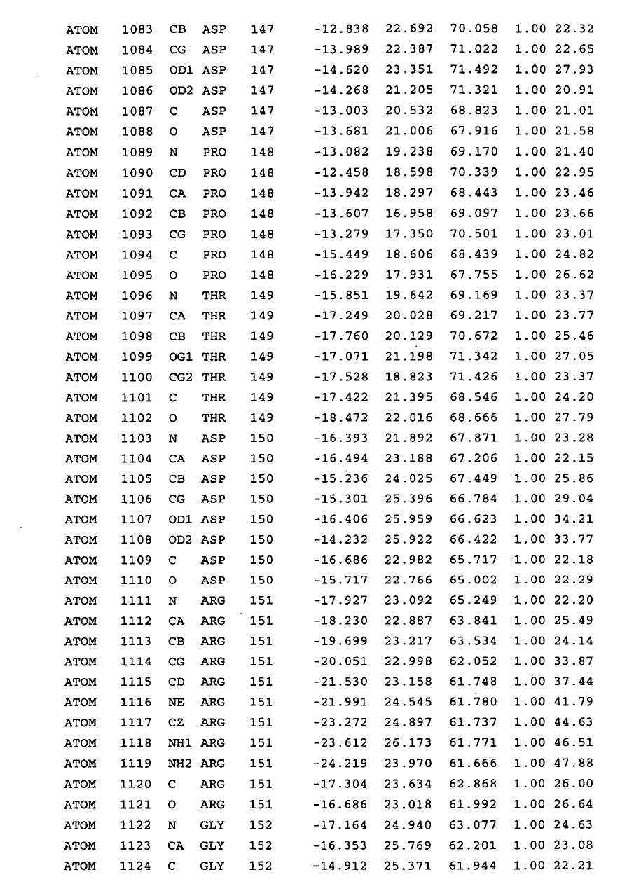

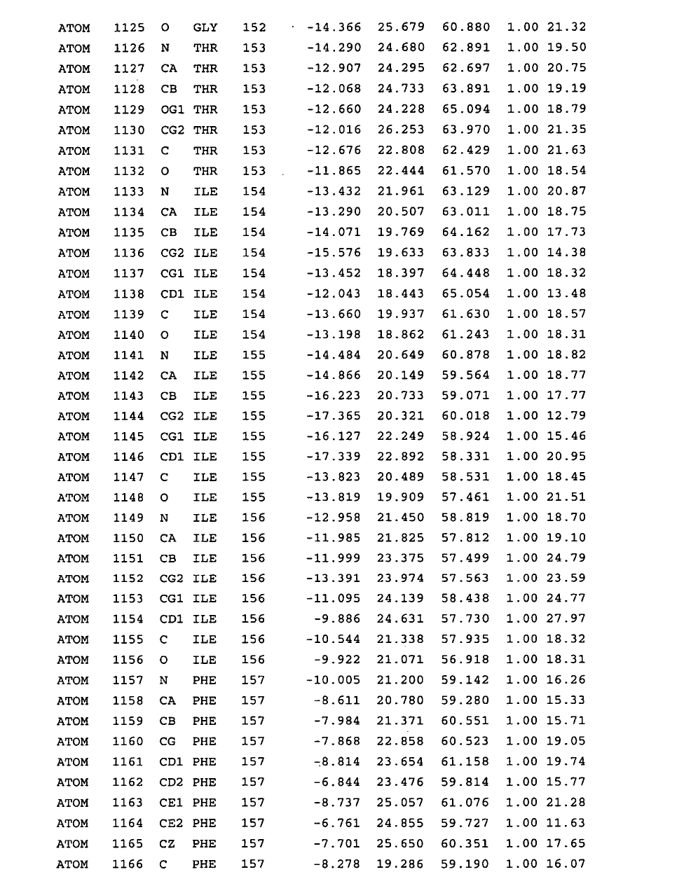

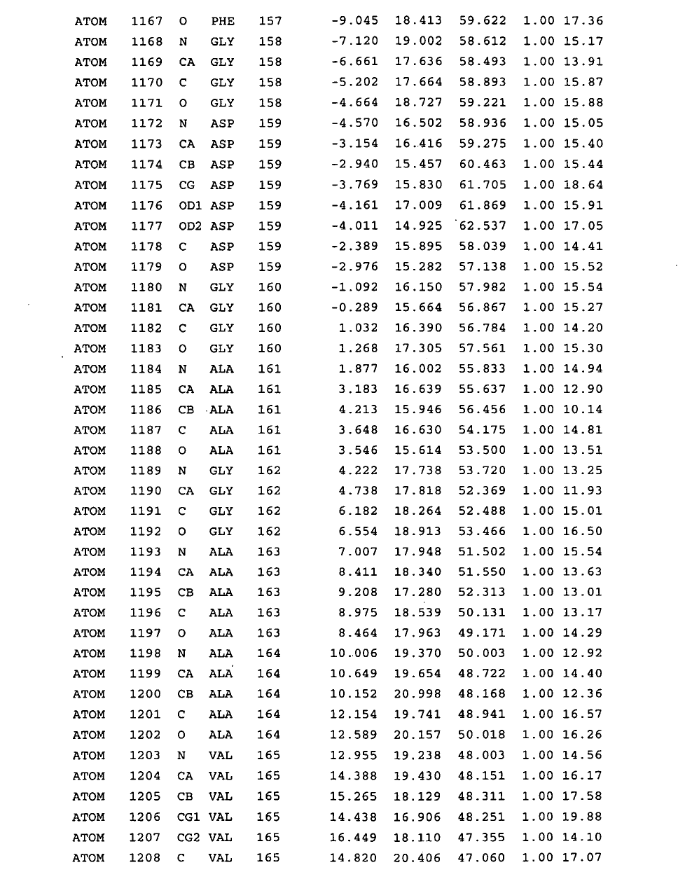

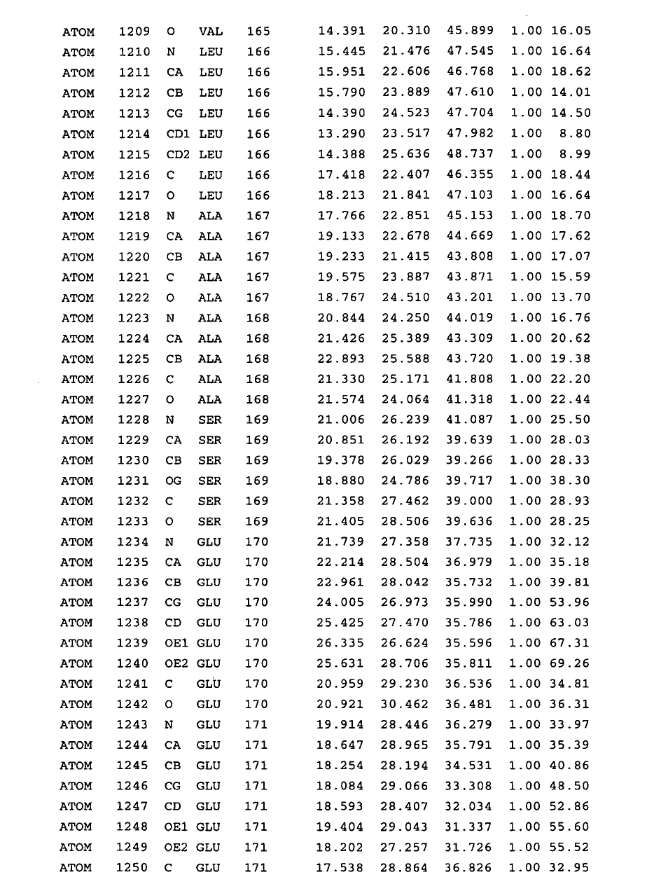

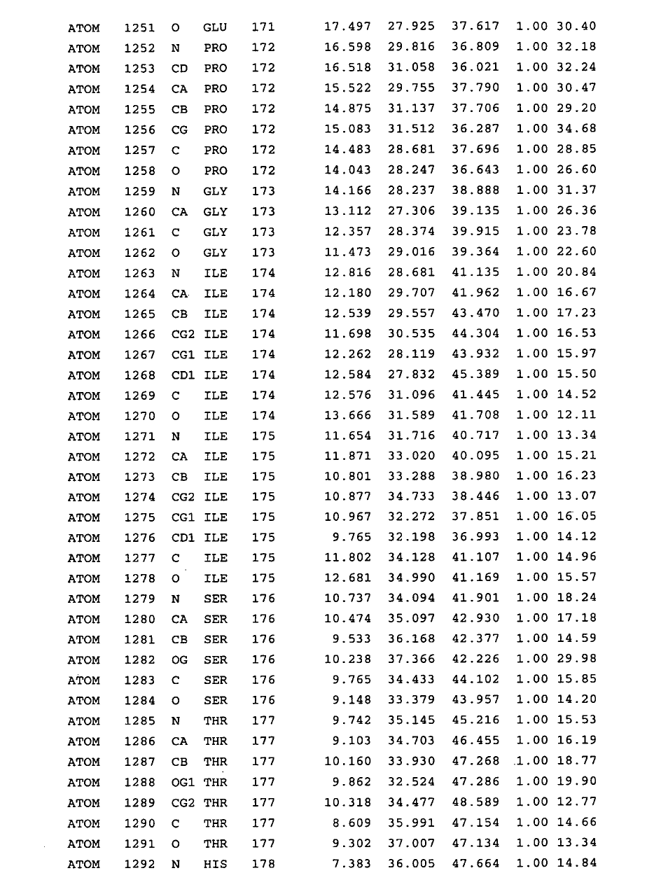

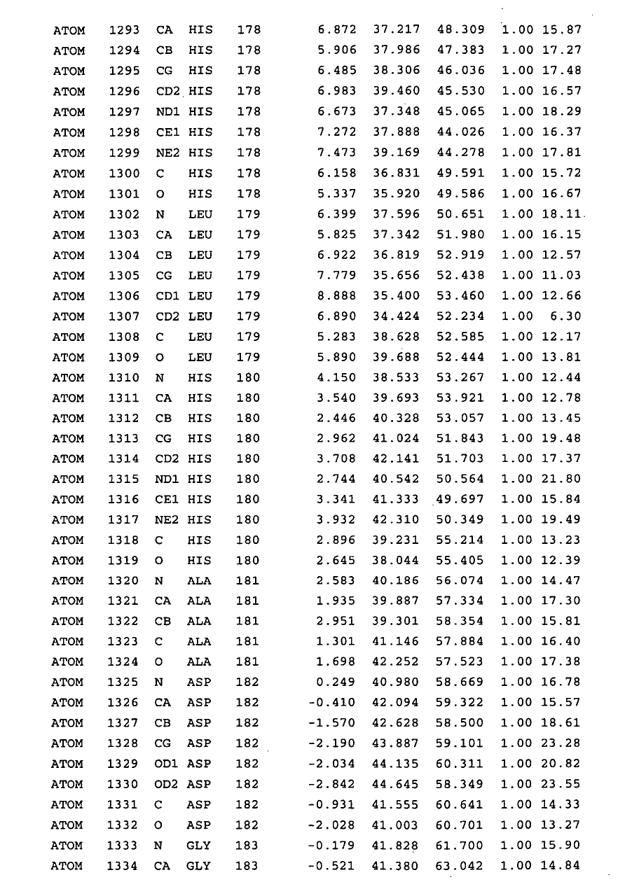

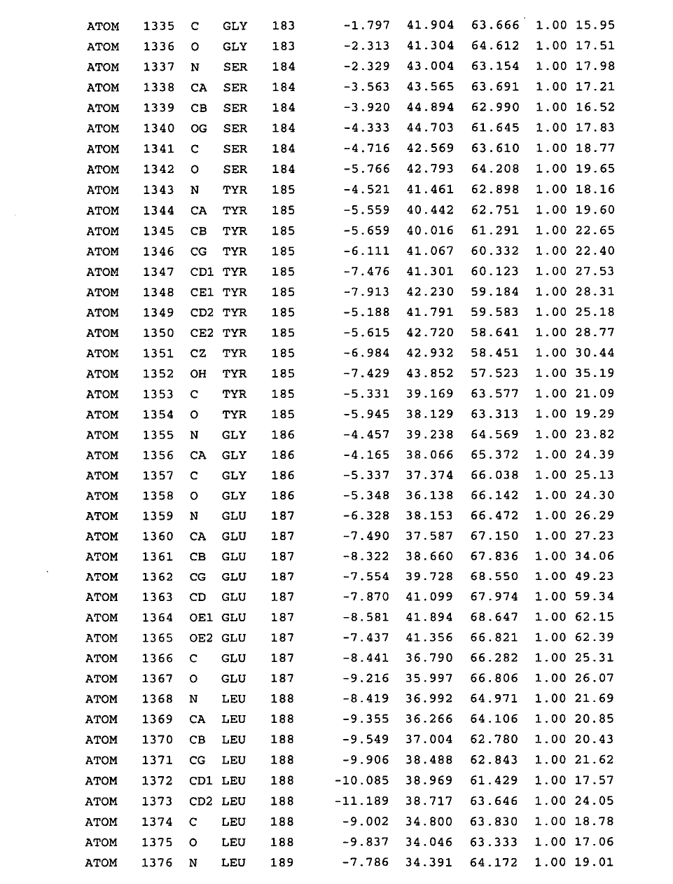

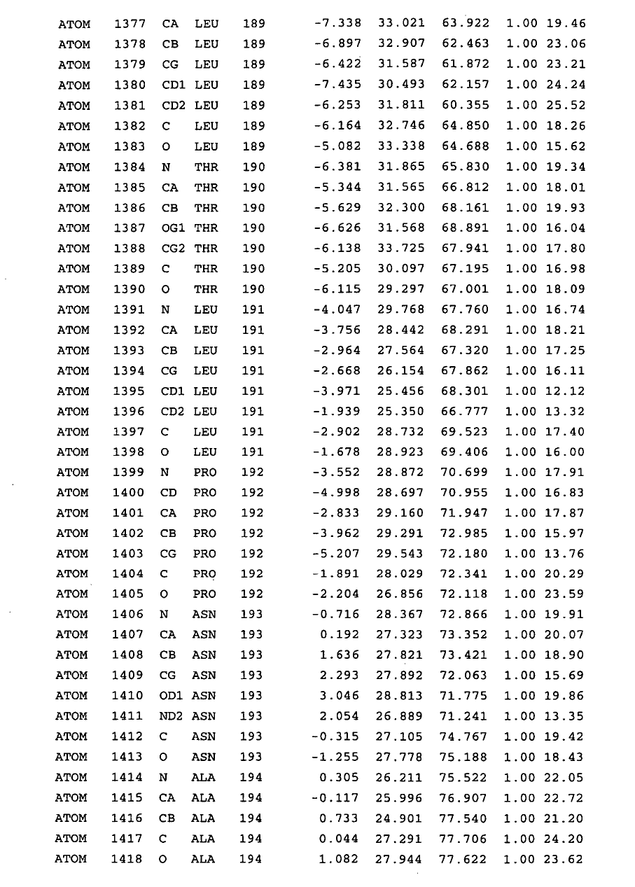

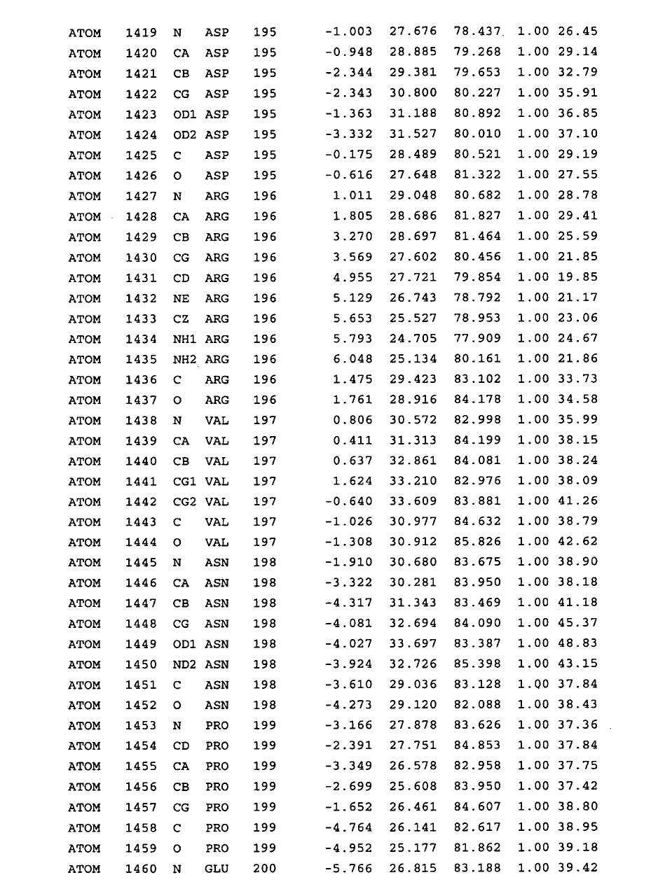

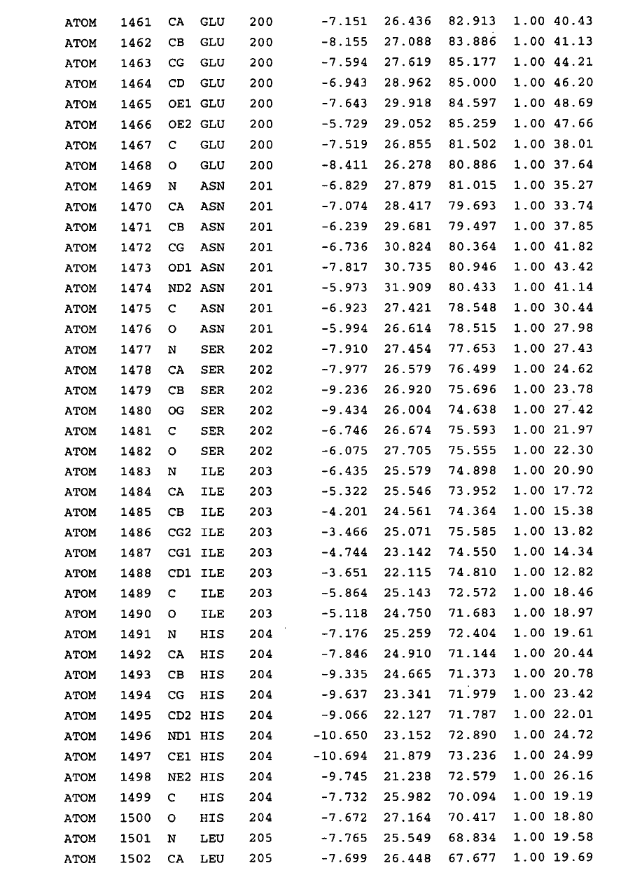

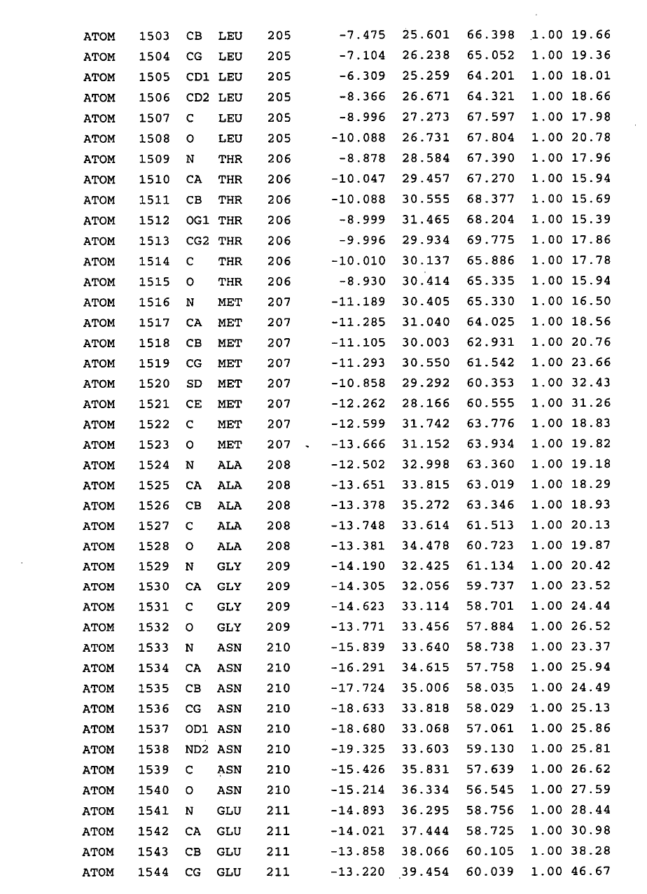

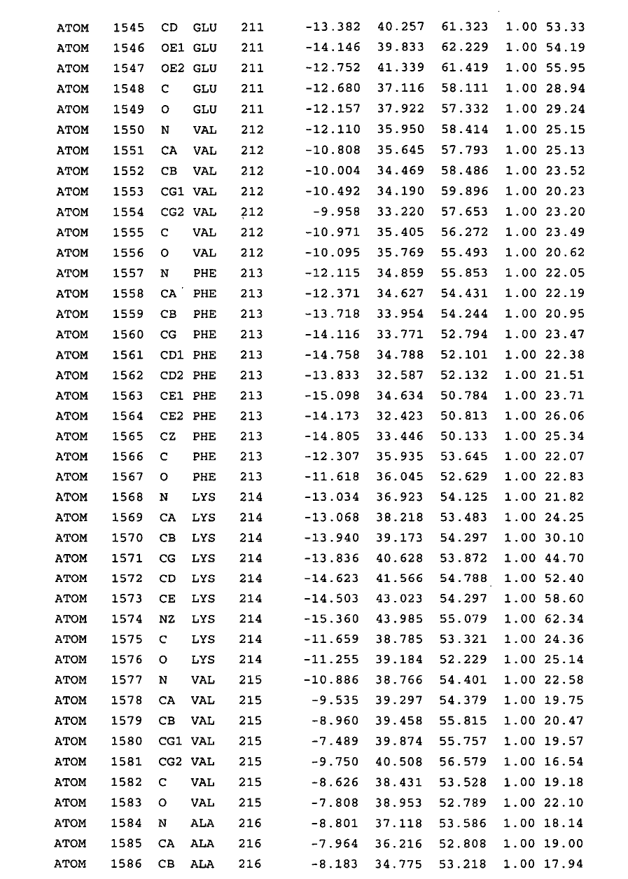

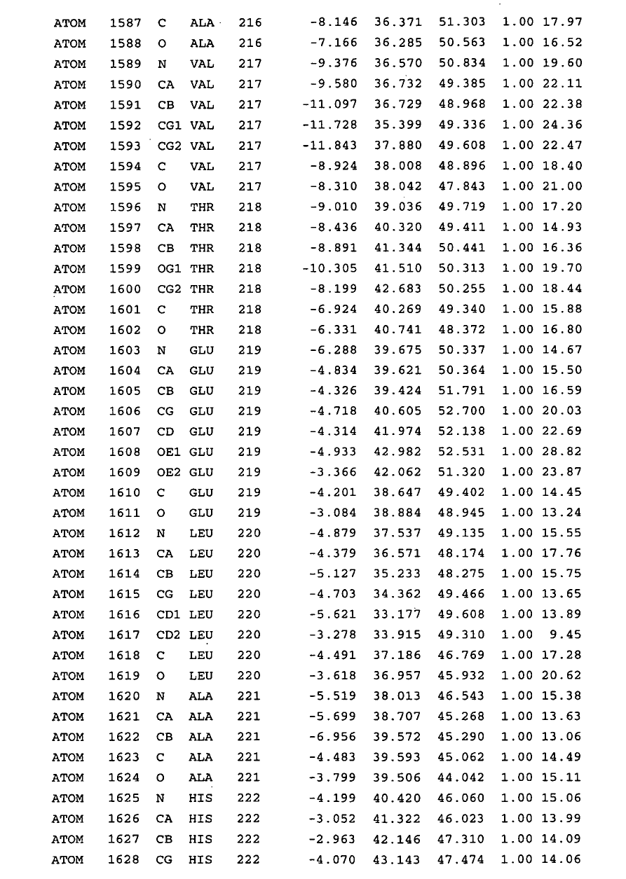

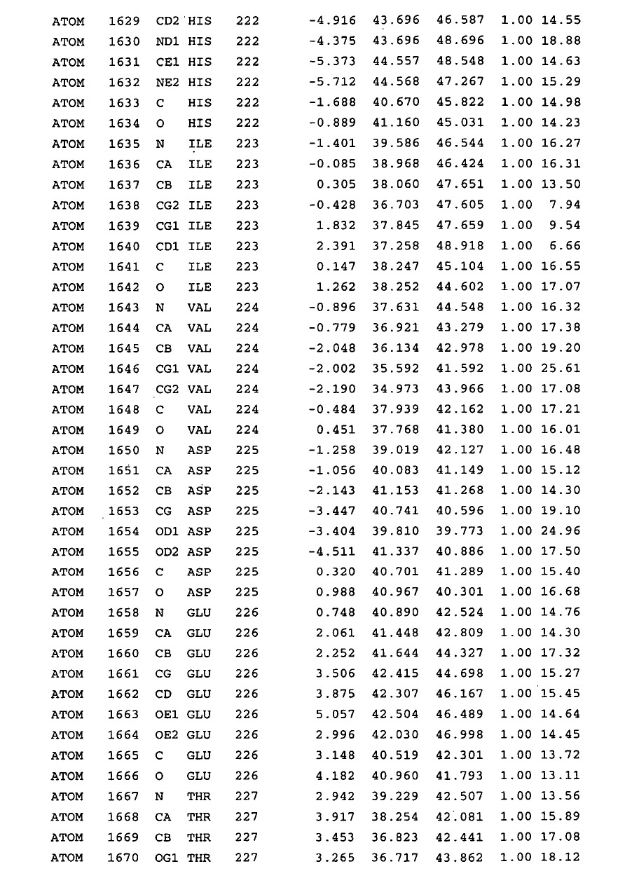

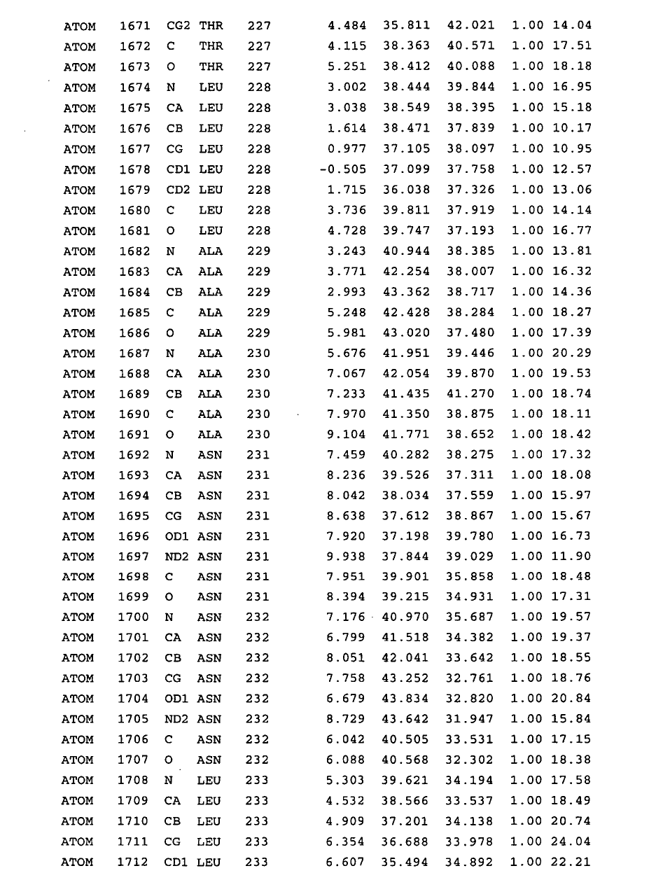

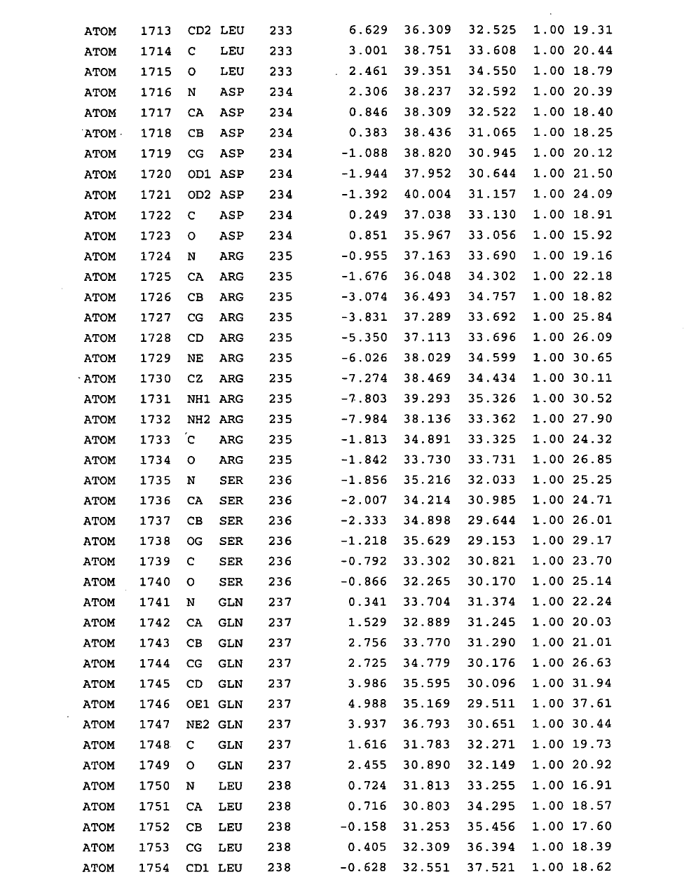

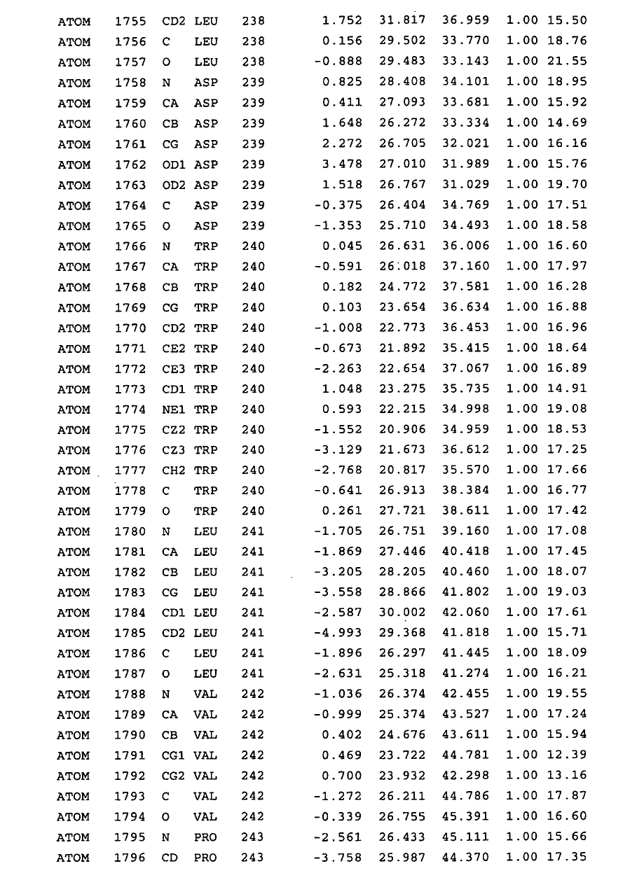

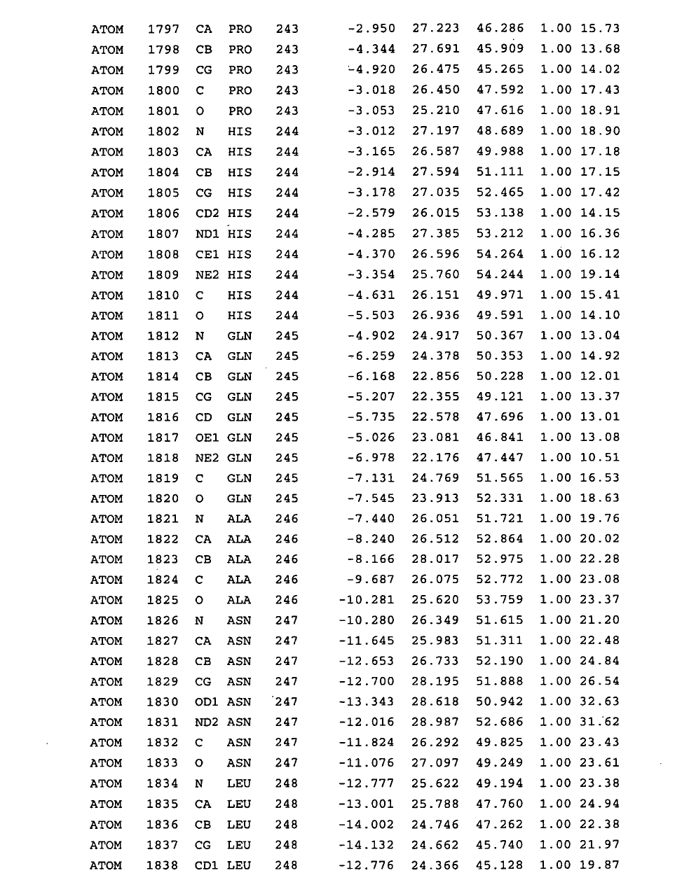

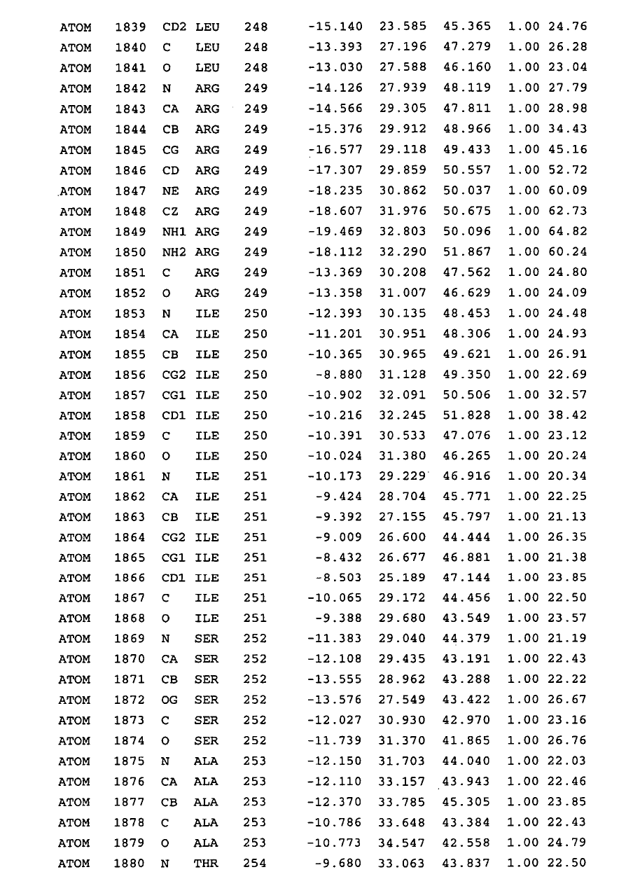

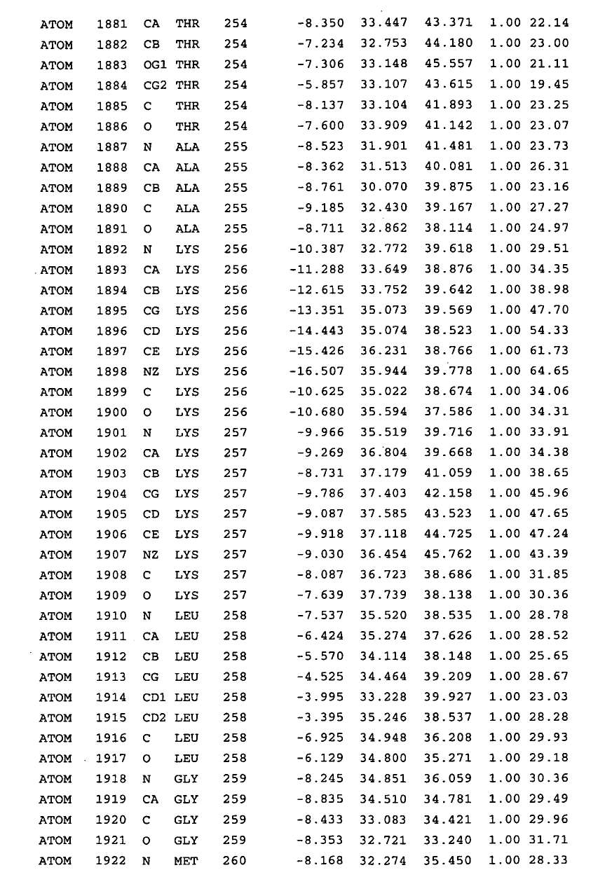

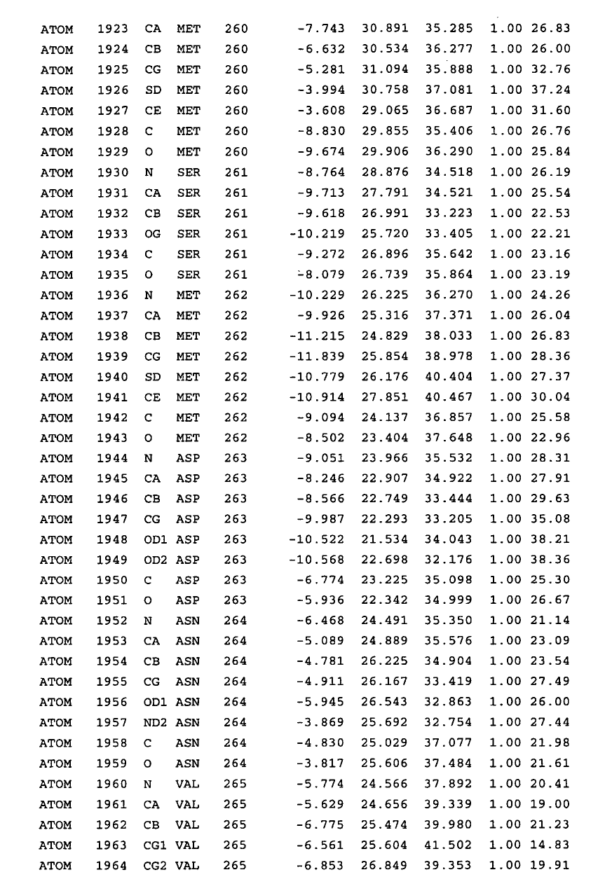

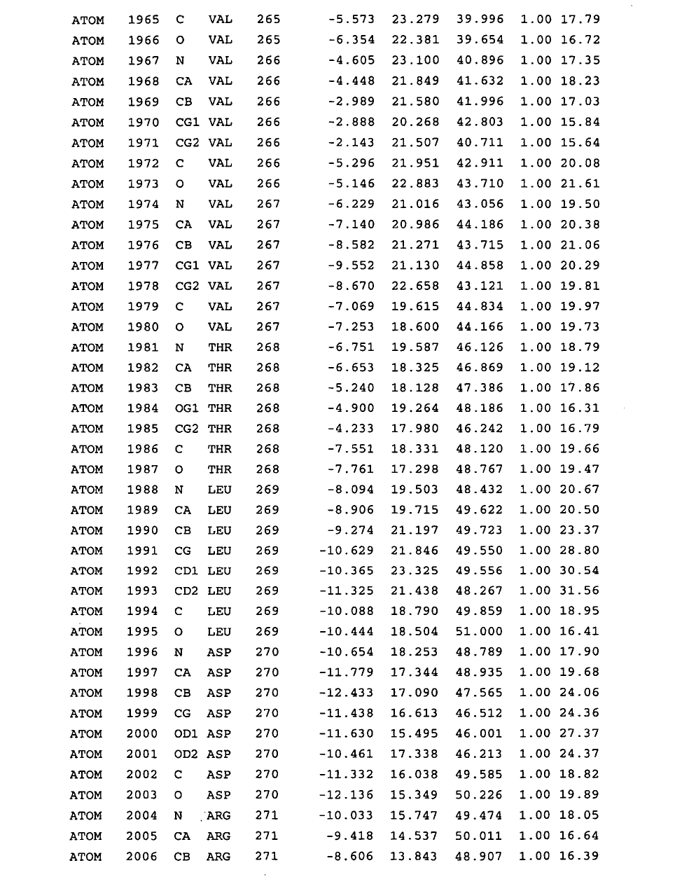

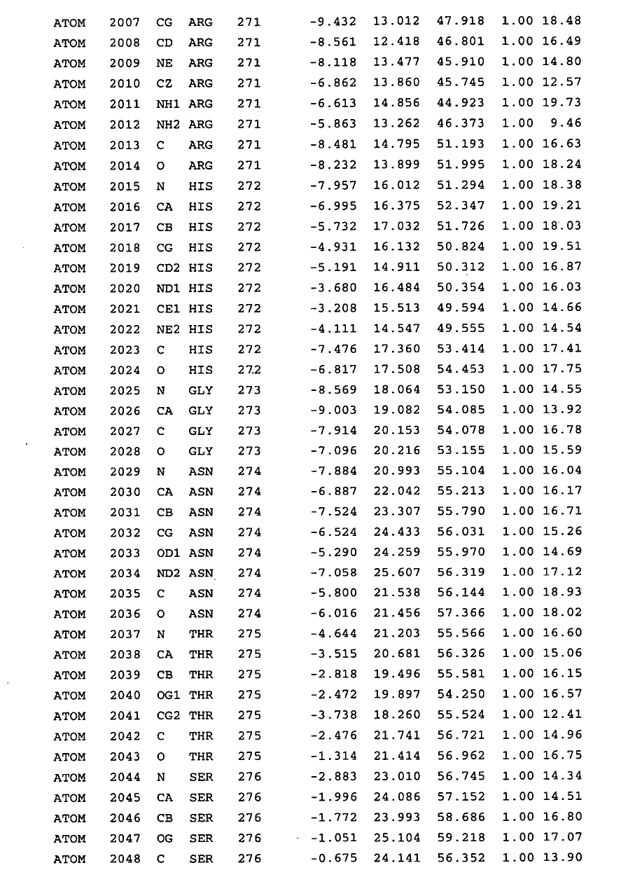

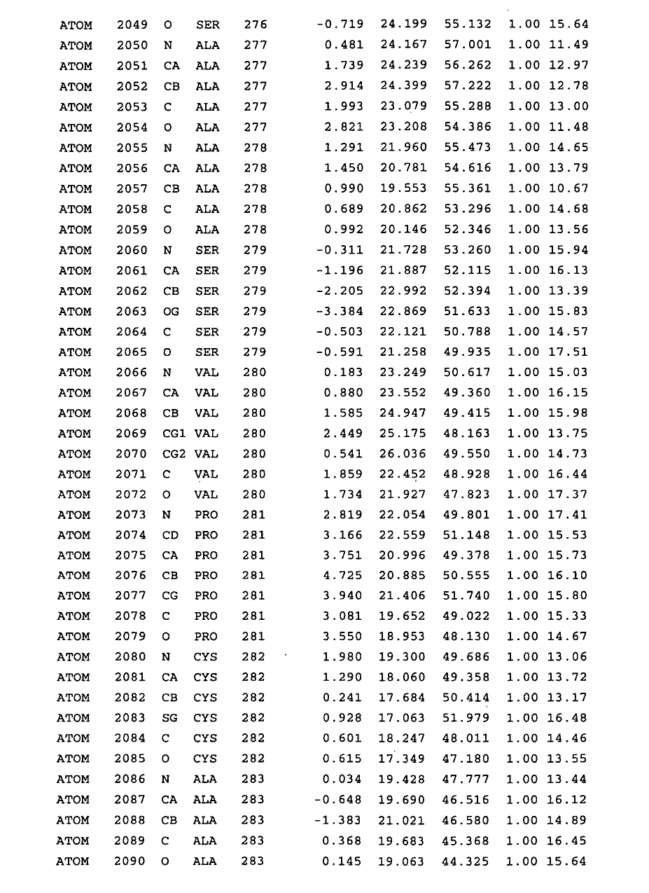

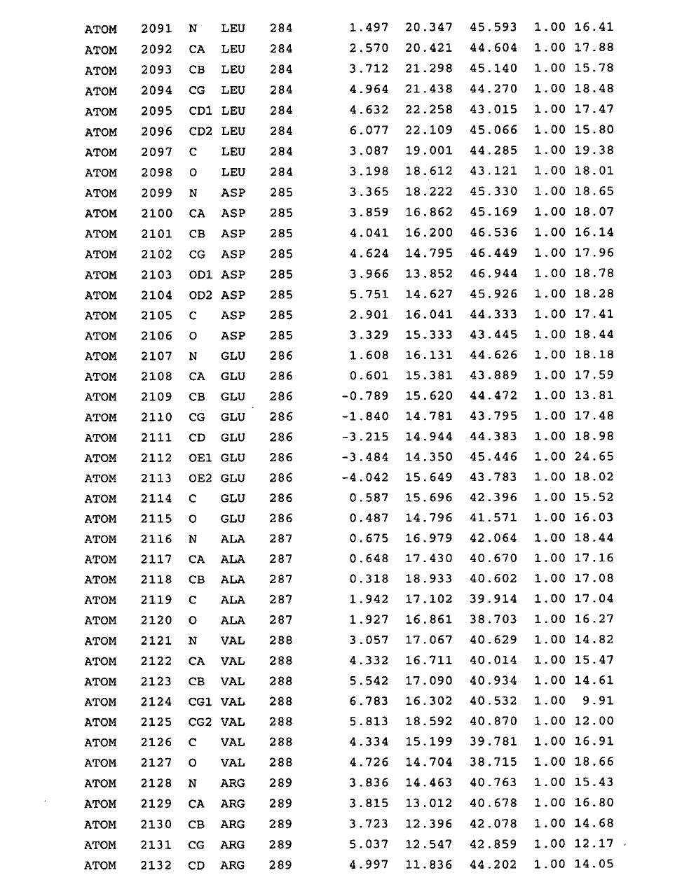

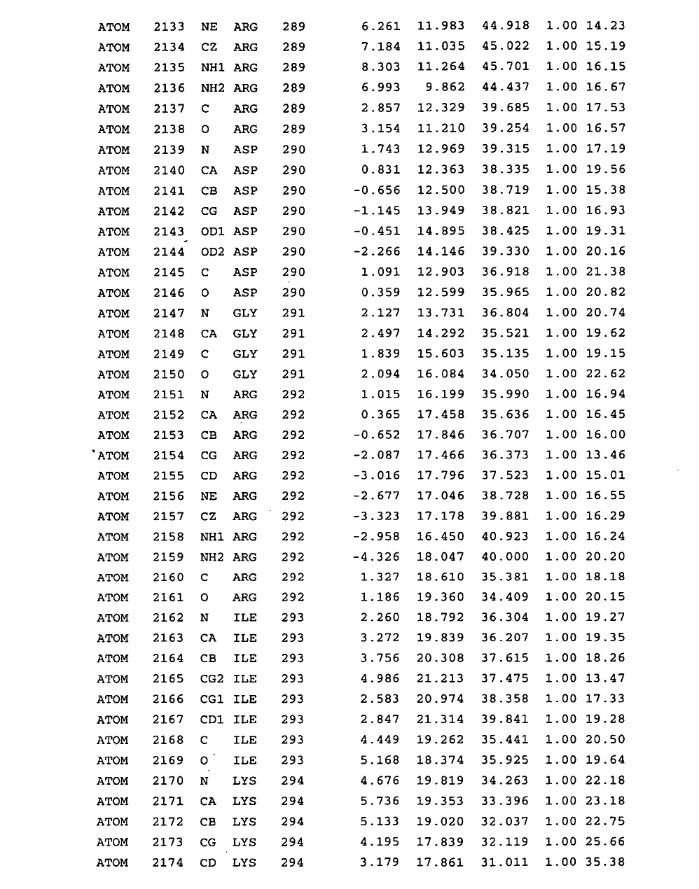

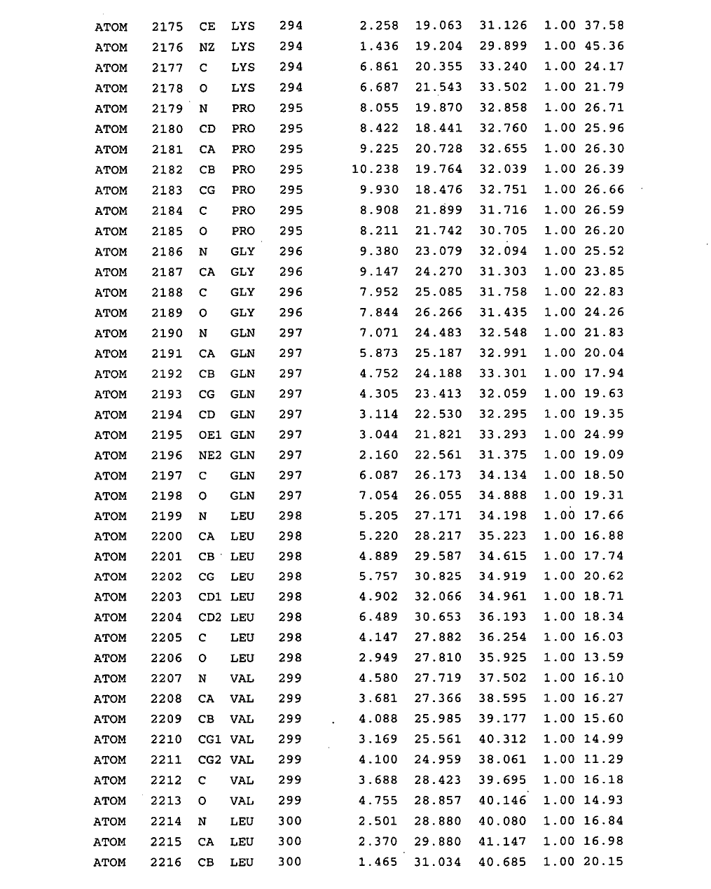

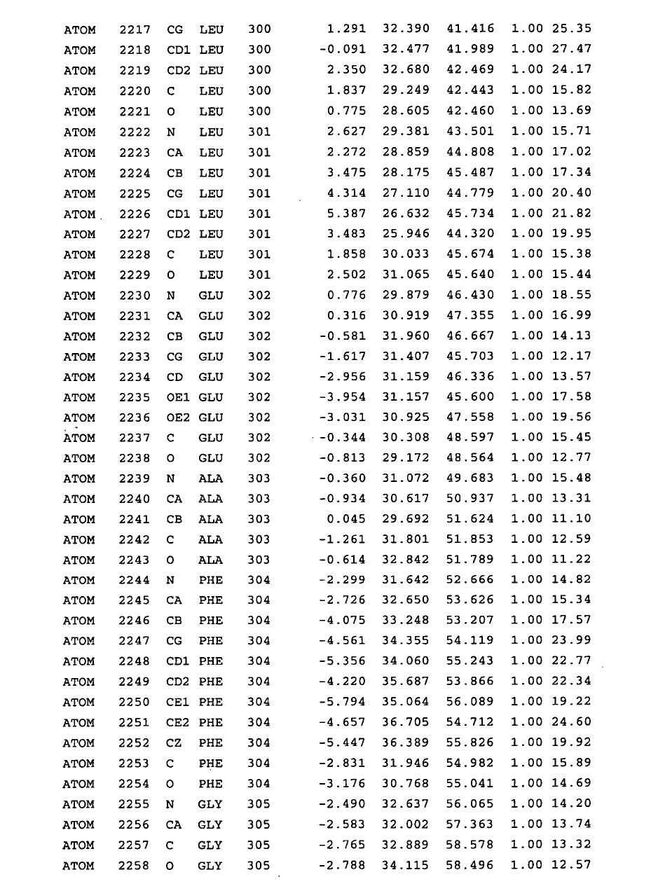

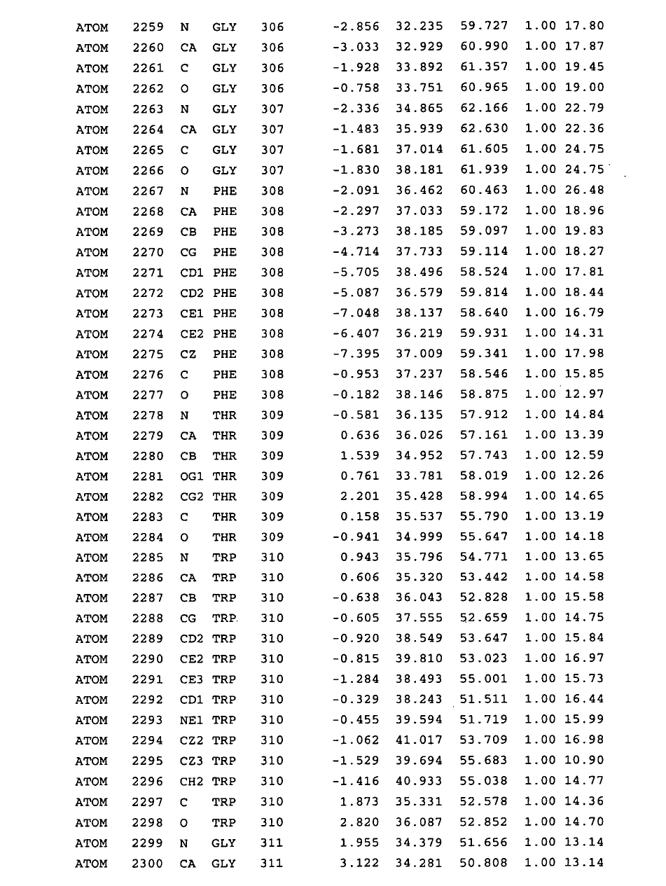

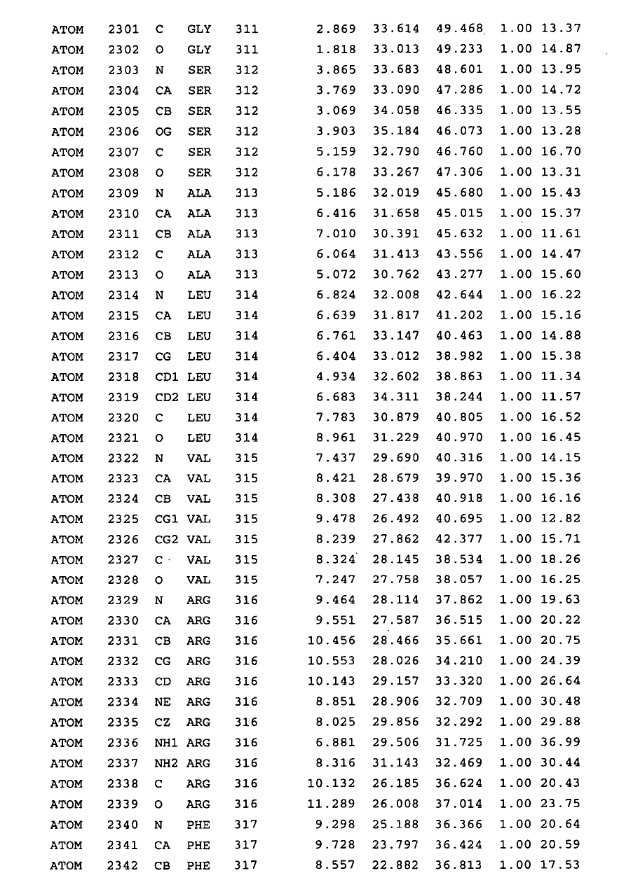

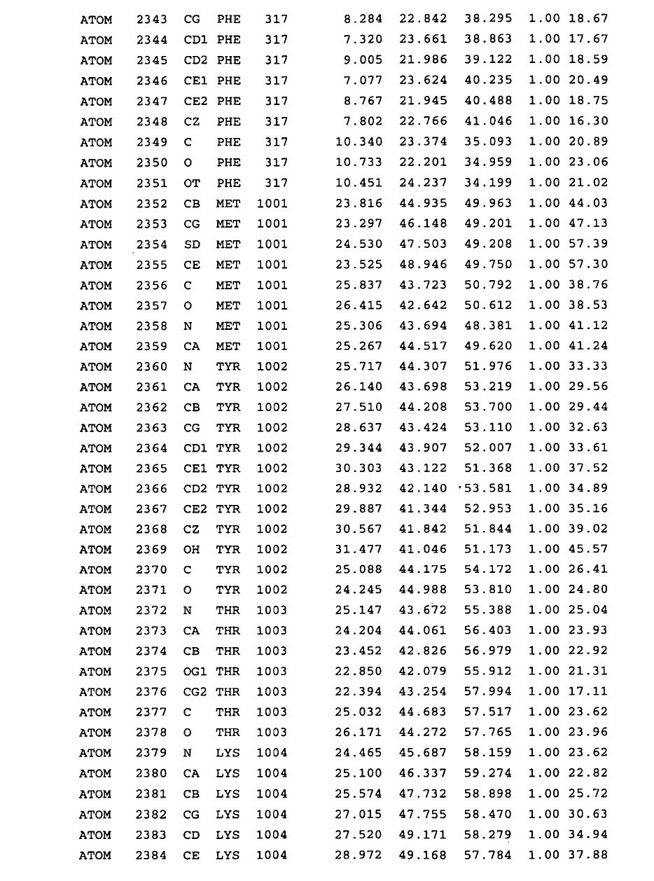

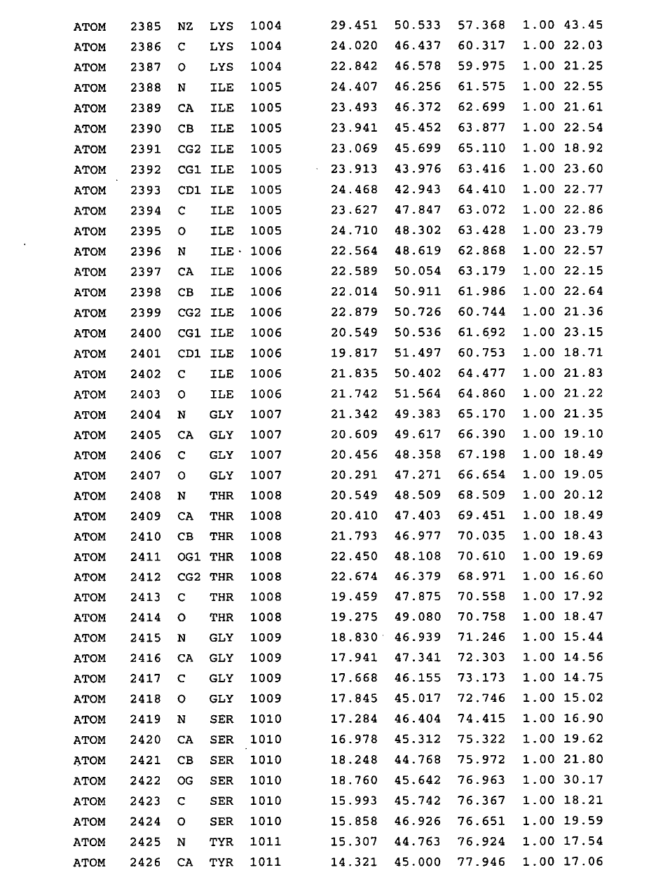

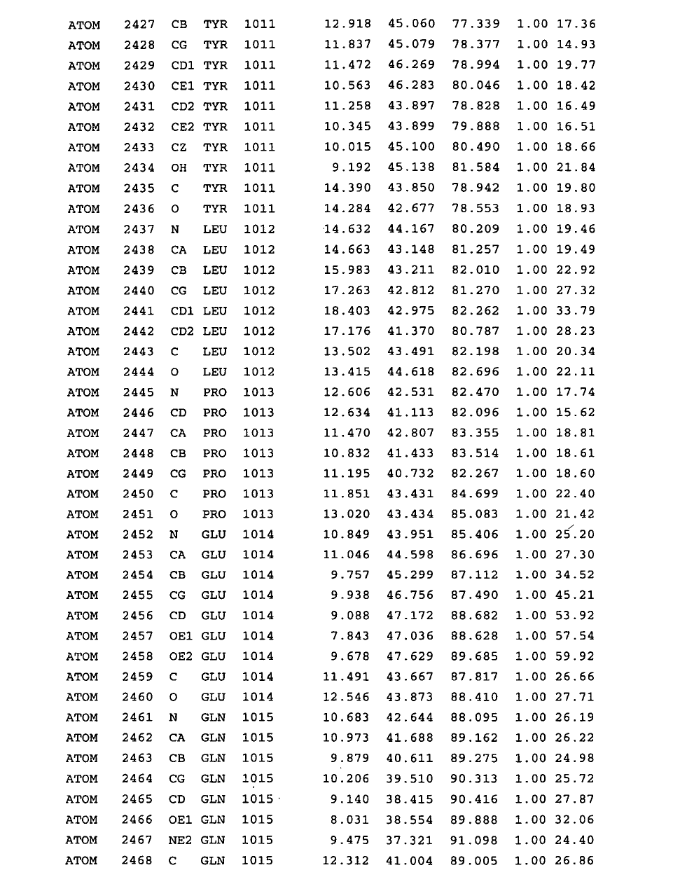

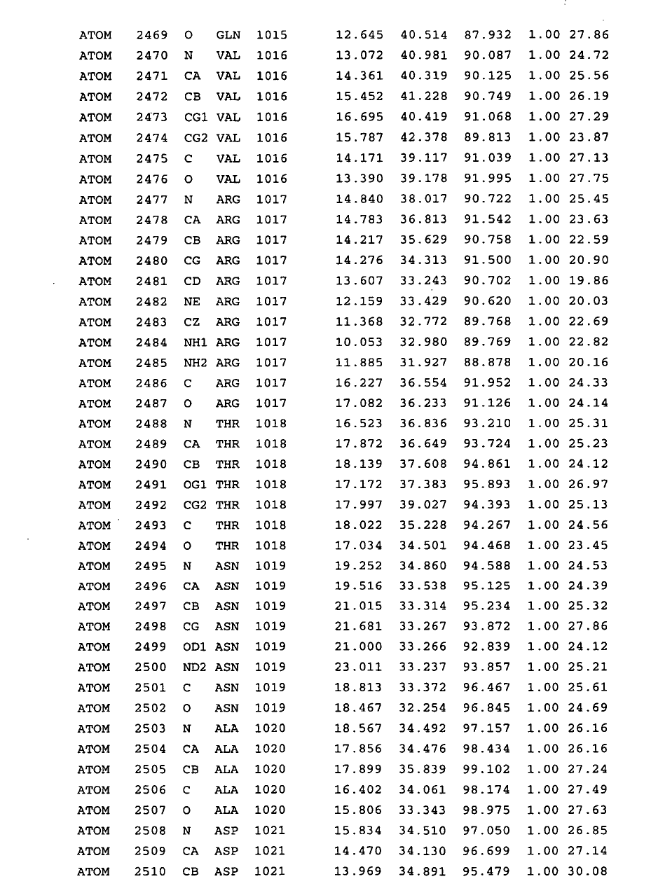

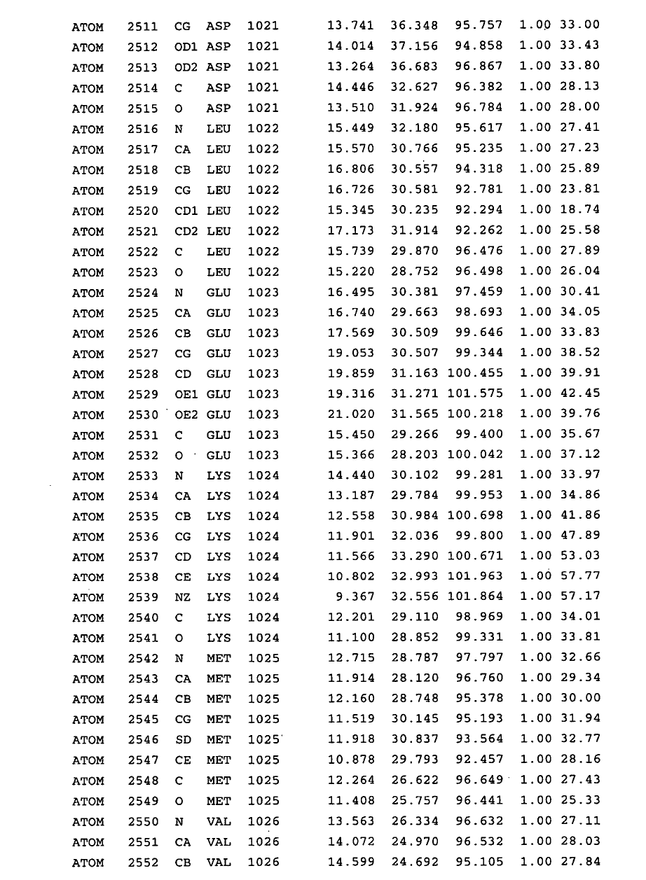

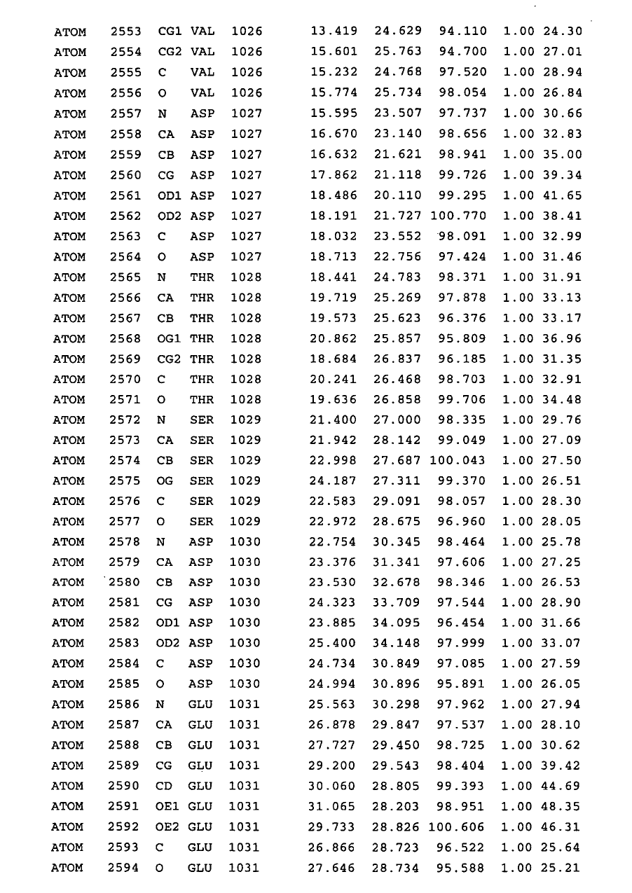

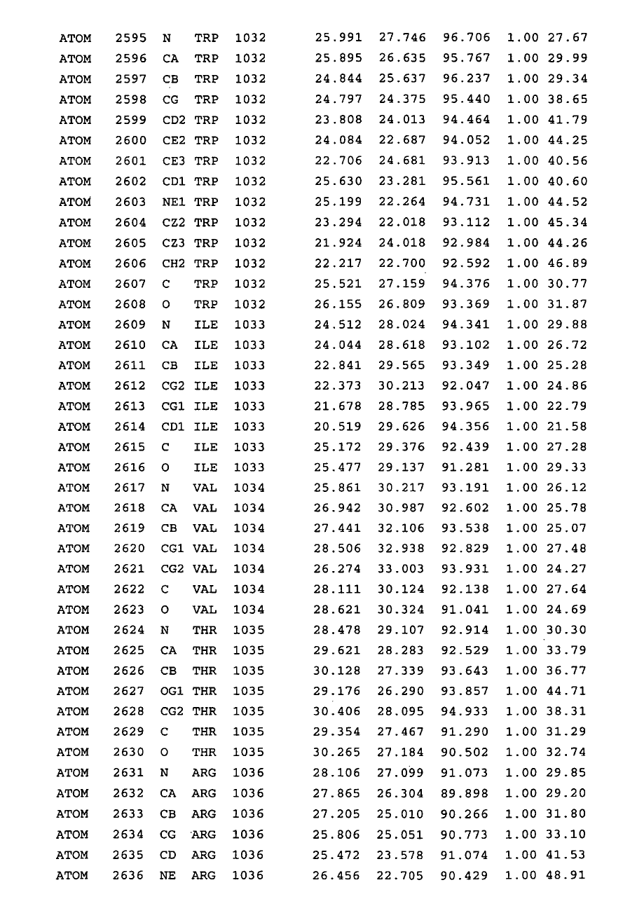

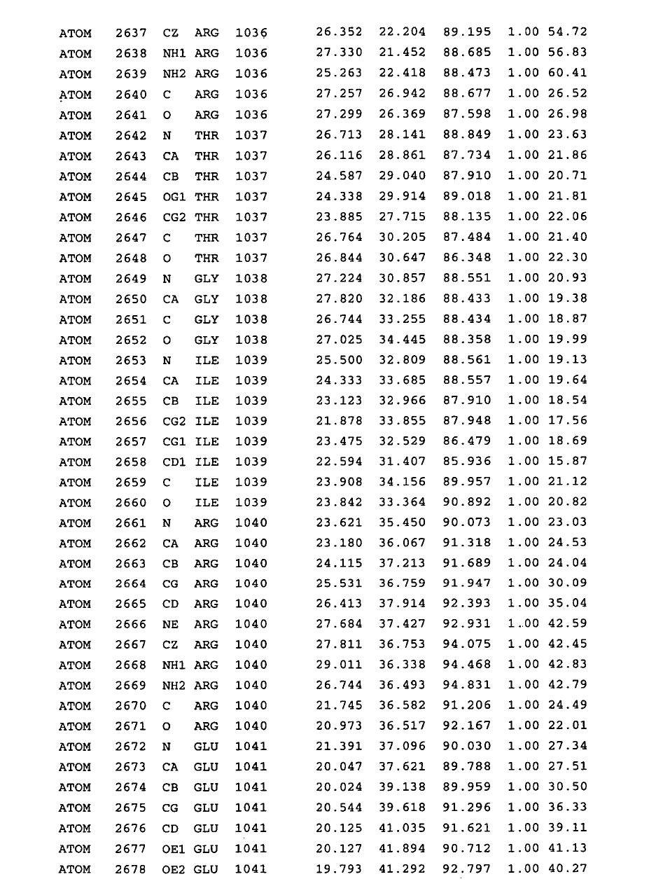

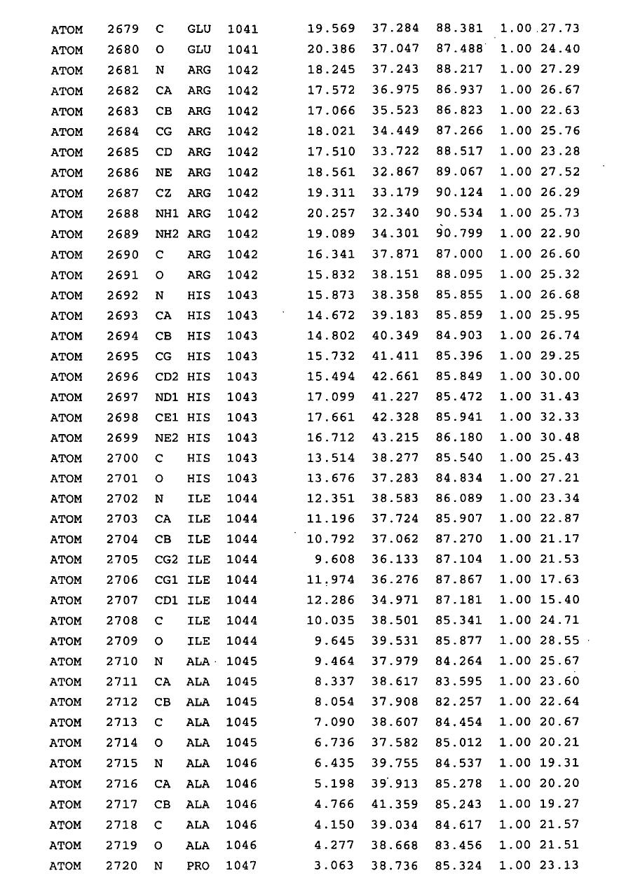

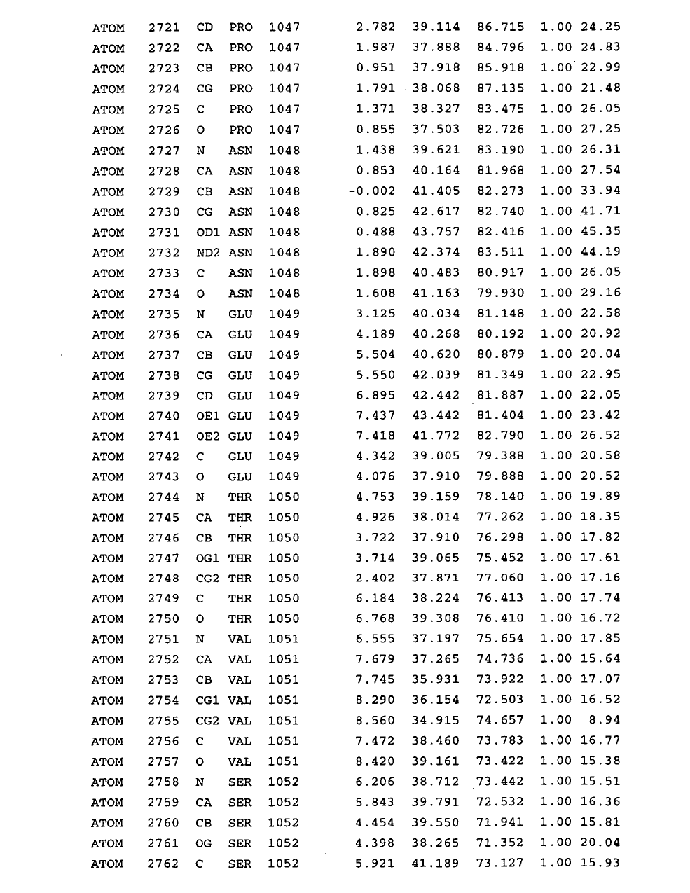

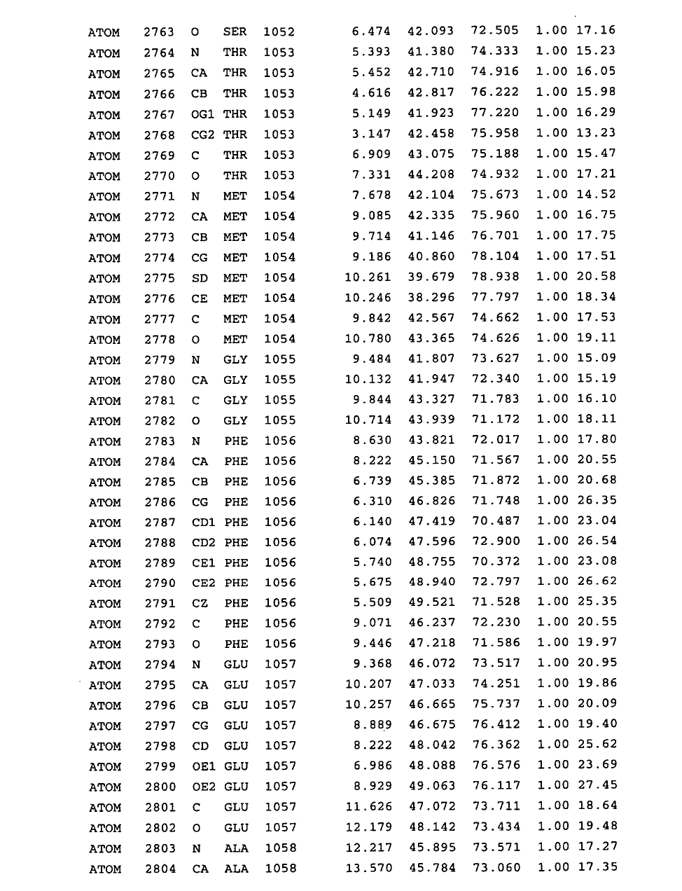

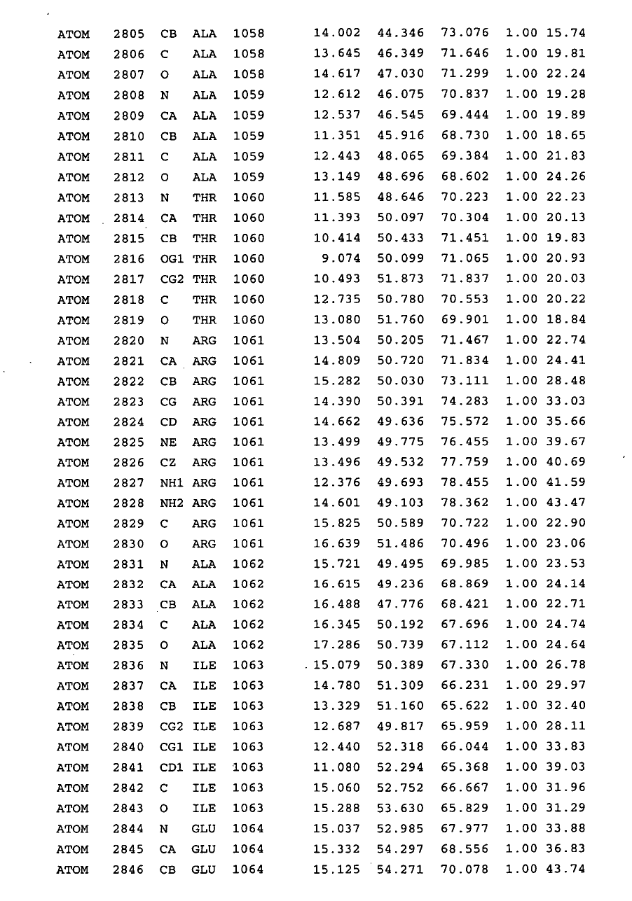

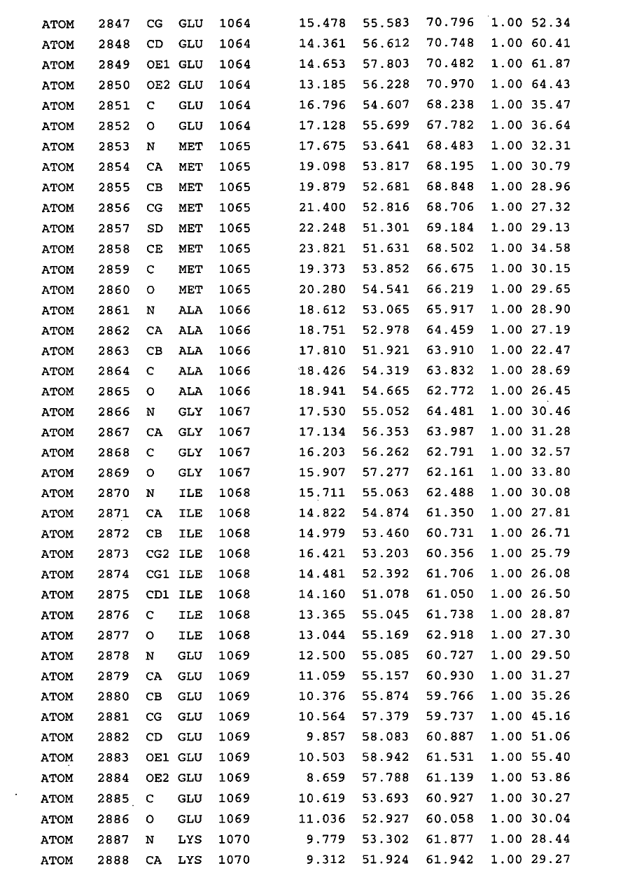

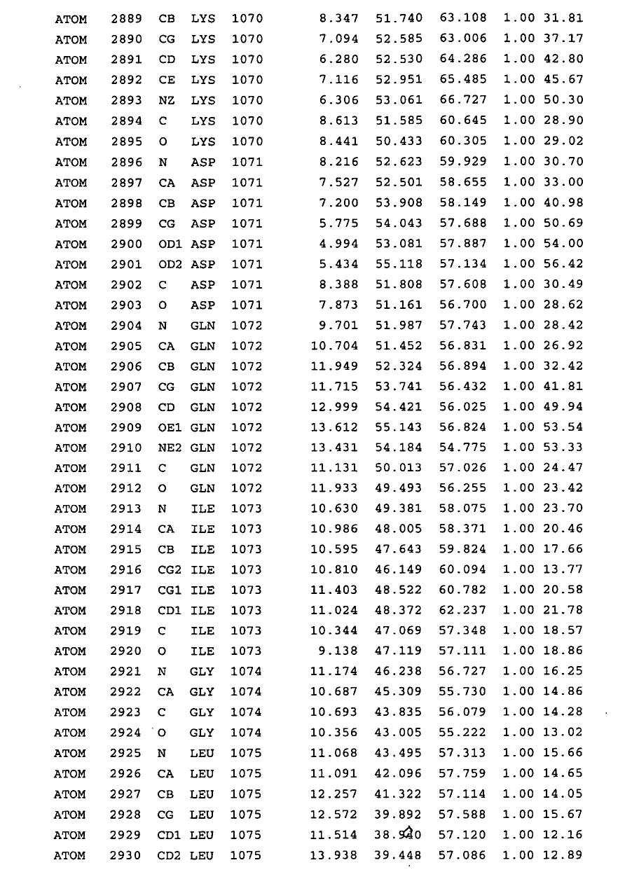

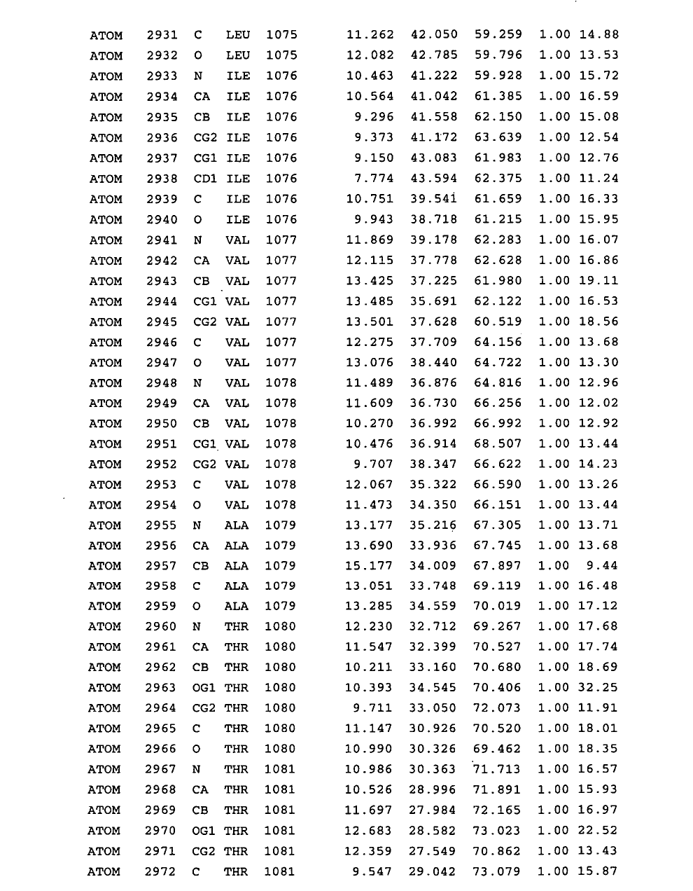

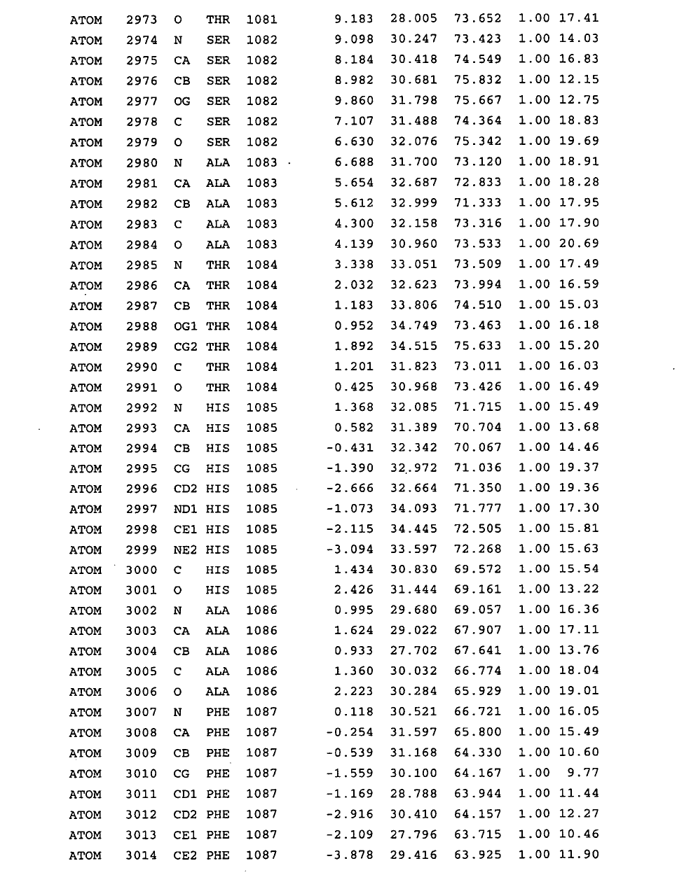

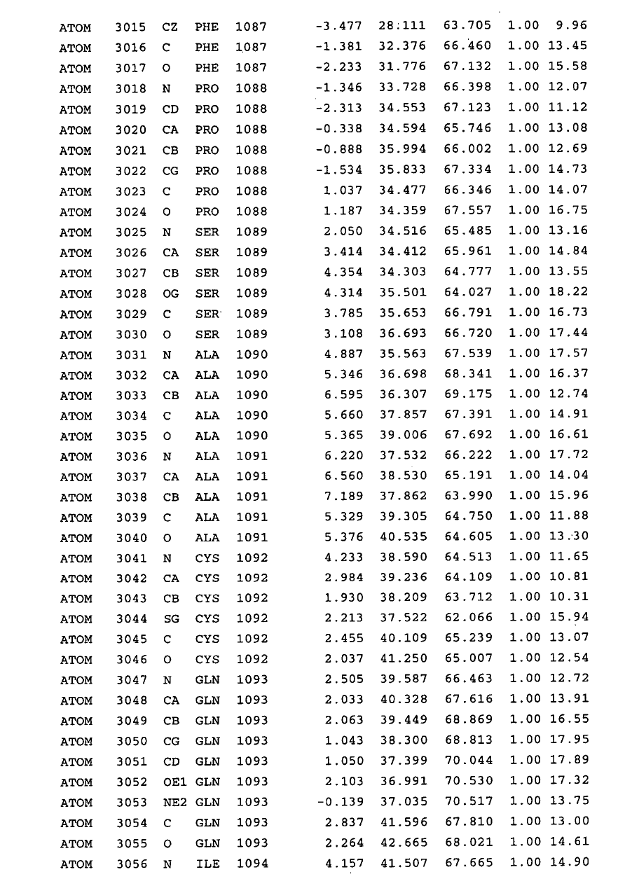

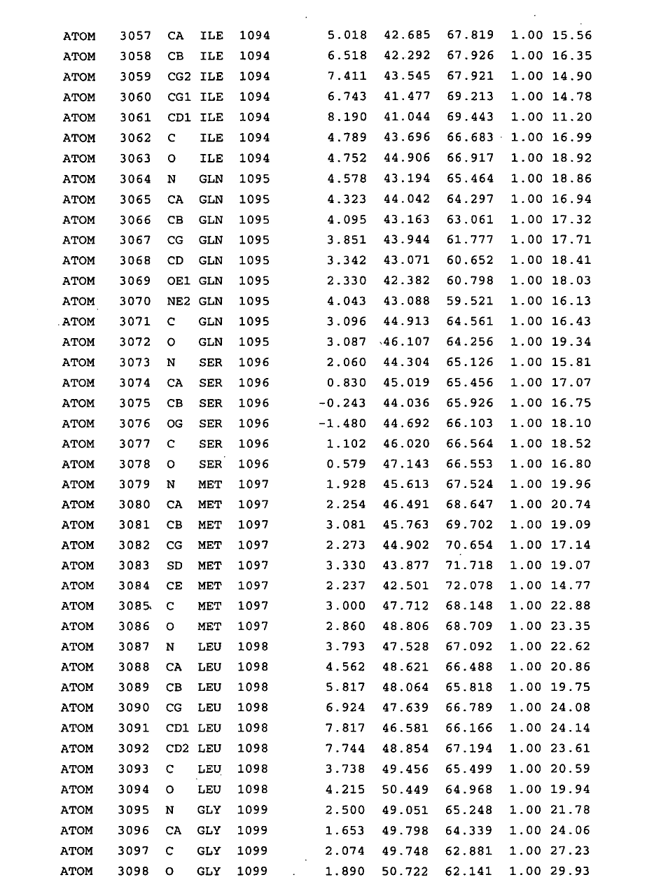

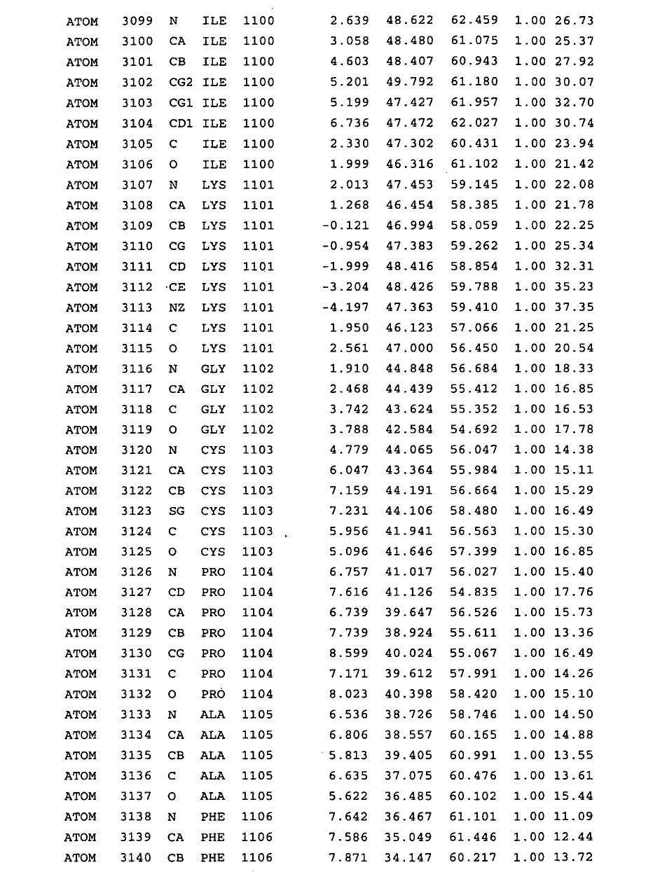

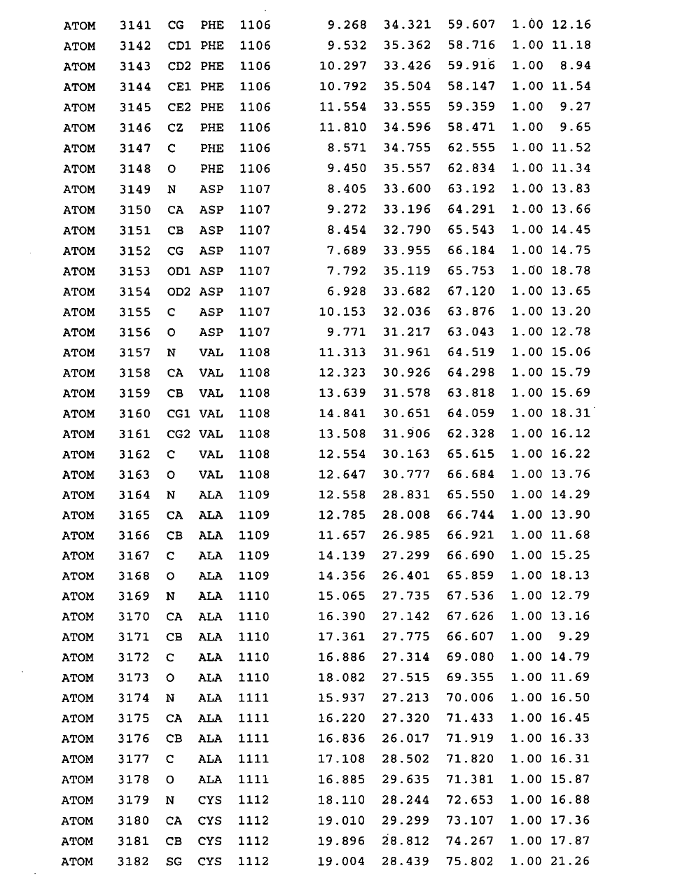

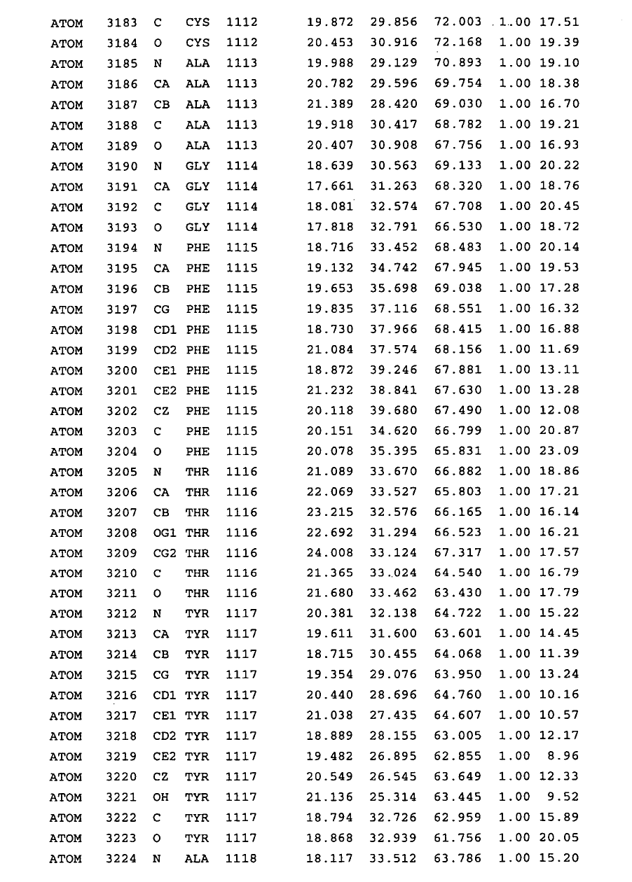

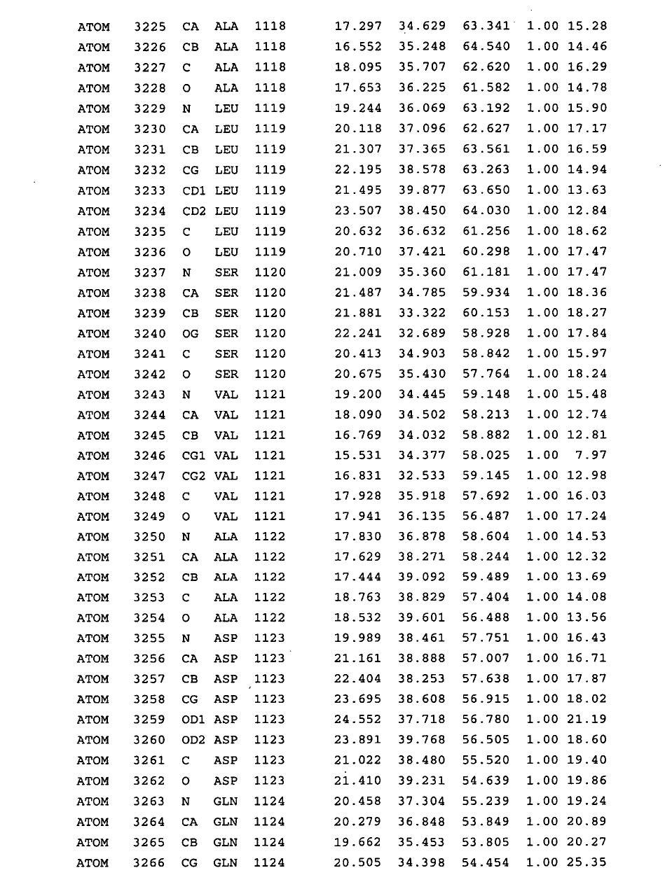

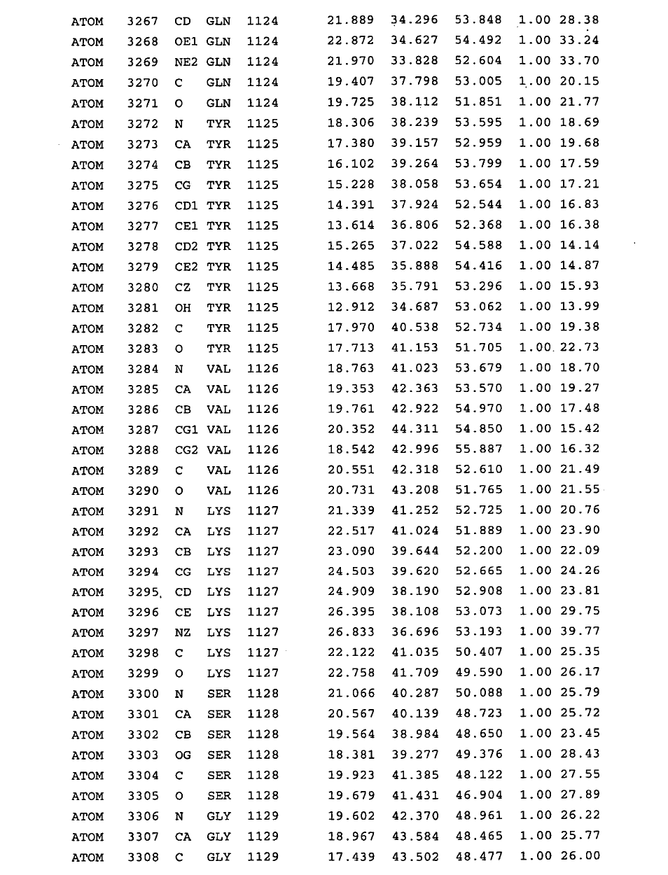

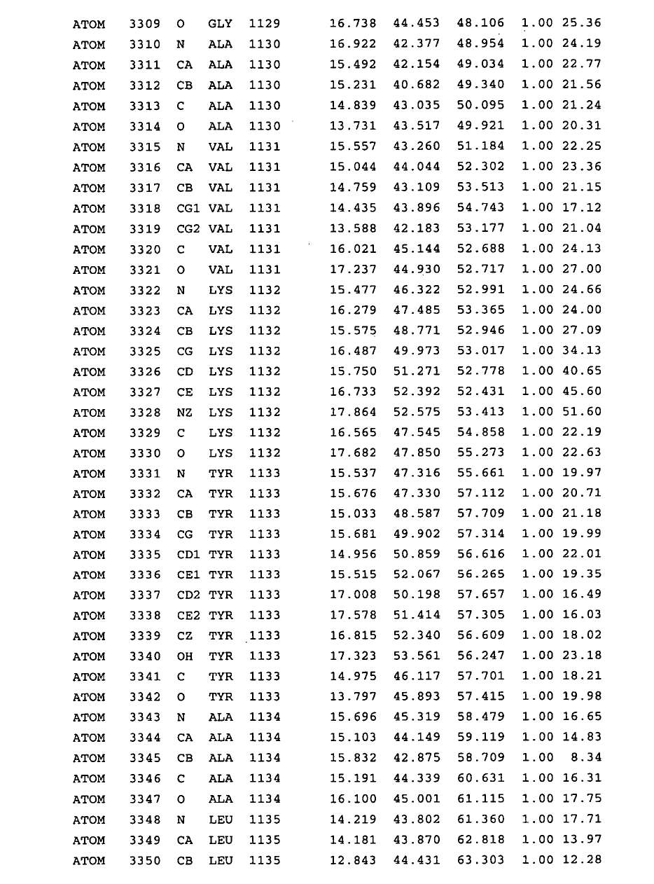

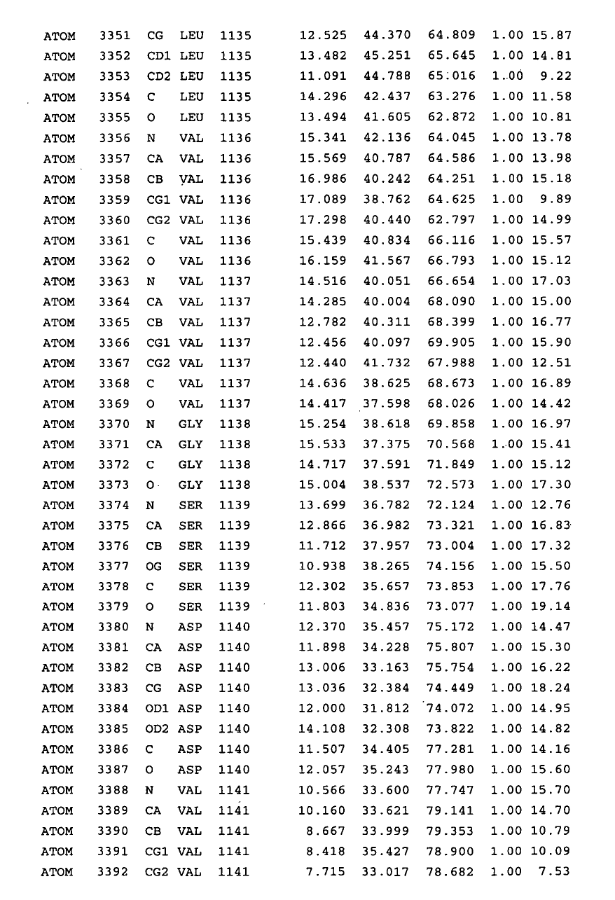

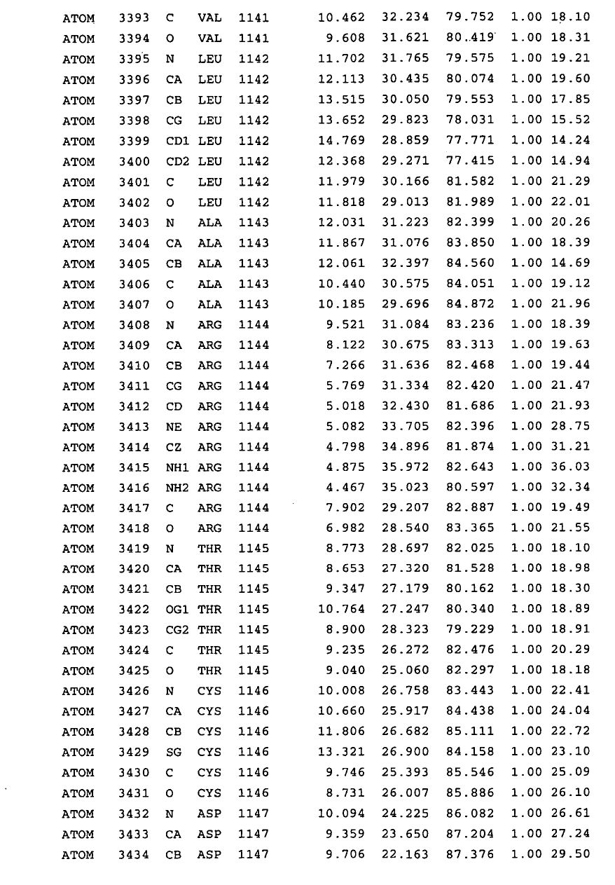

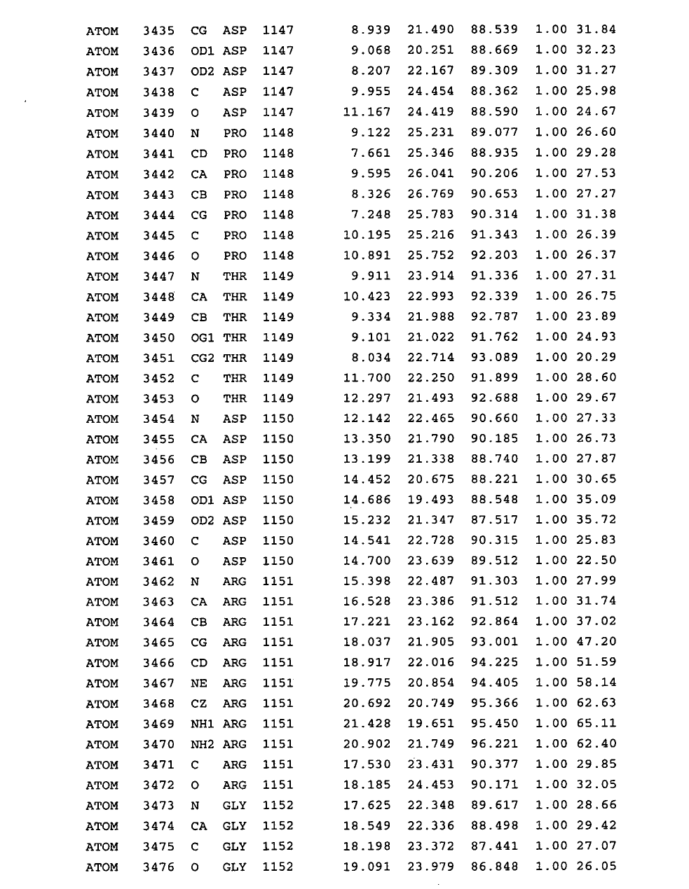

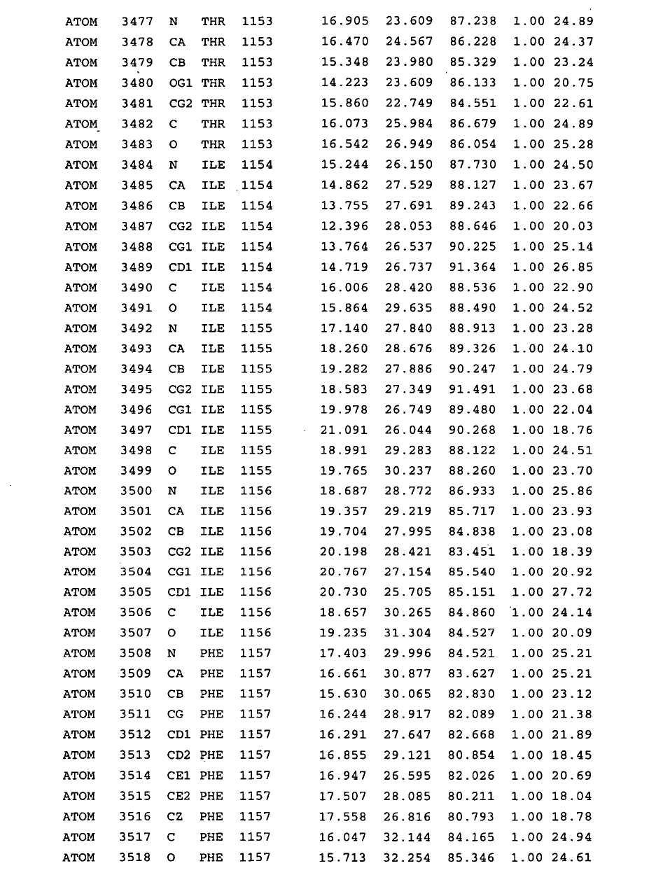

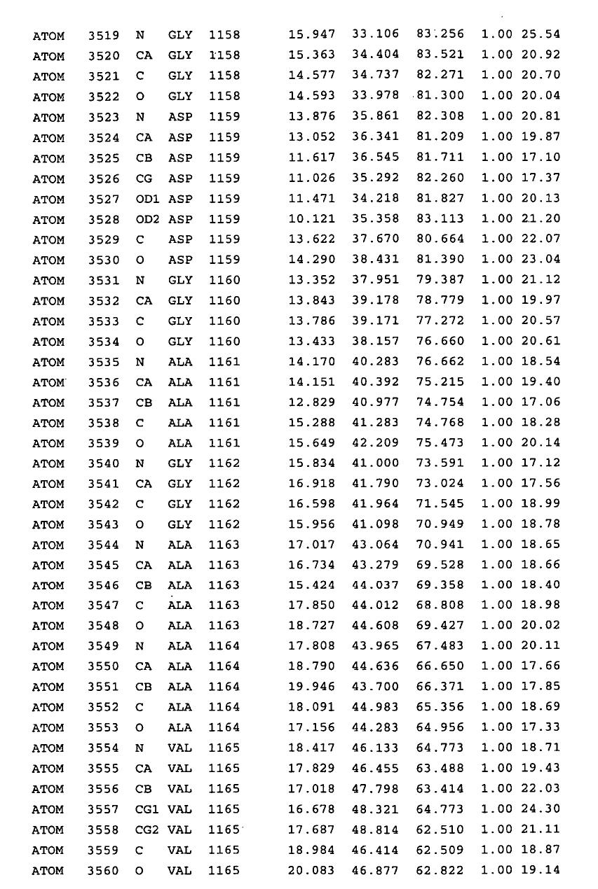

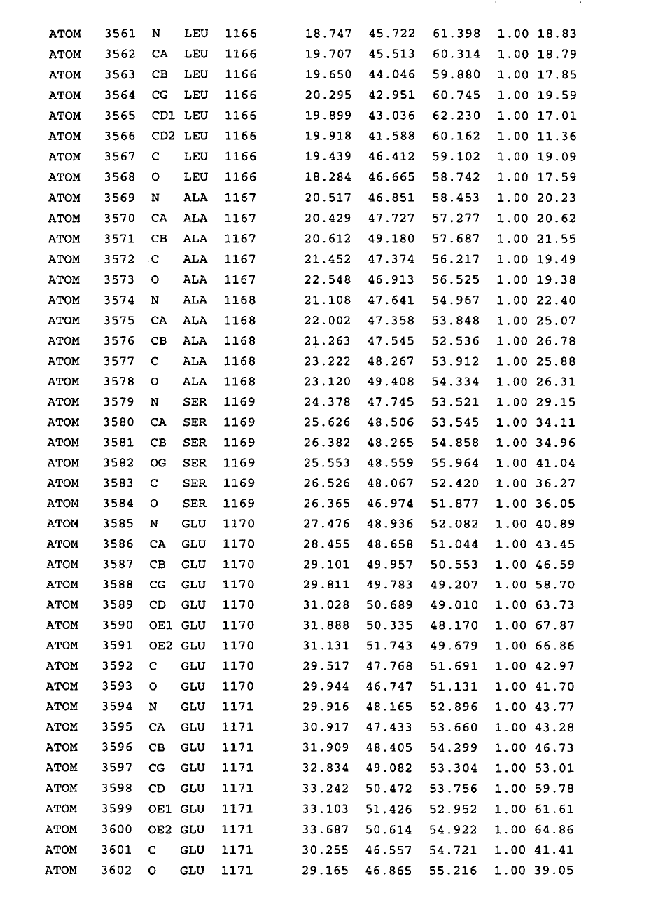

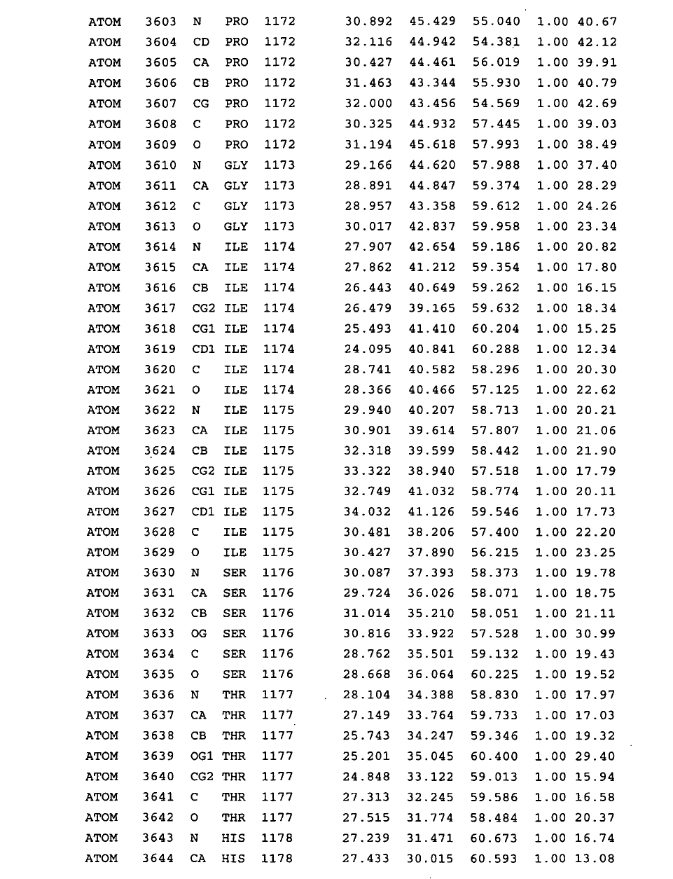

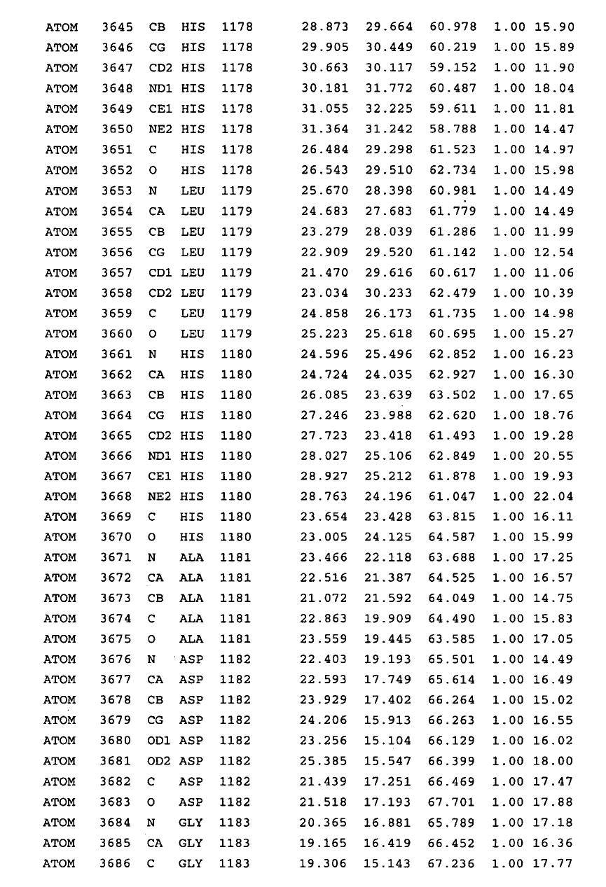

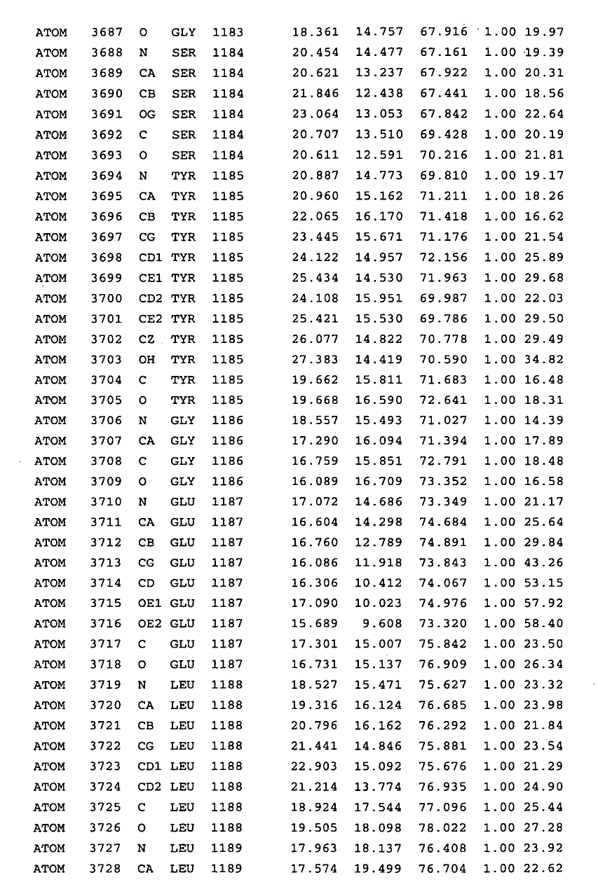

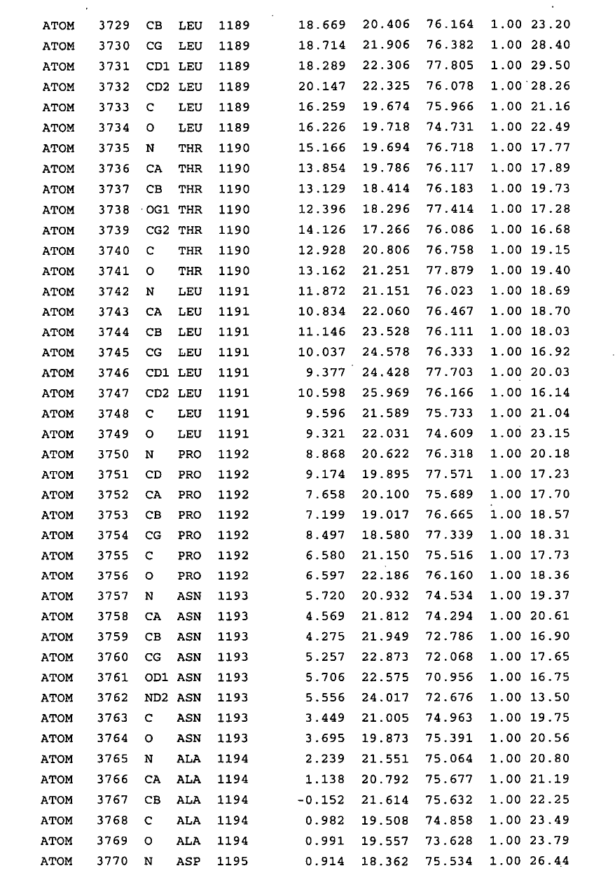

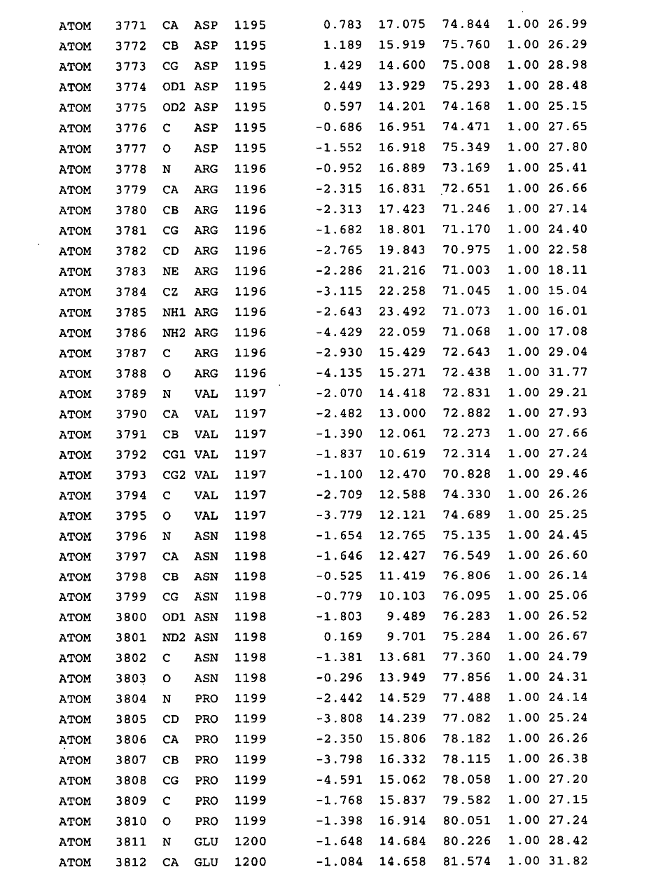

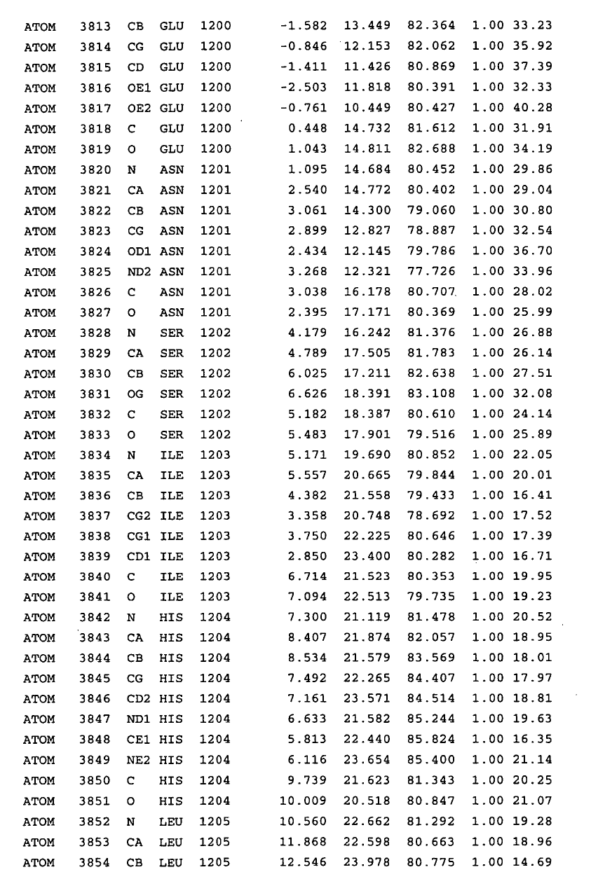

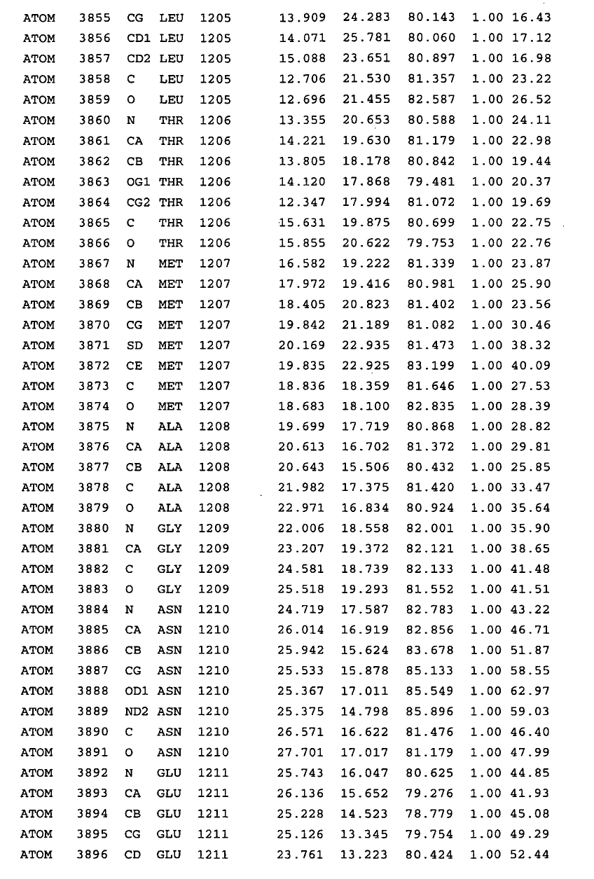

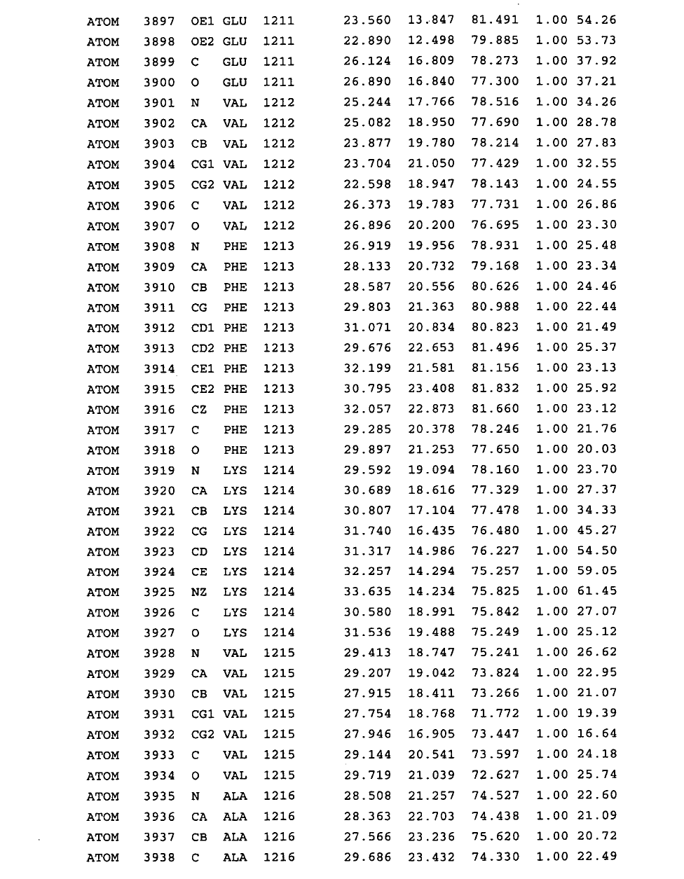

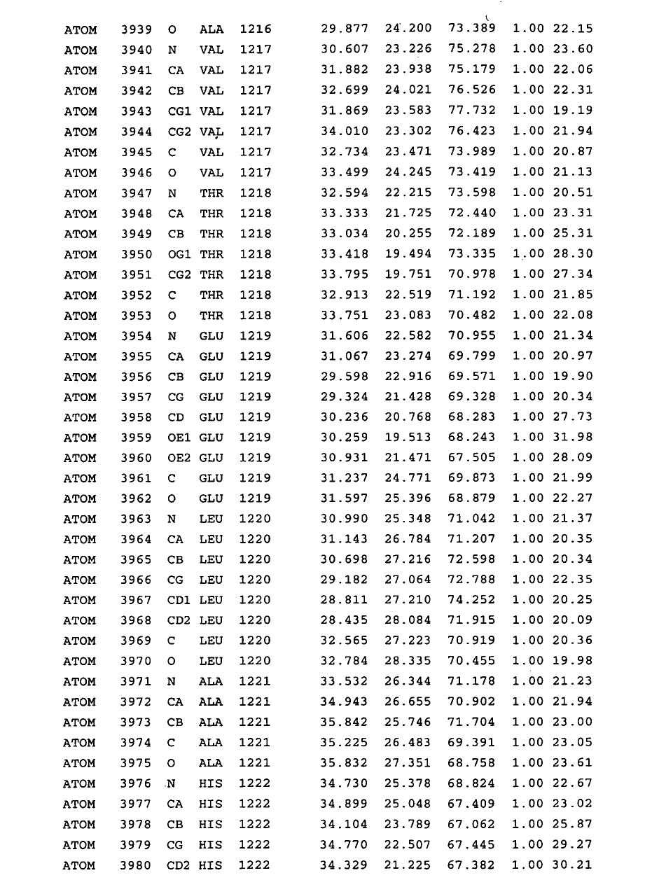

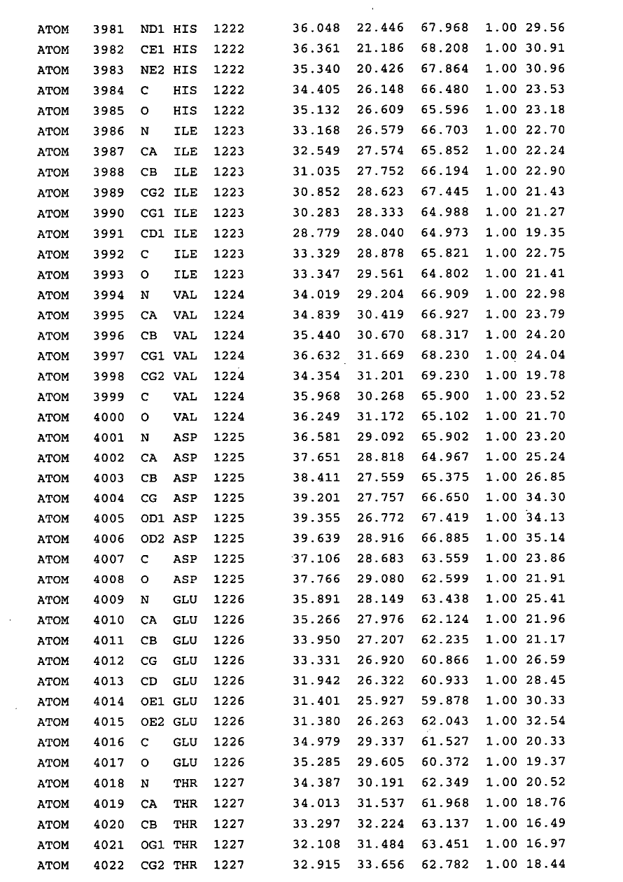

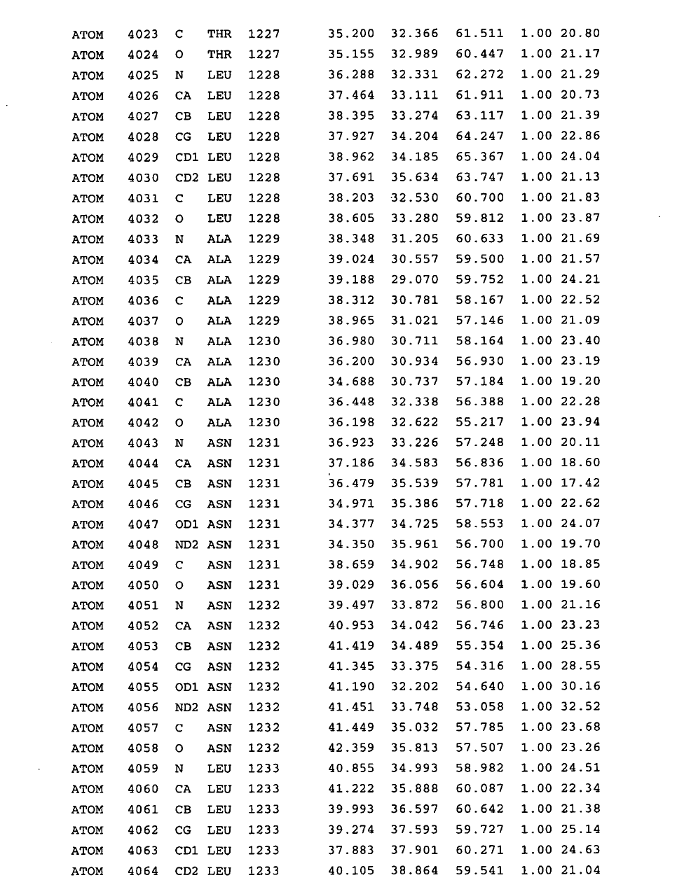

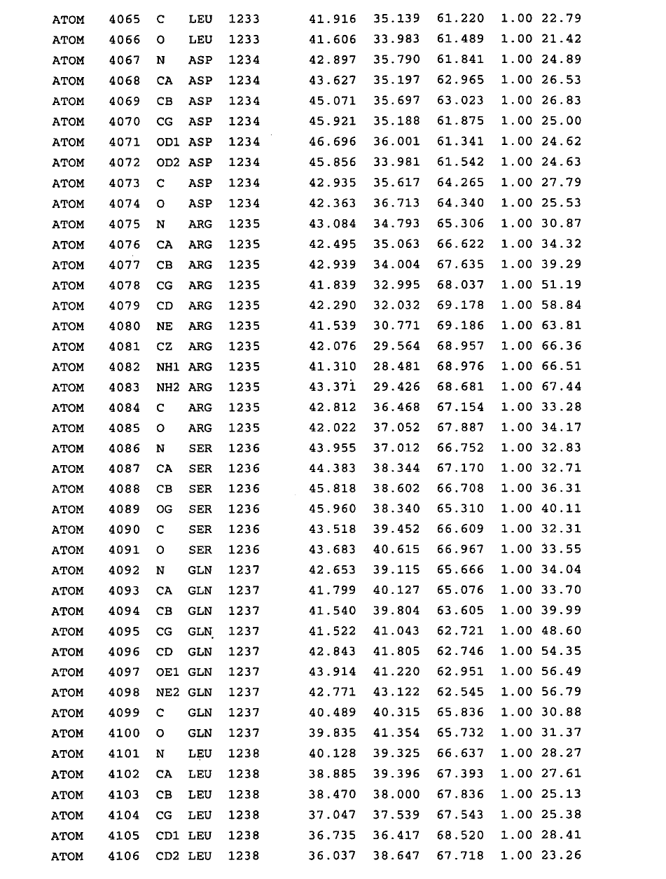

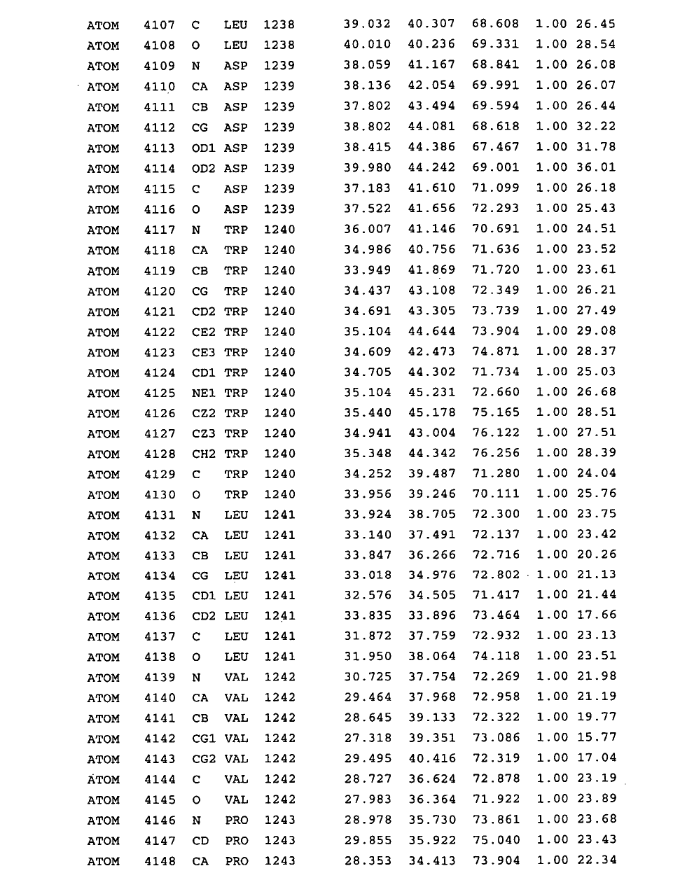

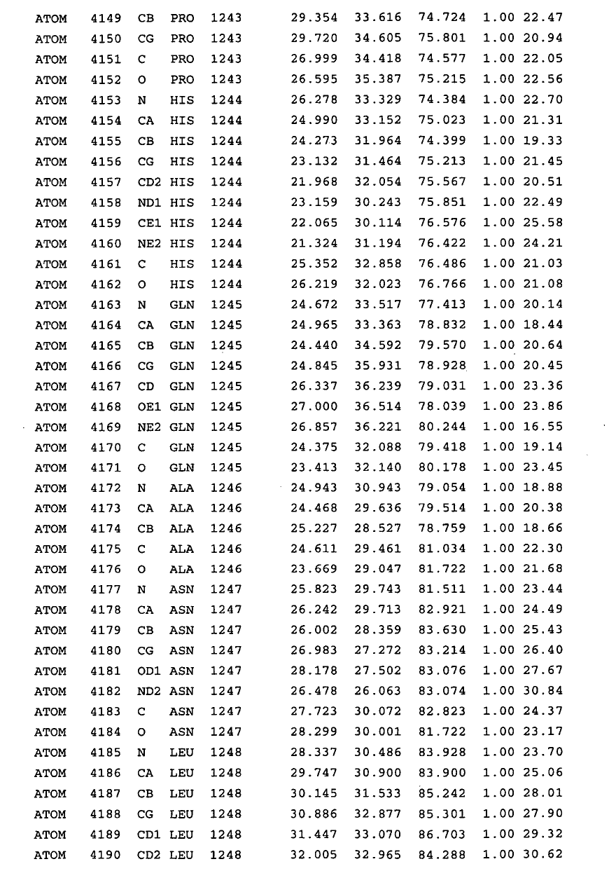

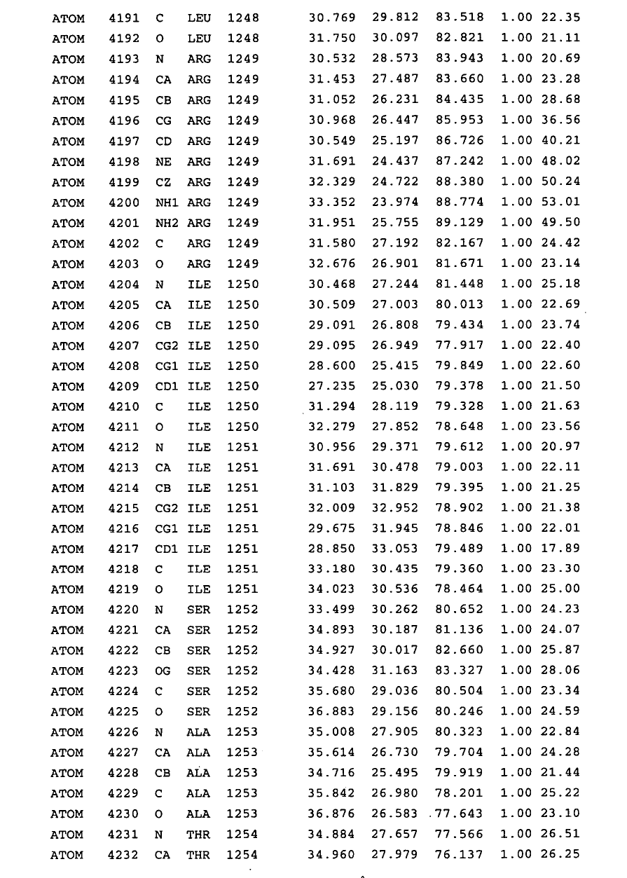

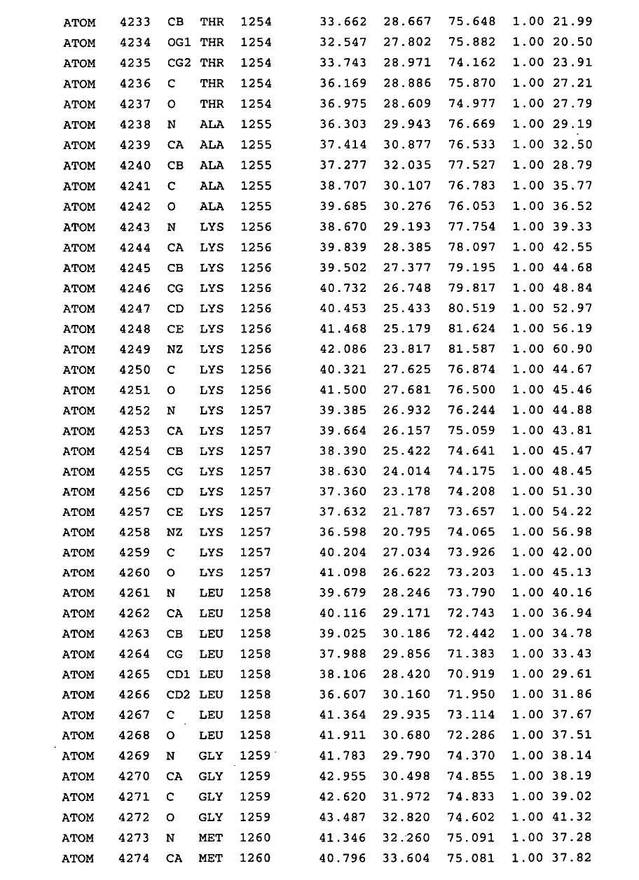

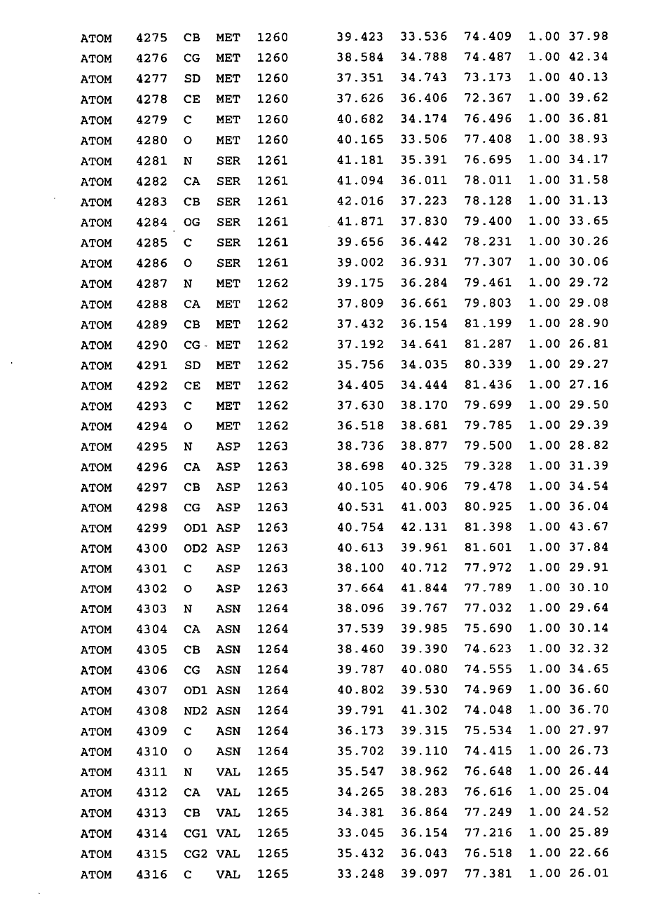

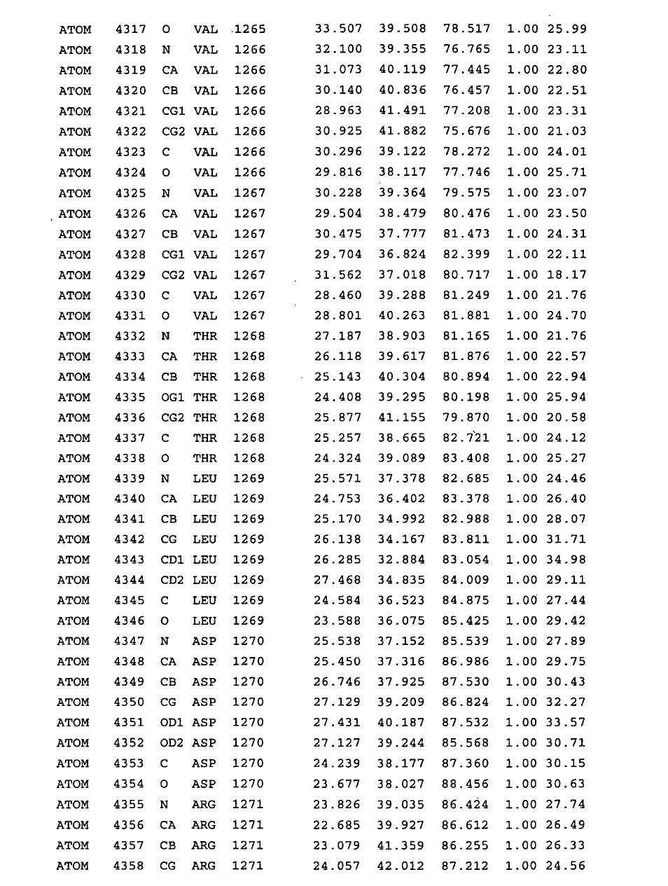

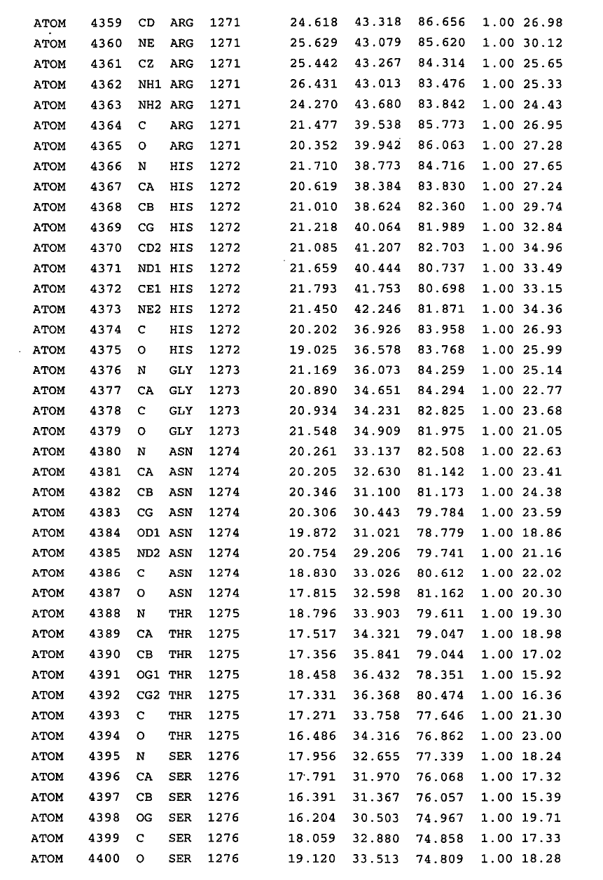

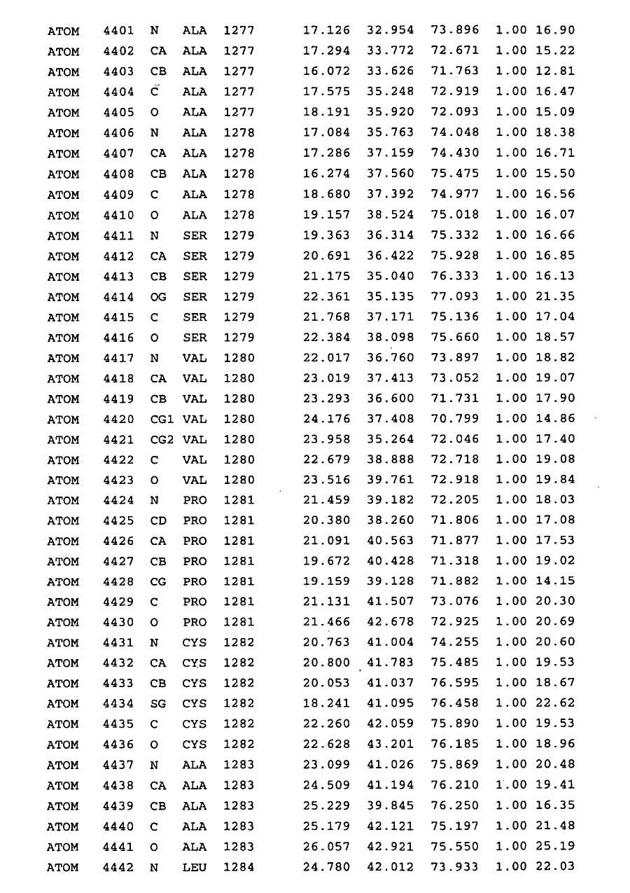

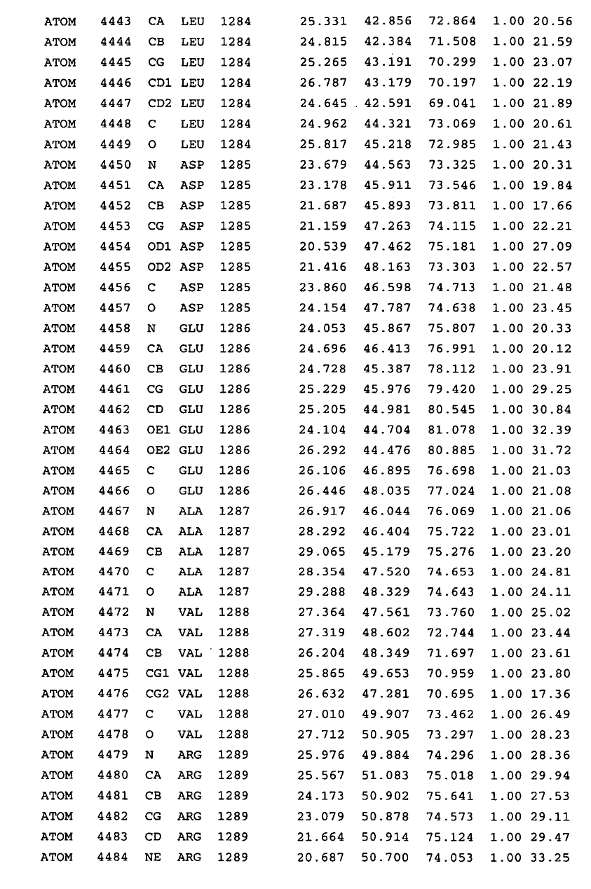

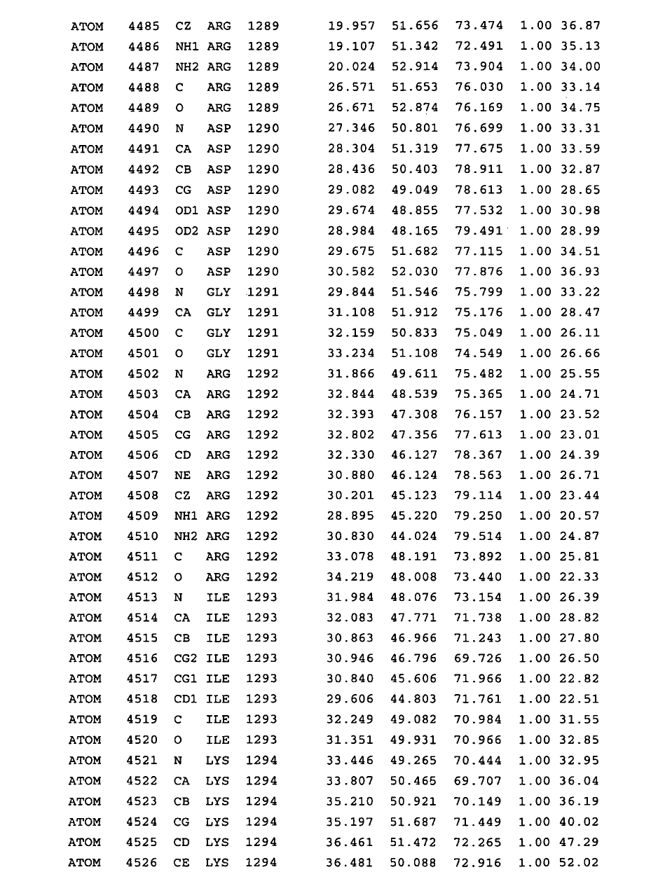

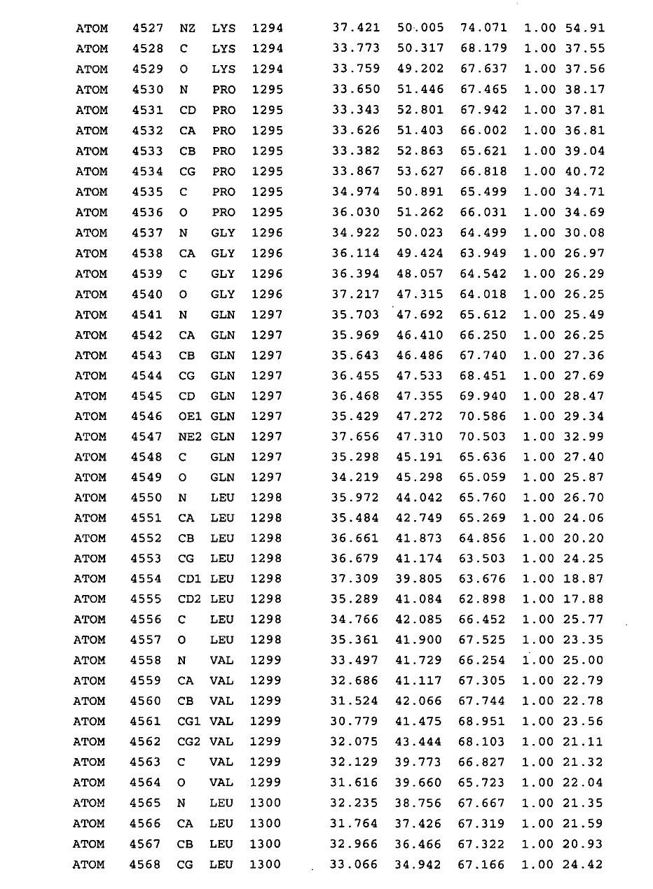

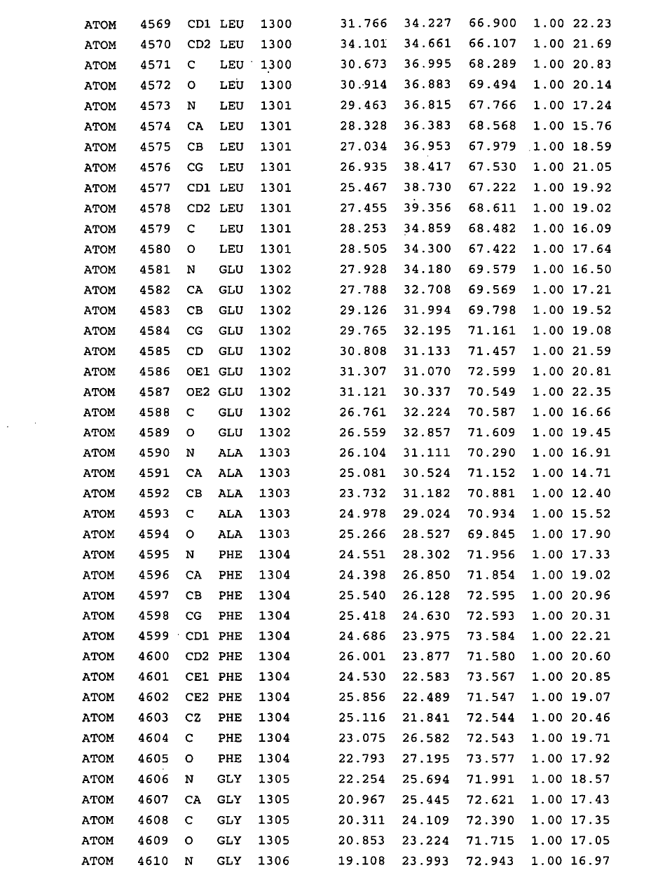

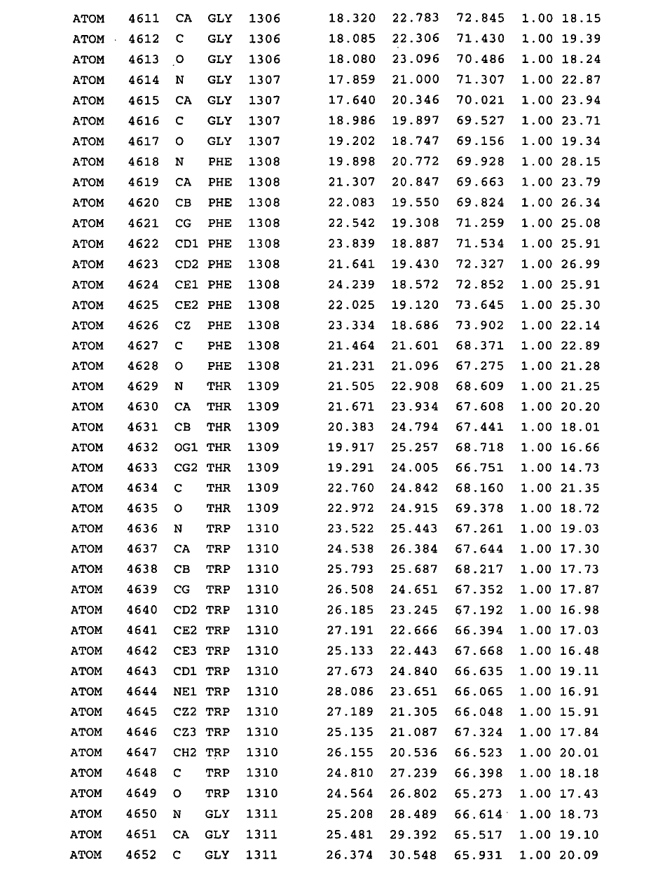

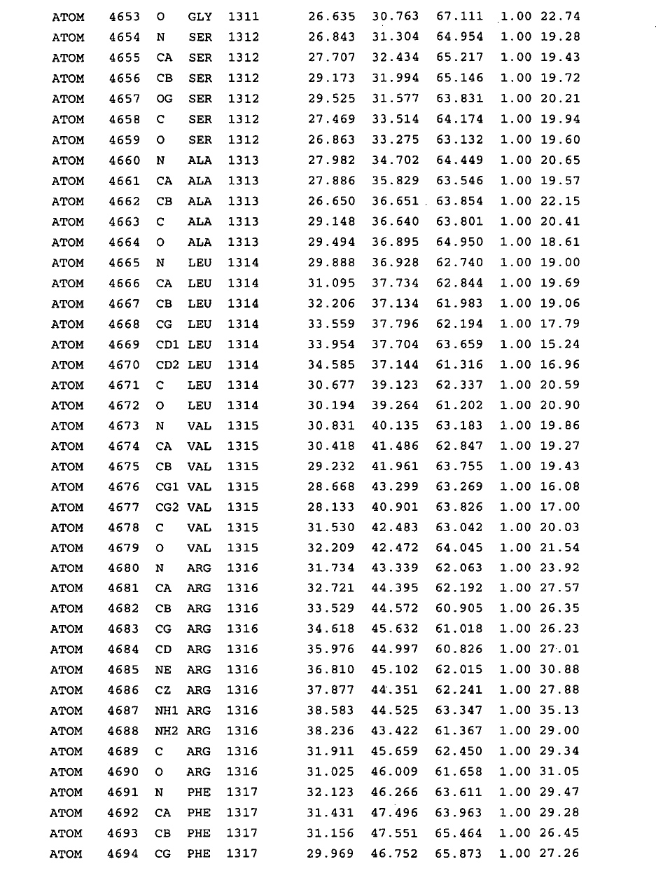

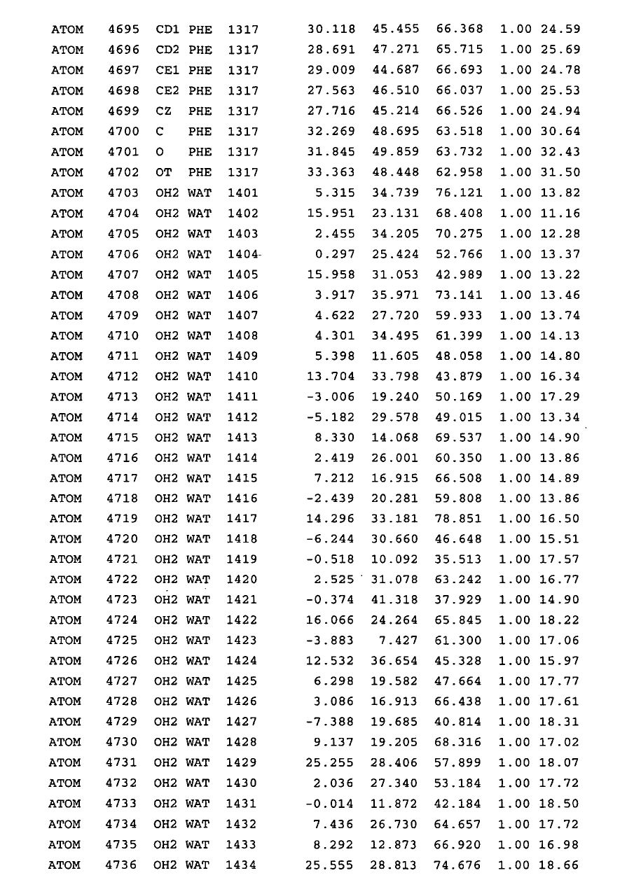

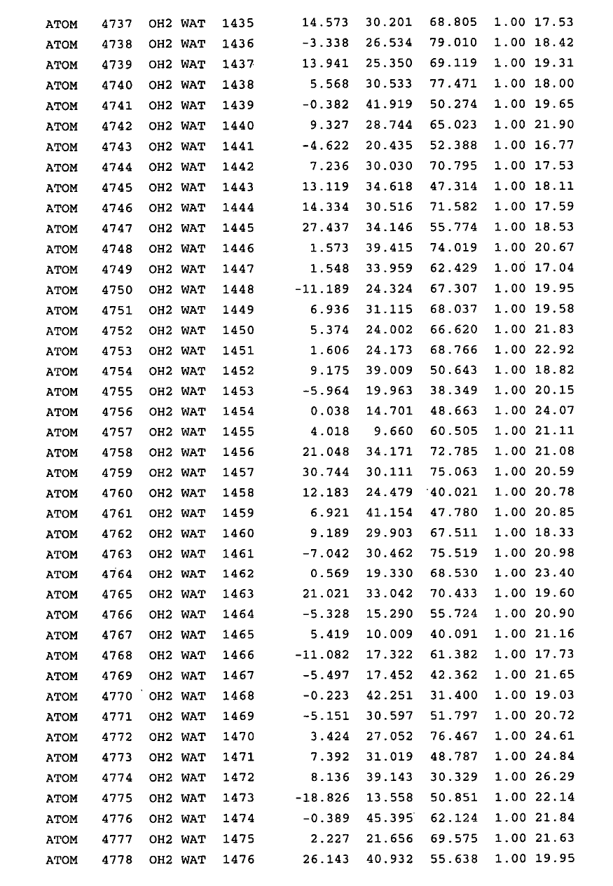

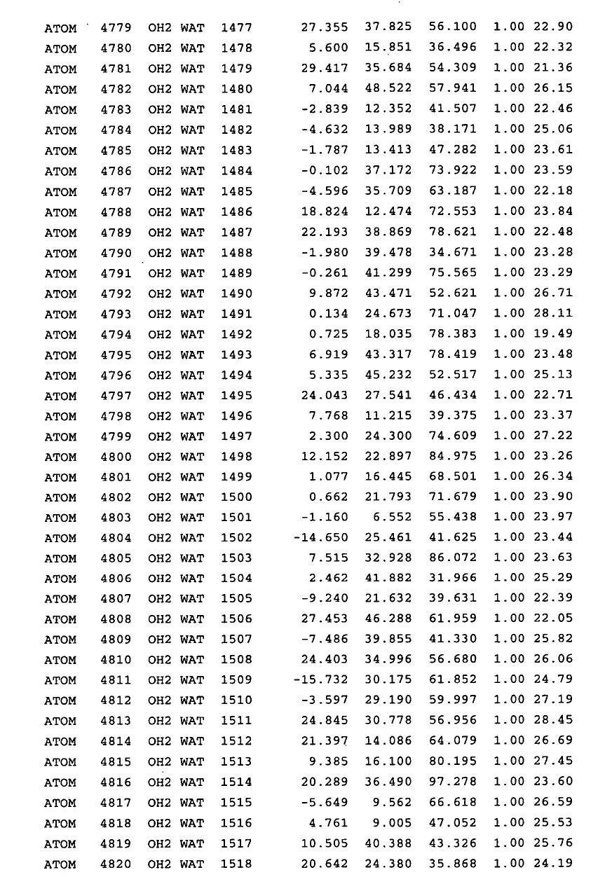

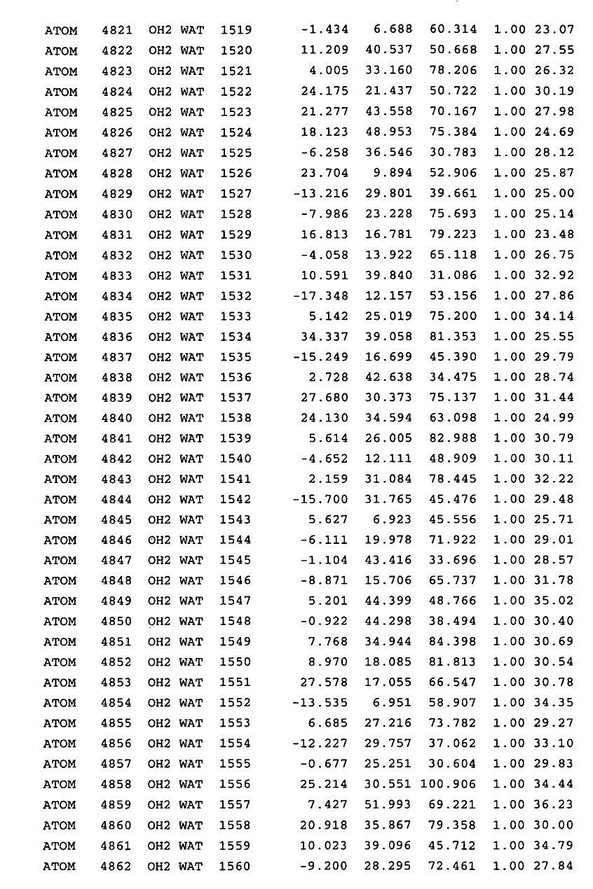

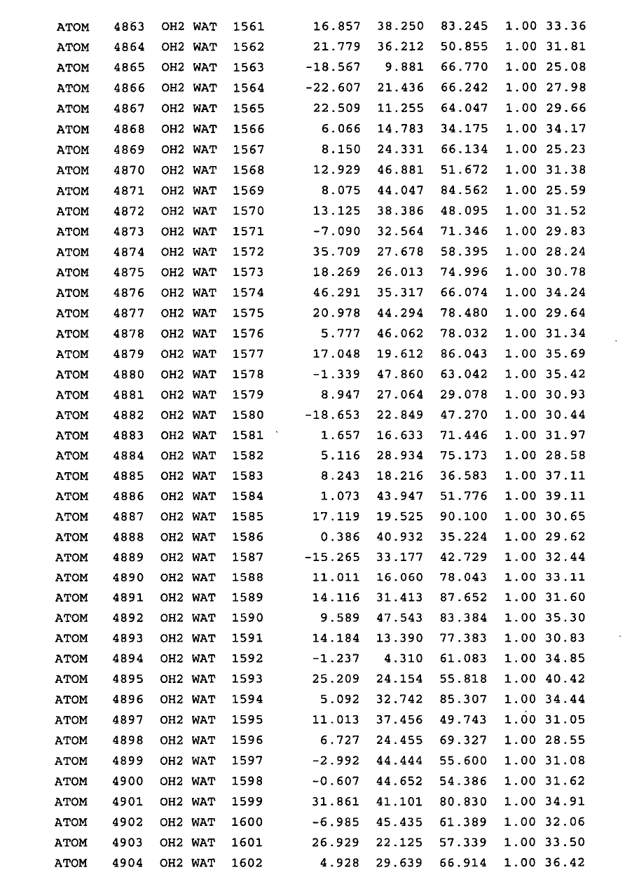

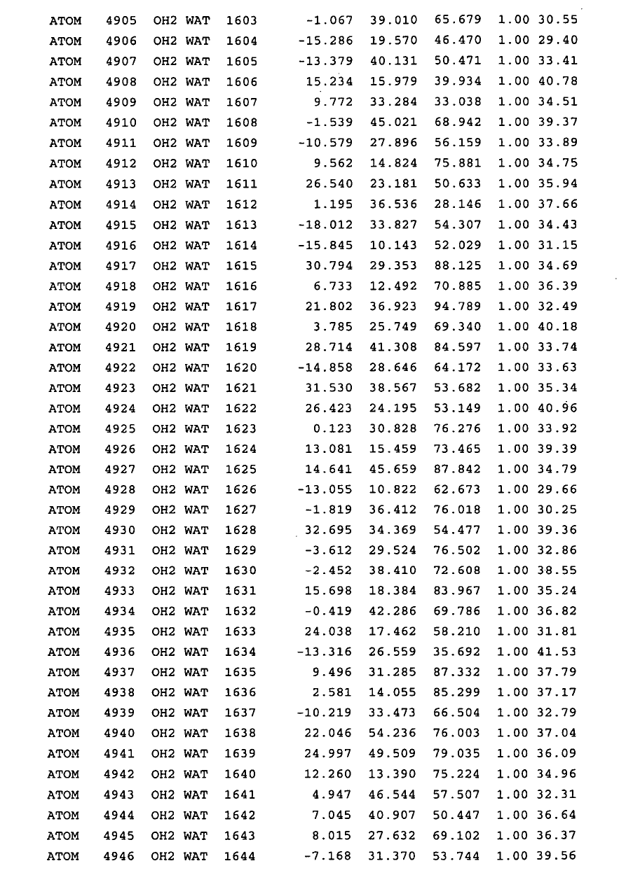

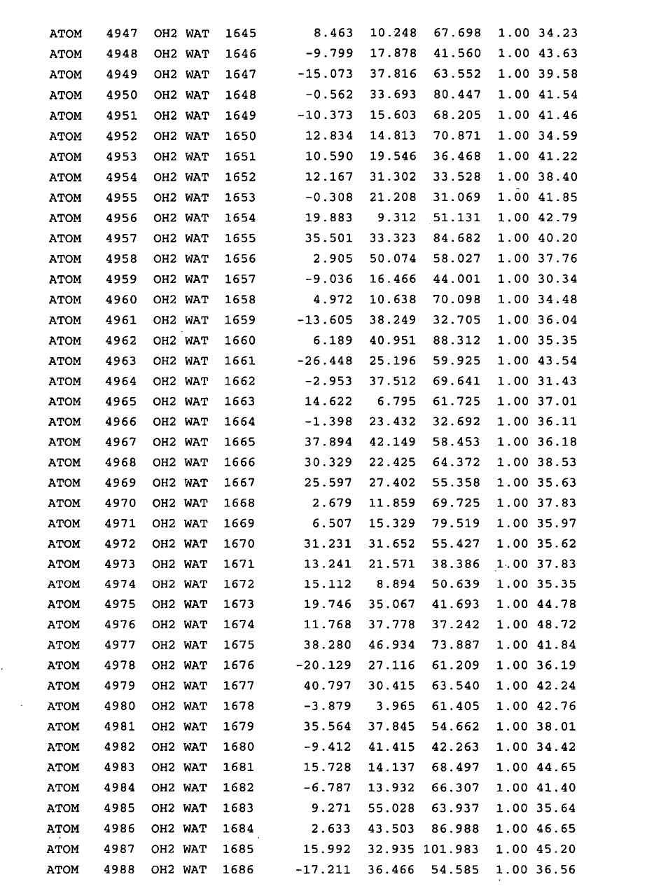

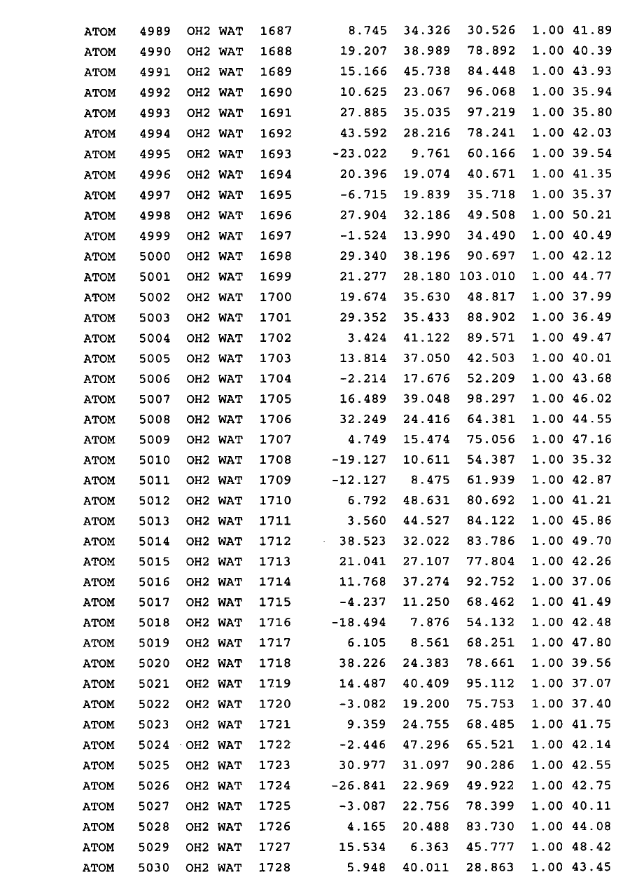

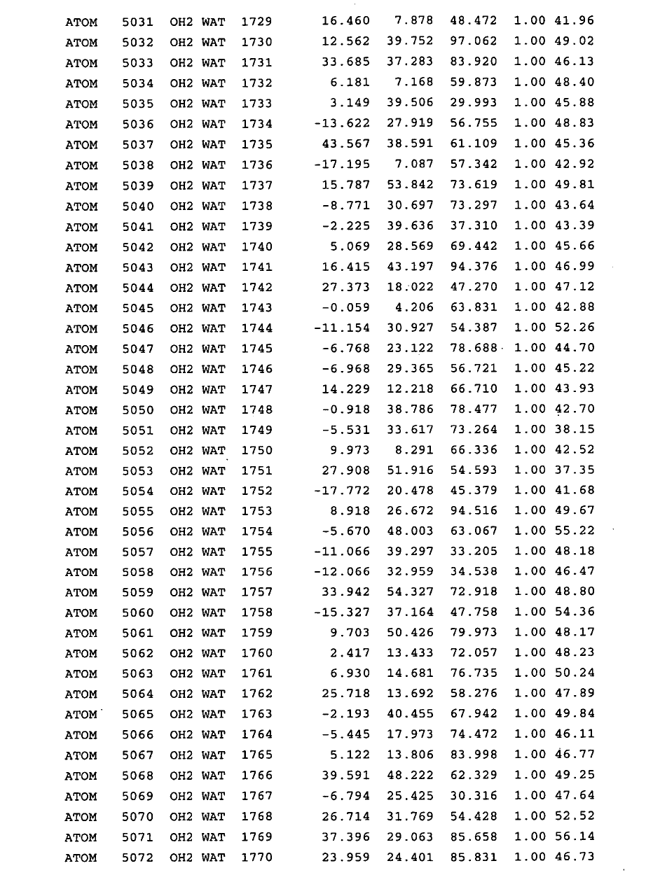

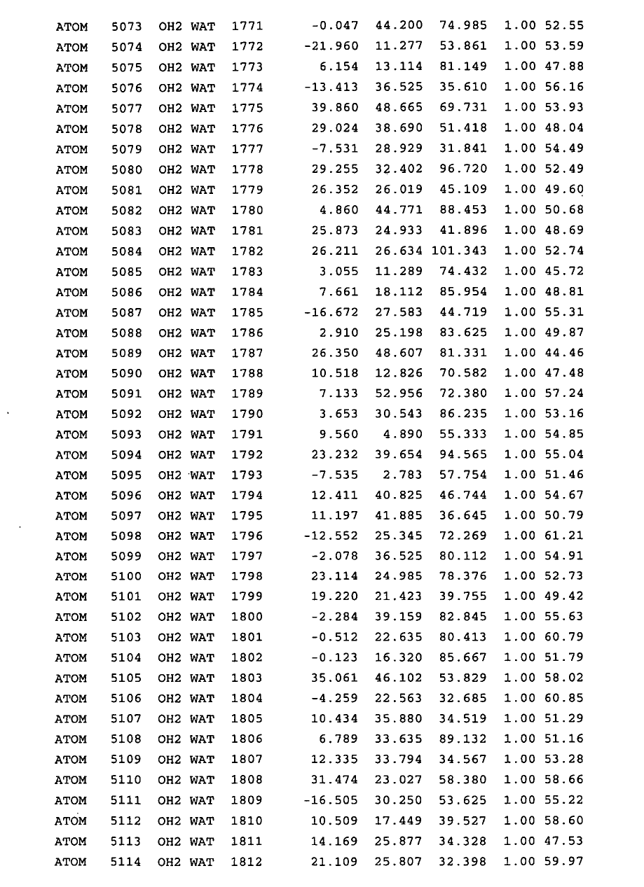

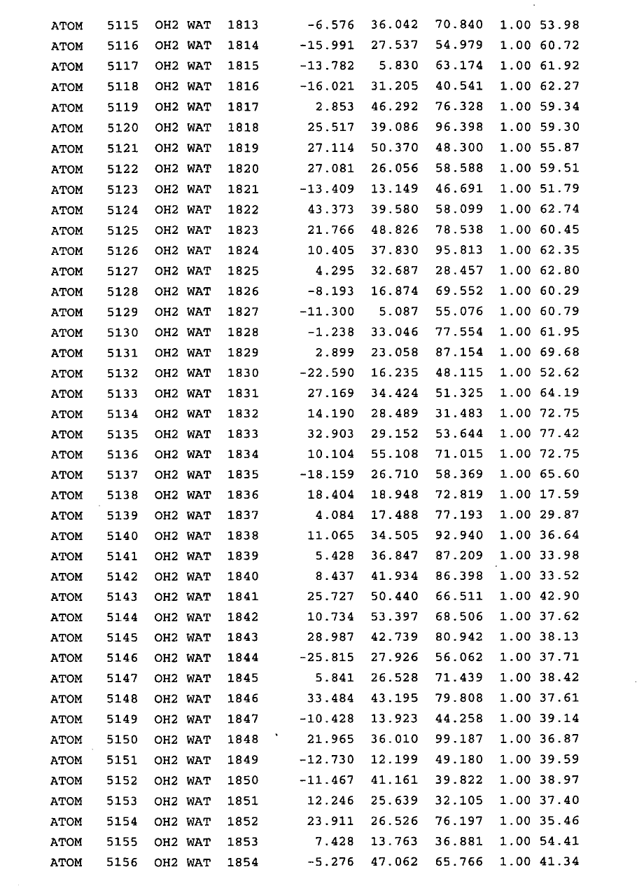

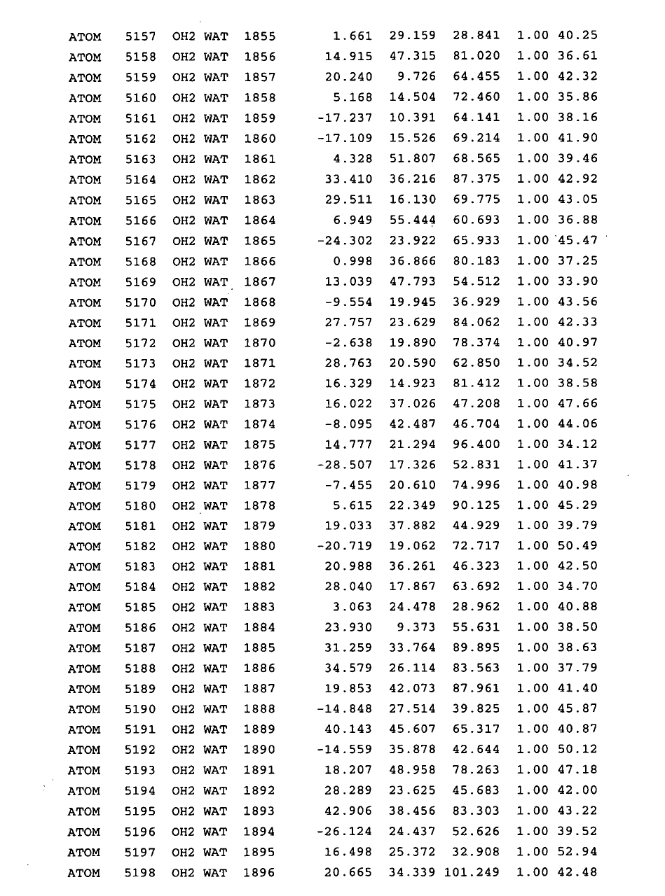

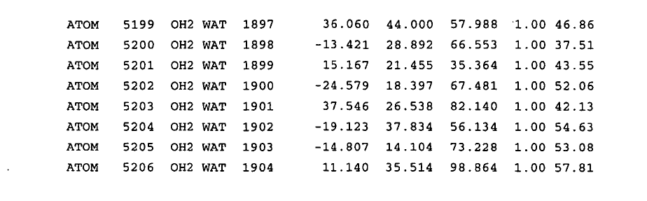

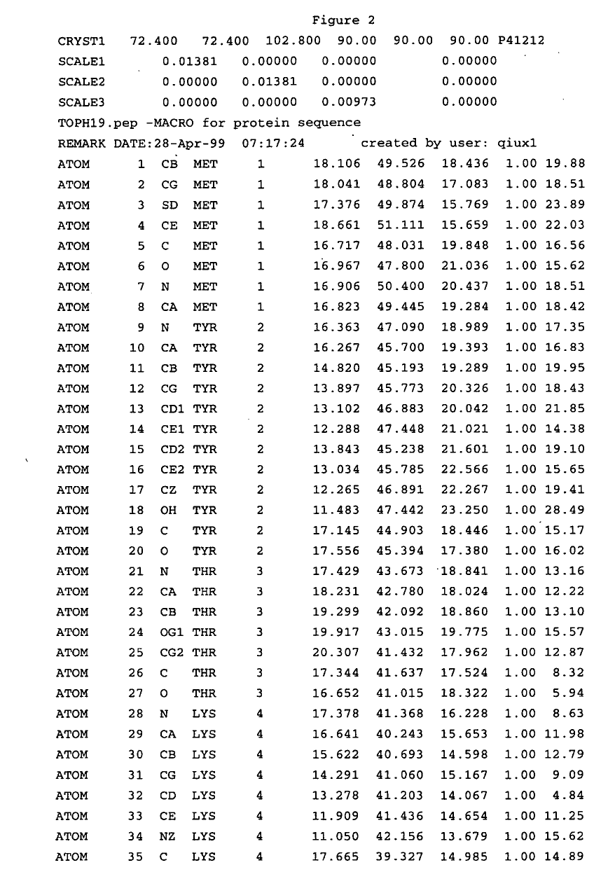

Further refinement of the atomic coordinates will change the numbers in FIG. 1-2 and Tables I-III, refinement of the crystal structure from another crystal form will result in a new set of coordinates. However, distances and angles in Tables II will remain the same within experimental errors, and relative conformation of residues in the active site will remain the same within experimental error. For example, the two independently determined monomers in our crystal form 1 and the monomer in crystal form 2 do not have identical numerical coordinates, but the structures of these three monomers have very similar structures, and the spatial relationship between amino acid residues are considered the same within experimental error. In fact, we would consider any structure that can be superimposed onto that of FabH with an rms error of less than 1.5 Å on α-carbon atoms being a close structural homologue and the same rms error but over all protein atoms being an identical structure. FIG. 1 provides the atomic coordinates of the E. coli FabH dimer, which contains 634 amino acids. FIG. 2 provides the atomic coordinates of the E. coli FabH monomer in complex with acetyl-CoA, which contains 317 amino acids. The FabH enzyme is characterized by an active site which preferably contains a binding site for the first substrate acetyl-CoA and the second substrate malonyl-ACP. The catalytic residues in FabH are Cys112, His244 and Asn274, compared to Cys163, His303 and His340 in FabF. The difference in catalytic residues is not only limited to their amino acid identity (His340 to Asn274 change), but also their relative spatial arrangement. While FabH Cys112 and Asn274 can be well superimposed onto FabF Cys163 and His340, His244 of FabH occupies a very different position from that of His303 of FabF. This indicated the catalytic mechanisms of the two enzymes are very different. The crystal structure described herein was solved in the presence and absence of acetyl-CoA. We identified that the catalytic Cys112 has been covalently aceytlated, and the product CoA is still bound to the active site. The bound CoA enabled us to identify the active site cavity, which is long and narrow and shaped nicely to bind the β-mercaptoethylamine-patotheinate arm of CoA. The structure of the acetyl-CoA complex also revealed all the key residues that are interacting with CoA and lining the active site, which is identified as a set of 33 amino acid residues listed in Table I. For example, the adenine part of CoA is sandwiched between the side chains of Arg151and Trp32. Our structures are determined in the absence of malonyl-ACP. However, the same acetyl-CoA binding cavity should bind malonyl-ACP as well because their active site binding regions are very similar and there is no apparent additional entrance to the active site. Moreover, while the FabH molecular surface in general negatively charged, a region just outside of the active site cavity is positively charge. This surface is mainly comprised of three α-helices (30-37, 209-231 and 248-258) and contains a number of positively charged amino acids (Arg36, Arg40, Lys214, His222, Arg235 Arg249, Lys256, Lys257). Since the acyl-carrier protein (ACP) is known to be very acidic or negatively charged, it is reasonable to assume this surface being the ACP binding surface.

Table I provides the the atomic coordinates of the apo E. coli FabH structure in the active site (in crystal form 1). Solvent molecules are omitted here for clarity, but can be found in FIG. 1. Residue 487 is Phe87 from the other monomer.

TABLE I

| ATOM | | RESIDUE | | X | Y | Z | Occ | B |
|---|---|---|---|---|---|---|---|---|
| 1 | N | THR | 28 | −24.151 | 18.846 | 61.990 | 1.00 | 36.45 |
| 2 | CA | THR | 28 | −23.735 | 19.054 | 60.610 | 1.00 | 36.69 |
| 3 | CB | THR | 28 | −22.196 | 19.086 | 60.565 | 1.00 | 32.66 |
| 4 | OG1 | THR | 28 | −21.760 | 20.076 | 59.636 | 1.00 | 33.79 |
| 5 | CG2 | THR | 28 | −21.645 | 17.737 | 60.183 | 1.00 | 27.40 |
| 6 | C | THR | 28 | −24.238 | 17.990 | 59.627 | 1.00 | 38.85 |
| 7 | O | THR | 28 | −24.732 | 16.923 | 60.023 | 1.00 | 42.97 |
| 8 | N | TRP | 32 | −24.091 | 20.068 | 53.681 | 1.00 | 30.06 |
| 9 | CA | TRP | 32 | −23.725 | 21.413 | 54.092 | 1.00 | 28.93 |
| 10 | CB | TRP | 32 | −24.277 | 21.708 | 55.486 | 1.00 | 29.27 |
| 11 | CG | TRP | 32 | −24.036 | 23.126 | 55.939 | 1.00 | 31.13 |
| 12 | CD2 | TRP | 32 | −22.895 | 23.622 | 56.644 | 1.00 | 32.44 |
| 13 | CE2 | TRP | 32 | −23.118 | 25.005 | 56.890 | 1.00 | 35.25 |
| 14 | CE3 | TRP | 32 | −21.707 | 23.038 | 57.096 | 1.00 | 32.45 |
| 15 | CD1 | TRP | 32 | −24.880 | 24.197 | 55.779 | 1.00 | 33.86 |
| 16 | NE1 | TRP | 32 | −24.333 | 25.331 | 56.351 | 1.00 | 35.49 |
| 17 | CZ2 | TRP | 32 | −22.200 | 25.800 | 57.565 | 1.00 | 35.24 |
| 18 | CZ3 | TRP | 32 | −20.793 | 23.832 | 57.765 | 1.00 | 34.43 |
| 19 | CH2 | TRP | 32 | −21.046 | 25.197 | 57.994 | 1.00 | 36.72 |
| 20 | C | TRP | 32 | −22.203 | 21.582 | 54.091 | 1.00 | 27.24 |
| 21 | O | TRP | 32 | −21.675 | 22.617 | 53.674 | 1.00 | 26.75 |
| 22 | N | ILE | 33 | −21.503 | 20.566 | 54.581 | 1.00 | 26.32 |
| 23 | CA | ILE | 33 | −20.042 | 20.617 | 54.642 | 1.00 | 25.89 |
| 24 | CB | ILE | 33 | −19.459 | 19.370 | 55.333 | 1.00 | 25.18 |
| 25 | CG2 | ILE | 33 | −17.925 | 19.444 | 55.366 | 1.00 | 26.64 |
| 26 | CG1 | ILE | 33 | −20.024 | 19.253 | 56.744 | 1.00 | 18.01 |
| 27 | CD1 | ILE | 33 | −19.621 | 18.008 | 57.421 | 1.00 | 19.10 |
| 28 | C | ILE | 33 | −19.432 | 20.755 | 53.258 | 1.00 | 24.76 |
| 29 | O | ILE | 33 | −18.630 | 21.650 | 53.022 | 1.00 | 23.20 |
| 30 | N | ARG | 36 | −20.198 | 24.159 | 51.621 | 1.00 | 26.35 |
| 31 | CA | ARG | 36 | −19.545 | 25.296 | 52.237 | 1.00 | 27.73 |
| 32 | CB | ARG | 36 | −20.083 | 25.473 | 53.649 | 1.00 | 34.96 |
| 33 | CG | ARG | 36 | −19.562 | 26.715 | 54.326 | 1.00 | 47.48 |
| 34 | CD | ARG | 36 | −20.581 | 27.250 | 55.290 | 1.00 | 56.04 |
| 35 | NE | ARG | 36 | −21.775 | 27.729 | 54.600 | 1.00 | 63.48 |
| 36 | CZ | ARG | 36 | −22.490 | 28.780 | 54.996 | 1.00 | 67.12 |
| 37 | NH1 | ARG | 36 | −23.564 | 29.153 | 54.303 | 1.00 | 67.75 |
| 38 | NH2 | ARG | 36 | −22.127 | 29.465 | 56.082 | 1.00 | 68.27 |
| 39 | C | ARG | 36 | −18.014 | 25.292 | 52.233 | 1.00 | 23.26 |
| 40 | O | ARG | 36 | −17.386 | 26.346 | 52.208 | 1.00 | 21.26 |
| 41 | N | THR | 37 | −17.423 | 24.103 | 52.214 | 1.00 | 20.79 |
| 42 | CA | THR | 37 | −15.973 | 23.969 | 52.258 | 1.00 | 19.72 |
| 43 | CB | THR | 37 | −15.549 | 23.164 | 53.509 | 1.00 | 20.01 |
| 44 | OG1 | THR | 37 | −16.014 | 21.812 | 53.384 | 1.00 | 17.59 |
| 45 | CG2 | THR | 37 | −16.157 | 23.752 | 54.765 | 1.00 | 18.21 |
| 46 | C | THR | 37 | −15.363 | 23.272 | 51.047 | 1.00 | 20.77 |
| 47 | O | THR | 37 | −14.234 | 23.571 | 50.657 | 1.00 | 20.98 |
| 48 | N | CYS | 112 | −0.698 | 28.695 | 58.467 | 1.00 | 12.58 |
| 49 | CA | CYS | 112 | −0.984 | 28.096 | 57.174 | 1.00 | 11.86 |
| 50 | CB | CYS | 112 | −2.457 | 28.264 | 56.808 | 1.00 | 10.86 |
| 51 | SG | CYS | 112 | −3.580 | 27.460 | 57.935 | 1.00 | 22.06 |
| 52 | C | CYS | 112 | −0.126 | 28.620 | 56.037 | 1.00 | 10.86 |
| 53 | O | CYS | 112 | −0.003 | 27.939 | 55.025 | 1.00 | 13.89 |
| 54 | N | LEU | 142 | −3.033 | 20.066 | 62.705 | 1.00 | 16.58 |
| 55 | CA | LEU | 142 | −4.063 | 20.954 | 63.207 | 1.00 | 17.95 |
| 56 | CB | LEU | 142 | −4.281 | 22.159 | 62.287 | 1.00 | 15.72 |
| 57 | CG | LEU | 142 | −3.100 | 23.125 | 62.126 | 1.00 | 18.13 |
| 58 | CD1 | LEU | 142 | −3.628 | 24.499 | 61.738 | 1.00 | 14.84 |
| 59 | CD2 | LEU | 142 | −2.246 | 23.204 | 63.415 | 1.00 | 12.26 |
| 60 | C | LEU | 142 | −5.396 | 20.321 | 63.598 | 1.00 | 17.45 |
| 61 | O | LEU | 142 | −6.111 | 20.883 | 64.417 | 1.00 | 17.68 |
| 62 | N | ARG | 151 | −17.927 | 23.092 | 65.249 | 1.00 | 22.20 |
| 63 | CA | ARG | 151 | −18.230 | 22.887 | 63.841 | 1.00 | 25.49 |
| 64 | CB | ARG | 151 | −19.699 | 23.217 | 63.534 | 1.00 | 24.14 |
| 65 | CG | ARG | 151 | −20.051 | 22.998 | 62.052 | 1.00 | 33.87 |
| 66 | CD | ARG | 151 | −21.530 | 23.158 | 61.748 | 1.00 | 37.44 |
| 67 | NE | ARG | 151 | −21.991 | 24.545 | 61.780 | 1.00 | 41.79 |
| 68 | CZ | ARG | 151 | −23.272 | 24.897 | 61.737 | 1.00 | 44.63 |
| 69 | NH1 | ARG | 151 | −23.612 | 26.173 | 61.771 | 1.00 | 46.51 |
| 70 | NH2 | ARG | 151 | −24.219 | 23.970 | 61.666 | 1.00 | 47.88 |
| 71 | C | ARG | 151 | −17.304 | 23.634 | 62.868 | 1.00 | 26.00 |
| 72 | O | ARG | 151 | −16.686 | 23.018 | 61.992 | 1.00 | 26.64 |
| 73 | N | GLY | 152 | −17.164 | 24.940 | 63.077 | 1.00 | 24.63 |
| 74 | CA | GLY | 152 | −16.353 | 25.769 | 62.201 | 1.00 | 23.08 |
| 75 | C | GLY | 152 | −14.912 | 25.371 | 61.944 | 1.00 | 22.21 |
| 76 | O | GLY | 152 | −14.366 | 25.679 | 60.880 | 1.00 | 21.32 |
| 77 | N | ILE | 155 | −14.484 | 20.649 | 60.878 | 1.00 | 18.82 |
| 78 | CA | ILE | 155 | −14.866 | 20.149 | 59.564 | 1.00 | 18.77 |
| 79 | CB | ILE | 155 | −16.223 | 20.733 | 59.071 | 1.00 | 17.77 |
| 80 | CG2 | ILE | 155 | −17.365 | 20.321 | 60.018 | 1.00 | 12.79 |
| 81 | CG1 | ILE | 155 | −16.127 | 22.249 | 58.924 | 1.00 | 15.46 |
| 82 | CD1 | ILE | 155 | −17.339 | 22.892 | 58.331 | 1.00 | 20.95 |
| 83 | C | ILE | 155 | −13.823 | 20.489 | 58.531 | 1.00 | 18.45 |
| 84 | O | ILE | 155 | −13.819 | 19.909 | 57.461 | 1.00 | 21.51 |
| 85 | N | ILE | 156 | −12.958 | 21.450 | 58.819 | 1.00 | 18.70 |
| 86 | CA | ILE | 156 | −11.985 | 21.825 | 57.812 | 1.00 | 19.10 |
| 87 | CB | ILE | 156 | −11.999 | 23.375 | 57.499 | 1.00 | 24.79 |
| 88 | CG2 | ILE | 156 | −13.391 | 23.974 | 57.563 | 1.00 | 23.59 |
| 89 | CG1 | ILE | 156 | −11.095 | 24.139 | 58.438 | 1.00 | 24.77 |
| 90 | CD1 | ILE | 156 | −9.886 | 24.631 | 57.730 | 1.00 | 27.97 |
| 91 | C | ILE | 156 | −10.544 | 21.338 | 57.935 | 1.00 | 18.32 |
| 92 | O | ILE | 156 | −9.922 | 21.071 | 56.918 | 1.00 | 18.31 |
| 93 | N | PHE | 157 | −10.005 | 21.200 | 59.142 | 1.00 | 16.26 |
| 94 | CA | PHE | 157 | −8.611 | 20.780 | 59.280 | 1.00 | 15.33 |
| 95 | CB | PHE | 157 | −7.984 | 21.371 | 60.551 | 1.00 | 15.71 |
| 96 | CG | PHE | 157 | −7.868 | 22.858 | 60.523 | 1.00 | 19.05 |
| 97 | CD1 | PHE | 157 | −8.814 | 23.654 | 61.158 | 1.00 | 19.74 |
| 98 | CD2 | PHE | 157 | −6.844 | 23.476 | 59.814 | 1.00 | 15.77 |
| 99 | CE1 | PHE | 157 | −8.737 | 25.057 | 61.076 | 1.00 | 21.28 |
| 100 | CE2 | PHE | 157 | −6.761 | 24.855 | 59.727 | 1.00 | 11.63 |
| 101 | CZ | PHE | 157 | −7.701 | 25.650 | 60.351 | 1.00 | 17.65 |
| 102 | C | PHE | 157 | −8.278 | 19.286 | 59.190 | 1.00 | 16.07 |
| 103 | O | PHE | 157 | −9.045 | 18.413 | 59.622 | 1.00 | 17.36 |
| 104 | N | LEU | 189 | −7.786 | 34.391 | 64.172 | 1.00 | 19.01 |
| 105 | CA | LEU | 189 | −7.338 | 33.021 | 63.922 | 1.00 | 19.46 |
| 106 | CB | LEU | 189 | −6.897 | 32.907 | 62.463 | 1.00 | 23.06 |
| 107 | CG | LEU | 189 | −6.422 | 31.587 | 61.872 | 1.00 | 23.21 |
| 108 | CD1 | LEU | 189 | −7.435 | 30.493 | 62.157 | 1.00 | 24.24 |
| 109 | CD2 | LEU | 189 | −6.253 | 31.811 | 60.355 | 1.00 | 25.52 |
| 110 | C | LEU | 189 | −6.164 | 32.746 | 64.850 | 1.00 | 18.26 |
| 111 | O | LEU | 189 | −5.082 | 33.338 | 64.688 | 1.00 | 15.62 |
| 112 | N | LEU | 205 | −7.765 | 25.549 | 68.834 | 1.00 | 19.58 |
| 113 | CA | LEU | 205 | −7.699 | 26.448 | 67.677 | 1.00 | 19.69 |
| 114 | CB | LEU | 205 | −7.475 | 25.601 | 66.398 | 1.00 | 19.66 |
| 115 | CG | LEU | 205 | −7.104 | 26.238 | 65.052 | 1.00 | 19.36 |
| 116 | CD1 | LEU | 205 | −6.309 | 25.259 | 64.201 | 1.00 | 18.01 |
| 117 | CD2 | LEU | 205 | −8.366 | 26.671 | 64.321 | 1.00 | 18.66 |
| 118 | C | LEU | 205 | −8.996 | 27.273 | 67.597 | 1.00 | 17.98 |
| 119 | O | LEU | 205 | −10.088 | 26.731 | 67.804 | 1.00 | 20.78 |
| 120 | N | MET | 207 | −11.189 | 30.405 | 65.330 | 1.00 | 16.50 |
| 121 | CA | MET | 207 | −11.285 | 31.040 | 64.025 | 1.00 | 18.56 |
| 122 | CB | MET | 207 | −11.105 | 30.003 | 62.931 | 1.00 | 20.76 |
| 123 | CG | MET | 207 | −11.293 | 30.550 | 61.542 | 1.00 | 23.66 |
| 124 | SD | MET | 207 | −10.858 | 29.292 | 60.353 | 1.00 | 32.43 |
| 125 | CE | MET | 207 | −12.262 | 28.166 | 60.555 | 1.00 | 31.26 |
| 126 | C | MET | 207 | −12.599 | 31.742 | 63.776 | 1.00 | 18.83 |
| 127 | O | MET | 207 | −13.666 | 31.152 | 63.934 | 1.00 | 19.82 |
| 128 | N | GLY | 209 | −14.190 | 32.425 | 61.134 | 1.00 | 20.42 |
| 129 | CA | GLY | 209 | −14.305 | 32.056 | 59.737 | 1.00 | 23.52 |
| 130 | C | GLY | 209 | −14.623 | 33.114 | 58.701 | 1.00 | 24.44 |
| 131 | O | GLY | 209 | −13.771 | 33.456 | 57.884 | 1.00 | 26.52 |
| 132 | N | ASN | 210 | −15.839 | 33.640 | 58.738 | 1.00 | 23.37 |
| 133 | CA | ASN | 210 | −16.291 | 34.615 | 57.758 | 1.00 | 25.94 |
| 134 | CB | ASN | 210 | −17.724 | 35.006 | 58.035 | 1.00 | 24.49 |
| 135 | CG | ASN | 210 | −18.633 | 33.818 | 58.029 | 1.00 | 25.13 |
| 136 | OD1 | ASN | 210 | −18.680 | 33.068 | 57.061 | 1.00 | 25.86 |
| 137 | ND2 | ASN | 210 | −19.325 | 33.603 | 59.130 | 1.00 | 25.81 |
| 138 | C | ASN | 210 | −15.426 | 35.831 | 57.639 | 1.00 | 26.62 |
| 139 | O | ASN | 210 | −15.214 | 36.334 | 56.545 | 1.00 | 27.59 |
| 140 | N | VAL | 212 | −12.110 | 35.950 | 58.414 | 1.00 | 25.15 |
| 141 | CA | VAL | 212 | −10.808 | 35.645 | 57.793 | 1.00 | 25.13 |
| 142 | CB | VAL | 212 | −10.004 | 34.469 | 58.486 | 1.00 | 23.52 |
| 143 | CG1 | VAL | 212 | −10.492 | 34.190 | 59.896 | 1.00 | 20.23 |
| 144 | CG2 | VAL | 212 | −9.958 | 33.220 | 57.653 | 1.00 | 23.20 |
| 145 | C | VAL | 212 | −10.971 | 35.405 | 56.272 | 1.00 | 23.49 |
| 146 | O | VAL | 212 | −10.095 | 35.769 | 55.493 | 1.00 | 20.62 |
| 147 | N | PHE | 213 | −12.115 | 34.859 | 55.853 | 1.00 | 22.05 |
| 148 | CA | PHE | 213 | −12.371 | 34.627 | 54.431 | 1.00 | 22.19 |
| 149 | CB | PHE | 213 | −13.718 | 33.954 | 54.244 | 1.00 | 20.95 |
| 150 | CG | PHE | 213 | −14.116 | 33.771 | 52.794 | 1.00 | 23.47 |
| 151 | CD1 | PHE | 213 | −14.758 | 34.788 | 52.101 | 1.00 | 22.38 |
| 152 | CD2 | PHE | 213 | −13.833 | 32.587 | 52.132 | 1.00 | 21.51 |
| 153 | CE1 | PHE | 213 | −15.098 | 34.634 | 50.784 | 1.00 | 23.71 |
| 154 | CE2 | PHE | 213 | −14.173 | 32.423 | 50.813 | 1.00 | 26.06 |
| 155 | CZ | PHE | 213 | −14.805 | 33.446 | 50.133 | 1.00 | 25.34 |
| 156 | C | PHE | 213 | −12.307 | 35.935 | 53.645 | 1.00 | 22.07 |
| 157 | O | PHE | 213 | −11.618 | 36.045 | 52.629 | 1.00 | 22.83 |
| 158 | N | ALA | 216 | −8.801 | 37.118 | 53.586 | 1.00 | 18.14 |
| 159 | CA | ALA | 216 | −7.964 | 36.216 | 52.808 | 1.00 | 19.00 |
| 160 | CB | ALA | 216 | −8.183 | 34.775 | 53.218 | 1.00 | 17.94 |
| 161 | C | ALA | 216 | −8.146 | 36.371 | 51.303 | 1.00 | 17.97 |
| 162 | O | ALA | 216 | −7.166 | 36.285 | 50.563 | 1.00 | 16.52 |
| 163 | N | LEU | 220 | −4.879 | 37.537 | 49.135 | 1.00 | 15.55 |
| 164 | CA | LEU | 220 | −4.379 | 36.571 | 48.174 | 1.00 | 17.76 |
| 165 | CB | LEU | 220 | −5.127 | 35.233 | 48.275 | 1.00 | 15.75 |
| 166 | CG | LEU | 220 | −4.703 | 34.362 | 49.466 | 1.00 | 13.65 |
| 167 | CD1 | LEU | 220 | −5.621 | 33.177 | 49.608 | 1.00 | 13.89 |
| 168 | CD2 | LEU | 220 | −3.278 | 33.915 | 49.310 | 1.00 | 9.45 |
| 169 | C | LEU | 220 | −4.491 | 37.186 | 46.769 | 1.00 | 17.28 |
| 170 | O | LEU | 220 | −3.618 | 36.957 | 45.932 | 1.00 | 20.62 |
| 171 | N | HIS | 244 | −3.012 | 27.197 | 48.689 | 1.00 | 18.90 |
| 172 | CA | HIS | 244 | −3.165 | 26.587 | 49.988 | 1.00 | 17.18 |
| 173 | CB | HIS | 244 | −2.914 | 27.594 | 51.111 | 1.00 | 17.15 |
| 174 | CG | HIS | 244 | −3.178 | 27.035 | 52.465 | 1.00 | 17.42 |
| 175 | CD2 | HIS | 244 | −2.579 | 26.015 | 53.138 | 1.00 | 14.15 |
| 176 | ND1 | HIS | 244 | −4.285 | 27.385 | 53.212 | 1.00 | 16.36 |
| 177 | CE1 | HIS | 244 | −4.370 | 26.596 | 54.264 | 1.00 | 16.12 |
| 178 | NE2 | HIS | 244 | −3.354 | 25.760 | 54.244 | 1.00 | 19.14 |
| 179 | C | HIS | 244 | −4.631 | 26.151 | 49.971 | 1.00 | 15.41 |
| 180 | O | HIS | 244 | −5.503 | 26.936 | 49.591 | 1.00 | 14.10 |
| 181 | N | ALA | 246 | −7.440 | 26.051 | 51.721 | 1.00 | 19.76 |
| 182 | CA | ALA | 246 | −8.240 | 26.512 | 52.864 | 1.00 | 20.02 |
| 183 | CB | ALA | 246 | −8.166 | 28.017 | 52.975 | 1.00 | 22.28 |
| 184 | C | ALA | 246 | −9.687 | 26.075 | 52.772 | 1.00 | 23.08 |
| 185 | O | ALA | 246 | −10.281 | 25.620 | 53.759 | 1.00 | 23.37 |
| 186 | N | ASN | 247 | −10.280 | 26.349 | 51.615 | 1.00 | 21.20 |
| 187 | CA | ASN | 247 | −11.645 | 25.983 | 51.311 | 1.00 | 22.48 |
| 188 | CB | ASN | 247 | −12.653 | 26.733 | 52.190 | 1.00 | 24.84 |
| 189 | CG | ASN | 247 | −12.700 | 28.195 | 51.888 | 1.00 | 26.54 |
| 190 | OD1 | ASN | 247 | −13.343 | 28.618 | 50.942 | 1.00 | 32.63 |
| 191 | ND2 | ASN | 247 | −12.016 | 28.987 | 52.686 | 1.00 | 31.62 |
| 192 | C | ASN | 247 | −11.824 | 26.292 | 49.825 | 1.00 | 23.43 |
| 193 | O | ASN | 247 | −11.076 | 27.097 | 49.249 | 1.00 | 23.61 |
| 194 | N | ARG | 249 | −14.126 | 27.939 | 48.119 | 1.00 | 27.79 |
| 195 | CA | ARG | 249 | −14.566 | 29.305 | 47.811 | 1.00 | 28.98 |
| 196 | CB | ARG | 249 | −15.376 | 29.912 | 48.966 | 1.00 | 34.43 |
| 197 | CG | ARG | 249 | −16.577 | 29.118 | 49.433 | 1.00 | 45.16 |
| 198 | CD | ARG | 249 | −17.307 | 29.859 | 50.557 | 1.00 | 52.72 |
| 199 | NE | ARG | 249 | −18.235 | 30.862 | 50.037 | 1.00 | 60.09 |
| 200 | CZ | ARG | 249 | −18.607 | 31.976 | 50.675 | 1.00 | 62.73 |
| 201 | NH1 | ARG | 249 | −19.469 | 32.803 | 50.096 | 1.00 | 64.82 |
| 202 | NH2 | ARG | 249 | −18.112 | 32.290 | 51.867 | 1.00 | 60.24 |
| 203 | C | ARG | 249 | −13.369 | 30.208 | 47.562 | 1.00 | 24.80 |
| 204 | O | ARG | 249 | −13.358 | 31.007 | 46.629 | 1.00 | 24.09 |
| 205 | N | ILE | 250 | −12.393 | 30.135 | 48.453 | 1.00 | 24.48 |
| 206 | CA | ILE | 250 | −11.201 | 30.951 | 48.306 | 1.00 | 24.93 |
| 207 | CB | ILE | 250 | −10.365 | 30.965 | 49.621 | 1.00 | 26.91 |
| 208 | CG2 | ILE | 250 | −8.880 | 31.128 | 49.350 | 1.00 | 22.69 |
| 209 | CG1 | ILE | 250 | −10.902 | 32.091 | 50.506 | 1.00 | 32.57 |
| 210 | CD1 | ILE | 250 | −10.216 | 32.245 | 51.828 | 1.00 | 38.42 |
| 211 | C | ILE | 250 | −10.391 | 30.533 | 47.076 | 1.00 | 23.12 |
| 212 | O | ILE | 250 | −10.024 | 31.380 | 46.265 | 1.00 | 20.24 |
| 213 | N | ASN | 274 | −7.884 | 20.993 | 55.104 | 1.00 | 16.04 |
| 214 | CA | ASN | 274 | −6.887 | 22.042 | 55.213 | 1.00 | 16.17 |
| 215 | CB | ASN | 274 | −7.524 | 23.307 | 55.790 | 1.00 | 16.71 |
| 216 | CG | ASN | 274 | −6.524 | 24.433 | 56.031 | 1.00 | 15.26 |
| 217 | OD1 | ASN | 274 | −5.290 | 24.259 | 55.970 | 1.00 | 14.69 |
| 218 | ND2 | ASN | 274 | −7.058 | 25.607 | 56.319 | 1.00 | 17.12 |
| 219 | C | ASN | 274 | −5.800 | 21.538 | 56.144 | 1.00 | 18.93 |
| 220 | O | ASN | 274 | −6.016 | 21.456 | 57.366 | 1.00 | 18.02 |
| 221 | N | SER | 276 | −2.883 | 23.010 | 56.745 | 1.00 | 14.34 |
| 222 | CA | SER | 276 | −1.996 | 24.086 | 57.152 | 1.00 | 14.51 |
| 223 | CB | SER | 276 | −1.772 | 23.993 | 58.686 | 1.00 | 16.80 |
| 224 | OG | SER | 276 | −1.051 | 25.104 | 59.218 | 1.00 | 17.07 |
| 225 | C | SER | 276 | −0.675 | 24.141 | 56.352 | 1.00 | 13.90 |
| 226 | O | SER | 276 | −0.719 | 24.199 | 55.132 | 1.00 | 15.64 |
| 227 | N | ALA | 303 | −0.360 | 31.072 | 49.683 | 1.00 | 15.48 |
| 228 | CA | ALA | 303 | −0.934 | 30.617 | 50.937 | 1.00 | 13.31 |
| 229 | CB | ALA | 303 | 0.045 | 29.692 | 51.624 | 1.00 | 11.10 |
| 230 | C | ALA | 303 | −1.261 | 31.801 | 51.853 | 1.00 | 12.59 |
| 231 | O | ALA | 303 | −0.614 | 32.842 | 51.789 | 1.00 | 11.22 |
| 232 | N | PHE | 304 | −2.299 | 31.642 | 52.666 | 1.00 | 14.82 |
| 233 | CA | PHE | 304 | −2.726 | 32.650 | 53.626 | 1.00 | 15.34 |
| 234 | CB | PHE | 304 | −4.075 | 33.248 | 53.207 | 1.00 | 17.57 |
| 235 | CG | PHE | 304 | −4.561 | 34.355 | 54.119 | 1.00 | 23.99 |
| 236 | CD1 | PHE | 304 | −5.356 | 34.060 | 55.243 | 1.00 | 22.77 |
| 237 | CD2 | PHE | 304 | −4.220 | 35.687 | 53.866 | 1.00 | 22.34 |
| 238 | CE1 | PHE | 304 | −5.794 | 35.064 | 56.089 | 1.00 | 19.22 |
| 239 | CE2 | PHE | 304 | −4.657 | 36.705 | 54.712 | 1.00 | 24.60 |
| 240 | CZ | PHE | 304 | −5.447 | 36.389 | 55.826 | 1.00 | 19.92 |
| 241 | C | PHE | 304 | −2.831 | 31.946 | 54.982 | 1.00 | 15.89 |
| 242 | O | PHE | 304 | −3.176 | 30.768 | 55.041 | 1.00 | 14.69 |
| 243 | N | GLY | 305 | −2.490 | 32.637 | 56.065 | 1.00 | 14.20 |
| 244 | CA | GLY | 305 | −2.583 | 32.002 | 57.363 | 1.00 | 13.74 |
| 245 | C | GLY | 305 | −2.765 | 32.889 | 58.578 | 1.00 | 13.32 |
| 246 | O | GLY | 305 | −2.788 | 34.115 | 58.496 | 1.00 | 12.57 |
| 247 | N | GLY | 306 | −2.856 | 32.235 | 59.727 | 1.00 | 17.80 |
| 248 | CA | GLY | 306 | −3.033 | 32.929 | 60.990 | 1.00 | 17.87 |
| 249 | C | GLY | 306 | −1.928 | 33.892 | 61.357 | 1.00 | 19.45 |
| 250 | O | GLY | 306 | −0.758 | 33.751 | 60.965 | 1.00 | 19.00 |
| 251 | N | PHE | 487 | 0.118 | 30.521 | 66.721 | 1.00 | 16.05 |
| 252 | CA | PHE | 487 | −0.254 | 31.597 | 65.800 | 1.00 | 15.49 |
| 253 | CB | PHE | 487 | −0.539 | 31.168 | 64.330 | 1.00 | 10.60 |
| 254 | CG | PHE | 487 | −1.559 | 30.100 | 64.167 | 1.00 | 9.77 |
| 255 | CD1 | PHE | 487 | −1.169 | 28.788 | 63.944 | 1.00 | 11.44 |
| 256 | CD2 | PHE | 487 | −2.916 | 30.410 | 64.157 | 1.00 | 12.27 |
| 257 | CE1 | PHE | 487 | −2.109 | 27.796 | 63.715 | 1.00 | 10.46 |
| 258 | CE2 | PHE | 487 | −3.878 | 29.416 | 63.925 | 1.00 | 11.90 |
| 259 | CZ | PHE | 487 | −3.477 | 28.111 | 63.705 | 1.00 | 9.96 |
| 260 | C | PHE | 487 | −1.381 | 32.376 | 66.460 | 1.00 | 13.45 |
| 261 | O | PHE | 487 | −2.233 | 31.776 | 67.132 | 1.00 | 15.58 |

Table II provides the distances between (D) atoms of the active site residues that are within 5.0 angstroms of one another as defined by Table 1.

TABLE II

| | | Distance | | | |
|---|---|---|---|---|---|
| | | Between | | | Between |
| Atom 1 | Atom 2 | (D=) | Atom 1 | Atom 2 | (D=) |
| 28CA | 151CD | 4.796 | 32N | 33CA | 4.198 |
| 28CB | 33CD1 | 4.204 | 32N | 33C | 4.728 |
| 28CB | 151CD | 4.292 | 32N | 33CB | 4.967 |
| 28CB | 33CG1 | 4.398 | 32CA | 33N | 2.428 |
| 28CB | 151CG | 4.703 | 32CA | 33CA | 3.808 |
| 28OG1 | 33CG1 | 3.472 | 32CA | 33C | 4.422 |
| 28OG1 | 33CD1 | 3.709 | 32CA | 33CB | 4.890 |
| 28OG1 | 151CD | 3.743 | 32CB | 33N | 3.133 |
| 28OG1 | 32CE3 | 3.902 | 32CB | 33CA | 4.454 |
| 28OG1 | 151CG | 4.159 | 32CG | 33N | 3.849 |
| 28OG1 | 32CZ3 | 4.306 | 32CG | 33CA | 4.892 |
| 28OG1 | 155CG2 | 4.418 | 32CD2 | 33N | 3.941 |
| 28OG1 | 32CD2 | 4.776 | 32CD2 | 36CB | 4.506 |
| 28OG1 | 33CB | 4.930 | 32CD2 | 36CD | 4.511 |
| 28OG1 | 151NE | 4.962 | 32CD2 | 33CA | 4.602 |
| 28CG2 | 33CD1 | 3.435 | 32CD2 | 36NE | 4.722 |
| 28CG2 | 33CG1 | 4.093 | 32CE2 | 36CD | 3.747 |
| 32N | 33N | 2.785 | 32CE2 | 36NE | 3.804 |
| 32CE2 | 36CZ | 4.270 | 32CZ3 | 33CG1 | 4.754 |
| 32CE2 | 36CB | 4.465 | 32CZ3 | 151CZ | 4.802 |
| 32CE2 | 36NH2 | 4.640 | 32CH2 | 36CD | 3.427 |
| 32CE2 | 36CG | 4.706 | 32CH2 | 151NE | 3.956 |
| 32CE2 | 151CZ | 4.851 | 32CH2 | 36CG | 4.238 |
| 32CE2 | 36NH1 | 4.909 | 32CH2 | 36NE | 4.297 |
| 32CE3 | 33N | 3.532 | 32CH2 | 151CD | 4.299 |
| 32CE3 | 33CA | 3.828 | 32CH2 | 151CZ | 4.365 |
| 32CE3 | 33CG1 | 4.157 | 32CH2 | 155CD1 | 4.378 |
| 32CE3 | 36CB | 4.522 | 32CN2 | 36CB | 4.459 |
| 32CE3 | 155CD1 | 4.542 | 32CH2 | 151NH1 | 4.669 |
| 32CE3 | 33CB | 4.649 | 32CH2 | 151CG | 4.722 |
| 32CE3 | 151CD | 4.657 | 32CH2 | 36NH2 | 4.800 |
| 32CE3 | 36CD | 4.719 | 32CH2 | 36CZ | 4.890 |
| 32CE3 | 151NE | 4.929 | 32C | 33CA | 2.430 |
| 32CD1 | 36NE | 4.848 | 32C | 33C | 3.009 |
| 32NE1 | 36NE | 3.919 | 32C | 33O | 3.730 |
| 32NE1 | 36CZ | 4.139 | 32C | 33CB | 3.737 |
| 32NE1 | 36CD | 4.346 | 32C | 36N | 4.094 |
| 32NE1 | 36NH1 | 4.404 | 32C | 33CG1 | 4.149 |
| 32NE1 | 36NH2 | 4.693 | 32C | 36CB | 4.453 |
| 32CZ2 | 36CD | 3.146 | 32C | 36CA | 4.929 |
| 32CZ2 | 36NE | 3.563 | 32C | 33CG2 | 4.950 |
| 32CZ2 | 36CZ | 3.945 | 32O | 33N | 2.249 |
| 32CZ2 | 36NH2 | 3.954 | 32O | 33CA | 2.757 |
| 32CZ2 | 36CG | 4.276 | 32O | 33C | 2.945 |
| 32CZ2 | 151CZ | 4.401 | 32O | 36N | 2.962 |
| 32CZ2 | 151NE | 4.403 | 32O | 33O | 3.261 |
| 32CZ2 | 15NH1 | 4.452 | 32O | 36CB | 3.270 |
| 32CZ2 | 36CB | 4.464 | 32O | 36CA | 3.712 |
| 32CZ2 | 36NH1 | 4.873 | 32O | 33CB | 4.267 |
| 32CZ2 | 151NH2 | 4.924 | 32O | 36CG | 4.657 |
| 32CZ2 | 151CD | 4.993 | 32O | 37N | 4.735 |
| 32CZ3 | 155CD1 | 3.624 | 32O | 36C | 4.758 |
| 32CZ3 | 151CD | 4.106 | 32O | 33CG1 | 4.844 |
| 32CZ3 | 36CD | 4.225 | 33N | 36N | 4.835 |
| 32CZ3 | 151NE | 4.250 | 33CA | 37OG1 | 4.386 |
| 32CZ3 | 151CG | 4.430 | 33CA | 36N | 4.658 |
| 32CZ3 | 36CB | 4.488 | 33CA | 36CB | 4.957 |
| 32CZ3 | 33CA | 4.545 | 33CA | 37N | 4.991 |
| 32CZ3 | 33N | 4.616 | 33CA | 37CG2 | 4.994 |
| 32CZ3 | 36CG | 4.653 | 33CB | 37OG1 | 4.651 |
| 33CG2 | 37OG1 | 3.631 | 36CB | 37CG2 | 4.430 |
| 33CG2 | 155CB | 4.276 | 36CB | 37CA | 4.592 |
| 33CG2 | 155CD1 | 4.585 | 36CG | 37N | 3.982 |
| 33CG2 | 155O | 4.633 | 36CG | 37CG2 | 4.535 |
| 33CG2 | 37CG2 | 4.695 | 36CG | 37CA | 4.970 |
| 33CG2 | 155CG2 | 4.767 | 36C | 37CA | 2.432 |
| 33CG2 | 37CB | 4.789 | 36C | 37CG2 | 3.497 |
| 33CG2 | 155CG1 | 4.874 | 36C | 37CB | 3.498 |
| 33CG1 | 155CG2 | 4.351 | 36C | 37C | 3.538 |
| 33CG1 | 155CB | 4.696 | 36C | 37OG1 | 4.176 |
| 33CG1 | 155CD1 | 4.793 | 36C | 37O | 4.442 |
| 33CD1 | 155CG2 | 4.145 | 36C | 249CD | 4.916 |
| 33CD1 | 155CB | 4.658 | 36C | 249CG | 4.954 |
| 33C | 37OG1 | 3.580 | 36O | 37N | 2.243 |
| 33C | 36N | 3.854 | 36O | 37CA | 2.766 |
| 33C | 37N | 4.042 | 36O | 37CG2 | 3.844 |
| 33C | 37CB | 4.576 | 36O | 37C | 3.859 |
| 33C | 36CA | 4.656 | 36O | 249CD | 3.882 |
| 33C | 37CG2 | 4.688 | 36O | 37CB | 3.898 |
| 33C | 36CB | 4.779 | 36O | 249CG | 4.005 |
| 33C | 37CA | 4.826 | 36O | 37O | 4.477 |
| 33C | 36C | 4.863 | 36O | 247CB | 4.749 |
| 33O | 37OG1 | 2.646 | 36O | 247OD1 | 4.807 |
| 33O | 37N | 2.851 | 36O | 37OG1 | 4.881 |
| 33O | 36N | 3.274 | 37CA | 247CB | 4.321 |
| 33O | 37CB | 3.467 | 37CA | 247CA | 4.867 |
| 33O | 37CA | 3.608 | 37CB | 156CG2 | 4.663 |
| 33O | 37CG2 | 3.684 | 37CB | 247CB | 4.782 |
| 33O | 36C | 3.777 | 37CG2 | 155CD1 | 3.854 |
| 33O | 36CA | 3.840 | 37CG2 | 156CG2 | 3.941 |
| 33O | 36CB | 4.138 | 37CG2 | 155CG1 | 4.422 |
| 33O | 37C | 4.148 | 37CG2 | 156CB | 4.991 |
| 33O | 36O | 4.926 | 37C | 247CB | 4.542 |
| 36N | 37N | 2.838 | 37C | 247CA | 4.609 |
| 36N | 37CA | 4.277 | 37C | 247C | 4.810 |
| 36N | 37C | 4.949 | 37O | 247CA | 3.598 |
| 36CA | 37N | 2.434 | 37O | 247C | 3.729 |
| 36CA | 37CA | 3.811 | 37O | 247CB | 3.853 |
| 36CA | 37CG2 | 4.500 | 37O | 247N | 4.926 |
| 36CA | 37CB | 4.704 | 37O | 247O | 4.938 |
| 36CA | 37C | 4.796 | 112N | 276OG | 3.686 |
| 36CB | 37N | 3.318 | 112N | 305CA | 3.963 |
| 112N | 306N | 4.333 | 112SG | 304O | 4.414 |
| 112N | 305C | 4.677 | 112SG | 157CE2 | 4.485 |
| 112N | 304O | 4.709 | 112SG | 274CG | 4.632 |
| 112N | 276CB | 4.828 | 112SG | 276N | 4.659 |
| 112N | 305N | 4.952 | 112SG | 305CA | 4.685 |
| 112N | 276CA | 4.966 | 112SG | 276C | 4.686 |
| 112CA | 276OG | 3.624 | 112SG | 244ND1 | 4.776 |
| 112CA | 276C | 4.051 | 112SG | 142CD1 | 4.820 |
| 112CA | 304O | 4.061 | 112C | 304O | 3.861 |
| 112CA | 276CA | 4.136 | 112C | 305CA | 4.386 |
| 112CA | 305CA | 4.225 | 112C | 304C | 4.415 |
| 112CA | 276O | 4.408 | 112C | 276C | 4.524 |
| 112CA | 244NE2 | 4.434 | 112C | 303CB | 4.545 |
| 112CA | 276CB | 4.443 | 112C | 276O | 4.551 |
| 112CA | 244CE1 | 4.710 | 112C | 244CD2 | 4.605 |
| 112CA | 304C | 4.800 | 112C | 305N | 4.661 |
| 112CA | 244CD2 | 4.813 | 112C | 244NE2 | 4.671 |
| 112CA | 305N | 4.911 | 112C | 276OG | 4.831 |
| 112CB | 304O | 3.148 | 112C | 244CG | 4.958 |
| 112CB | 244CE1 | 3.594 | 112O | 244CD2 | 3.728 |
| 112CB | 244NE2 | 3.694 | 112O | 276O | 3.809 |
| 112CB | 305CA | 3.781 | 112O | 303CB | 3.827 |
| 112CB | 304C | 4.127 | 112O | 244NE2 | 4.073 |
| 112CB | 244ND1 | 4.129 | 112O | 276C | 4.079 |
| 112CB | 276OG | 4.216 | 112O | 244CG | 4.177 |
| 112CB | 276CA | 4.217 | 112O | 304O | 4.251 |
| 112CB | 244CD2 | 4.306 | 112O | 244CE1 | 4.632 |
| 112CB | 305N | 4.436 | 112O | 244ND1 | 4.683 |
| 112CB | 276C | 4.515 | 112O | 276CA | 4.831 |
| 112CB | 244CG | 4.571 | 112O | 244CB | 4.890 |
| 112CB | 276CB | 4.716 | 112O | 304C | 4.905 |
| 112CB | 276O | 4.728 | 112O | 304N | 4.955 |
| 112CB | 306N | 4.945 | 112O | 303CA | 4.975 |
| 112CB | 305C | 4.962 | 142CA | 157CB | 4.754 |
| 112CB | 274OD1 | 4.977 | 142CA | 205CD1 | 4.956 |
| 112SG | 276OG | 3.687 | 142CB | 157CD2 | 3.797 |
| 112SG | 276CA | 3.809 | 142CB | 157CG | 4.058 |
| 112SG | 244CE1 | 3.853 | 142CB | 157CB | 4.165 |
| 112SG | 276CB | 3.982 | 142CB | 205CD1 | 4.170 |
| 112SG | 244NE2 | 4.070 | 142CB | 157CE2 | 4.469 |
| 112SG | 274OD1 | 4.127 | 142CB | 276CB | 4.757 |
| 112SG | 274ND2 | 4.259 | 142CB | 157CD1 | 4.905 |
| 142CG | 276CB | 3.788 | 151C | 155N | 4.563 |
| 142CG | 276OG | 4.071 | 151C | 155CD1 | 4.597 |
| 142CG | 205CD1 | 4.377 | 151C | 155CB | 4.899 |
| 142CG | 157CD2 | 4.414 | 151O | 152N | 2.258 |
| 142CG | 157CE2 | 4.706 | 151O | 152CA | 2.779 |
| 142CD1 | 276CB | 3.608 | 151O | 152C | 2.947 |
| 142CD1 | 276OG | 3.655 | 151O | 155CG1 | 3.212 |
| 142CD1 | 205CD1 | 3.719 | 151O | 155CG2 | 3.411 |
| 142CD1 | 157CE2 | 3.740 | 151O | 155N | 3.421 |
| 142CD1 | 157CD2 | 3.885 | 151O | 152O | 3.701 |
| 142CD1 | 487CZ | 4.116 | 151O | 155CD1 | 3.721 |
| 142CD1 | 487CE1 | 4.134 | 151O | 155CB | 3.737 |
| 142CD1 | 157CZ | 4.454 | 151O | 155CA | 4.176 |
| 142CD1 | 157CG | 4.706 | 152CA | 155CG1 | 4.815 |
| 142CD1 | 276CA | 4.885 | 152CA | 155CD1 | 4.922 |
| 142CD2 | 487CE1 | 4.604 | 152C | 207CE | 4.094 |
| 142CD2 | 205CD1 | 4.620 | 152C | 155CG1 | 4.510 |
| 142CD2 | 276OG | 4.759 | 152C | 156CG2 | 4.843 |
| 142CD2 | 276CB | 4.818 | 152C | 155N | 4.860 |
| 142C | 157CB | 4.133 | 152O | 207CE | 3.274 |
| 142C | 157CG | 4.691 | 152O | 156CG2 | 3.855 |
| 142O | 157CB | 4.323 | 152O | 155CG1 | 4.323 |
| 142O | 205CD1 | 4.386 | 152O | 156CG1 | 4.363 |
| 142O | 157CG | 4.706 | 152O | 156CB | 4.727 |
| 151N | 152N | 2.952 | 152O | 155CD1 | 4.807 |
| 151N | 152CA | 4.351 | 152O | 156N | 4.911 |
| 151CA | 152N | 2.436 | 155N | 156N | 2.685 |
| 151CA | 152CA | 3.810 | 155N | 156CA | 4.127 |
| 151CA | 152C | 4.558 | 155N | 156CG2 | 4.821 |
| 151CA | 155CG2 | 4.685 | 155N | 157N | 4.835 |
| 151CB | 152N | 3.099 | 155N | 156C | 4.966 |
| 151CB | 152CA | 4.414 | 155CA | 156N | 2.427 |
| 151CG | 152N | 3.627 | 155CA | 156CA | 3.765 |
| 151CG | 155CG2 | 4.303 | 155CA | 156CG2 | 4.562 |
| 151CG | 155CD1 | 4.606 | 155CA | 156C | 4.769 |
| 151CG | 152CA | 4.623 | 155CA | 156CB | 4.784 |
| 151CD | 152N | 4.899 | 155CA | 157N | 4.991 |
| 151C | 152CA | 2.431 | 155CB | 156N | 3.352 |
| 151C | 152C | 3.097 | 155CB | 156CA | 4.554 |
| 151C | 152O | 4.095 | 155CB | 156CG2 | 4.561 |
| 151C | 155CG1 | 4.343 | 155CG2 | 156N | 4.705 |
| 151C | 155CG2 | 4.371 | 155CG1 | 156N | 3.270 |
| 155CG1 | 156CG2 | 3.509 | 156CG1 | 157CE2 | 4.578 |
| 155CG1 | 156CA | 4.310 | 156CG1 | 157CB | 4.670 |
| 155CG1 | 156CB | 4.510 | 156CG1 | 207CE | 4.697 |
| 155CD1 | 156CG2 | 4.165 | 156CG1 | 274ND2 | 4.790 |
| 155CD1 | 156N | 4.638 | 156CG1 | 246O | 4.975 |
| 155C | 156CA | 2.383 | 156CD1 | 274ND2 | 3.308 |
| 155C | 156C | 3.439 | 156CD1 | 274CB | 3.331 |
| 155C | 156CB | 3.567 | 156CD1 | 157CZ | 3.561 |
| 155C | 156CG2 | 3.643 | 156CD1 | 157CE1 | 3.563 |
| 155C | 157N | 3.931 | 156CD1 | 157N | 3.712 |
| 155C | 156O | 4.261 | 156CD1 | 157CE2 | 3.715 |
| 155C | 156CG1 | 4.558 | 156CD1 | 157CD1 | 3.722 |
| 155O | 156N | 2.227 | 156CD1 | 274CG | 3.772 |
| 155O | 156CA | 2.675 | 156CD1 | 157CD2 | 3.864 |
| 155O | 156C | 3.604 | 156CD1 | 157CG | 3.875 |
| 155O | 156CB | 3.915 | 156CD1 | 246O | 4.111 |
| 155O | 156CG2 | 4.089 | 156CD1 | 157CA | 4.343 |
| 155O | 156O | 4.103 | 156CD1 | 274CA | 4.694 |
| 155O | 157N | 4.363 | 156CD1 | 157CB | 4.712 |
| 156N | 157N | 2.981 | 156CD1 | 274N | 4.913 |
| 156N | 157CA | 4.422 | 156CD1 | 274OD1 | 4.936 |
| 156CA | 157N | 2.466 | 156C | 157N | 1.329 |
| 156CA | 157CA | 3.825 | 156C | 157CA | 2.420 |
| 156CA | 157C | 4.700 | 156C | 157C | 3.305 |
| 156CA | 157O | 4.854 | 156C | 157CB | 3.660 |
| 156CA | 157CB | 4.870 | 156C | 157O | 3.694 |
| 156CA | 157CD1 | 4.959 | 156C | 274N | 3.900 |
| 156CA | 274N | 4.980 | 156C | 157CG | 4.021 |
| 156CB | 157N | 3.377 | 156C | 274CB | 4.195 |
| 156CB | 157CA | 4.624 | 156C | 157CD1 | 4.329 |
| 156CB | 246O | 4.688 | 156C | 274O | 4.565 |
| 156CB | 274CB | 4.791 | 156C | 274CA | 4.613 |
| 156CB | 157CD1 | 4.859 | 156C | 157CD2 | 4.668 |
| 156CG2 | 157N | 4.653 | 156O | 157N | 2.229 |
| 156CG1 | 157N | 3.213 | 156O | 157CA | 2.717 |
| 156CG1 | 157CD1 | 3.583 | 156O | 274N | 2.729 |
| 156CG1 | 157CE1 | 3.655 | 156O | 157C | 3.324 |
| 156CG1 | 157CG | 4.050 | 156O | 274CB | 3.467 |
| 156CG1 | 157CZ | 4.179 | 156O | 274CA | 3.614 |
| 156CG1 | 157CA | 4.262 | 156O | 157O | 3.892 |
| 156CG1 | 157CD2 | 4.517 | 156O | 274O | 3.950 |
| 156CG1 | 274CB | 4.523 | 156O | 157CB | 4.129 |
| 156O | 274C | 4.220 | 157CZ | 205CD1 | 4.113 |
| 156O | 157CG | 4.518 | 157CZ | 205CD2 | 4.153 |
| 156O | 274CG | 4.862 | 157CZ | 274CG | 4.640 |
| 156O | 157CD2 | 4.863 | 157CZ | 205CG | 4.775 |
| 157N | 274O | 4.374 | 157CZ | 207SD | 4.820 |
| 157N | 274N | 4.566 | 157C | 274O | 3.627 |
| 157N | 274CB | 4.672 | 157C | 274N | 4.446 |
| 157CA | 274O | 3.295 | 157C | 274C | 4.527 |
| 157CA | 274N | 4.244 | 157O | 274O | 4.850 |
| 157CA | 274C | 4.279 | 189N | 207CA | 4.847 |
| 157CA | 274CB | 4.444 | 189CA | 207CA | 4.417 |
| 157CA | 274CA | 4.594 | 189CA | 207N | 4.864 |
| 157CB | 274O | 3.745 | 189CA | 207CB | 4.928 |
| 157CB | 274C | 4.921 | 189CA | 487CE2 | 4.997 |
| 157CG | 274O | 3.919 | 189CB | 306CA | 4.135 |
| 157CG | 205CD1 | 4.661 | 189CB | 212CG1 | 4.600 |
| 157CG | 274CB | 4.767 | 189CB | 487CE2 | 4.841 |
| 157CG | 274CG | 4.946 | 189CB | 306N | 4.926 |
| 157CD1 | 205CD1 | 4.256 | 189CB | 487CD2 | 4.995 |
| 157CD1 | 205CD2 | 4.394 | 189CG | 306CA | 3.750 |
| 157CD1 | 205CG | 4.976 | 189CG | 487CE2 | 3.924 |
| 157CD2 | 274O | 3.280 | 189CG | 306N | 4.212 |
| 157CD2 | 274CG | 3.915 | 189CG | 487CD2 | 4.347 |
| 157CD2 | 274CB | 4.085 | 189CG | 487CZ | 4.911 |
| 157CD2 | 274ND2 | 4.099 | 189CG | 207CG | 4.991 |
| 157CD2 | 274OD1 | 4.220 | 189CD1 | 207CB | 3.783 |
| 157CD2 | 274C | 4.280 | 189CD1 | 207CG | 3.907 |
| 157CD2 | 205CD1 | 4.766 | 189CD1 | 207SD | 4.051 |
| 157CD2 | 274CA | 4.819 | 189CD1 | 487CE2 | 4.116 |
| 157CE1 | 205CD2 | 3.643 | 189CD1 | 207CA | 4.314 |
| 157CE1 | 205CD1 | 3.963 | 189CD1 | 205CD2 | 4.490 |
| 157CE1 | 205CG | 4.458 | 189CD1 | 487CZ | 4.872 |
| 157CE1 | 207CE | 4.729 | 189CD1 | 207N | 4.916 |
| 157CE1 | 207SD | 4.791 | 189CD1 | 487CD2 | 4.942 |
| 157CE2 | 274ND2 | 3.503 | 189CD2 | 306CA | 3.467 |
| 157CE2 | 274CG | 3.728 | 189CD2 | 306N | 3.480 |
| 157CE2 | 274OD1 | 4.078 | 189CD2 | 305C | 4.060 |
| 157CE2 | 274O | 4.205 | 189CD2 | 305O | 4.557 |
| 157CE2 | 274CB | 4.299 | 189CD2 | 305CA | 4.739 |
| 157CE2 | 205CD1 | 4.515 | 189CD2 | 212CG2 | 4.797 |
| 157CE2 | 274C | 4.976 | 189CD2 | 212CG1 | 4.883 |
| 157CZ | 274ND2 | 4.083 | 189CD2 | 306C | 4.903 |
| 189CD2 | 487CE2 | 4.911 | 209N | 212N | 4.914 |
| 189CD2 | 212CB | 4.963 | 209CA | 210N | 2.421 |
| 189C | 487CD2 | 4.060 | 209CA | 210CA | 3.796 |
| 189C | 487CE2 | 4.144 | 209CA | 212CG1 | 4.372 |
| 189C | 487O | 4.648 | 209CA | 210C | 4.462 |
| 189C | 306CA | 4.974 | 209CA | 212N | 4.662 |
| 189O | 487CD2 | 3.681 | 209CA | 210CB | 4.826 |
| 189O | 487O | 4.066 | 209CA | 212CG2 | 4.959 |
| 189O | 487CE2 | 4.173 | 209CA | 210CG | 4.975 |
| 189O | 487C | 4.215 | 209C | 210CA | 2.434 |
| 189O | 306CA | 4.247 | 209C | 210C | 3.026 |
| 189O | 306C | 4.621 | 209C | 210CB | 3.693 |
| 189O | 487CG | 4.813 | 209C | 212N | 3.800 |
| 205CG | 487CZ | 4.299 | 209C | 210O | 3.920 |
| 205CG | 487CE2 | 4.667 | 209C | 210CG | 4.126 |
| 205CD1 | 487CZ | 4.050 | 209C | 213N | 4.177 |
| 205CD1 | 487CE2 | 4.824 | 209C | 210OD1 | 4.376 |
| 205CD1 | 487CE1 | 4.931 | 209C | 212CG1 | 4.433 |
| 205CD2 | 207CB | 4.532 | 209C | 213CB | 4.625 |
| 205CD2 | 207N | 4.789 | 209C | 212CA | 4.667 |
| 205C | 207N | 4.445 | 209C | 210ND2 | 4.747 |
| 205O | 207N | 4.564 | 209C | 212CG2 | 4.782 |
| 207CA | 209N | 4.326 | 209C | 212CB | 4.818 |
| 207CB | 209N | 4.314 | 209C | 212C | 4.948 |
| 207CB | 209CA | 4.966 | 209O | 210N | 2.245 |
| 207CG | 209N | 3.475 | 209O | 210CA | 2.777 |
| 207CG | 209CA | 3.821 | 209O | 210C | 2.905 |
| 207CG | 212CG1 | 4.074 | 209O | 213N | 2.972 |
| 207CG | 212CG2 | 4.903 | 209O | 212N | 3.043 |
| 207SD | 209CA | 4.461 | 209O | 210O | 3.487 |
| 207SD | 209N | 4.640 | 209O | 213CB | 3.674 |
| 207SD | 212CG2 | 4.851 | 209O | 212CA | 3.685 |
| 207SD | 212CG1 | 4.933 | 209O | 212C | 3.773 |
| 207CE | 209CA | 4.469 | 209O | 212CG2 | 3.827 |
| 207CE | 209N | 4.711 | 209O | 213CA | 3.906 |
| 207C | 209N | 3.159 | 209O | 212CG1 | 3.916 |
| 207C | 209CA | 4.396 | 209O | 212CB | 3.947 |
| 207O | 209N | 3.120 | 209O | 210CB | 4.249 |
| 207O | 209CA | 4.341 | 209O | 210CG | 4.878 |
| 209N | 210N | 3.152 | 209O | 212O | 4.958 |
| 209N | 212CG1 | 4.281 | 209O | 210OD1 | 4.993 |
| 209N | 210CA | 4.540 | 210N | 212N | 4.398 |
| 210N | 213N | 4.866 | 212C | 216CA | 4.658 |
| 210N | 213CB | 4.979 | 212C | 213CG | 4.966 |
| 210CA | 213CB | 4.405 | 212O | 213N | 2.245 |
| 210CA | 212N | 4.438 | 212O | 216N | 2.670 |
| 210CA | 213N | 4.596 | 212O | 213CA | 2.759 |
| 210C | 213N | 3.886 | 212O | 213C | 2.887 |
| 210C | 213CB | 4.239 | 212O | 216CB | 3.134 |
| 210C | 213CA | 4.591 | 212O | 213O | 3.255 |
| 210C | 212CA | 4.624 | 212O | 216CA | 3.457 |
| 210C | 212C | 4.679 | 212O | 213CB | 4.240 |
| 210O | 213N | 3.501 | 212O | 304CE1 | 4.399 |
| 210O | 213CB | 3.633 | 212O | 216C | 4.660 |
| 210O | 212N | 3.644 | 212O | 304CZ | 4.701 |
| 210O | 213CA | 3.933 | 213N | 216N | 4.607 |
| 210O | 213C | 4.126 | 213N | 216CB | 4.734 |
| 210O | 212C | 4.352 | 213CA | 250CD1 | 4.134 |
| 210O | 212CA | 4.631 | 213CA | 216CB | 4.363 |
| 210O | 213CG | 4.674 | 213CA | 216N | 4.434 |
| 210O | 213CD1 | 4.727 | 213CA | 250CG1 | 4.898 |
| 212N | 213N | 2.784 | 213CA | 216CA | 4.958 |
| 212N | 213CA | 4.205 | 213CB | 250CD1 | 4.585 |
| 212N | 213C | 4.773 | 213CG | 250CG1 | 4.288 |
| 212N | 213CB | 4.895 | 213CG | 250CD1 | 4.298 |
| 212CA | 213N | 2.468 | 213CG | 249NH2 | 4.361 |
| 212CA | 213CA | 3.845 | 213CD1 | 249NH2 | 4.189 |
| 212CA | 213C | 4.420 | 213CD1 | 250CG1 | 4.969 |
| 212CA | 216N | 4.888 | 213CD1 | 249CZ | 4.976 |
| 212CA | 213CB | 4.891 | 213CD2 | 250CG1 | 3.388 |
| 212CB | 213N | 3.397 | 213CD2 | 250CD1 | 3.646 |
| 212CB | 213CA | 4.698 | 213CD2 | 247ND2 | 4.070 |
| 212CB | 304CE1 | 4.881 | 213CD2 | 247OD1 | 4.172 |
| 212CG1 | 213N | 4.408 | 213CD2 | 249NH2 | 4.297 |
| 212CG2 | 213N | 3.253 | 213CD2 | 249CB | 4.423 |
| 212CG2 | 213CA | 4.264 | 213CD2 | 247CG | 4.542 |
| 212CG2 | 304CE1 | 4.815 | 213CD2 | 250CB | 4.579 |
| 212C | 213N | 1.335 | 213CD2 | 250N | 4.650 |
| 212C | 213CA | 2.440 | 213CD2 | 249CD | 4.689 |
| 212C | 213C | 2.994 | 213CD2 | 250CA | 4.924 |
| 212C | 213CB | 3.710 | 213CE1 | 249NH2 | 3.969 |
| 212C | 213O | 3.755 | 213CE1 | 249CZ | 4.403 |
| 212C | 216N | 3.855 | 213CE1 | 249NH1 | 4.789 |
| 212C | 216CB | 4.183 | 213CE1 | 250CG1 | 4.914 |
| 213CE1 | 249NE | 4.963 | 213O | 213N | 3.471 |
| 213CE2 | 250CG1 | 3.302 | 213O | 216CA | 3.662 |
| 213CE2 | 249CB | 3.341 | 213O | 216CB | 3.709 |
| 213CE2 | 250N | 3.738 | 213O | 216C | 3.731 |
| 213CE2 | 213CB | 3.785 | 213O | 250CD1 | 4.129 |
| 213CE2 | 247OD1 | 3.897 | 213O | 250CG1 | 4.545 |
| 213CE2 | 249C | 4.015 | 213O | 216O | 4.914 |
| 213CE2 | 249CD | 4.057 | 216N | 304CZ | 4.099 |
| 213CE2 | 249NH2 | 4.080 | 216N | 304CE2 | 4.314 |
| 213CE2 | 250CD1 | 4.089 | 216N | 304CE1 | 4.419 |
| 213CE2 | 250CA | 4.157 | 216N | 304CD2 | 4.807 |
| 213CE2 | 250CB | 4.248 | 216N | 304CD1 | 4.895 |
| 213CE2 | 249CG | 4.314 | 216CA | 304CE2 | 3.847 |
| 213CE2 | 249CA | 4.346 | 216CA | 304CD2 | 3.926 |
| 213CE2 | 249NE | 4.420 | 216CA | 304CZ | 3.934 |
| 213CE2 | 249CZ | 4.459 | 216CA | 304CG | 4.094 |
| 213CE2 | 247ND2 | 4.468 | 216CA | 304CE1 | 4.099 |
| 213CE2 | 249O | 4.492 | 216CA | 304CD1 | 4.169 |
| 213CE2 | 247CG | 4.604 | 216CA | 250CD1 | 4.669 |
| 213CZ | 249CB | 3.765 | 216CA | 304CB | 4.908 |
| 213CZ | 249NH2 | 3.909 | 216CA | 220N | 4.975 |
| 213CZ | 249CZ | 4.112 | 216CA | 220CD1 | 4.997 |
| 213CZ | 250CG1 | 4.148 | 216CB | 250CD1 | 3.531 |
| 213CZ | 249NE | 4.295 | 216CB | 304CD1 | 3.550 |
| 213CZ | 249C | 4.377 | 216CB | 304CE1 | 3.746 |
| 213CZ | 249CD | 4.394 | 216CB | 304CG | 3.756 |
| 213CZ | 250N | 4.428 | 216CB | 304CZ | 4.110 |
| 213CZ | 249O | 4.508 | 216CB | 304CD2 | 4.118 |
| 213CZ | 249NH1 | 4.708 | 216CB | 304CE2 | 4.288 |
| 213CZ | 249CG | 4.729 | 216CB | 304CB | 4.383 |
| 213CZ | 250CA | 4.749 | 216CB | 250CG1 | 4.685 |
| 213CZ | 249CA | 4.754 | 216CB | 220CD1 | 4.706 |
| 213C | 216N | 3.701 | 216C | 220N | 4.091 |
| 213C | 216CB | 4.305 | 216C | 220CG | 4.389 |
| 213C | 216CA | 4.432 | 216C | 220CD1 | 4.410 |
| 213C | 250CD1 | 4.614 | 216C | 220CB | 4.425 |
| 213C | 216C | 4.795 | 216C | 250CD1 | 4.646 |
| 213O | 216N | 3.163 | 216C | 304CD2 | 4.738 |
| 213O | 212O | 3.255 | 216C | 304CE2 | 4.889 |
| 213O | 213CB | 3.375 | 216C | 220CA | 4.901 |
| 213O | 213CG | 3.382 | 216C | 304CG | 4.985 |
| 213O | 213CD1 | 3.423 | 216O | 220N | 2.973 |
| 216O | 220CB | 3.240 | 244CG | 303CB | 4.261 |
| 216O | 220CG | 3.312 | 244CG | 246N | 4.437 |
| 216O | 220CD1 | 3.600 | 244CG | 303CA | 4.495 |
| 216O | 220CA | 3.682 | 244CG | 304O | 4.536 |
| 216O | 304CD2 | 4.466 | 244CG | 276O | 4.605 |
| 216O | 220CD2 | 4.723 | 244CG | 304N | 4.694 |
| 216O | 220C | 4.729 | 244CG | 274OD1 | 4.945 |
| 216O | 304CG | 4.812 | 244CD2 | 276O | 3.276 |
| 216O | 304CE2 | 4.867 | 244CD2 | 276C | 4.179 |
| 220CG | 304CB | 3.954 | 244CD2 | 274OD1 | 4.296 |
| 220CG | 304CD2 | 4.620 | 244CD2 | 276CA | 4.491 |
| 220CG | 304CG | 4.655 | 244CD2 | 276N | 4.705 |
| 220CG | 304N | 4.839 | 244CD2 | 303CB | 4.764 |
| 220CG | 303C | 4.910 | 244ND1 | 246N | 3.736 |
| 220CG | 304CA | 4.914 | 244ND1 | 246CB | 3.939 |
| 220CG | 303O | 4.942 | 244ND1 | 304O | 4.002 |
| 220CD1 | 250CG2 | 3.858 | 244ND1 | 246CA | 4.065 |
| 220CD1 | 304CB | 3.918 | 244ND1 | 274OD1 | 4.288 |
| 220CD1 | 304N | 4.769 | 244ND1 | 274ND2 | 4.528 |
| 220CD1 | 304CG | 4.781 | 244ND1 | 274CG | 4.656 |
| 220CD1 | 304CA | 4.980 | 244ND1 | 304N | 4.729 |
| 220CD2 | 303O | 3.794 | 244CE1 | 274OD1 | 3.036 |
| 220CD2 | 303C | 3.874 | 244CE1 | 274ND2 | 3.525 |
| 220CD2 | 304CB | 4.033 | 244CE1 | 274CG | 3.527 |
| 220CD2 | 303N | 4.091 | 244CE1 | 246N | 4.024 |
| 220CD2 | 304N | 4.170 | 244CE1 | 246CA | 4.116 |
| 220CD2 | 303CA | 4.361 | 244CE1 | 246CB | 4.253 |
| 220CD2 | 304CA | 4.531 | 244CE1 | 304O | 4.409 |
| 220CD2 | 304CD2 | 4.978 | 244CE1 | 276O | 4.453 |
| 220CD2 | 304CG | 4.997 | 244CE1 | 276CA | 4.503 |
| 244N | 303CA | 4.590 | 244CE1 | 276N | 4.607 |
| 244N | 303N | 4.800 | 244CE1 | 274CB | 4.806 |
| 244N | 303CB | 4.918 | 244CE1 | 276C | 4.903 |
| 244CA | 246N | 4.644 | 244NE2 | 274OD1 | 2.997 |
| 244CA | 303CA | 4.703 | 244NE2 | 276O | 3.189 |
| 244CA | 303CB | 4.756 | 244NE2 | 276CA | 3.620 |
| 244CB | 303CA | 3.618 | 244NE2 | 276N | 3.747 |
| 244CB | 303CB | 3.663 | 244NE2 | 276C | 3.774 |
| 244CB | 304N | 4.380 | 244NE2 | 274CG | 3.873 |
| 244CB | 303N | 4.545 | 244NE2 | 274ND2 | 4.248 |
| 244CB | 303C | 4.581 | 244NE2 | 246N | 4.811 |
| 244CB | 246N | 4.821 | 244C | 246N | 3.311 |
| 244C | 246CA | 4.639 | 246C | 274CG | 4.829 |
| 244O | 246N | 3.012 | 246O | 247N | 2.265 |
| 244O | 246CA | 4.288 | 246O | 246CA | 2.400 |
| 244O | 246CB | 4.440 | 246O | 247CA | 2.826 |
| 246N | 247N | 2.858 | 246O | 247CB | 3.054 |
| 246N | 247CA | 4.225 | 246O | 247ND2 | 3.937 |
| 246N | 247O | 4.519 | 246O | 247CG | 3.998 |
| 246N | 274ND2 | 4.635 | 246O | 274ND2 | 4.116 |
| 246N | 274CG | 4.694 | 246O | 274CB | 4.132 |
| 246N | 247C | 4.783 | 246O | 247C | 4.279 |
| 246N | 274CB | 4.908 | 246O | 274CG | 4.548 |
| 246CA | 247N | 2.398 | 246O | 247O | 4.812 |
| 246CA | 274ND2 | 3.762 | 247CA | 249N | 4.491 |
| 246CA | 247CA | 3.780 | 247CB | 249N | 4.494 |
| 246CA | 274CG | 4.159 | 247CG | 250N | 3.957 |
| 246CA | 274CB | 4.398 | 247CG | 249N | 4.038 |
| 246CA | 247CB | 4.470 | 247CG | 250CB | 4.274 |
| 246CA | 247ND2 | 4.518 | 247CG | 249CB | 4.318 |
| 246CA | 247O | 4.632 | 247CG | 250CG1 | 4.508 |
| 246CA | 247C | 4.704 | 247CG | 249CA | 4.619 |
| 246CA | 274OD1 | 4.840 | 247CG | 249CG | 4.681 |
| 246CA | 247CG | 4.866 | 247CG | 250CD1 | 4.751 |
| 246CB | 247N | 3.017 | 247CG | 250CA | 4.762 |
| 246CB | 247ND2 | 3.981 | 247CG | 249C | 4.818 |
| 246CB | 274ND2 | 4.268 | 247OD1 | 249N | 3.007 |
| 246CB | 247CA | 4.360 | 247OD1 | 250N | 3.066 |
| 246CB | 247CG | 4.666 | 247OD1 | 249CB | 3.116 |
| 246CB | 247CB | 4.733 | 247OD1 | 249CA | 3.431 |
| 246CB | 247O | 4.816 | 247OD1 | 249CG | 3.604 |
| 246CB | 250CG2 | 4.830 | 247OD1 | 249C | 3.735 |
| 246CB | 250CD1 | 4.837 | 247OD1 | 250CB | 4.015 |
| 246CB | 250CB | 4.978 | 247OD1 | 250CA | 4.121 |
| 246CB | 274CG | 4.988 | 247OD1 | 249CD | 4.172 |
| 246C | 247CA | 2.445 | 247OD1 | 250CG1 | 4.267 |
| 246C | 247CB | 3.093 | 247OD1 | 250CD1 | 4.870 |
| 246C | 247C | 3.647 | 247OD1 | 249O | 4.930 |
| 246C | 247ND2 | 3.730 | 247ND2 | 250CD1 | 3.820 |
| 246C | 247CG | 3.789 | 247ND2 | 250CG1 | 3.953 |
| 246C | 247O | 3.922 | 247ND2 | 250CB | 4.004 |
| 246C | 274ND2 | 4.440 | 247ND2 | 250N | 4.402 |
| 246C | 274CB | 4.631 | 247ND2 | 250CA | 4.869 |
| 246C | 247OD1 | 4.815 | 247C | 249N | 3.305 |
| 247C | 250N | 4.120 | 274O | 276CA | 4.809 |
| 247C | 249CA | 4.545 | 303N | 304CA | 4.862 |
| 247C | 249C | 4.779 | 303CA | 304N | 2.430 |
| 247C | 250CB | 4.900 | 303CA | 304CA | 3.818 |
| 247C | 250CA | 4.940 | 303CA | 304C | 4.661 |
| 247O | 249N | 3.360 | 303CA | 304O | 4.679 |
| 247O | 250N | 3.406 | 303CA | 304CB | 4.684 |
| 247O | 250CB | 3.950 | 303CB | 304N | 3.222 |
| 247O | 250CA | 3.970 | 303CB | 304CA | 4.521 |
| 247O | 250C | 4.123 | 303CB | 304O | 4.818 |
| 247O | 249C | 4.217 | 303CB | 304C | 4.963 |
| 247O | 249CA | 4.373 | 303C | 304CA | 2.452 |
| 247O | 250CG2 | 4.591 | 303C | 304CB | 3.442 |
| 249N | 250N | 2.817 | 303C | 304C | 3.504 |
| 249N | 250CA | 4.203 | 303C | 304O | 3.860 |
| 249N | 250C | 4.666 | 303C | 305N | 4.467 |
| 249CA | 250N | 2.413 | 303C | 304CG | 4.748 |
| 249CA | 250CA | 3.779 | 303O | 304N | 2.247 |
| 249CA | 250C | 4.413 | 303O | 304CA | 2.806 |
| 249CA | 250CB | 4.866 | 303O | 304CB | 3.762 |
| 249CB | 250N | 3.035 | 303O | 304C | 3.989 |
| 249CB | 250CA | 4.353 | 303O | 304O | 4.630 |
| 249CG | 250N | 4.416 | 303O | 305N | 4.674 |
| 249C | 250CA | 2.410 | 303O | 304CG | 4.827 |
| 249C | 250C | 3.035 | 304N | 305N | 3.547 |
| 249C | 250CB | 3.720 | 304N | 305CA | 4.719 |
| 249C | 250O | 3.774 | 304CA | 305N | 2.450 |
| 249C | 250CG1 | 4.278 | 304CA | 305CA | 3.795 |
| 249C | 250CG2 | 4.919 | 304CA | 305C | 4.958 |
| 249O | 250N | 2.240 | 304CB | 305N | 3.325 |
| 249O | 250CA | 2.733 | 304CG | 305N | 3.321 |
| 249O | 250C | 3.038 | 304CG | 305CA | 4.469 |
| 249O | 250O | 3.374 | 304CG | 305O | 4.729 |
| 249O | 250CB | 4.232 | 304CD1 | 305N | 3.304 |
| 249O | 250CG1 | 4.716 | 304CD1 | 305CA | 4.052 |
| 274OD1 | 276CB | 4.452 | 304CD1 | 305O | 4.145 |
| 274OD1 | 276C | 4.632 | 304CD1 | 305C | 4.383 |
| 274OD1 | 276O | 4.648 | 304CD2 | 305N | 4.139 |
| 274ND2 | 276N | 4.935 | 304CE1 | 305O | 3.966 |
| 274C | 276N | 3.322 | 304CE1 | 305N | 4.100 |
| 274C | 276CA | 4.688 | 304CE1 | 305C | 4.483 |
| 274O | 276N | 3.552 | 304CE1 | 305CA | 4.616 |
| 304CE2 | 305N | 4.804 | | | |
| 304CE2 | 305O | 4.952 | | | |
| 304CZ | 305O | 4.401 | | | |
| 304CZ | 305N | 4.783 | | | |
| 304C | 305N | 1.329 | | | |
| 304C | 305CA | 2.395 | | | |
| 304C | 305C | 3.718 | | | |
| 304C | 305O | 4.130 | | | |
| 304C | 306N | 4.754 | | | |
| 304O | 305N | 2.239 | | | |
| 304O | 305CA | 2.696 | | | |
| 304O | 305C | 4.145 | | | |
| 304O | 305O | 4.826 | | | |
| 304O | 306N | 4.921 | | | |
| 305N | 306N | 3.702 | | | |
| 305N | 306CA | 4.963 | | | |
| 305CA | 306N | 2.391 | | | |
| 305CA | 306CA | 3.771 | | | |
| 305CA | 306O | 4.400 | | | |
| 305CA | 306C | 4.467 | | | |
| 305C | 306CA | 2.427 | | | |
| 305C | 306C | 3.071 | | | |
| 305C | 306O | 3.236 | | | |
| 305O | 306N | 2.248 | | | |
| 305O | 306CA | 2.772 | | | |
| 305O | 306C | 2.996 | | | |
| 305O | 306O | 3.217 | | | |
| 306N | 487CD2 | 4.792 | | | |
| 306CA | 487CD2 | 4.048 | | | |
| 306CA | 487CG | 4.502 | | | |
| 306CA | 487CB | 4.525 | | | |
| 306CA | 487CE2 | 4.655 | | | |
| 306C | 487CB | 4.265 | | | |
| 306C | 487CD2 | 4.576 | | | |
| 306C | 487CG | 4.734 | | | |
| 306O | 487CB | 4.248 | | | |
| 306O | 487CG | 4.922 | | | |

Table III provides the the atomic coordinates of the acetyl-CoA complex structure in the active site. Solvent molecules are omitted here for clarity, but can be found in FIG. 2. Residue 487 is Phe87 from the other monomer. Residue CAC is acetylated cysteine, and COA is the bound CoA molecule.

TABLE III

| ATOM | | RESIDUE | | X | Y | Z | Occ | B |
|---|---|---|---|---|---|---|---|---|
| 1 | N | THR | 28 | 32.909 | 0.319 | 26.935 | 1.00 | 14.64 |
| 2 | CA | THR | 28 | 31.524 | 0.759 | 27.053 | 1.00 | 16.73 |
| 3 | CB | THR | 28 | 31.399 | 2.311 | 26.861 | 1.00 | 18.66 |
| 4 | OG1 | THR | 28 | 30.140 | 2.771 | 27.368 | 1.00 | 21.07 |
| 5 | CG2 | THR | 28 | 31.523 | 2.702 | 25.394 | 1.00 | 14.87 |
| 6 | C | THR | 28 | 30.671 | −0.021 | 26.041 | 1.00 | 15.95 |
| 7 | O | THR | 28 | 31.196 | −0.755 | 25.190 | 1.00 | 14.39 |
| 8 | N | TRP | 32 | 24.685 | 1.112 | 27.156 | 1.00 | 18.61 |
| 9 | CA | TRP | 32 | 24.896 | 1.996 | 28.316 | 1.00 | 17.67 |
| 10 | CB | TRP | 32 | 26.253 | 1.657 | 28.999 | 1.00 | 18.46 |
| 11 | CG | TRP | 32 | 26.543 | 2.508 | 30.252 | 1.00 | 14.22 |
| 12 | CD2 | TRP | 32 | 26.947 | 3.865 | 30.325 | 1.00 | 16.45 |
| 13 | CE2 | TRP | 32 | 27.044 | 4.089 | 31.715 | 1.00 | 13.95 |
| 14 | CE3 | TRP | 32 | 27.232 | 4.916 | 29.444 | 1.00 | 14.91 |
| 15 | CD1 | TRP | 32 | 26.405 | 1.970 | 31.509 | 1.00 | 19.11 |
| 16 | NE1 | TRP | 32 | 26.722 | 2.948 | 32.369 | 1.00 | 17.55 |
| 17 | CZ2 | TRP | 32 | 27.417 | 5.348 | 32.222 | 1.00 | 16.49 |
| 18 | CZ3 | TRP | 32 | 27.602 | 6.164 | 29.953 | 1.00 | 8.45 |
| 19 | CH2 | TRP | 32 | 27.698 | 6.373 | 31.321 | 1.00 | 11.56 |
| 20 | C | TRP | 32 | 24.917 | 3.414 | 27.781 | 1.00 | 16.08 |
| 21 | O | TRP | 32 | 24.363 | 4.325 | 28.378 | 1.00 | 17.69 |
| 22 | N | ILE | 33 | 25.536 | 3.534 | 26.593 | 1.00 | 16.72 |
| 23 | CA | ILE | 33 | 25.591 | 4.911 | 26.052 | 1.00 | 17.89 |
| 24 | CB | ILE | 33 | 26.670 | 5.169 | 24.944 | 1.00 | 20.24 |
| 25 | CG2 | ILE | 33 | 26.790 | 6.671 | 24.704 | 1.00 | 18.87 |
| 26 | CG1 | ILE | 33 | 28.038 | 4.571 | 25.295 | 1.00 | 16.21 |
| 27 | CD1 | ILE | 33 | 28.930 | 4.480 | 24.013 | 1.00 | 24.09 |
| 28 | C | ILE | 33 | 24.196 | 5.403 | 25.732 | 1.00 | 18.98 |
| 29 | O | ILE | 33 | 23.877 | 6.540 | 26.194 | 1.00 | 18.61 |
| 30 | N | ARG | 36 | 22.046 | 6.096 | 28.836 | 1.00 | 20.61 |
| 31 | CA | ARG | 36 | 22.587 | 7.077 | 29.780 | 1.00 | 20.93 |
| 32 | CB | ARG | 36 | 23.940 | 6.602 | 30.339 | 1.00 | 19.27 |
| 33 | CG | ARG | 36 | 23.882 | 5.328 | 31.146 | 1.00 | 20.40 |
| 34 | CD | ARG | 36 | 23.627 | 5.619 | 32.605 | 1.00 | 22.27 |
| 35 | NE | ARG | 36 | 23.511 | 4.396 | 33.393 | 1.00 | 27.02 |
| 36 | CZ | ARG | 36 | 23.867 | 4.298 | 34.670 | 1.00 | 25.93 |
| 37 | NH1 | ARG | 36 | 23.734 | 3.152 | 35.315 | 1.00 | 26.63 |
| 38 | NH2 | ARG | 36 | 24.330 | 5.355 | 35.318 | 1.00 | 23.35 |
| 39 | C | ARG | 36 | 22.702 | 8.517 | 29.247 | 1.00 | 18.28 |
| 40 | O | ARG | 36 | 22.703 | 9.462 | 30.029 | 1.00 | 17.41 |
| 41 | N | THR | 37 | 22.798 | 8.697 | 27.936 | 1.00 | 18.97 |
| 42 | CA | THR | 37 | 22.932 | 10.050 | 27.405 | 1.00 | 21.02 |
| 43 | CB | THR | 37 | 24.388 | 10.371 | 26.949 | 1.00 | 18.78 |
| 44 | OG1 | THR | 37 | 24.793 | 9.461 | 25.925 | 1.00 | 17.72 |
| 45 | CG2 | THR | 37 | 25.347 | 10.293 | 28.084 | 1.00 | 21.35 |
| 46 | C | THR | 37 | 22.048 | 10.362 | 26.222 | 1.00 | 20.16 |
| 47 | O | THR | 37 | 21.914 | 11.534 | 25.839 | 1.00 | 25.43 |
| 48 | N | CAC | 112 | 30.456 | 25.709 | 28.104 | 1.00 | 10.38 |
| 49 | CA | CAC | 112 | 29.270 | 25.229 | 27.412 | 1.00 | 14.44 |
| 50 | CB | CAC | 112 | 28.799 | 23.888 | 27.980 | 1.00 | 17.69 |
| 51 | SG | CAC | 112 | 29.712 | 22.439 | 27.254 | 1.00 | 17.65 |
| 52 | CD | CAC | 112 | 32.183 | 21.508 | 28.594 | 1.00 | 24.17 |
| 53 | CE | CAC | 112 | 30.937 | 22.403 | 28.616 | 1.00 | 21.28 |
| 54 | OE | CAC | 112 | 30.752 | 23.125 | 29.602 | 1.00 | 25.29 |
| 55 | C | CAC | 112 | 28.167 | 26.294 | 27.295 | 1.00 | 11.81 |
| 56 | O | CAC | 112 | 27.369 | 26.232 | 26.368 | 1.00 | 10.19 |
| 57 | N | LEU | 142 | 35.611 | 19.985 | 21.261 | 1.00 | 10.22 |
| 58 | CA | LEU | 142 | 35.860 | 19.347 | 22.539 | 1.00 | 13.06 |
| 59 | CB | LEU | 142 | 34.735 | 19.597 | 23.555 | 1.00 | 12.36 |
| 60 | CG | LEU | 142 | 34.583 | 20.999 | 24.171 | 1.00 | 11.62 |
| 61 | CD1 | LEU | 142 | 33.937 | 20.919 | 25.543 | 1.00 | 5.06 |
| 62 | CD2 | LEU | 142 | 35.940 | 21.651 | 24.300 | 1.00 | 10.88 |
| 63 | C | LEU | 142 | 36.175 | 17.851 | 22.433 | 1.00 | 13.55 |
| 64 | O | LEU | 142 | 36.786 | 17.299 | 23.322 | 1.00 | 19.07 |
| 65 | N | ARG | 151 | 36.295 | 6.724 | 29.164 | 1.00 | 23.03 |
| 66 | CA | ARG | 151 | 34.919 | 6.417 | 28.730 | 1.00 | 23.11 |
| 67 | CB | ARG | 151 | 34.470 | 5.004 | 29.175 | 1.00 | 16.86 |
| 68 | CG | ARG | 151 | 34.348 | 4.774 | 30.666 | 1.00 | 15.32 |
| 69 | CD | ARG | 151 | 33.926 | 3.335 | 30.928 | 1.00 | 7.13 |
| 70 | NE | ARG | 151 | 33.779 | 3.086 | 32.349 | 1.00 | 10.71 |
| 71 | CZ | ARG | 151 | 33.378 | 1.927 | 32.869 | 1.00 | 3.91 |
| 72 | NH1 | ARG | 151 | 33.268 | 1.783 | 34.179 | 1.00 | 4.61 |
| 73 | NH2 | ARG | 151 | 33.078 | 0.930 | 32.071 | 1.00 | 10.10 |
| 74 | C | ARG | 151 | 33.873 | 7.478 | 29.120 | 1.00 | 17.49 |
| 75 | O | ARG | 151 | 33.012 | 7.828 | 28.317 | 1.00 | 17.71 |
| 76 | N | GLY | 152 | 34.016 | 8.044 | 30.309 | 1.00 | 17.52 |
| 77 | CA | GLY | 152 | 33.070 | 9.045 | 30.776 | 1.00 | 16.37 |
| 78 | C | GLY | 152 | 33.062 | 10.401 | 30.082 | 1.00 | 15.84 |
| 79 | O | GLY | 152 | 32.246 | 11.248 | 30.439 | 1.00 | 21.56 |
| 80 | N | ILE | 155 | 32.443 | 9.844 | 25.187 | 1.00 | 7.71 |
| 81 | CA | ILE | 155 | 31.083 | 9.426 | 24.707 | 1.00 | 12.55 |
| 82 | CB | ILE | 155 | 30.385 | 8.425 | 25.708 | 1.00 | 11.77 |
| 83 | CG2 | ILE | 155 | 31.197 | 7.148 | 25.866 | 1.00 | 11.90 |
| 84 | CG1 | ILE | 155 | 30.158 | 9.085 | 27.088 | 1.00 | 12.15 |
| 85 | CD1 | ILE | 155 | 29.158 | 8.276 | 27.966 | 1.00 | 11.79 |
| 86 | C | ILE | 155 | 30.193 | 10.622 | 24.373 | 1.00 | 10.55 |
| 87 | O | ILE | 155 | 29.530 | 10.593 | 23.314 | 1.00 | 14.21 |
| 88 | N | ILE | 156 | 30.115 | 11.601 | 25.228 | 1.00 | 15.15 |
| 89 | CA | ILE | 156 | 29.284 | 12.781 | 24.971 | 1.00 | 13.87 |
| 90 | CB | ILE | 156 | 28.912 | 13.460 | 26.383 | 1.00 | 18.45 |
| 91 | CG2 | ILE | 156 | 27.632 | 12.860 | 26.931 | 1.00 | 23.09 |
| 92 | CG1 | ILE | 156 | 30.082 | 13.252 | 27.370 | 1.00 | 15.34 |
| 93 | CD1 | ILE | 156 | 29.617 | 12.611 | 28.714 | 1.00 | 19.30 |
| 94 | C | ILE | 156 | 29.845 | 13.826 | 24.026 | 1.00 | 13.98 |
| 95 | O | ILE | 156 | 29.049 | 14.365 | 23.211 | 1.00 | 9.76 |
| 96 | N | PHE | 157 | 31.114 | 14.104 | 24.000 | 1.00 | 10.77 |
| 97 | CA | PHE | 157 | 31.656 | 15.152 | 23.157 | 1.00 | 7.33 |
| 98 | CB | PHE | 157 | 32.859 | 15.790 | 23.759 | 1.00 | 4.54 |
| 99 | CG | PHE | 157 | 32.560 | 16.451 | 25.090 | 1.00 | 7.66 |
| 100 | CD1 | PHE | 157 | 32.946 | 15.788 | 26.255 | 1.00 | 3.98 |
| 101 | CD2 | PHE | 157 | 31.915 | 17.650 | 25.184 | 1.00 | 5.65 |
| 102 | CE1 | PHE | 157 | 32.660 | 16.349 | 27.491 | 1.00 | 9.88 |
| 103 | CE2 | PHE | 157 | 31.630 | 18.205 | 26.422 | 1.00 | 4.05 |
| 104 | CZ | PHE | 157 | 32.018 | 17.536 | 27.588 | 1.00 | 6.80 |
| 105 | C | PHE | 157 | 31.810 | 14.851 | 21.690 | 1.00 | 10.70 |
| 106 | O | PHE | 157 | 32.380 | 13.859 | 21.257 | 1.00 | 13.03 |
| 107 | N | LEU | 189 | 34.231 | 20.663 | 36.441 | 1.00 | 15.69 |
| 108 | CA | LEU | 189 | 34.309 | 20.542 | 34.989 | 1.00 | 15.11 |
| 109 | CB | LEU | 189 | 32.983 | 20.982 | 34.350 | 1.00 | 10.07 |
| 110 | CG | LEU | 189 | 32.807 | 20.922 | 32.844 | 1.00 | 7.51 |
| 111 | CD1 | LEU | 189 | 33.311 | 19.593 | 32.263 | 1.00 | 10.35 |
| 112 | CD2 | LEU | 189 | 31.343 | 21.142 | 32.523 | 1.00 | 7.61 |
| 113 | C | LEU | 189 | 35.464 | 21.418 | 34.538 | 1.00 | 15.40 |
| 114 | O | LEU | 189 | 35.452 | 22.612 | 34.812 | 1.00 | 16.51 |
| 115 | N | LEU | 205 | 40.306 | 17.390 | 29.143 | 1.00 | 13.16 |
| 116 | CA | LEU | 205 | 39.050 | 17.802 | 29.770 | 1.00 | 15.27 |
| 117 | CB | LEU | 205 | 37.963 | 17.874 | 28.694 | 1.00 | 12.62 |
| 118 | CG | LEU | 205 | 36.505 | 18.215 | 29.034 | 1.00 | 14.99 |
| 119 | CD1 | LEU | 205 | 35.817 | 18.527 | 27.706 | 1.00 | 15.12 |
| 120 | CD2 | LEU | 205 | 35.773 | 17.085 | 29.762 | 1.00 | 11.51 |
| 121 | C | LEU | 205 | 38.658 | 16.793 | 30.846 | 1.00 | 15.81 |
| 122 | O | LEU | 205 | 38.675 | 15.588 | 30.594 | 1.00 | 20.20 |
| 123 | N | MET | 207 | 35.792 | 15.888 | 34.121 | 1.00 | 18.42 |
| 124 | CA | MET | 207 | 34.419 | 16.232 | 34.463 | 1.00 | 16.18 |
| 125 | CB | MET | 207 | 33.555 | 16.227 | 33.174 | 1.00 | 17.87 |
| 126 | CG | MET | 207 | 32.024 | 16.237 | 33.467 | 1.00 | 17.17 |
| 127 | SD | MET | 207 | 30.990 | 16.464 | 32.044 | 2.00 | 17.60 |
| 128 | CE | MET | 207 | 31.340 | 14.797 | 31.582 | 1.00 | 22.99 |
| 129 | C | MET | 207 | 33.790 | 15.238 | 35.466 | 1.00 | 16.62 |
| 130 | O | MET | 207 | 33.726 | 14.046 | 35.222 | 1.00 | 18.22 |
| 131 | N | GLY | 209 | 30.811 | 14.103 | 36.169 | 1.00 | 12.42 |
| 132 | CA | GLY | 209 | 29.492 | 14.040 | 35.588 | 1.00 | 16.72 |
| 133 | C | GLY | 209 | 28.358 | 14.011 | 36.516 | 1.00 | 19.06 |
| 134 | O | GLY | 209 | 27.487 | 14.883 | 36.423 | 1.00 | 20.59 |
| 135 | N | ASN | 210 | 28.284 | 13.037 | 37.418 | 1.00 | 21.24 |
| 136 | CA | ASN | 210 | 27.150 | 13.010 | 38.362 | 1.00 | 24.44 |
| 137 | CB | ASN | 210 | 27.198 | 11.753 | 39.171 | 1.00 | 25.49 |
| 138 | CG | ASN | 210 | 27.160 | 11.958 | 40.631 | 1.00 | 33.50 |
| 139 | OD1 | ASN | 210 | 26.177 | 11.619 | 41.309 | 1.00 | 34.80 |
| 140 | ND2 | ASN | 210 | 28.217 | 12.429 | 41.247 | 1.00 | 32.41 |
| 141 | C | ASN | 210 | 26.970 | 14.201 | 39.196 | 1.00 | 25.55 |
| 142 | O | ASN | 210 | 25.858 | 14.799 | 39.270 | 1.00 | 27.11 |
| 143 | N | VAL | 212 | 27.967 | 17.255 | 38.323 | 1.00 | 18.86 |
| 144 | CA | VAL | 212 | 27.657 | 18.365 | 37.397 | 1.00 | 19.45 |
| 145 | CB | VAL | 212 | 28.483 | 18.363 | 36.115 | 1.00 | 13.66 |
| 146 | CG1 | VAL | 212 | 28.142 | 19.417 | 35.091 | 1.00 | 10.49 |
| 147 | CG2 | VAL | 212 | 29.921 | 18.642 | 36.480 | 1.00 | 11.31 |
| 148 | C | VAL | 212 | 26.176 | 18.359 | 36.977 | 1.00 | 25.20 |
| 149 | O | VAL | 212 | 25.455 | 19.359 | 36.929 | 1.00 | 27.15 |
| 150 | N | PHE | 213 | 25.738 | 17.114 | 36.763 | 1.00 | 24.63 |
| 151 | CA | PHE | 213 | 24.361 | 16.813 | 36.336 | 1.00 | 25.87 |
| 152 | CB | PHE | 213 | 24.203 | 15.287 | 36.398 | 1.00 | 23.74 |
| 153 | CG | PHE | 213 | 22.788 | 14.958 | 36.099 | 1.00 | 23.97 |
| 154 | CD1 | PHE | 213 | 22.533 | 14.398 | 34.810 | 1.00 | 27.08 |
| 155 | CD2 | PHE | 213 | 21.752 | 14.909 | 36.974 | 1.00 | 22.61 |
| 156 | CE1 | PHE | 213 | 21.275 | 13.964 | 34.464 | 1.00 | 23.26 |
| 157 | CE2 | PHE | 213 | 20.480 | 14.482 | 36.625 | 1.00 | 23.27 |
| 158 | CZ | PHE | 213 | 20.223 | 13.976 | 35.335 | 1.00 | 21.74 |
| 159 | C | PHE | 213 | 23.356 | 17.458 | 37.319 | 1.00 | 26.46 |
| 160 | O | PHE | 213 | 22.395 | 18.091 | 36.945 | 1.00 | 28.12 |
| 161 | N | ALA | 216 | 23.435 | 21.215 | 37.390 | 1.00 | 21.80 |
| 162 | CA | ALA | 216 | 22.949 | 21.675 | 36.100 | 1.00 | 19.74 |
| 163 | CB | ALA | 216 | 23.464 | 20.861 | 34.933 | 1.00 | 18.25 |
| 164 | C | ALA | 216 | 21.440 | 21.811 | 36.028 | 1.00 | 20.86 |
| 165 | O | ALA | 216 | 20.936 | 22.882 | 35.612 | 1.00 | 14.72 |
| 166 | N | HIS | 244 | 21.005 | 23.509 | 25.764 | 1.00 | 14.90 |
| 167 | CA | HIS | 244 | 22.348 | 23.098 | 25.390 | 1.00 | 17.43 |
| 168 | CB | HIS | 244 | 23.328 | 23.551 | 26.478 | 1.00 | 17.97 |
| 169 | CG | HIS | 244 | 24.644 | 22.836 | 26.459 | 1.00 | 18.58 |
| 170 | CD2 | HIS | 244 | 25.582 | 22.714 | 25.488 | 1.00 | 18.43 |
| 171 | ND1 | HIS | 244 | 25.123 | 22.136 | 27.546 | 1.00 | 18.75 |
| 172 | CE1 | HIS | 244 | 26.295 | 21.608 | 27.243 | 1.00 | 21.88 |
| 173 | NE2 | HIS | 244 | 26.597 | 21.944 | 26.000 | 1.00 | 17.34 |
| 174 | C | HIS | 244 | 22.190 | 21.563 | 25.366 | 1.00 | 17.94 |
| 175 | O | HIS | 244 | 21.579 | 20.979 | 26.286 | 1.00 | 18.08 |
| 176 | N | ALA | 246 | 23.569 | 18.461 | 26.118 | 1.00 | 19.92 |
| 177 | CA | ALA | 246 | 24.594 | 17.753 | 26.886 | 1.00 | 22.75 |
| 178 | CB | ALA | 246 | 24.851 | 18.474 | 28.207 | 1.00 | 20.40 |
| 179 | C | ALA | 246 | 24.197 | 16.301 | 27.174 | 1.00 | 25.65 |
| 180 | O | ALA | 246 | 24.941 | 15.364 | 26.869 | 1.00 | 27.18 |
| 181 | N | ASN | 247 | 23.035 | 16.122 | 27.793 | 1.00 | 26.14 |
| 182 | CA | ASN | 247 | 22.545 | 14.795 | 28.146 | 1.00 | 26.38 |
| 183 | CB | ASN | 247 | 22.964 | 14.464 | 29.587 | 1.00 | 28.11 |
| 184 | CG | ASN | 247 | 22.574 | 13.044 | 30.019 | 1.00 | 32.46 |
| 185 | OD1 | ASN | 247 | 21.552 | 12.486 | 29.583 | 1.00 | 30.09 |
| 186 | ND2 | ASN | 247 | 23.371 | 12.470 | 30.912 | 1.00 | 31.19 |
| 187 | C | ASN | 247 | 21.021 | 14.827 | 28.020 | 1.00 | 26.38 |
| 188 | O | ASN | 247 | 20.381 | 15.783 | 28.497 | 1.00 | 24.78 |
| 189 | N | ARG | 249 | 18.806 | 13.418 | 29.619 | 1.00 | 26.17 |
| 190 | CA | ARG | 249 | 18.082 | 13.368 | 30.918 | 1.00 | 30.19 |
| 191 | CB | ARG | 249 | 18.684 | 12.450 | 31.892 | 1.00 | 35.11 |
| 192 | CG | ARG | 249 | 20.149 | 12.514 | 32.084 | 1.00 | 40.00 |
| 193 | CD | ARG | 249 | 20.737 | 11.377 | 33.040 | 1.00 | 40.00 |
| 194 | NE | ARG | 249 | 19.770 | 10.270 | 32.981 | 1.00 | 40.00 |
| 195 | CZ | ARG | 249 | 20.131 | 8.991 | 32.720 | 1.00 | 40.00 |
| 196 | NH1 | ARG | 249 | 19.206 | 8.005 | 32.728 | 1.00 | 40.00 |
| 197 | NH2 | ARG | 249 | 21.400 | 8.728 | 32.469 | 1.00 | 40.00 |
| 198 | C | ARG | 249 | 17.883 | 14.720 | 31.505 | 1.00 | 30.27 |
| 199 | O | ARG | 249 | 16.848 | 15.128 | 31.999 | 1.00 | 29.21 |
| 200 | N | ILE | 250 | 19.022 | 15.485 | 31.317 | 1.00 | 31.13 |
| 201 | CA | ILE | 250 | 18.989 | 16.891 | 31.777 | 1.00 | 30.26 |
| 202 | CB | ILE | 250 | 20.417 | 17.557 | 31.646 | 1.00 | 31.49 |
| 203 | CG2 | ILE | 250 | 20.269 | 19.060 | 31.848 | 1.00 | 27.57 |
| 204 | CG1 | ILE | 250 | 21.391 | 16.967 | 32.703 | 1.00 | 27.31 |
| 205 | CD1 | ILE | 250 | 22.804 | 17.587 | 32.626 | 1.00 | 29.25 |
| 206 | C | ILE | 250 | 17.878 | 17.652 | 31.051 | 1.00 | 29.32 |
| 207 | O | ILE | 250 | 17.014 | 18.274 | 31.667 | 1.00 | 30.29 |
| 208 | N | ASN | 274 | 27.325 | 16.399 | 22.555 | 1.00 | 13.47 |
| 209 | CA | ASN | 274 | 27.474 | 17.720 | 23.155 | 1.00 | 13.39 |
| 210 | CB | ASN | 274 | 27.818 | 17.530 | 24.622 | 1.00 | 15.56 |
| 211 | CG | ASN | 274 | 27.960 | 18.816 | 25.366 | 1.00 | 17.87 |
| 212 | OD1 | ASN | 274 | 28.135 | 19.881 | 24.780 | 1.00 | 24.67 |
| 213 | ND2 | ASN | 274 | 27.890 | 18.729 | 26.689 | 1.00 | 19.64 |
| 214 | C | ASN | 274 | 28.638 | 18.414 | 22.458 | 1.00 | 14.33 |
| 215 | O | ASN | 274 | 29.770 | 17.971 | 22.613 | 1.00 | 12.94 |
| 216 | N | SER | 276 | 29.549 | 21.633 | 22.863 | 1.00 | 7.51 |
| 217 | CA | SER | 276 | 29.823 | 22.861 | 23.613 | 1.00 | 13.37 |
| 218 | CB | SER | 276 | 31.354 | 23.045 | 23.758 | 1.00 | 16.08 |
| 219 | OG | SER | 276 | 31.709 | 24.178 | 24.552 | 1.00 | 13.44 |
| 220 | C | SER | 276 | 29.132 | 24.114 | 23.029 | 1.00 | 13.89 |
| 221 | O | SER | 276 | 27.945 | 24.062 | 22.700 | 1.00 | 11.72 |
| 222 | N | PHE | 304 | 24.334 | 25.567 | 30.088 | 1.00 | 14.66 |
| 223 | CA | PHE | 304 | 25.107 | 25.471 | 31.332 | 1.00 | 17.36 |
| 224 | CB | PHE | 304 | 24.396 | 24.476 | 32.274 | 1.00 | 14.19 |
| 225 | CG | PHE | 304 | 25.035 | 24.321 | 33.630 | 1.00 | 14.80 |
| 226 | CD1 | PHE | 304 | 26.179 | 23.562 | 33.795 | 1.00 | 13.55 |
| 227 | CD2 | PHE | 304 | 24.464 | 24.909 | 34.748 | 1.00 | 18.11 |
| 228 | CE1 | PHE | 304 | 26.751 | 23.388 | 35.053 | 1.00 | 13.17 |
| 229 | CE2 | PHE | 304 | 25.024 | 24.744 | 36.014 | 1.00 | 19.56 |
| 230 | CZ | PHE | 304 | 26.175 | 23.977 | 36.166 | 1.00 | 18.61 |
| 231 | C | PHE | 304 | 26.495 | 24.936 | 30.934 | 1.00 | 18.48 |
| 232 | O | PHE | 304 | 26.597 | 24.072 | 30.048 | 1.00 | 19.82 |
| 233 | N | GLY | 305 | 27.546 | 25.411 | 31.603 | 1.00 | 20.16 |
| 234 | CA | GLY | 305 | 28.889 | 24.966 | 31.272 | 1.00 | 18.15 |
| 235 | C | GLY | 305 | 29.950 | 25.008 | 32.367 | 1.00 | 15.06 |
| 236 | O | GLY | 305 | 29.701 | 25.407 | 33.507 | 1.00 | 11.78 |
| 237 | N | GLY | 306 | 31.145 | 24.556 | 31.988 | 1.00 | 16.84 |
| 238 | CA | GLY | 306 | 32.290 | 24.514 | 32.875 | 1.00 | 16.87 |
| 239 | C | GLY | 306 | 32.529 | 25.856 | 33.525 | 1.00 | 19.36 |
| 240 | O | GLY | 306 | 32.236 | 26.899 | 32.934 | 1.00 | 17.63 |
| 241 | N | PHE | 487 | 38.425 | 26.469 | 30.807 | 1.00 | 13.64 |
| 242 | CA | PHE | 487 | 37.277 | 26.474 | 31.704 | 1.00 | 12.94 |
| 243 | CB | PHE | 487 | 35.953 | 26.064 | 31.031 | 1.00 | 16.12 |
| 244 | CG | PHE | 487 | 35.967 | 24.728 | 30.332 | 1.00 | 10.50 |
| 245 | CD1 | PHE | 487 | 36.055 | 24.668 | 28.952 | 1.00 | 11.96 |
| 246 | CD2 | PHE | 487 | 35.776 | 23.548 | 31.043 | 1.00 | 14.59 |
| 247 | CE1 | PHE | 487 | 35.943 | 23.450 | 28.275 | 1.00 | 11.67 |
| 248 | CE2 | PHE | 487 | 35.665 | 22.321 | 30.373 | 1.00 | 10.83 |
| 249 | CZ | PHE | 487 | 35.748 | 22.283 | 28.989 | 1.00 | 9.41 |
| 250 | C | PHE | 487 | 37.606 | 25.574 | 32.861 | 1.00 | 13.75 |
| 251 | O | PHE | 487 | 38.217 | 24.529 | 32.661 | 1.00 | 18.53 |
| 252 | AO6 | COA | 350 | 25.886 | 9.541 | 33.559 | 1.00 | 40.00 |
| 253 | AP2 | COA | 350 | 25.938 | 8.466 | 34.779 | 1.00 | 40.00 |
| 254 | AO4 | COA | 350 | 25.984 | 7.033 | 34.193 | 1.00 | 40.00 |
| 255 | AO5 | COA | 350 | 24.688 | 8.689 | 35.674 | 1.00 | 40.00 |
| 256 | AO3 | COA | 350 | 27.383 | 8.800 | 35.491 | 1.00 | 40.00 |
| 257 | AP1 | COA | 350 | 27.959 | 7.998 | 36.780 | 1.00 | 40.00 |
| 258 | AO1 | COA | 350 | 26.887 | 7.993 | 37.879 | 1.00 | 40.00 |
| 259 | AO2 | COA | 350 | 29.237 | 8.653 | 37.296 | 1.00 | 40.00 |
| 260 | AO5* | COA | 350 | 28.201 | 6.460 | 36.164 | 1.00 | 40.00 |
| 261 | AC5* | COA | 350 | 27.718 | 5.279 | 36.817 | 1.00 | 39.18 |
| 262 | AC4* | COA | 350 | 28.472 | 4.019 | 36.378 | 1.00 | 37.65 |
| 263 | AO4* | COA | 350 | 28.702 | 4.012 | 34.931 | 1.00 | 35.45 |
| 264 | AC3* | COA | 350 | 29.898 | 3.856 | 36.965 | 1.00 | 37.54 |
| 265 | AO3* | COA | 350 | 30.205 | 2.474 | 37.178 | 1.00 | 40.00 |
| 266 | AP3* | COA | 350 | 31.518 | 2.029 | 38.160 | 1.00 | 40.00 |
| 267 | AO7 | COA | 350 | 32.888 | 2.220 | 37.337 | 1.00 | 40.00 |
| 268 | AO8 | COA | 350 | 31.503 | 3.018 | 39.420 | 1.00 | 40.00 |
| 269 | AO9 | COA | 350 | 31.296 | 0.500 | 38.522 | 1.00 | 40.00 |
| 270 | AC2* | COA | 350 | 30.688 | 4.469 | 35.850 | 1.00 | 32.65 |
| 271 | AO2* | COA | 350 | 32.112 | 4.433 | 35.932 | 1.00 | 24.96 |
| 272 | AC1* | COA | 350 | 30.098 | 3.815 | 34.584 | 1.00 | 27.72 |
| 273 | AN9 | COA | 350 | 30.429 | 4.564 | 33.382 | 1.00 | 20.99 |
| 274 | AC8 | COA | 350 | 30.840 | 5.878 | 33.186 | 1.00 | 21.31 |
| 275 | AN7 | COA | 350 | 30.992 | 6.002 | 31.788 | 1.00 | 18.53 |
| 276 | AC5 | COA | 350 | 30.700 | 4.873 | 31.234 | 1.00 | 12.67 |
| 277 | AC6 | COA | 350 | 30.698 | 4.501 | 29.898 | 1.00 | 12.21 |
| 278 | AN6 | COA | 350 | 31.039 | 5.381 | 28.963 | 1.00 | 15.81 |
| 279 | AN1 | COA | 350 | 30.338 | 3.249 | 29.672 | 1.00 | 17.72 |
| 280 | AC2 | COA | 350 | 30.014 | 2.442 | 30.654 | 1.00 | 11.38 |
| 281 | AN3 | COA | 350 | 29.997 | 2.743 | 31.973 | 1.00 | 15.08 |
| 282 | AC4 | COA | 350 | 30.341 | 3.964 | 32.268 | 1.00 | 15.56 |
| 283 | PS1 | COA | 350 | 27.926 | 20.647 | 30.314 | 1.00 | 40.00 |
| 284 | PC2 | COA | 350 | 26.439 | 19.897 | 31.045 | 1.00 | 40.00 |
| 285 | PC3 | COA | 350 | 26.760 | 18.654 | 31.835 | 1.00 | 40.00 |
| 286 | PN4 | COA | 350 | 26.965 | 17.518 | 30.873 | 1.00 | 40.00 |
| 287 | PC5 | COA | 350 | 27.350 | 16.338 | 31.273 | 1.00 | 40.00 |
| 288 | PO5 | COA | 350 | 27.542 | 15.370 | 30.476 | 1.00 | 40.00 |
| 289 | PC6 | COA | 350 | 27.580 | 16.199 | 32.745 | 1.00 | 40.00 |
| 290 | PC7 | COA | 350 | 26.255 | 15.801 | 33.363 | 1.00 | 40.00 |
| 291 | PN8 | COA | 350 | 26.292 | 14.370 | 33.634 | 1.00 | 40.00 |
| 292 | PC9 | COA | 350 | 26.176 | 13.440 | 32.669 | 1.00 | 37.37 |
| 293 | PO9 | COA | 350 | 25.948 | 13.691 | 31.437 | 1.00 | 31.87 |
| 294 | PC10 | COA | 350 | 26.320 | 11.982 | 33.151 | 1.00 | 38.48 |
| 295 | PC10 | COA | 350 | 26.849 | 11.940 | 34.496 | 1.00 | 37.07 |
| 296 | PC11 | COA | 350 | 27.172 | 11.057 | 32.178 | 1.00 | 40.00 |
| 297 | PC13 | COA | 350 | 28.667 | 11.476 | 32.189 | 1.00 | 40.00 |
| 298 | PC14 | COA | 350 | 26.632 | 11.101 | 30.745 | 1.00 | 40.00 |
| 299 | PC12 | COA | 350 | 26.933 | 9.588 | 32.579 | 1.00 | 40.00 |

Mutants and Derivatives

The invention further provides homologues, co-complexes and mutants of the E. coli FabH crystal structure of the invention.

The term “homologue” means a protein having at least 30% amino acid sequence identity with E. coli FabH or any of its functional domains.

The term “co-complex” means FabH or a mutant or homologue of FabH in covalent or non-covalent association with a chemical entity or compound.

The term “mutant” refers to a FabH polypeptide, i.e., a polypeptide displaying the biological activity of wild-type FabH activity, characterized by the replacement of at least one amino acid from the wild-type FabH sequence. Such a mutant may be prepared, for example, by expression of E. coli FabH cDNA previously altered in its coding sequence by oligonucleotide-directed mutagenesis.

E. coli FabH mutants may also be generated by site-specific incorporation of unnatural amino acids into the FabH proteins using the general biosynthetic method of C. J. Noren et al, Science, 244:182-188 (1989). In this method, the codon encoding the amino acid of interest in wild-type FabH enzyme is replaced by a “blank” nonsense codon, TAG, using oligonucleotide-directed mutagenesis. A suppressor tRNA directed against this codon is then chemically aminoacylated in vitro with the desired unnatural amino acid. The aminoacylated tRNA is then added to an in vitro translation system to yield a mutant FabH enzyme with the site-specific incorporated unnatural amino acid.

Selenomethionine may be incorporated into wild-type or mutant FabH by expression of E. coli FabH-encoding cDNAs in auxotrophic or non-auxotrophic E. coli strains [W. A. Hendrickson et al, EMBO J., 9(5):1665-1672 (1990)]. In this method, the wild-type or mutagenized FabH cDNA may be expressed in a host organism on a growth medium depleted of either natural methionine but enriched in selenomethionine. The location(s) of the Se atom(s) can be determined by X-ray diffraction analysis at three or four different wavelengths. This information, in turn, is used to generate the phase information used to construct three-dimensional structure of the enzyme.

II. Methods of Identifying Inhibitors of the Novel FabH Crystalline Structure

Another aspect of this invention involves a method for identifying inhibitors of a FabH enzyme characterized by the crystal structure and novel active site described herein, and the inhibitors themselves. The novel E. coli FabH crystalline structure of the invention, or the structure of E. coli FabH homologue, permits the identification of inhibitors of FabH activity. Such inhibitors may be competitive, binding to all or a portion of the active site of FabH; or non-competitive and bind to and inhibit FabH whether or not it is bound to another chemical entity.

One design approach is to probe the FabH crystal of the invention with molecules composed of a variety of different chemical entities to determine optimal sites for interaction between candidate inhibitors and the enzyme. For example, high resolution X-ray diffraction data collected from crystals saturated with solvent allows the determination of where each type of solvent molecule sticks. Small molecules that bind tightly to those sites can then be designed and synthesized and tested for their FabH inhibitor activity [J. Travis, Science, 262:1374 (1993)].

This invention also enables the development of compounds that can isomerize to short-lived reaction intermediates in the chemical reaction of a substrate or other compound that binds to or with FabH. Thus, the time-dependent analysis of structural changes in FabH during its interaction with other molecules is permitted. The reaction intermediates of FabH can also be deduced from the reaction product in co-complex with FabH. Such information is useful to design improved analogues of known FabH inhibitors or to design novel classes of inhibitors based on the reaction intermediates of the enzyme and enzyme-inhibitor co-complex. This provides a novel route for designing FabH inhibitors with both high specificity and stability.

Another approach made possible by this invention, is to screen computationally small molecule data bases for chemical entities or compounds that can bind in whole, or in part, to the FabH enzyme. In this screening, the quality of fit of such entities or compounds to the binding site may be judged either by shape complementarity or by estimated interaction energy [E. C. Meng et al, J. Comp. Chem., 13:505-524 (1992)].

Because FabH may crystallize in more than one crystal form, the structure coordinates of FabH, or portions thereof, as provided by this invention are particularly useful to solve the structure of those other crystal forms of FabH. They may also be used to solve the structure of FabH mutant co-complexes, or of the crystalline form of any other protein with significant amino acid sequence homology to any functional domain of FabH.

One method that may be employed for this purpose is molecular replacement. In this method, the unknown crystal structure, whether it is another crystal form of FabH, a FabH mutant, a FabH co-complex, a FabH from a different bacterial species, or the crystal of some other protein with significant amino acid sequence homology to any domain of FabH, may be determined using the FabH structure coordinates of this invention as provided in FIG. 1 and Tables I-III. This method will provide an accurate structural form for the unknown crystal more quickly and efficiently than attempting to determine such information ab initio.

Thus, the FabH structure provided herein permits the screening of known molecules and/or the designing of new molecules which bind to the structure, particularly at the active site or substrate binding site, via the use of computerized evaluation systems. For example, computer modeling systems are available in which the sequence of the FabH, and the FabH structure (i.e., the atomic coordinates, bond distances between atoms in the active site region, etc. as provided by FIGS. 1-2 and Tables I-III herein) may be input. Thus, a machine readable medium may be encoded with data representing the coordinates of FIGS. 1-2 and Tables I-III. The computer then generates structural details of the site into which a test compound should bind, thereby enabling the determination of the complementary structural details of said test compound.

More particularly, the design of compounds that bind to or inhibit FabH according to this invention generally involves consideration of two factors. First, the compound must be capable of physically and structurally associating with FabH. Non-covalent molecular interactions important in the association of FabH with its substrate include hydrogen bonding, van der Waals and hydrophobic interactions.

Second, the compound must be able to assume a conformation that allows it to associate with FabH. Although certain portions of the compound will not directly participate in this association with FabH, those portions may still influence the overall conformation of the molecule. This, in turn, may have a significant impact on potency. Such conformational requirements include the overall three-dimensional structure and orientation of the chemical entity or compound in relation to all or a portion of the binding site, e.g., active site or substrate binding sites of FabH, or the spacing between functional groups of a compound comprising several chemical entities that directly interact with FabH.

The potential inhibitory or binding effect of a chemical compound on FabH may be analyzed prior to its actual synthesis and testing by the use of computer modelling techniques. If the theoretical structure of the given compound suggests insufficient interaction and association between it and FabH, synthesis and testing of the compound is obviated. However, if computer modelling indicates a strong interaction, the molecule may then be synthesized and tested for its ability to bind to FabH and inhibit using a suitable assay. In this manner, synthesis of inoperative compounds may be avoided.

An inhibitory or other binding compound of FabH may be computationally evaluated and designed by means of a series of steps in which chemical entities or fragments are screened and selected for their ability to associate with the individual binding pockets or other areas of FabH.

One skilled in the art may use one of several methods to screen chemical entities or fragments for their ability to associate with FabH and more particularly with the individual binding pockets of the FabH active site or accessory binding sites. This process may begin by visual inspection of, for example, the active site on the computer screen based on the FabH coordinates in FIGS. 1-2 and Tables I-III. Selected fragments or chemical entities may then be positioned in a variety of orientations, or docked, within a binding pocket of FabH. Docking may be accomplished using software such as Quanta and Sybyl, followed by energy minimization and molecular dynamics with standard molecular mechanics force fields, such as CHARMM and AMBER.

Specialized computer programs may also assist in the process of selecting fragments or chemical entities. These include:

1. GRID [P. J. Goodford, “A Computational Procedure for Determining Energetically Favorable Binding Sites on Biologically Important Macromolecules”, J. Med. Chem., 28:849-857 (1985)]. GRID is available from Oxford University, Oxford, UK.

2. MCSS [A. Miranker and M. Karplus, “Functionality Maps of Binding Sites: A Multiple Copy Simultaneous Search Method”, Proteins: Structure. Function and Genetics, 11:29-34 (1991)]. MCSS is available from Molecular Simulations, Burlington, Mass.

3. AUTODOCK [D. S. Goodsell and A. J. Olsen, “Automated Docking of Substrates to Proteins by Simulated Annealing”, Proteins: Structure, Function, and Genetics, 8:195-202 (1990)]. AUTODOCK is available from Scripps Research Institute, La Jolla, Calif.

4. DOCK [I. D. Kuntz et al, “A Geometric Approach to Macromolecule-Ligand Interactions”, J. Mol. Biol., 161:269-288 (1982)]. DOCK is available from University of California, San Francisco, Calif.

Once suitable chemical entities or fragments have been selected, they can be assembled into a single compound or inhibitor. Assembly may be proceed by visual inspection of the relationship of the fragments to each other on the three-dimensional image displayed on a computer screen in relation to the structure coordinates of FabH. This would be followed by manual model building using software such as Quanta or Sybyl.

Useful programs to aid one of skill in the art in connecting the individual chemical entities or fragments include:

1. CAVEAT [P. A. Bartlett et al, “CAVEAT: A Program to Facilitate the Structure-Derived Design of Biologically Active Molecules”, in Molecular Recognition in Chemical and Biological Problems”, Special Pub., Royal Chem. Soc. 78, pp. 182-196 (1989)]. CAVEAT is available from the University of California, Berkeley, Calif.

2. 3D Database systems such as MACCS-3D (MDL Information Systems, San Leandro, Calif.). This area is reviewed in Y. C. Martin, “3D Database Searching in Drug Design”, J. Med. Chem., 35:2145-2154 (1992).

3. HOOK (available from Molecular Simulations, Burlington, Mass.).

Instead of proceeding to build a FabH inhibitor in a step-wise fashion one fragment or chemical entity at a time as described above, inhibitory or other FabH binding compounds may be designed as a whole or “de novo” using either an empty active site or optionally including some portion(s) of a known inhibitor(s). These methods include:

1. LUDI [H.-J. Bohm, “The Computer Program LUDI: A New Method for the De Novo Design of Enzyme Inhibitors”, J. Comp. Aid. Molec. Design, 6:61-78 (1992)]. LUDI is available from Biosym Technologies, San Diego, Calif.

2. LEGEND [Y. Nishibata and A. Itai, Tetrahedron, 47:8985 (1991)]. LEGEND is available from Molecular Simulations, Burlington, Mass.

3. LeapFrog (available from Tripos Associates, St. Louis, Mo.).

Other molecular modelling techniques may also be employed in accordance with this invention. See, e.g., N. C. Cohen et al, “Molecular Modeling Software and Methods for Medicinal Chemistry”, J. Med. Chem., 33:883-894 (1990). See also, M. A. Navia and M. A. Murcko, “The Use of Structural Information in Drug Design”, Current Opinions in Structural Biology, 2:202-210 (1992). For example, where the structures of test compounds are known, a model of the test compound may be superimposed over the model of the structure of the invention. Numerous methods and techniques are known in the art for performing this step, any of which may be used. See, e.g., P. S. Farmer, Drug Design, Ariens, E. J., ed., Vol. 10, pp 119-143 (Academic Press, New York, 1980); U.S. Pat. No. 5,331,573; U.S. Pat. No. 5,500,807; C. Verlinde, Curr. Biol., 2:577-587 (1994); and I. D. Kuntz, Science, 257:1078-1082 (1992). The model building techniques and computer evaluation systems described herein are not a limitation on the present invention.

Thus, using these computer evaluation systems, a large number of compounds may be quickly and easily examined and expensive and lengthy testing avoided. Moreover, the need for actual synthesis of many compounds is effectively eliminated.

Once identified by the modelling techniques, the FabH inhibitor may be tested for bioactivity using standard techniques. For example, structure of the invention may be used in binding assays using conventional formats to screen inhibitors. One particularly suitable assay format includes the enzyme-linked immunosorbent assay (ELISA). Other assay formats may be used; these assay formats are not a limitation on the present invention.

In another aspect, the FabH structure of the invention permit the design and identification of synthetic compounds and/or other molecules which are characterized by the conformation of FabH of the invention. Using known computer systems, the coordinates of the FabH structure of the invention may be provided in machine readable form, the test compounds designed and/or screened and their conformations superimposed on the structure of the FabH of the invention. Subsequently, suitable candidates identified as above may be screened for the desired FabH inhibitory bioactivity, stability, and the like.

Once identified and screened for biological activity, these inhibitors may be used therapeutically or prophylactically to block FabH activity, and thus, bacterial replication.

The following examples illustrate various aspects of this invention. These examples do not limit the scope of this invention which is defined by the appended claims.

### EXAMPLE 1

### The Expression of FabH from Escherichia coli in Escherichia coli

The strategy for the expression of the FabH from Escherichia coli, using Escherichia coli as a host was based on the PCR amplification of the fabH gene and the introduction of suitable restriction sites that allowed the cloning of the fabH-containing DNA fragment in the pET29 expression vector. After the PCR amplification the insert of the resultant recombinant plasmid, (pET29c hereafter), was sequenced to verify the absence of artifacts introduced by the Taq polymerase reaction. Expression was monitored by SDS-polyacrylamide gel analysis.

A. Bacterial Strains, Plasmids and Medium

The Escherichia coli strains used were: MAXEfficiency DH10B Competent Cells Genotype: F⁻ mcrA Δ(mrr-hsdRMS-mcrBC) φ80dlacZΔM15 ΔlacX74 deoR recA1 araD139 Δ(ara, leu)7697 galU galK λ⁻ rpsL nupG. E. coli cells were grown at 37° C. in Luria Bertani broth (LB). These strains may all be obtained from commercial sources. BL21(DE3) competent cells for protein expression purchased by Novagen. The protocol used to make them electroporation-competent was the one provided by Invitrogen.

The plasmid used was pET29 [Novagen]. A detailed description of pET29 is provided in FIG. 2. Briefly, pET29 is an expression vector of E.coli which is based on the T7 promoter-driven system and allows the selection of the recombinant clones by kanamycin resistance.

LB Medium. Per liter:

| | |
|---|---|
| Bacto-tryptone | 10 g |
| Bacto-yeast extract | 5 g |
| NaCl | 5 g |

For plasmid propagation 0.1 mg/ml of kanamycin was added to the medium.

B. DNA Manipulations

Plasmid DNA was prepared by the rapid alkaline method (Sambrook et al, 1989). Transformations of E. coli cells were carried out according to the protocol provided with the DH10B or the electroporation method. The plasmids for sequencing were purified using QIAGEN mini-prep kit [QIAGEN]. DNA sequencing was carried out on supercoiled plasmid DNA by the dideoxy chain-termination method using the Thermo Sequenase cycle sequencing kit [ABI, BigDye, Applied BioSystems, USA]. Synthetic oligonucleotides [ordered in-house] were used as primers. Restriction enzymes and T4DNA ligase were obtained from Gibco BRL (Life Technologies) and the experiments were carried out following the instructions provided by the suppliers.

The fabH gene from E.coli cloned in the pET29 vector was amplified by PCR using the primers:

| | |
|---|---|
| (5′-TATACATATGTATACGAAGATTATTGGT-3′; | SEQ ID |
| | NO: 2) |
| and: | |
| (5′-ATATGGATCCCTAGAAACGAACCAGCGCGG-3′;. | SEQ ID |
| | NO: 3) |

NdeI and BamHI restriction sites were incorporated at the 5′ and 3′ ends respectively of each primer to facilitate ligation of the amplified DNA into vectors. Plasmid DNA (480 ng) was amplified in 100 ul of PCR mixture containing 200 uM deoxynucleotide triphosphates (dNTPs), 0.20 mM oligonucleotide primers, the manufacturer's buffer and 2.5 U of pfu (Stratagene). The following cycling parameters were used:

94° C. 4 min

94° C. 1 min, 55° C. 40 sec, 72° C. 1 min (30 cycles)

72° C. 2 min

4° C.

Polymerase chain reaction (PCR) was performed using the GeneAmp, PCR System 2400 [Perkin Elmer Cetus Co]. PCR-amplified DNA fragments were purified using Qiaquick PCR Purification kit for Rapid Purification of DNA Fragments [Qiagen].

C. Cloning of the fabH Gene of E. coli in the Expression Vector pET29 of E. coli.

The cloning strategy is shown in FIG. 2. PCR amplification of the fabH gene from E. coli using the primers AKK2 and AKK3 resulted in a DNA fragment of about 960 bp. This fragment was purified with Qiaquick PCR purification kit protocol (Qiagen) digested with NdeI and BamHI, purified, ligated overnight to the NdeI and BamHI sites of already digested vector pET29 to obtain the recombinant plasmid pET29c. The ligation mix was used to transform E. coli DH10B competent cells. The construction of pET29c was initially confirmed by restriction analysis of the plasmid purified from the transformants. The amplification with Taq DNA polymerase made the sequencing of the fabH of pET29c an obligatory step to confirm that no changes were introduced due to the low fidelity of this enzyme. Sequence analysis was accomplished by using T7 promoter and terminator primers. The sequencing of both strands showed that no artifacts had been introduced during the amplification process.

D. Small-Scale Production of FabH from E. coli in E. coli

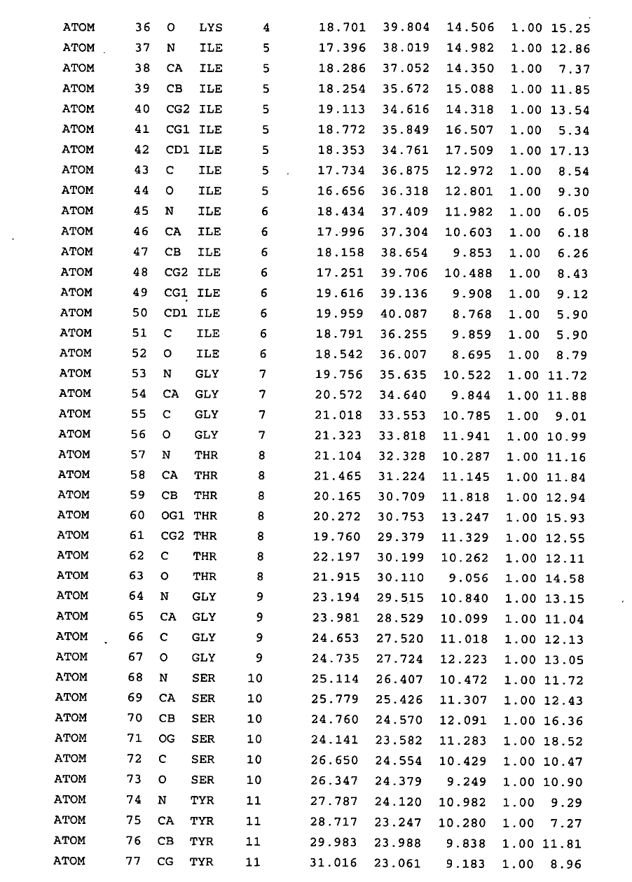

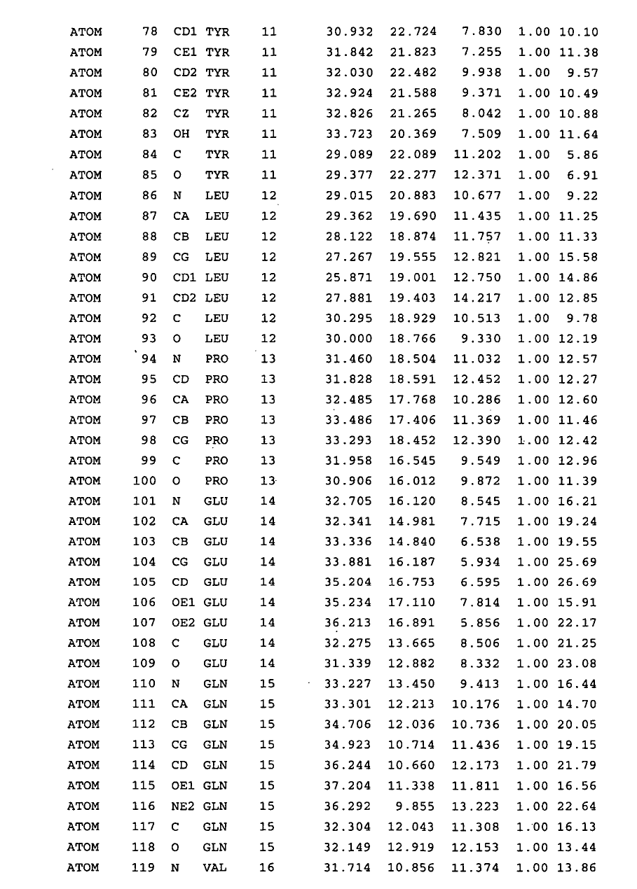

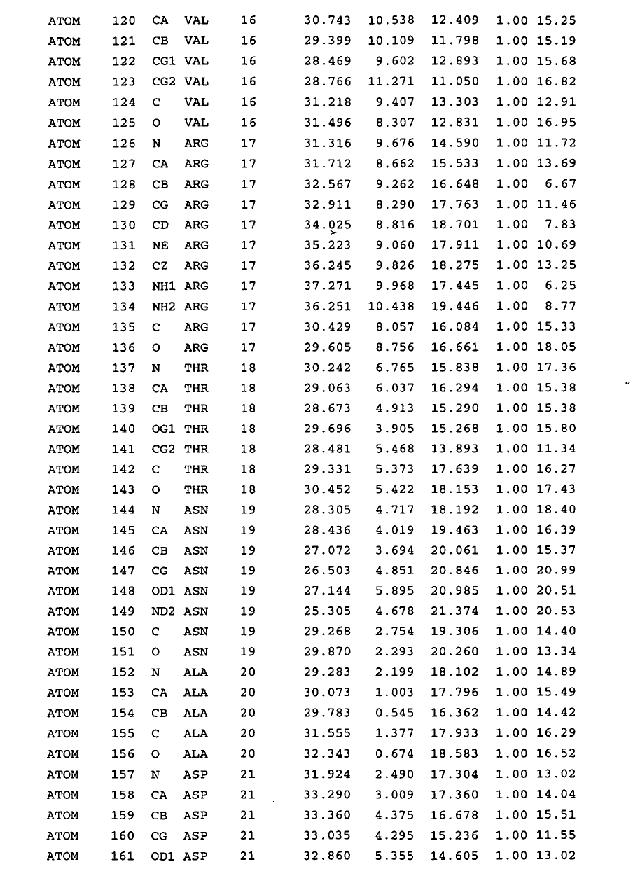

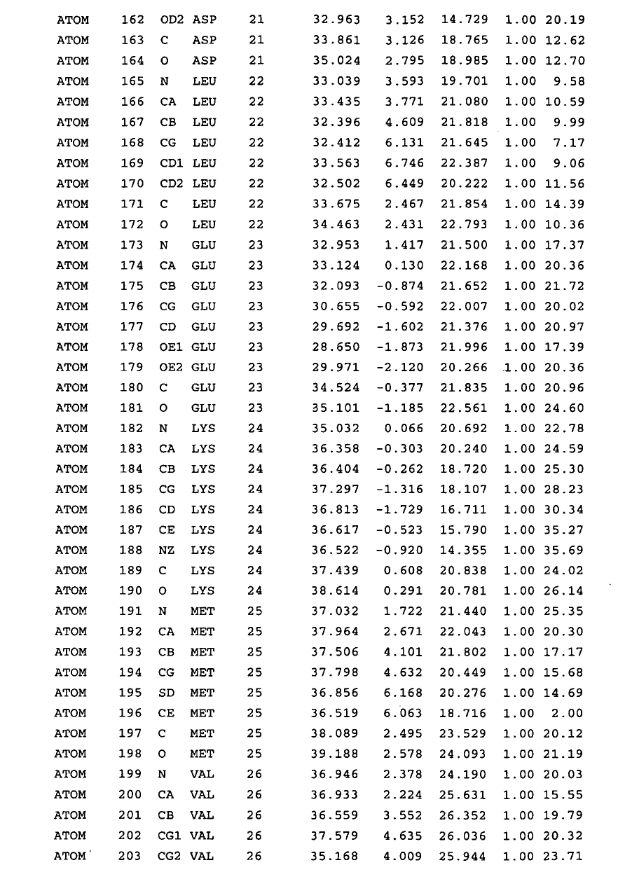

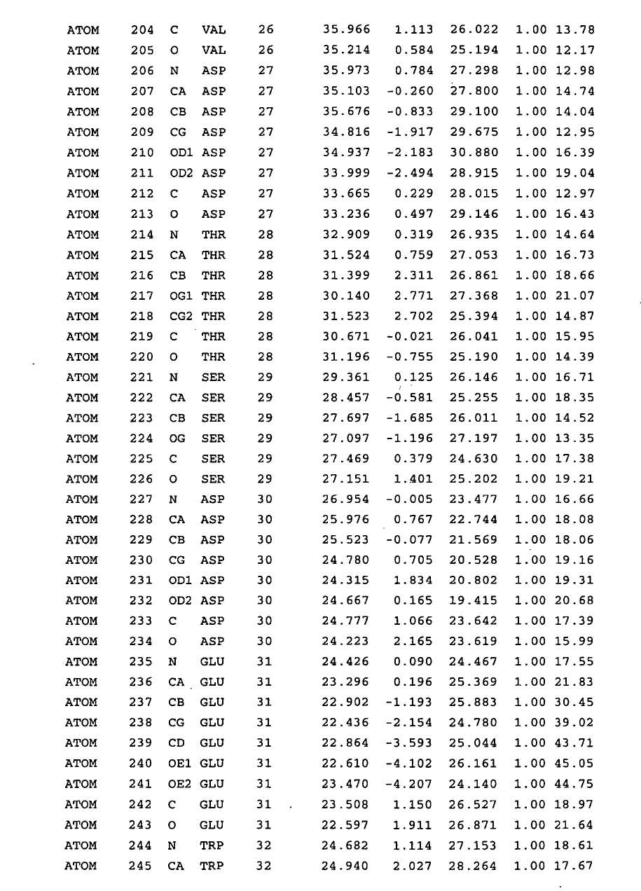

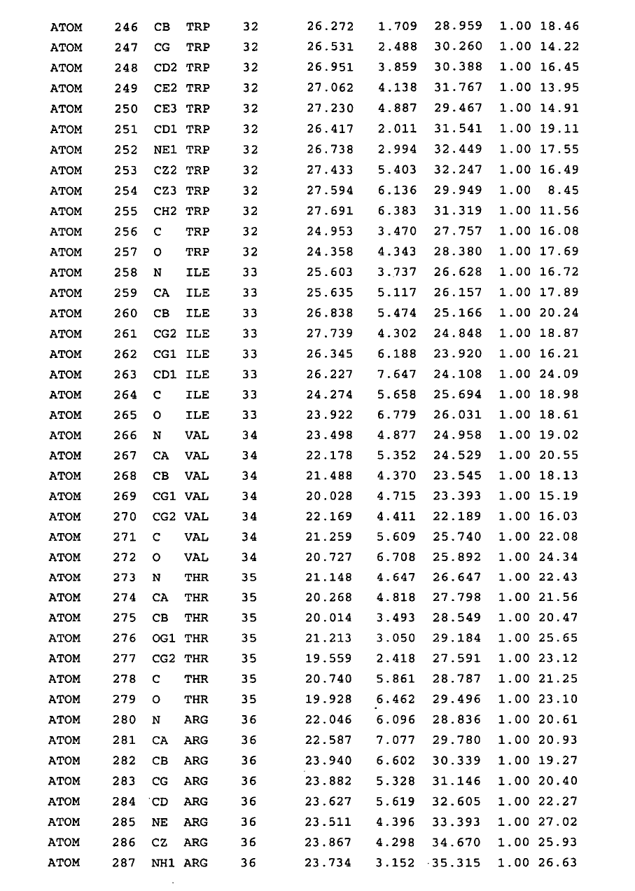

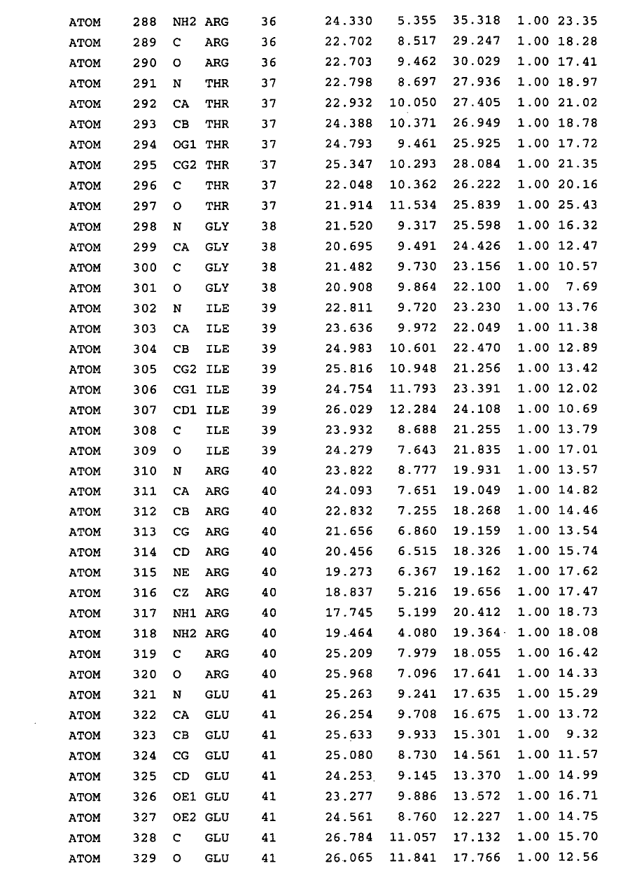

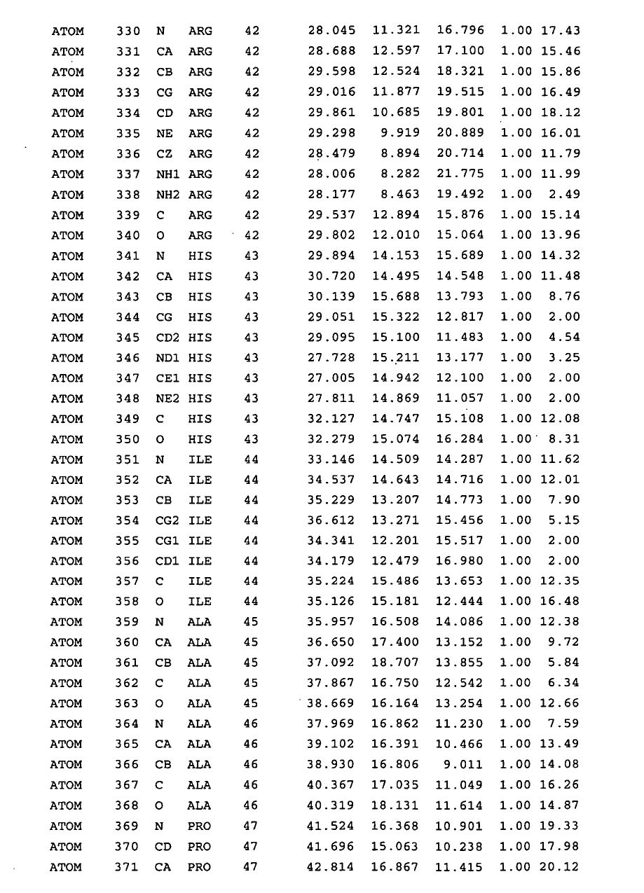

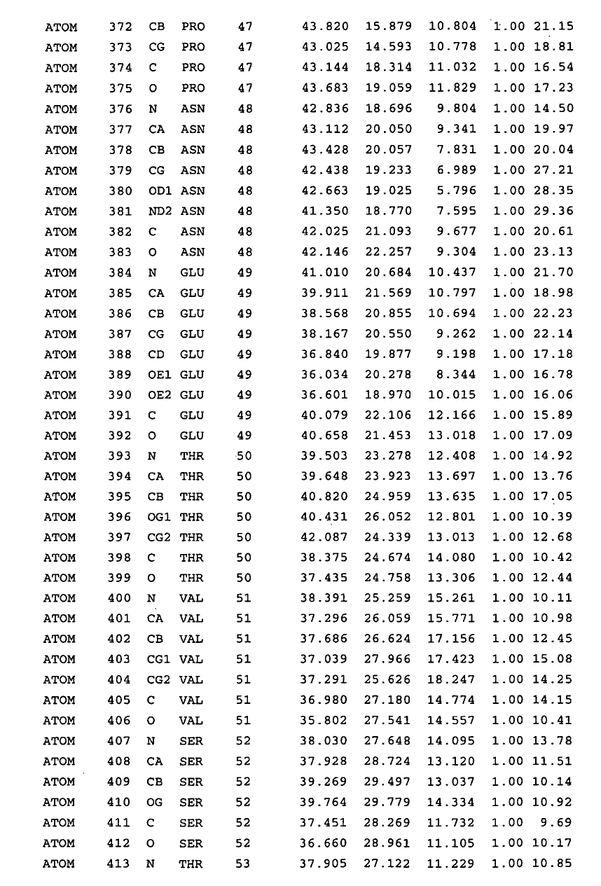

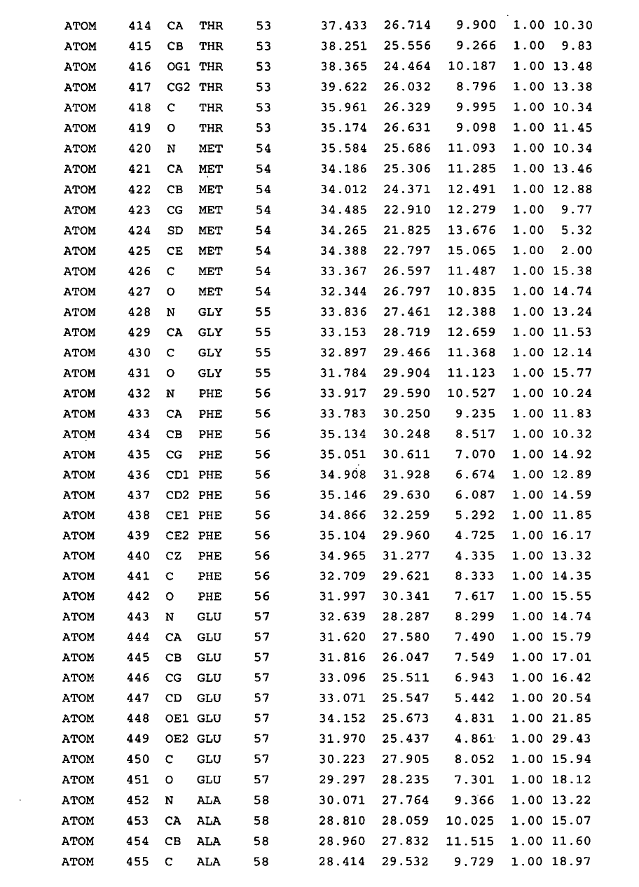

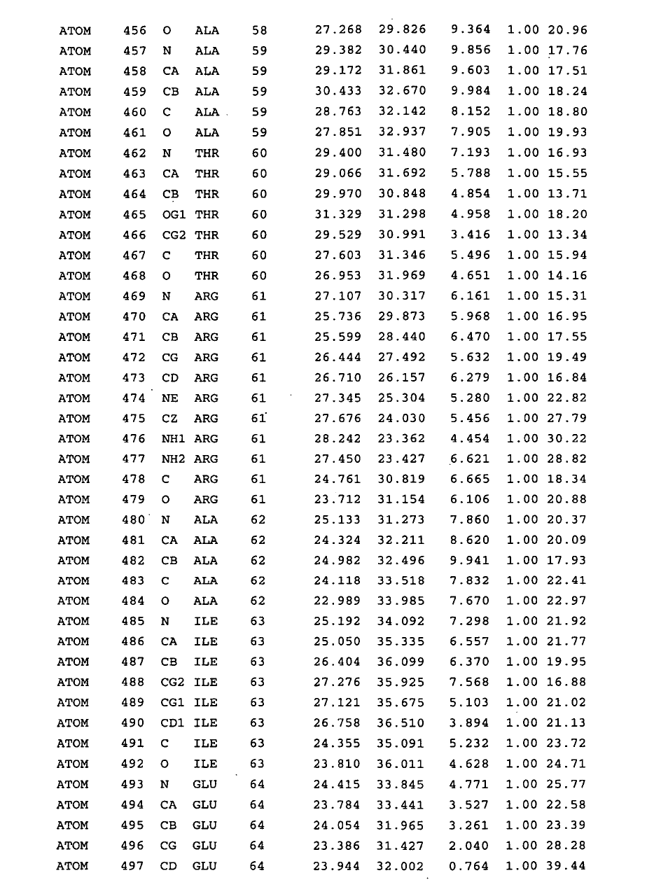

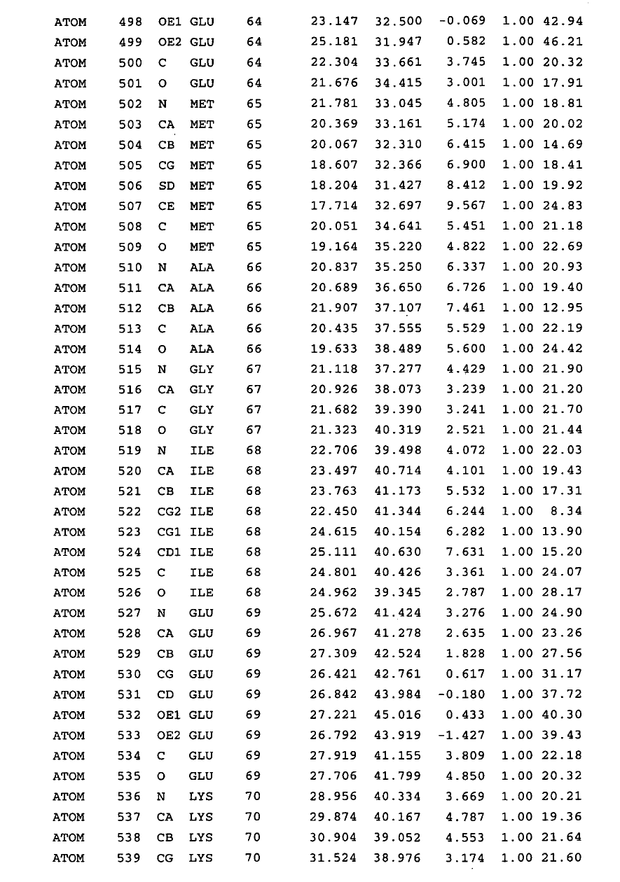

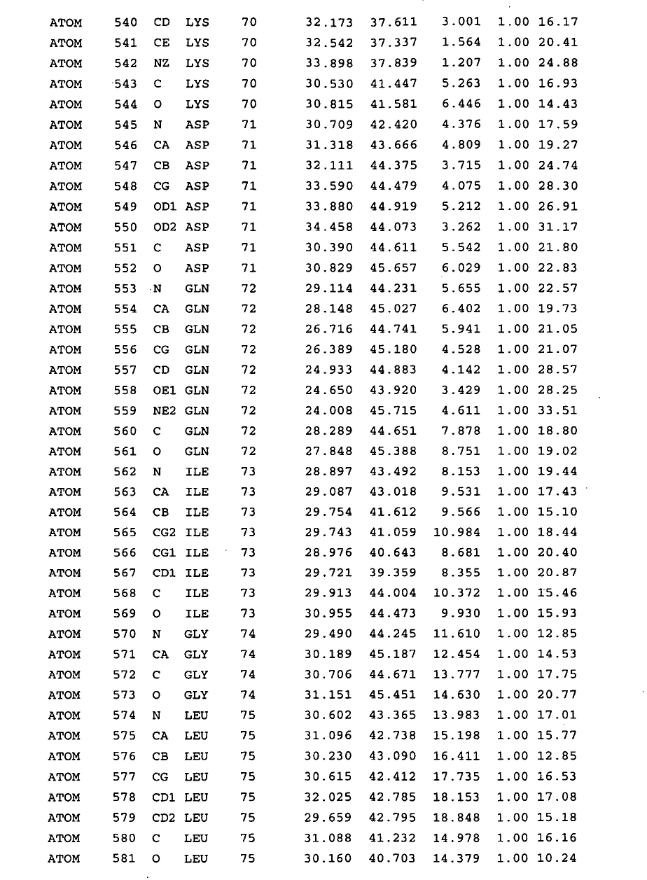

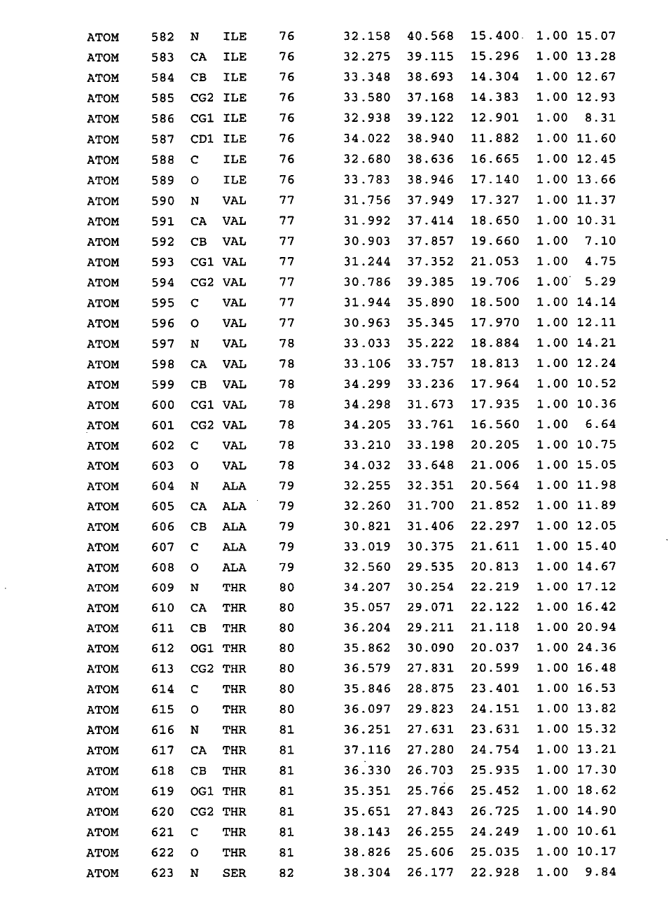

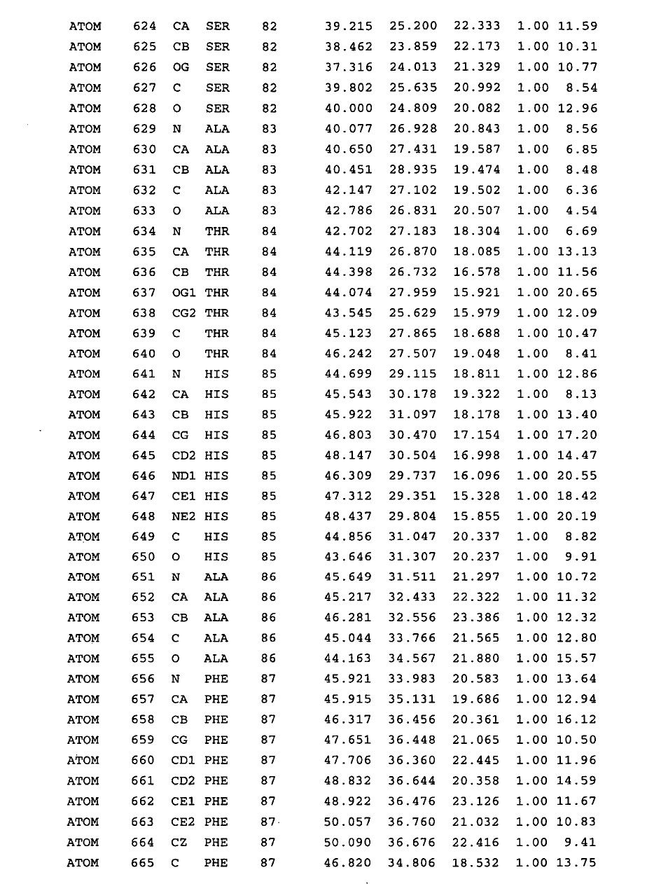

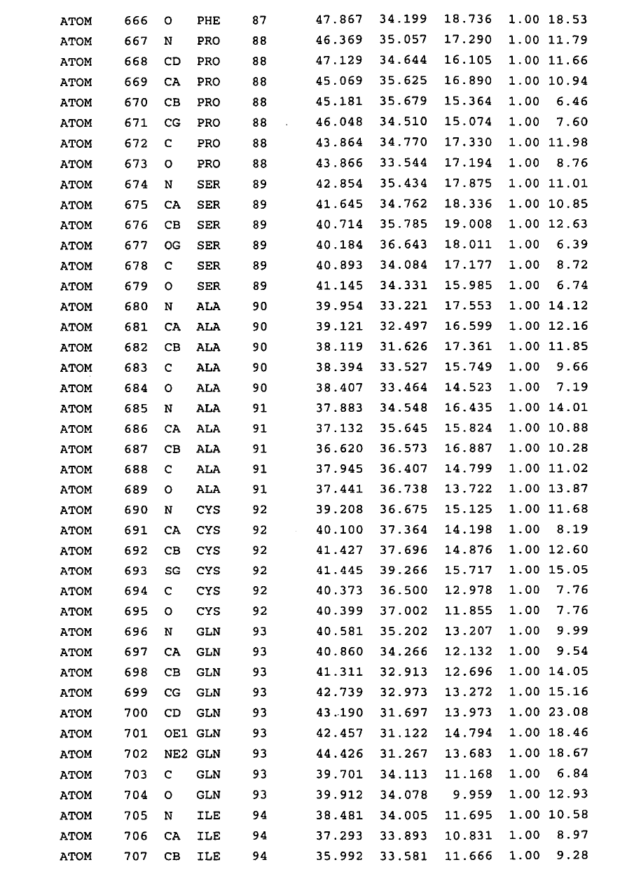

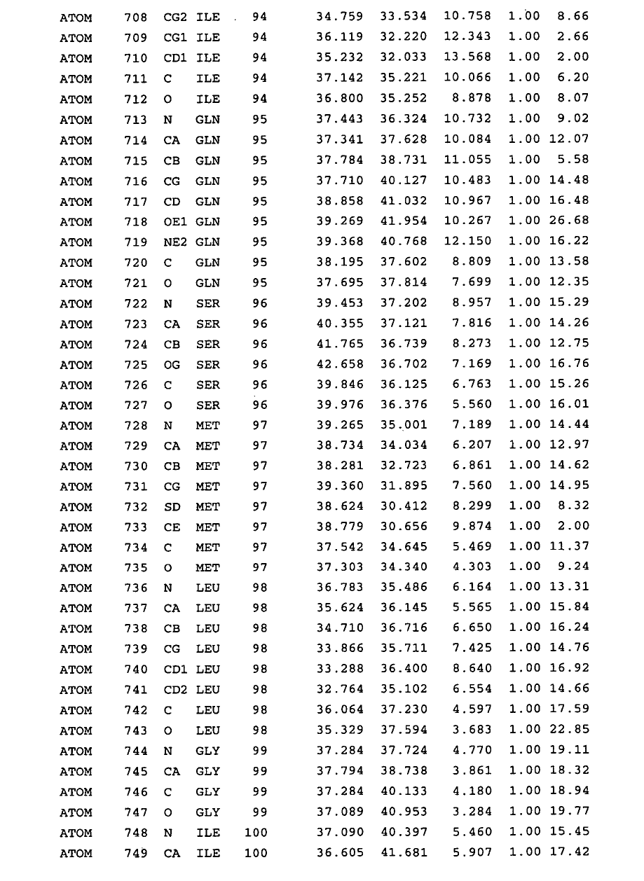

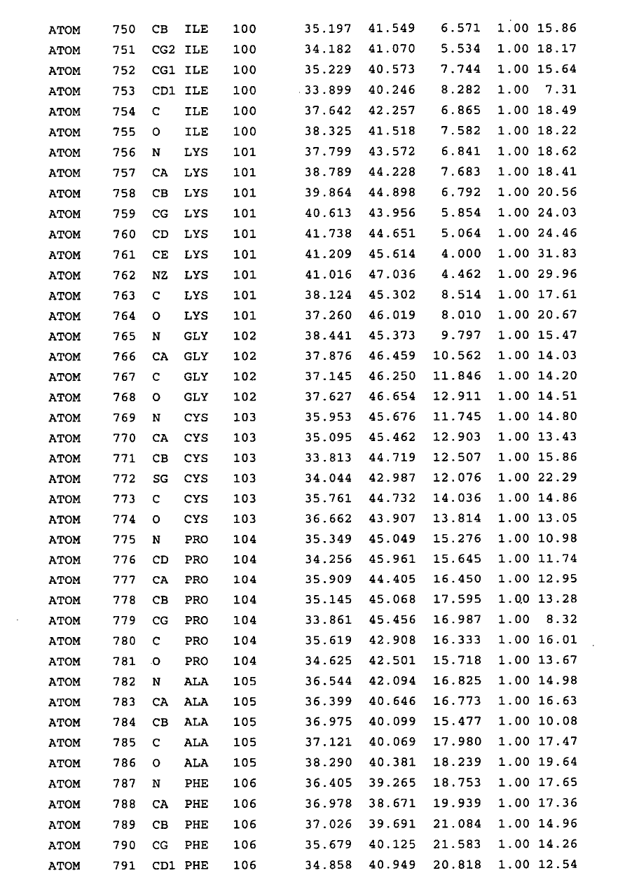

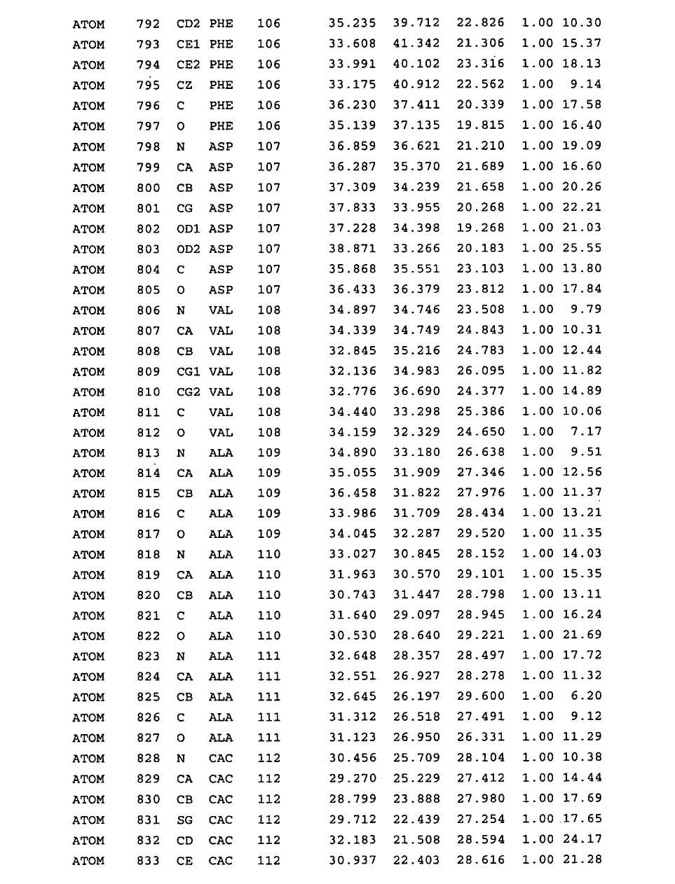

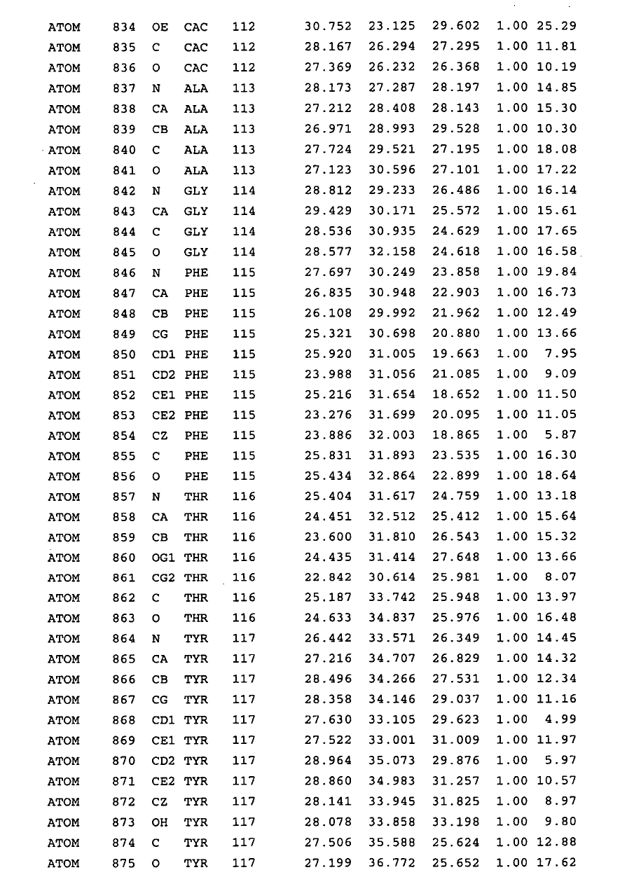

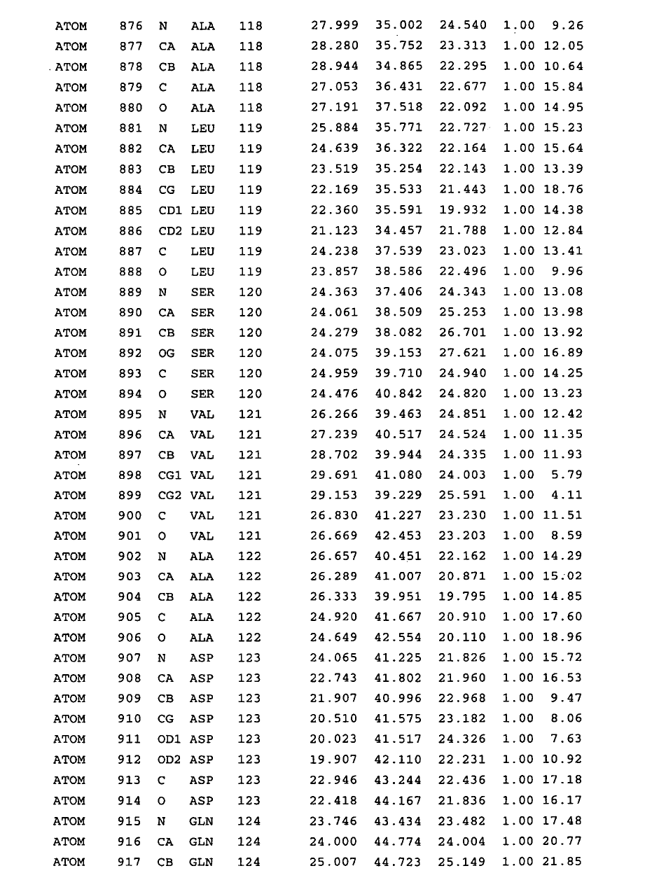

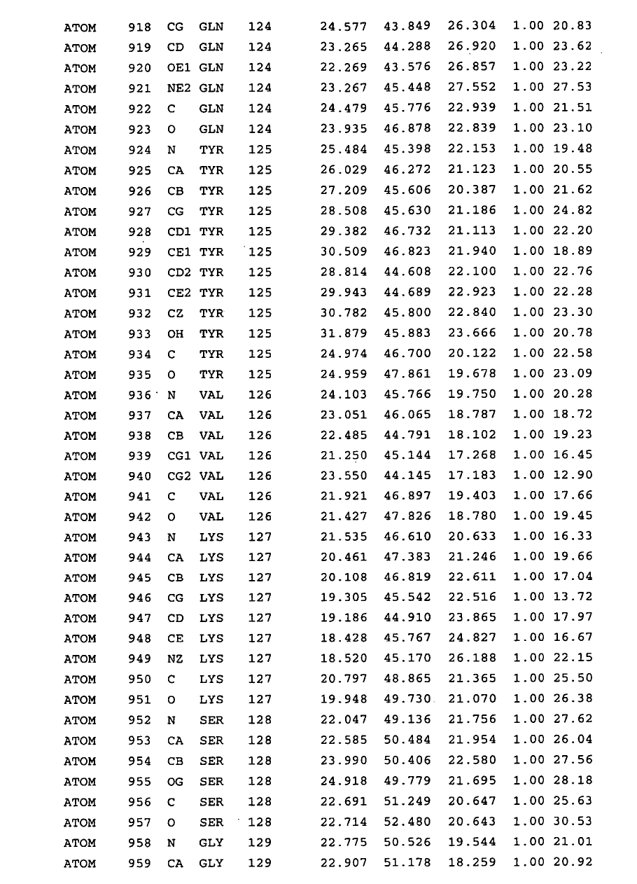

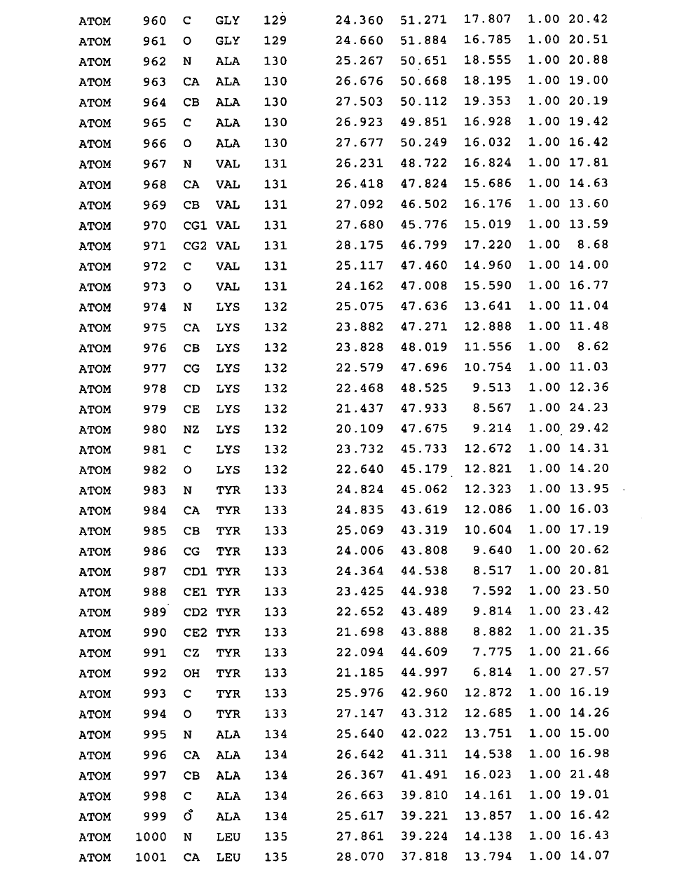

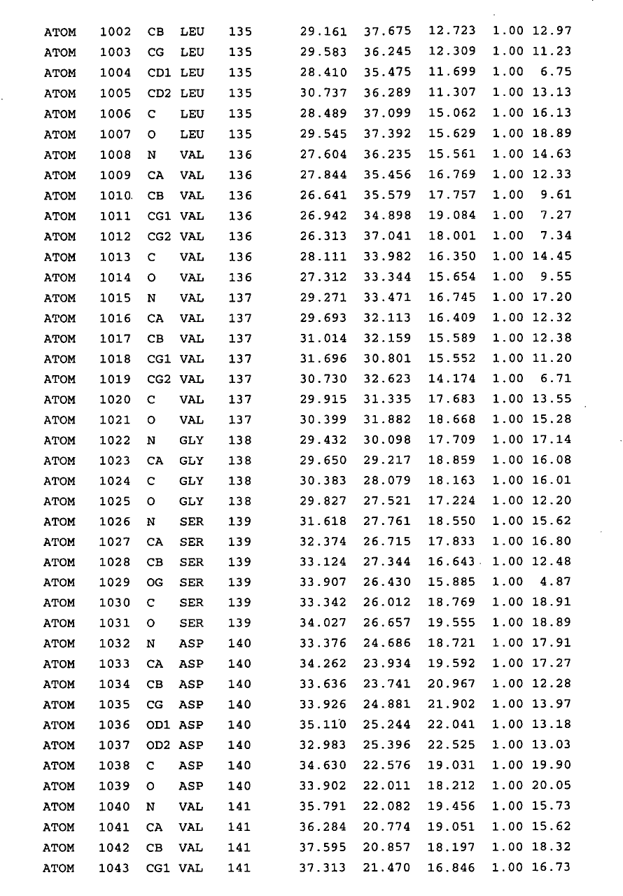

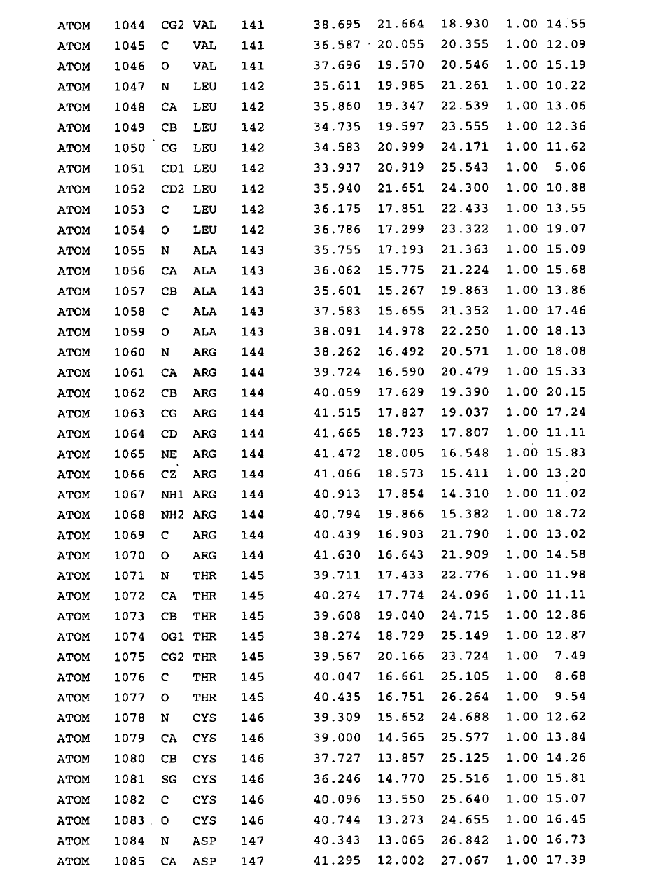

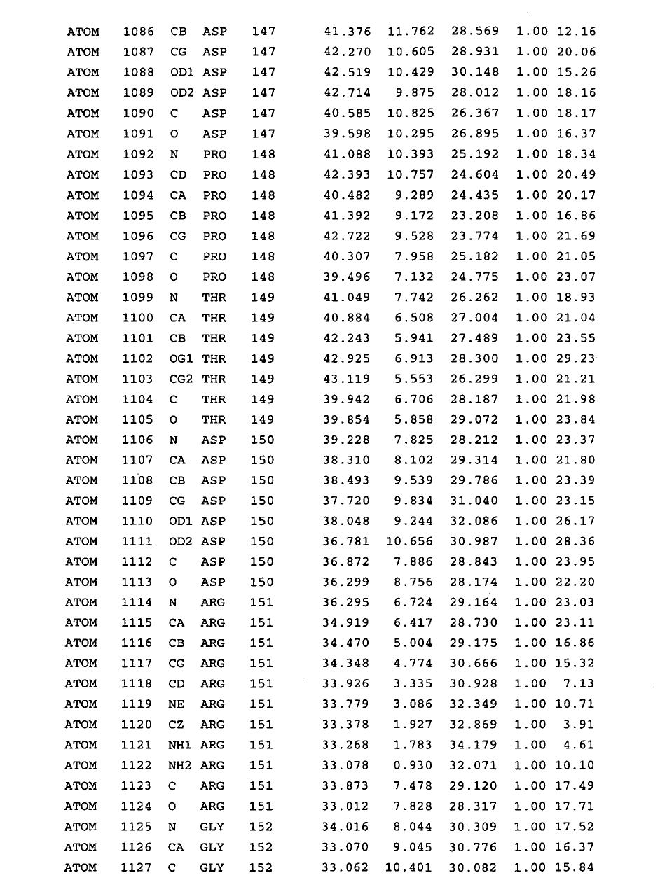

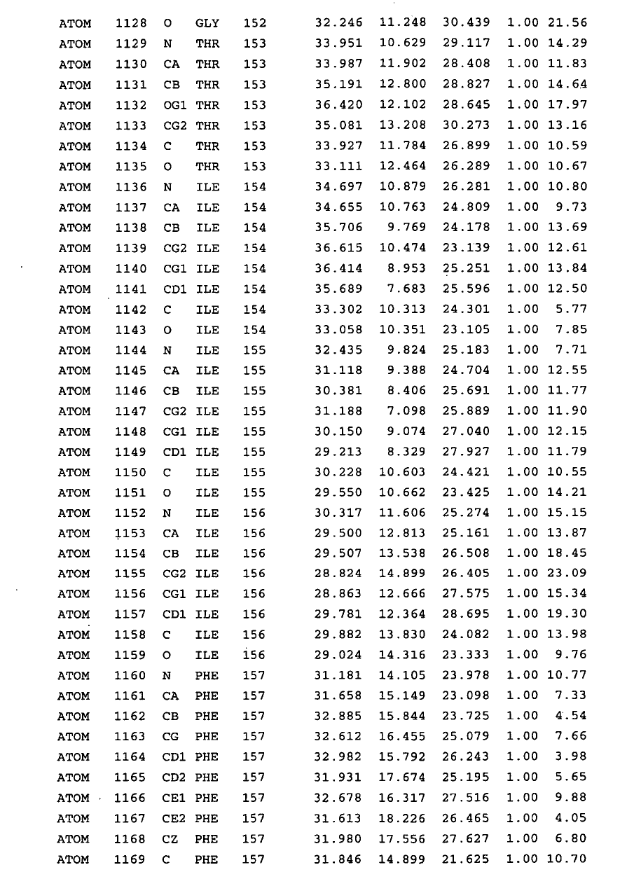

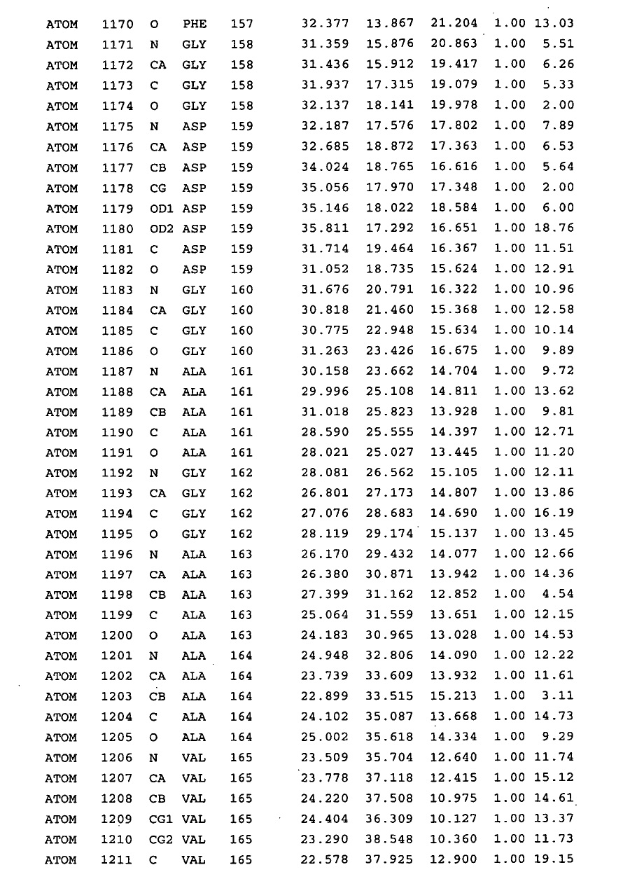

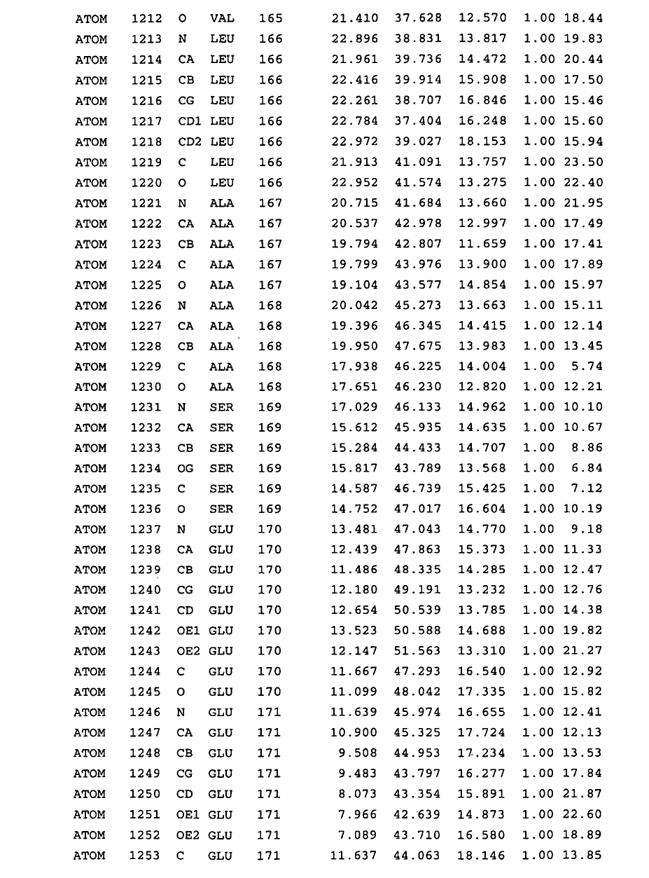

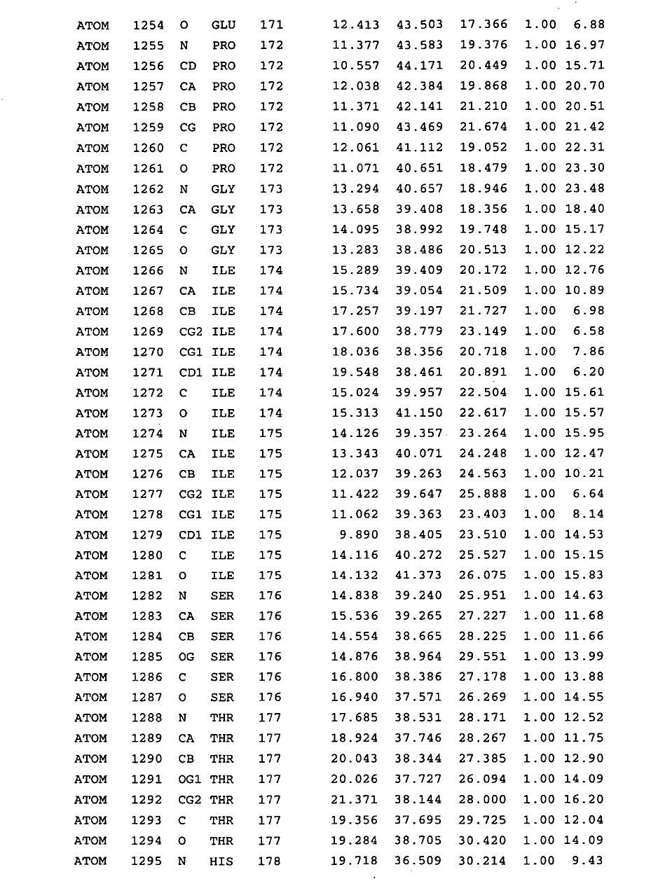

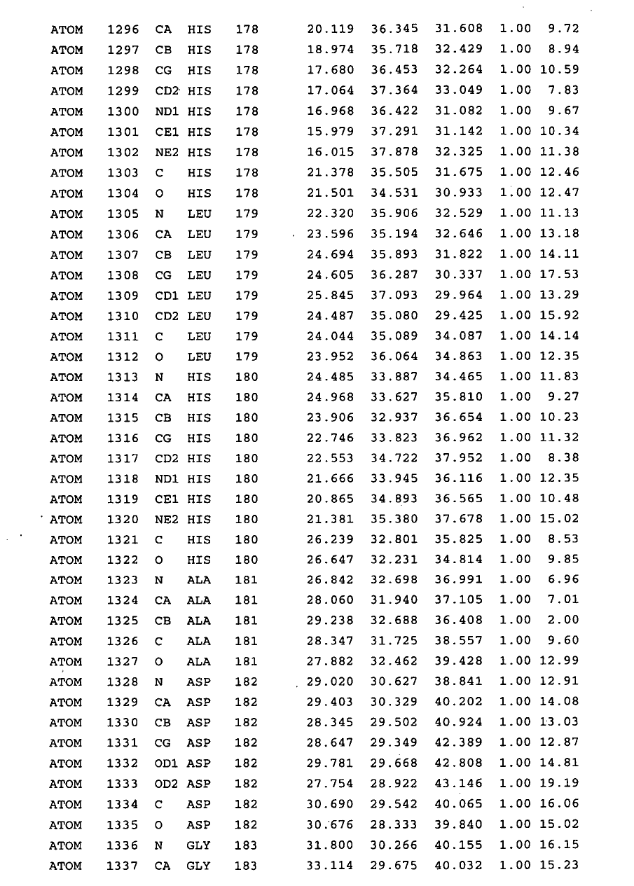

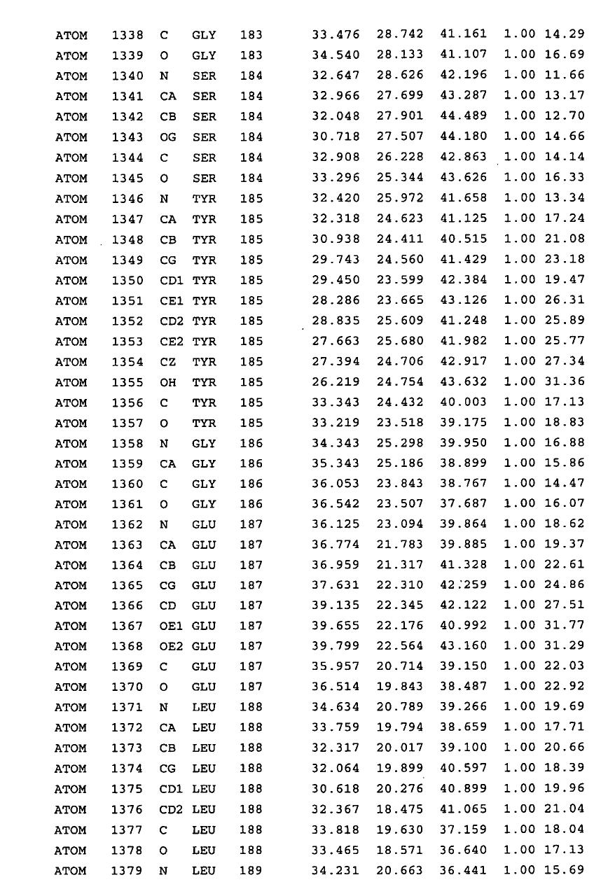

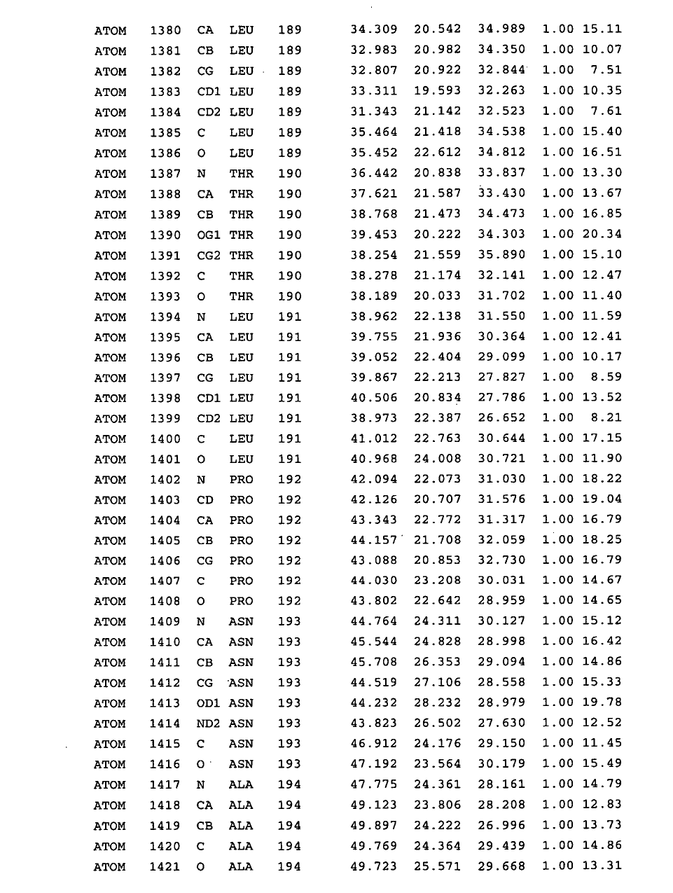

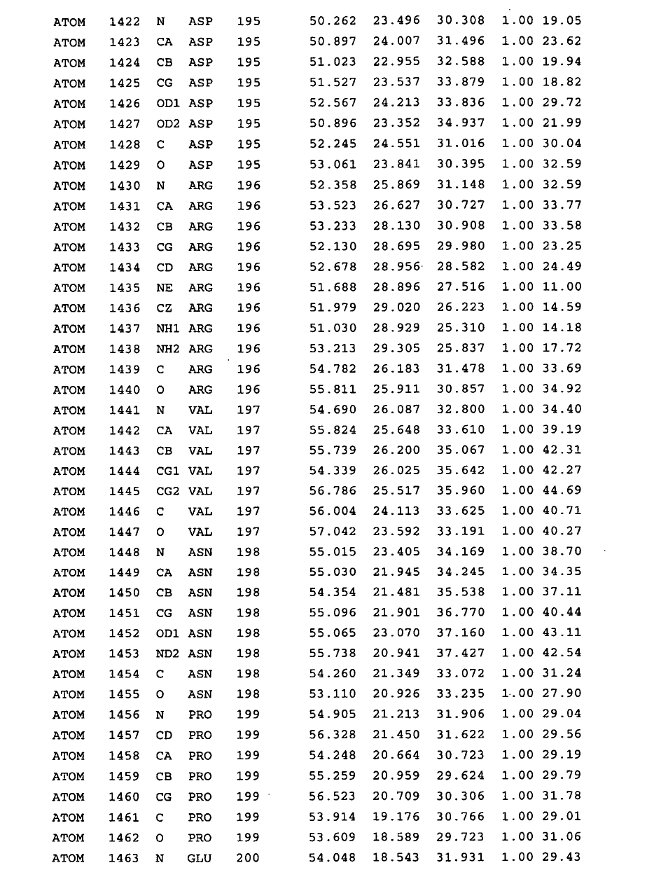

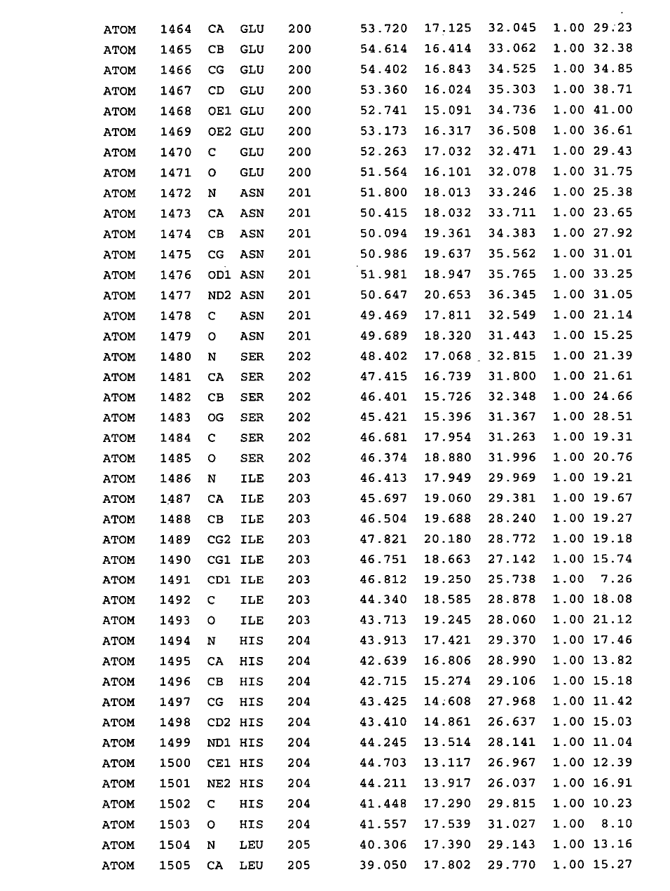

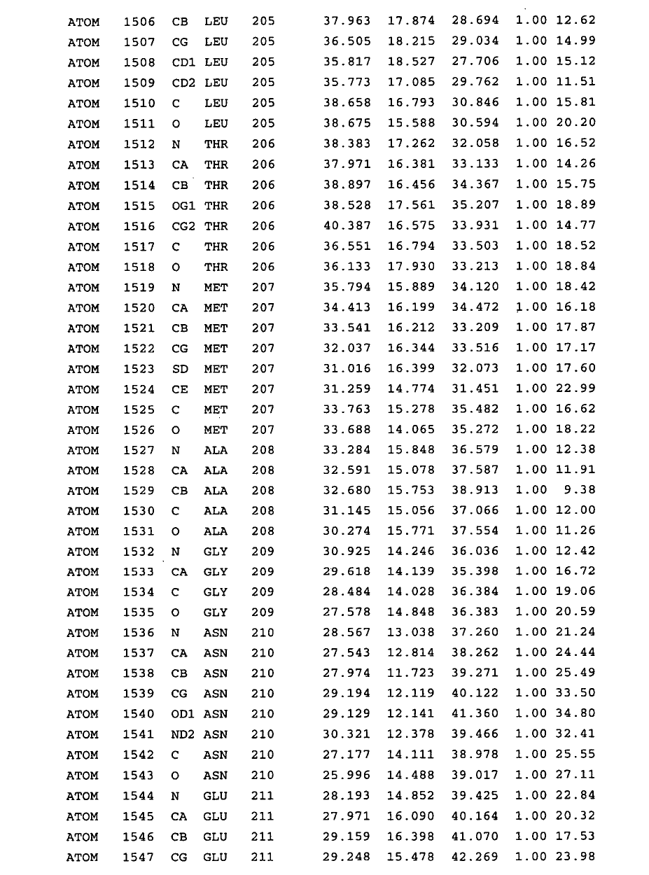

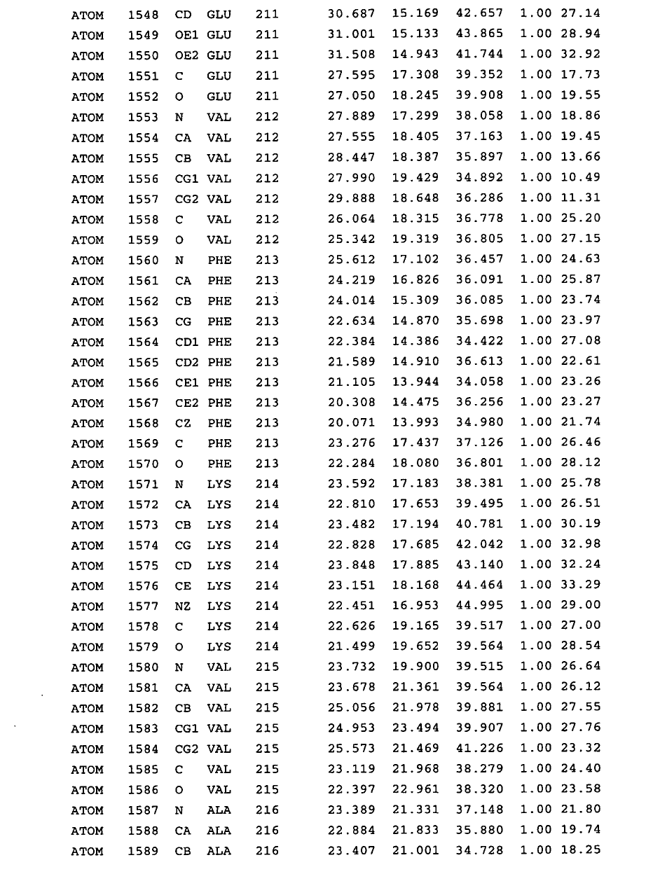

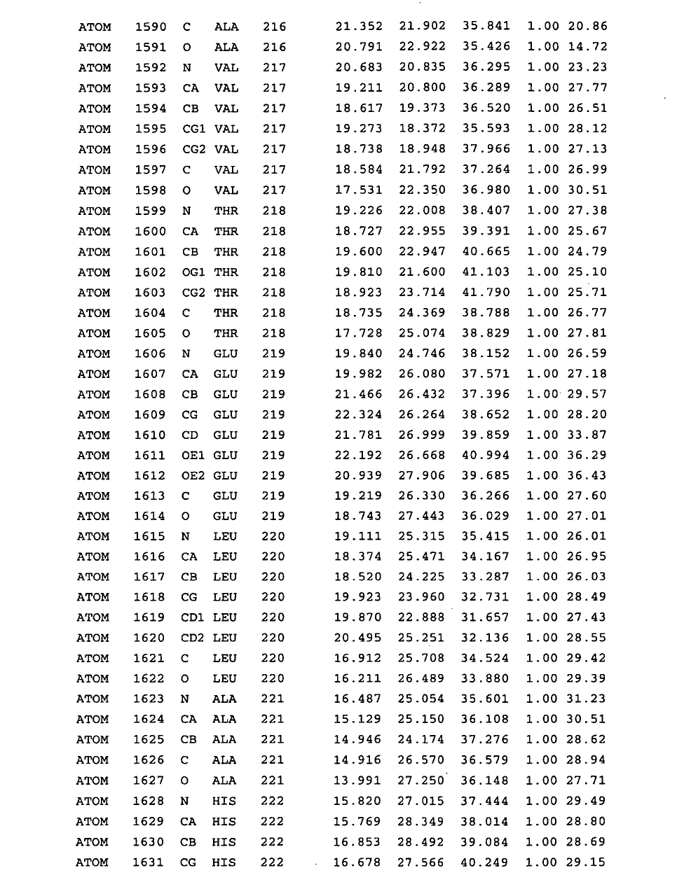

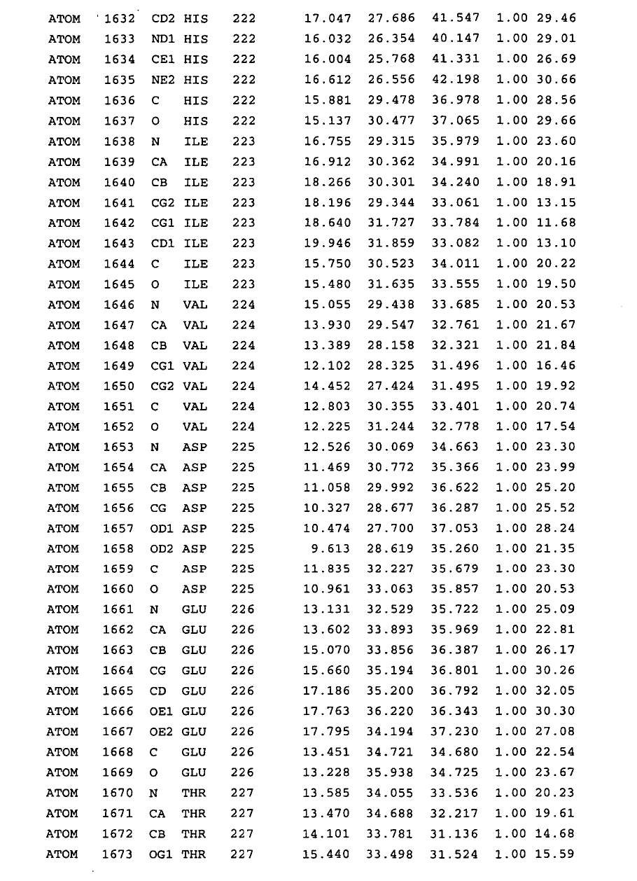

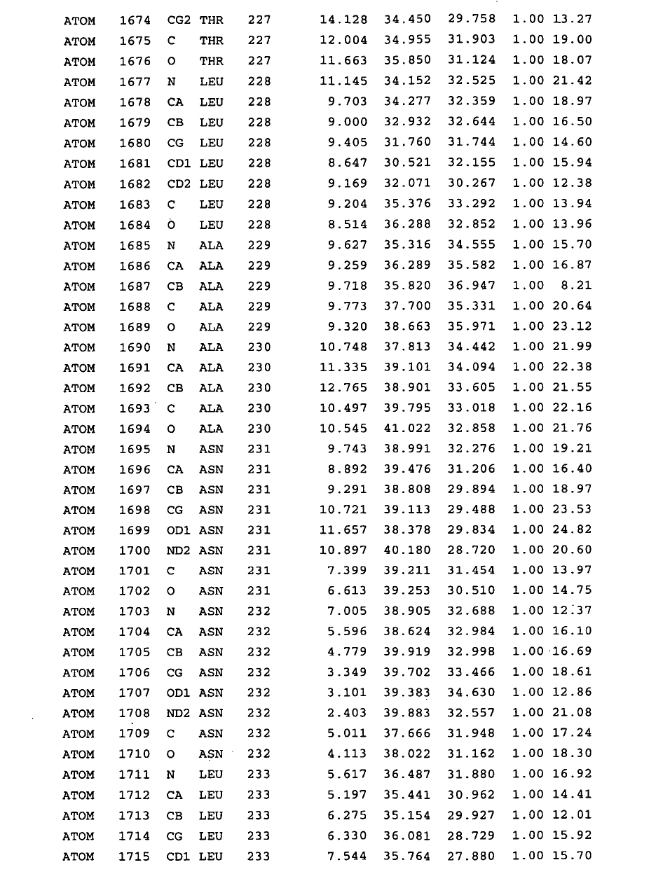

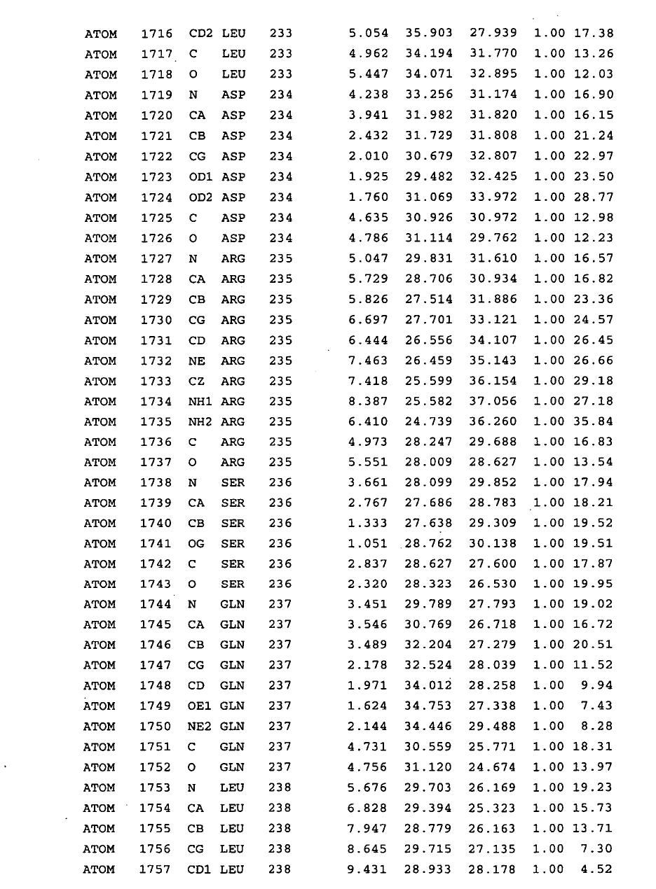

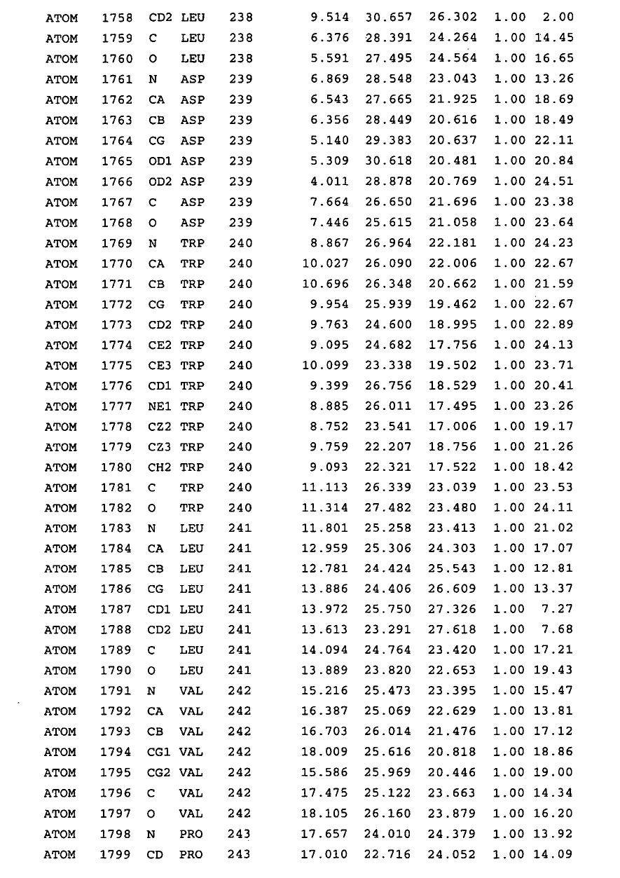

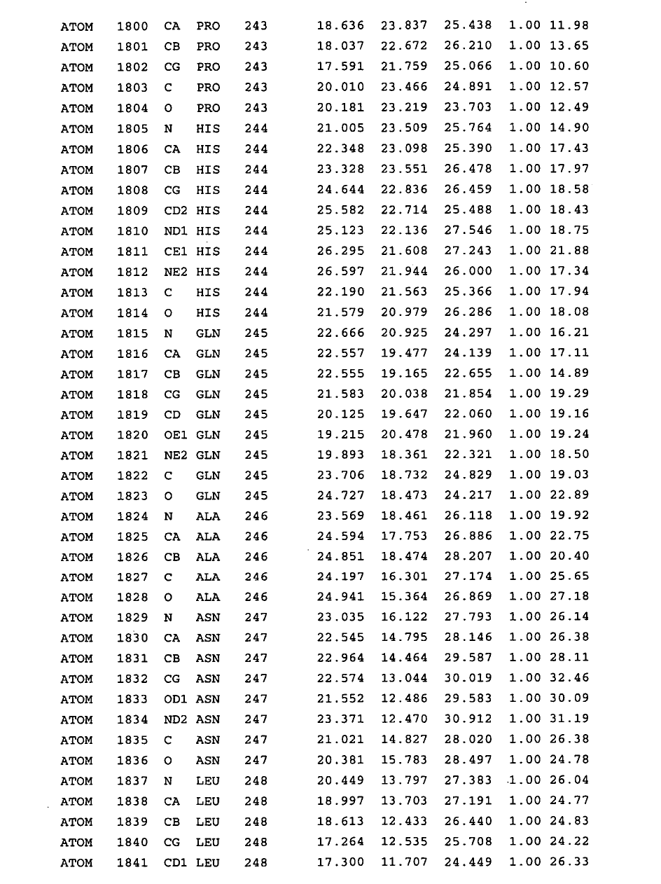

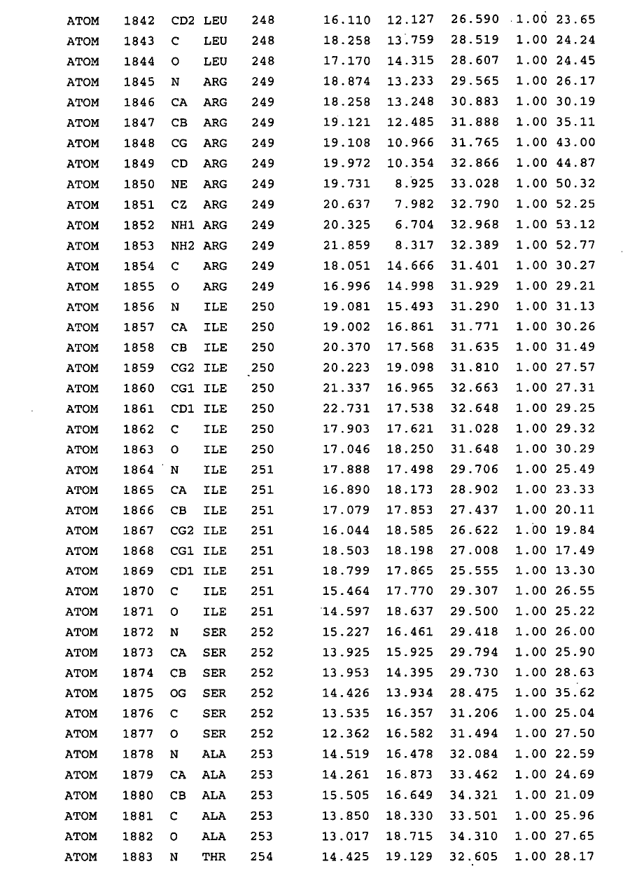

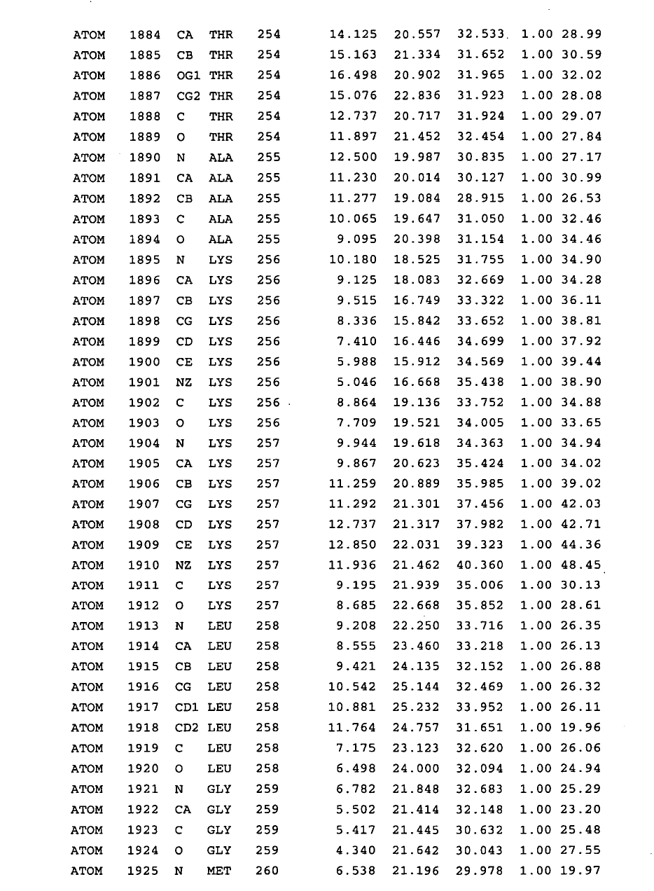

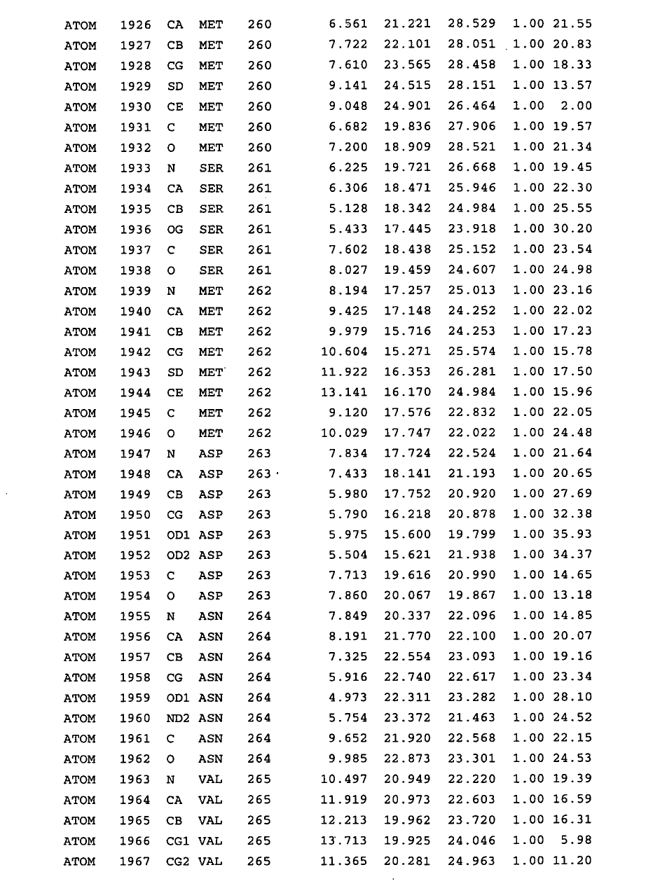

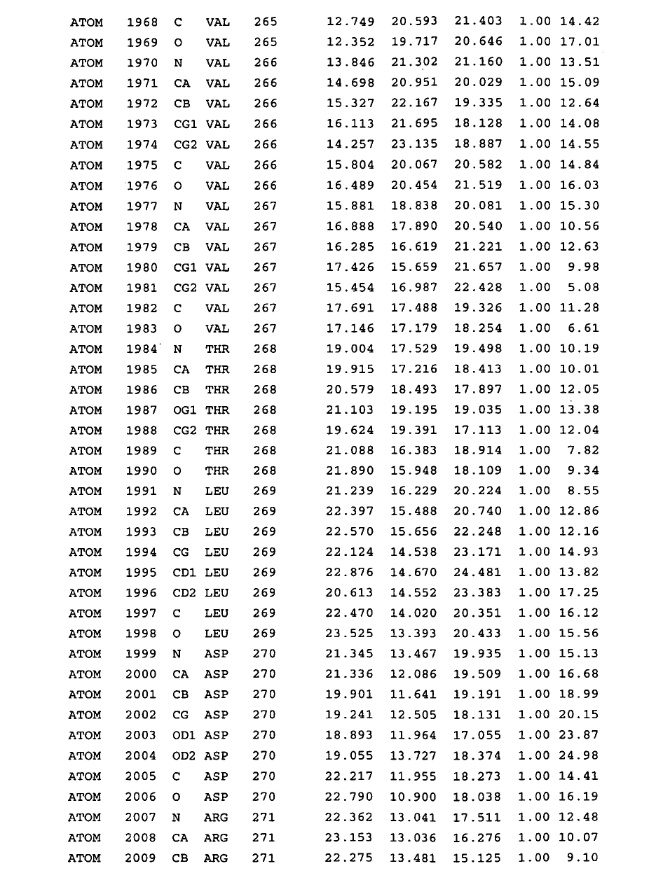

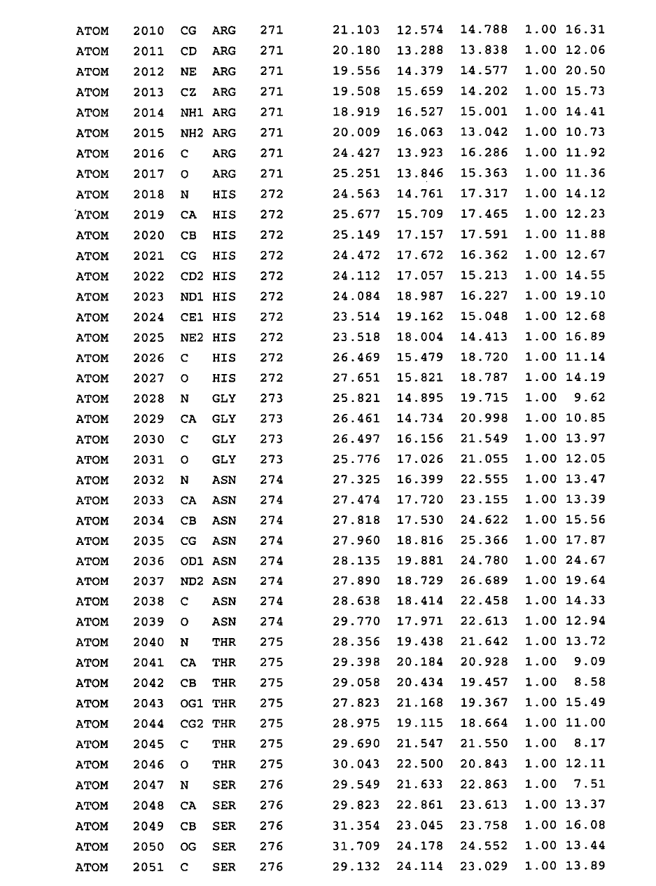

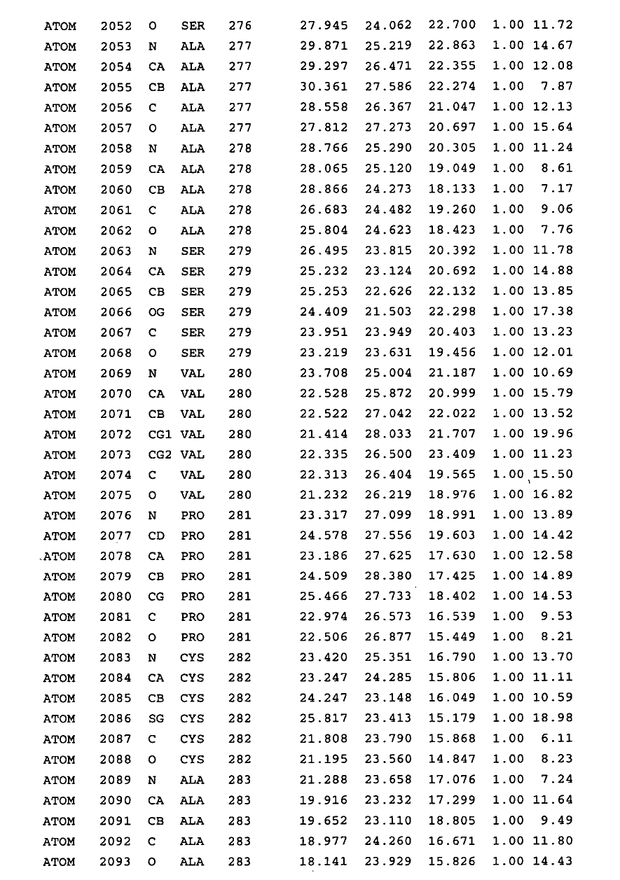

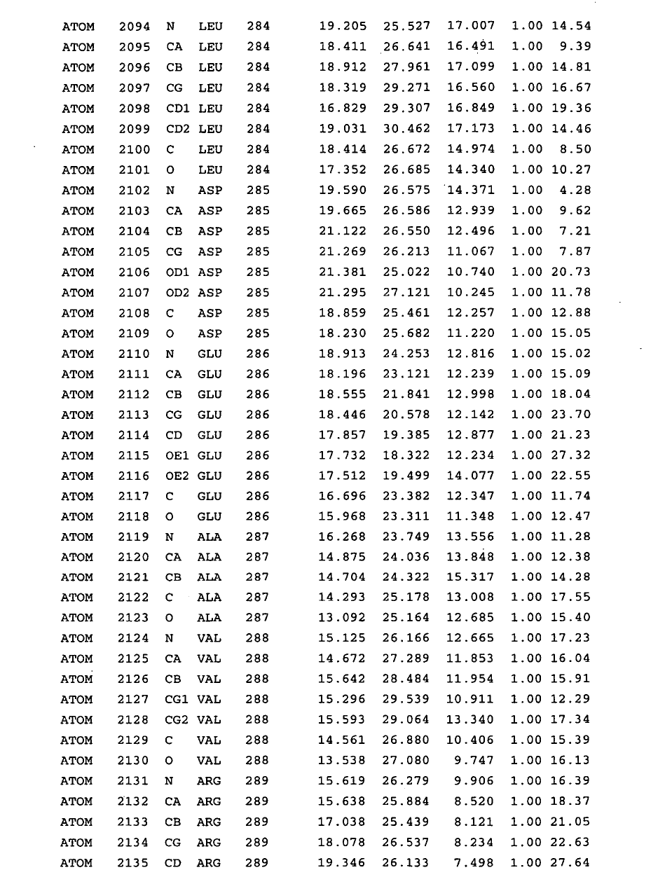

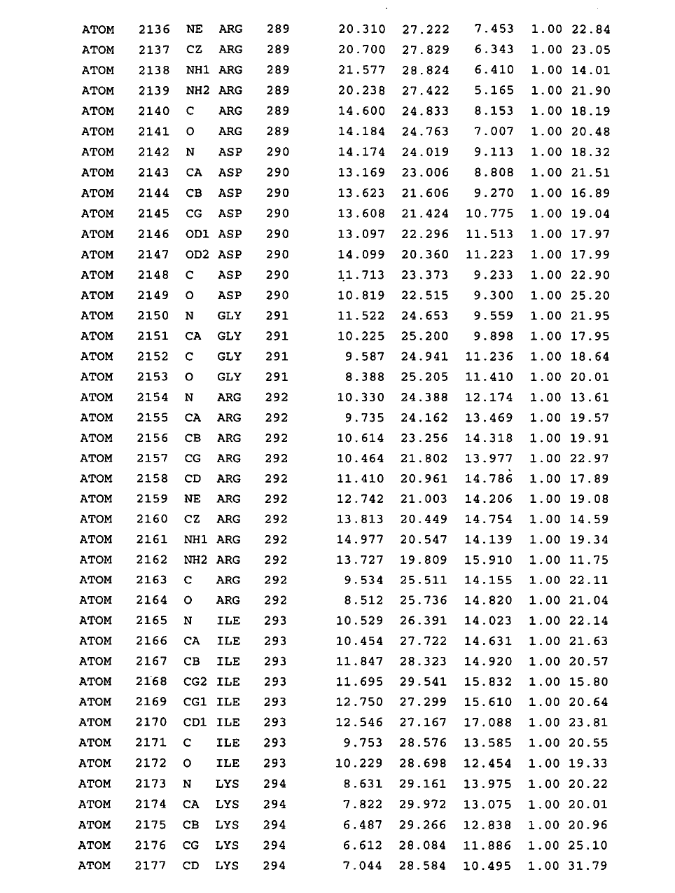

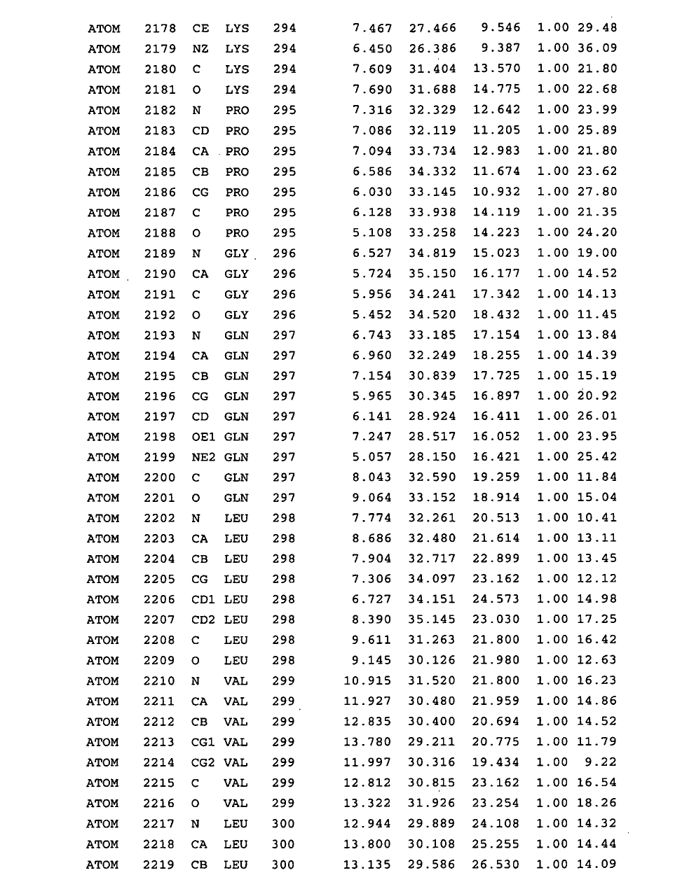

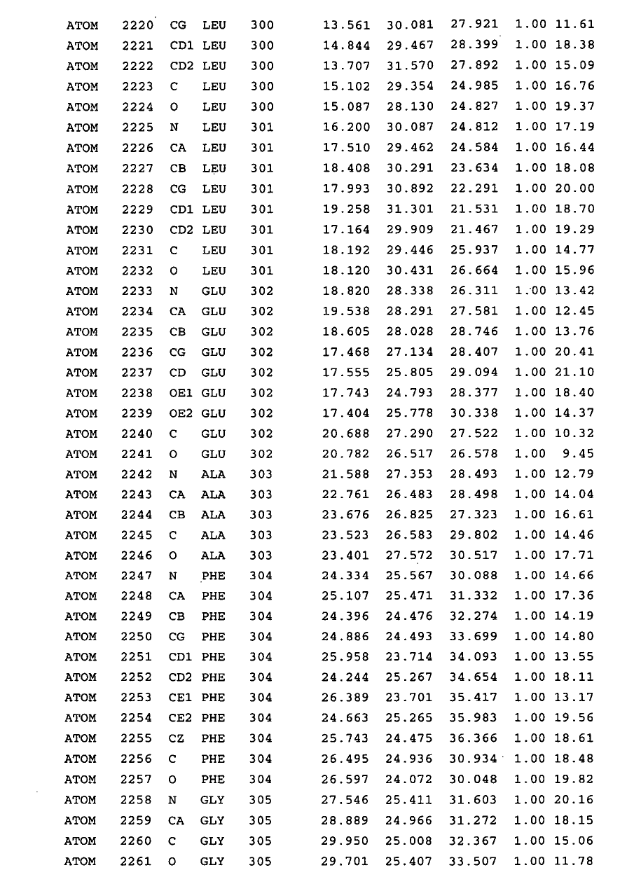

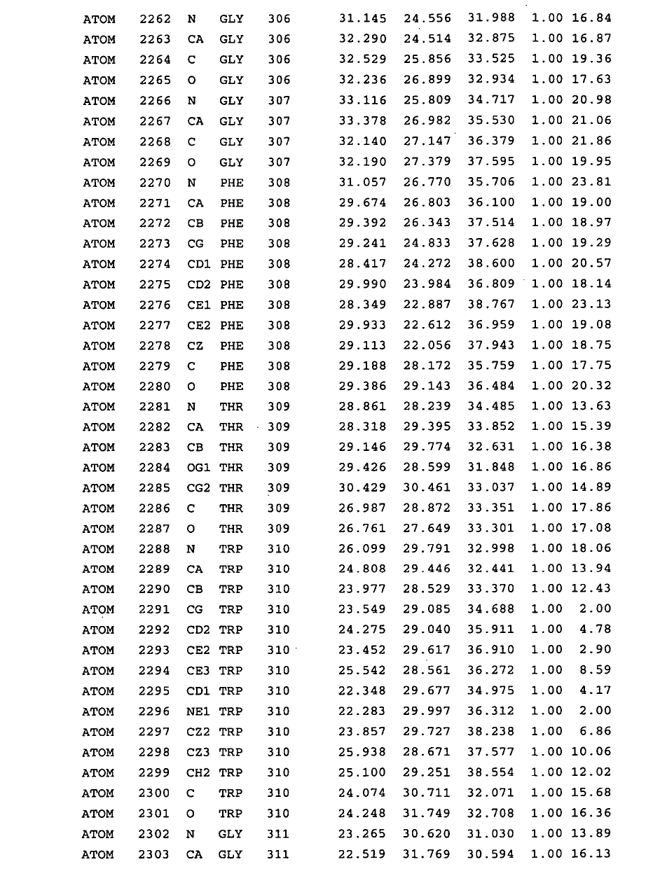

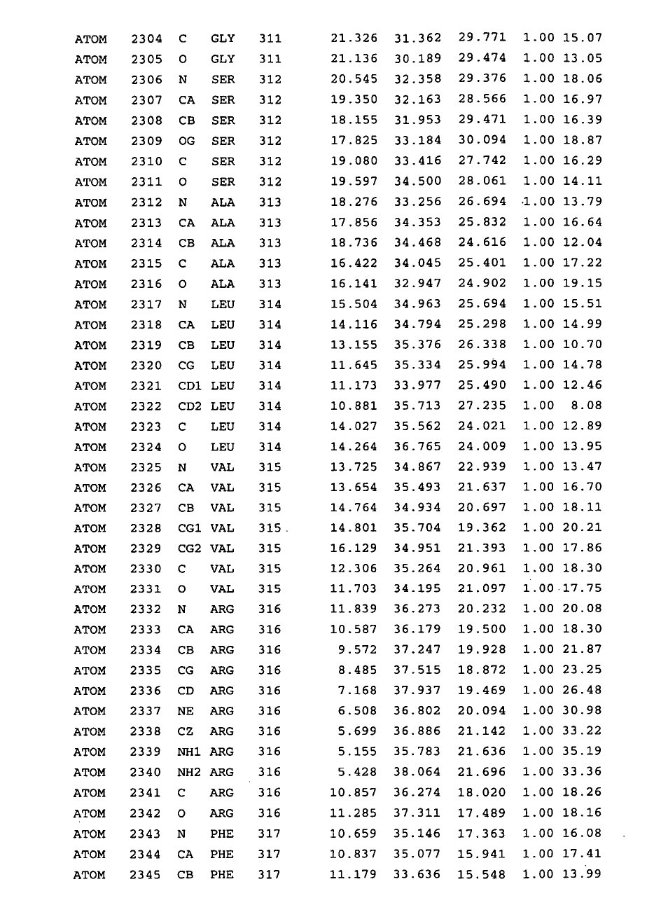

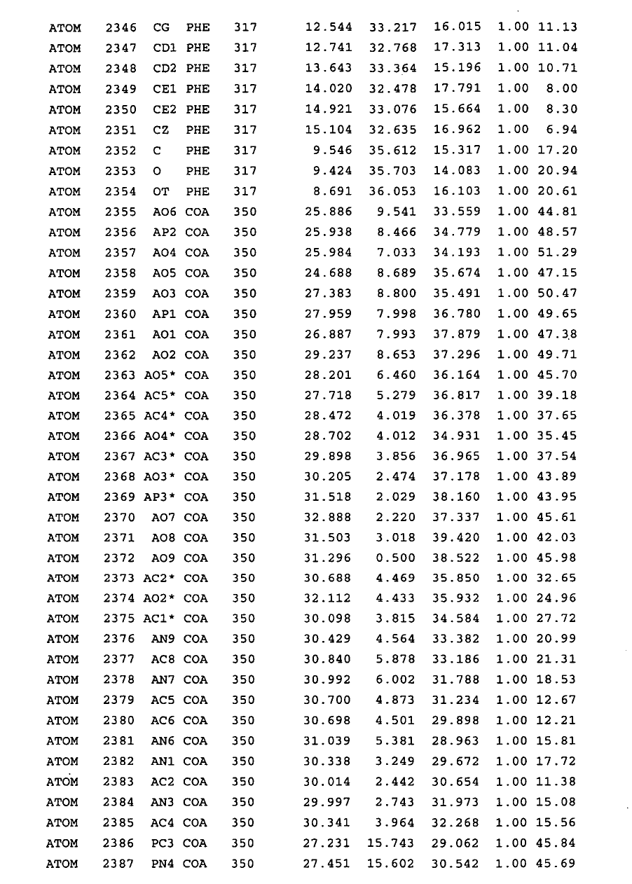

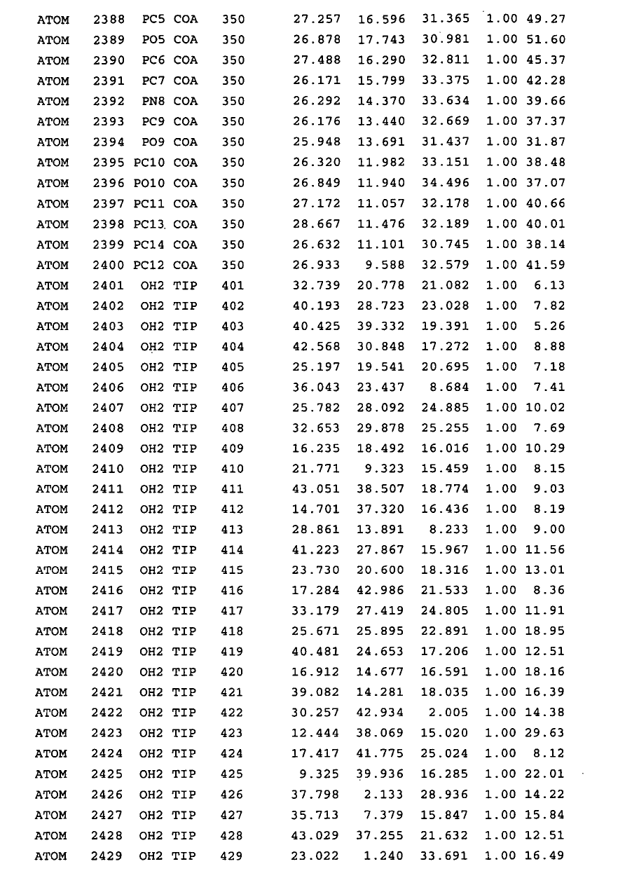

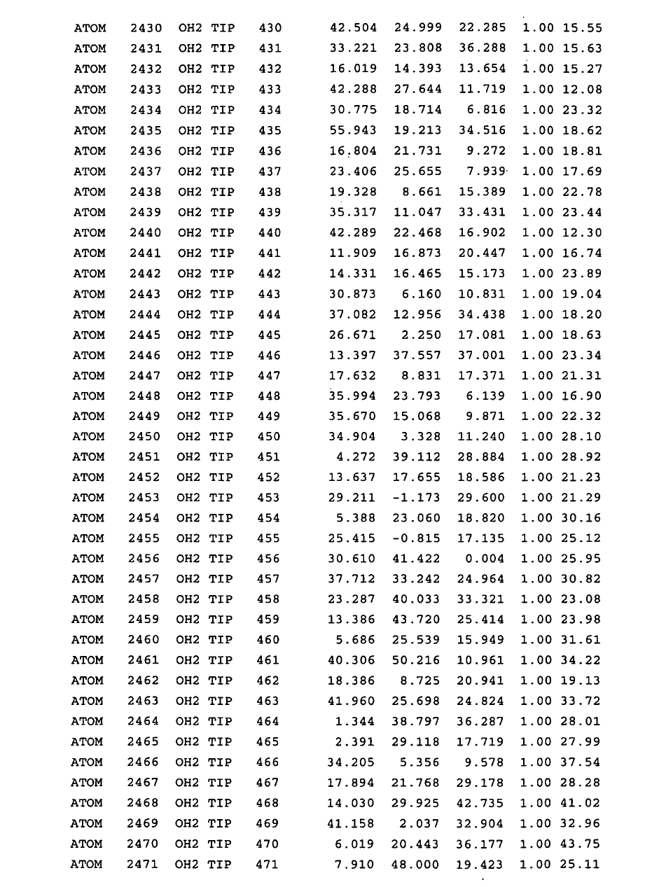

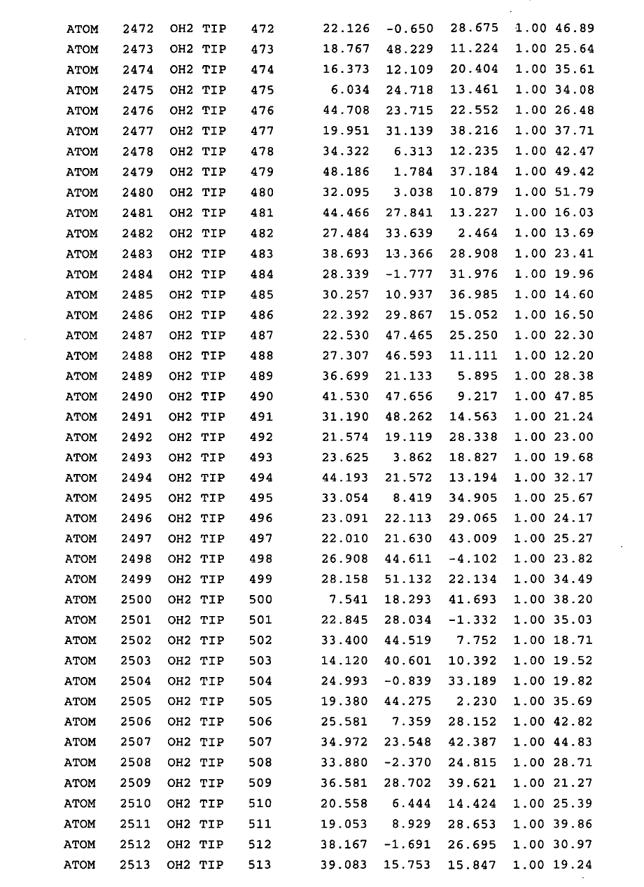

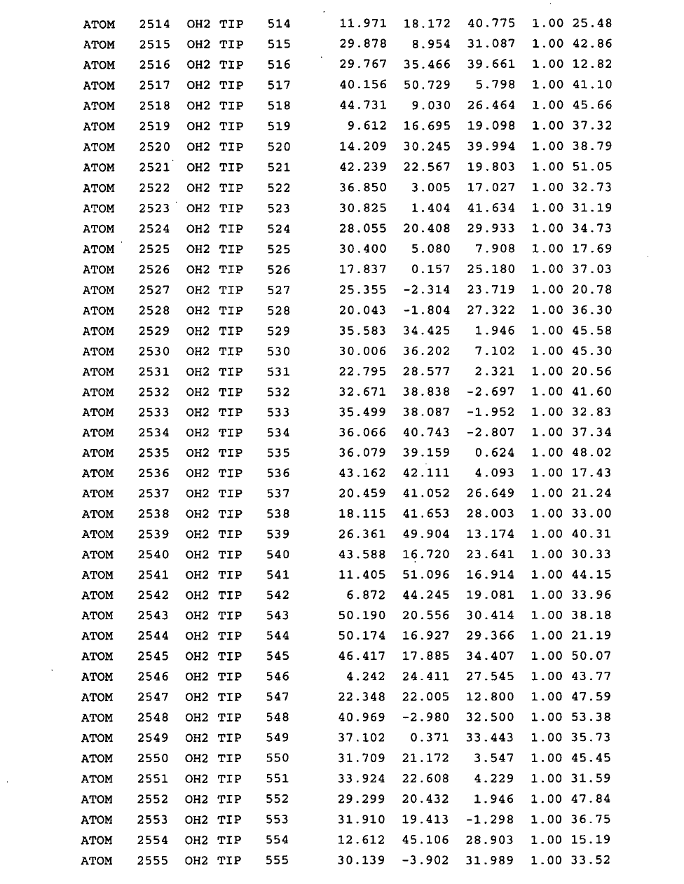

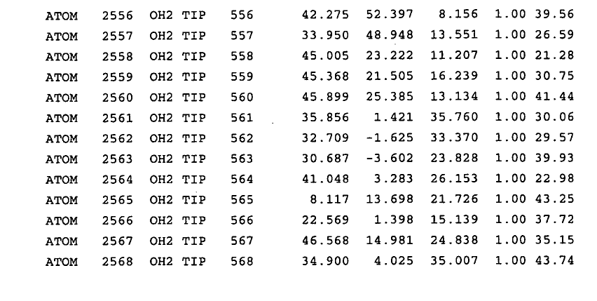

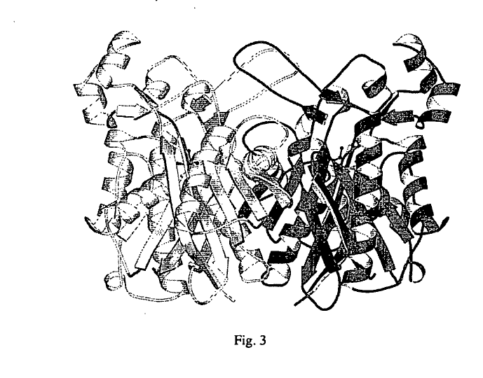

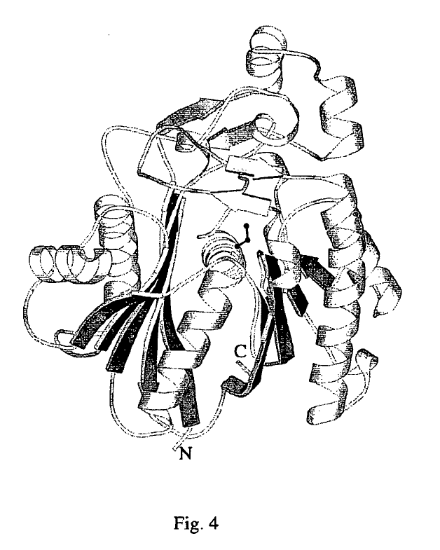

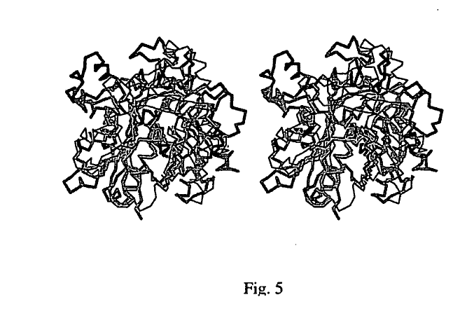

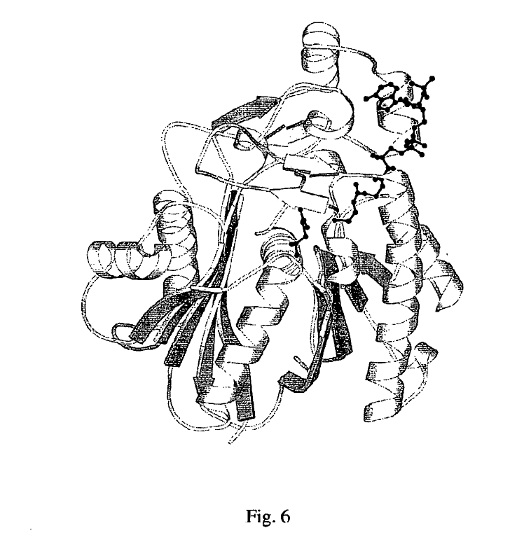

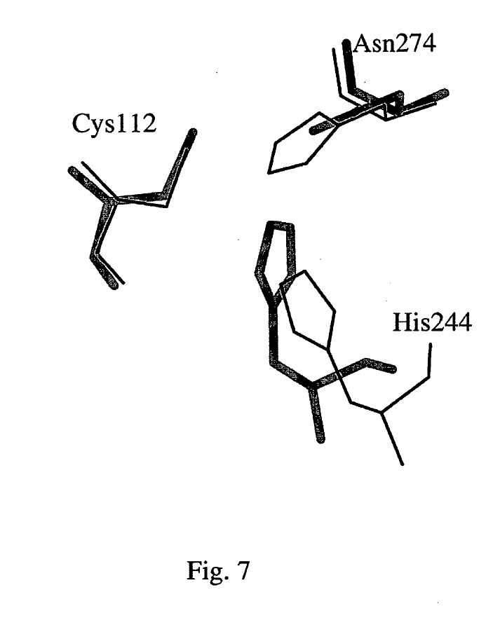

The plasmid pET29c and the negative control pET29 (vector without insert) were used to transform the E. coli BL21(DE3) host strain. Single clones of BL21(DE3): pET29c cells were grown overnight at 37° C. in 2 ml of LB medium in the presence of 0.1 mg/ml kanamycin. The cells were then diluted 100-fold in 10 ml LB with kanamycin. When the cultures reached a value of 0.5 at OD₆₀₀ the fabH expression was induced by addition of isopropyl-thio-galactoside (IPTG) at 0.5 mM of final concentration. After this induction 2 ml samples were taken at different times (1 and 2 hours). The cells were harvested in a microfuge for 3 min, the pellets were washed with 20 mM Tris-HCl pH 8, 1 mM PMSF and resuspended in 100 ul of SDS-PAGE gel-loading buffer. The cells were broken by sonication (15 seconds). The samples were then boiled for 10 minutes and after one spin, 10 ml fractions were analyzed by SDS-PAGE according to the methods of Laemmli [U. K. Laemmli, Nature 227, 680-685 (1970)]. The 4-12% polyacrylamide gels [NOVEX] were stained with Coomassie blue. As shown in FIG. 3 good expression levels were detected from the early stages after induction with IPTG. The evidence was the presence of a prominent band (lanes 2, 4 and 6 in FIG. 3) that was in good agreement with the Mᵣ predicted from the primary sequence. The FabH protein has a theoretical molecular weight of about 33,514 Da.

### EXAMPLE 2

### Large Scale Growth and Purification of FabH

A. Large Scale Growth

A 4 liter fermentation of E.coli BL21(DE3): pET29c was carried out in Luria Bertani medium (LB), containing 100 ug/ml kanamycin. The 8×500 ml flasks were inoculated at 1% (v/v) from an overnight secondary seed culture in single strength LB medium, containing 100 mg/ml kanamycin. The flasks were incubated at 37° C., agitated at 250 rpm in a benchtop shaker. The OD at 600 nm was monitored, and at 0.5 absorbance units, FabH expression was induced with the addition of isopropyl-thiogalactosidase to 0.5 mM and the cells harvested by centrifugation in a Sorval CSA rotor, 2 hours post induction. A total of 20 grams of cell paste was recovered.

LB Medium, per liter, contains the following components. The medium was supplied by the in-house laboratory.

Single Strength

| | |
|---|---|
| Bacto Tryptone | 10 g |
| Bacto Yeast Extract | 5 g |
| Sodium Chloride | 5 g |

B. Purification

1) Lysis

12.5 g of cells of E. coli overexpressing E. coli FabH obtained as described above, were resuspended in 140 ml of 20 mM Tris pH 7.9, 50 mM NaCl, 1 mM EDTA, 1 mM DTT, 10% glycerol, 1 mM PMSF (buffer A). Lysozyme ( Sigma Chemicals: hen egg) was added to a final concentration of 1 mg/ml. Cells were incubated at 37° C. for 20 min. The cells were then lysed by sonication in an ice water bath (4×30 sec). DNAse (Sigma; bovine pancreas type 1) was added along with MgCl₂ and held at 37° C. for 5 minutes. The solution was centrifuged in a Beckman JA-HS centrifuge at 14,000 g for 60 minutes using a Beckman JA-14 rotor.

2) Anion Exchange

All chromatography was performed on a Pharmacia chromatography system, fitted with a UV detector (Pharmacia, Monitor UV-1). UV (at 280 nm) was monitored during all operations. All operations were performed at 4° C.

The supernatant from 1) was loaded onto a Q-Sepharose high performance (Pharmacia) column of 50 ml packed into a Pharmacia XK-26 column. The column was equilibrated with buffer A prior to loading. The column was then washed with buffer A (250 ml) at 4 ml/min, and eluted with a linear gradient of buffer A to 1M NaCl in buffer A over 80 minutes at 4 ml/min. The eluate was fractionated into 8 ml fractions using a Pharmacia FRAC 200.

The eluted fractions were assayed for FabH activity by measurement of incorporation of [¹⁴C]Acetyl-CoA to Malonyl-ACP and , and for protein by the Bradford method. Active fractions were analyzed by reducing SDS-PAGE (NOVEX, NuPAGE Bis-Tris 4-12% gradient gel). Active fractions pooled together and dialyzed against Buffer A.

3) Anion Exchange Chromatography

The dialyzed material was loaded onto a MonoQ column equilibrated with buffer A (Pharmacia, 5/5). The column was washed with 20 ml of the equilibration buffer until 280 nm absorbance returned to base line and then eluted with a linear gradient of equilibration buffer to buffer A over 90 minutes at 0.5 ml/min. Fractions were pooled together, collected, assayed for FabH activity.

4) Hydroxyapatite/Buffer Exchange

Eluted fractions are collected (1 ml fraction) and assayed for FabH activity and protein. Active fractions are pooled and the volume was doubled with Buffer B [20 mM Tris-HCl pH 7.4, 50 mM NaCl, 1 mM DTT and 10% glycerol] to reduce the salt concentration in half. The active eluate was loaded in a hydroxyapatite column and eluted with 0.5 M Potassium Phosphate pH 7.4. The active enzyme was buffer exchanged with 20 mM Tris pH 7.4, 50 mM NaCl 2 mM DTT. This product was greater than 97% purity by SDS PAGE and the activity showed an overall process yield of 60% from 1). N-terminal amino acid analysis confirmed identity.

### EXAMPLE 3

### Fermentation, Purification and Characterization of Seleno-methionine derivative of Escherichia coli FabH

A. Fermentation

To obtain soluble selmet-FabH for purification and crystallization studies, E. coli strain BL21 (DE3) was transformed with pET29c FabH. 50 ul of the seed culture expressing FabH gene product was inoculated into 100 ml of Luria broth, containing kanamycin (50 ug/ml) and glucose (0.6%). On reaching target density of 2 OD, the cells from the seed culture were isolated by centrifugation, resuspended in 100 ml of M9 minimal medium containing 1 mM CaCl₂, 1 mM MgSO₄, kanamycin ( 50 ug/ml) and glucose (0.6% w/v). The resuspended pellets were then added to 900 ml of the same medium and the cells were grown at 37 C to mid-log phase, A₆₀₀ of 1.5, at which point lysine, phenylalanine, threonine at 100 mg/l each, and selenomethionine, isoleucine, leucine, and valine at 50 mg/l were added. The culture was shaken for 15 minutes, and then induced with 0.5 mM isopropyl b-D-thiogalactopyranoside (IPTG). The culture was grown for 13 hours, and harvested by centrifugation (speed). 5 ml aliquots were taken prior to and during induction to monitor the expression of selenomethionine FabH. 12 g of cell paste (wet wt) was recovered from 5L. In addition, to compare the expression of selenomethionine derivative to that of wt FabH, a one 1 culture was prepared under identical conditions except that the cells were grown in LB media.

B. Purification

All lysis and purification steps were carried out using degassed buffers in a cold room or on ice. 4.5 g of E. coli cells over expressing Fab H were resuspended in 50 ml of 20 mM Tris, 50 mM sodium chloride, 10% glycerol, 0.2 mM PMSF, 2 mM DTT, pH 7.9 (buffer A). Cells were lysed twice at 10,000 psi using Microfluidizer (Microfluidics Corporation, Mass.). Cell debris was removed by centrifugation (Sorvall RC-5B) at 35,000 g for 30 min. The supernatant was applied to a 2.6×4 cm Source Q column (Pharmacia) equilibrated in buffer A. The column was washed with 10 column volumes of buffer A, and eluted with a 10 column volume gradient of 0 to 1.0 M NaCl in buffer A. Eluted fractions were analyzed by 10% SDS-PAGE under reducing conditions. Fab H eluted at 0.2-0.25 M NaCl. Fab H containing fractions were pooled and applied to a 2.6×6 cm Hydroxyapatite column (Bio-Rad, Type I, 40 u) equilibrated in buffer A. The column was eluted with a 30 column volume linear gradient of buffer A to 400 mM potassium phosphate, 10% glycerol, 2 mM DTT. Fab H, which eluted at 80-180 mM potassium phosphate, was diluted 1:2 with 50 mM Tris, 200 mM NaCl, 10% glycerol, 2 mM DTT, pH 7.5 (buffer B) and applied to a 1.6×7.5 cm Blue Sepharose column (Pharmacia) equilibrated in buffer B. The column was eluted with buffer B containing 1 M NaCl. Blue Sepharose eluted Fab H fractions were next applied to a 2.6×60 cm Superdex 200 size exclusion column (Pharmacia) equilibrated in 20 mM Tris, 50 mM NaCl, 2 mM DTT, pH 7.4. A total of 23 mg of Fab H was recovered which was concentrated to 13 mg/ml using Amicon 3 filtration device.

C. Characterization

i). Mass Spectoscopy

Matrix-assisted laser desorption ionization mass spectrometry (MALDI-MS) data were obtained on a PerSeptive Biosystems Voyager RP laser desorption time-of-flight mass spectrometer. Protein samples were prepared for analysis by diluting analyte 1:5 with 3,5-Dimethoxy-4-hydroxy-cinnamic acid (10 mg/ml in 2:1 0.1% trifluoroacetic acid/acetonitrile) for a final concentration of 1-10 pmol/ul. Bovine Beta lactoglobulin A (Sigma) was included as an internal calibrant (MH⁺18364 Da). Desorption/ionization was accomplished using photon irradiation from a 337-nm pulsed nitrogen laser and 30-keV accelerating energy. Spectra were averaged over ca. 100 laser scans.

The predicted molecular mass for native FabH is 33516 Da. MALDI-MS analysis of the selenomethionyl incoporated FabH protein construct provided a mass of 33,889 Da. This is in close agreement with the predicted +375Da shift in mass expected for the sulfur to selenium side-chain substitution of eight methionine residues within the protein (33,891 Da theoretical).

ii). N-terminal Sequence Analysis

Sequence analysis was performed on a Hewlett-Packard G1000A N-terminal Protein Sequencer with on-line PTH identification using an HP1100 HPLC. Samples were applied directly to biphasic sequencing cartridges and standard 3.1 sequencing method cycles were used.

N-terminal sequencing results showed negligible native methionine in the first residue. Instead, a unique PTH (phenylthiohydantoin) derivative was observed which eluted 1.6 minutes later than PTH-methionine, and did not coelute with any natural PTH-amino acid derivatives. The increase in hydrophobicity is consistent with the direct detection of the PTH-selenomethionyl amino acid derivative.

D. Measurement of β-ketoacyl-ACP Synthase III Activity.

The enzyme catalyses the condensation of acetyl-CoA with malonyl-ACP to form acetoacetyl-ACP. The reaction can be described by three distinct steps: (a) the acyl group of acetyl-CoA is transferred to the active site cysteine resulting in a acyl-enzyme thioester; (b) carbanion formation by the decarboxylation of malonyl-ACP; and (c) carbon-carbon bond formation by nucleophillic attack of the carbanion onto the carbonyl carbon atom of the acyl-enzyme thioester to yield the acetoacetyl-ACP product.

This reaction can be assayed in order to characterize the enzyme or identify specific inhibitors of its activity in two ways:

(1) Radiolabeled acetoacetyl-ACP formation can be specifically determined using [3H]-acetyl-CoA and malonyl-ACP. The [3H]-acetyl-CoA substrate is soluble in 10% TCA while the resulting [3H]-acetoacetyl-ACP is not. A reaction mixture containing 100 mM sodium phosphate buffer pH7.0, 1 mM DTT, 34 uM acetyl-CoA, 0.15 uM [3H]-acetyl-CoA (specific activity 25 Ci/mmol), and 7 uM malonyl-ACP is prepared and transfered to a microtiter plate with or without inhibitors already added. The enzyme is added last to start the reaction and the plate is incubated at 37 degrees C. Ten percent TCA is added to stop the reaction, and then 50 ug of BSA as a carrier. Stopped reactions are filtered and washed 2 times with 10% TCA on Wallac GF/A filtermats using a TomTec harvester. The filtermats are dried at 60 degrees C and the radioactivity quantified using Wallac Betaplate scintillation cocktail and a Wallac Microbeta 1450 liquid scintillation counter. IC50s are generated using Grafit 4.0 software and solved using the equation v=Vmax/(1+I/IC50).

(2) FabG coupling can also be used to measure FabH production of acetoacetyl-ACP by following NADPH consumption at 340 nm. FabG (β-ketoacyl-ACP reductase) will specifically reduce the C3 carbonyl of acetoacetyl-ACP to form β-hydroxybutyryl-ACP. This reduction requires the conversion of NADPH to form NADP+ which can be monitored by following the optical density at wavelength 340 nm.

(3) FabD coupling is an available assay option in the absence of purified malonyl-ACP. FabD (Malonyl-CoA:ACP transacylase) is responsible for malonic acid transfer from malonyl-CoA to ACP to form malonyl-ACP. This activity can be exploited by applying the techniques described in (1) above together with de novo malonyl-ACP from the FabD reaction.

E. Ligand Binding to FabH.

It is also possible to define ligand interactions with FabH in experiments that are not dependent upon enzyme catalyzed turnover of substrates. This type of experiment can be done in a number of ways:

(1) Effects of Ligand Binding Upon Enzyme Intrinsic Fluorescence (e.g. of Tryptophan). Binding of either natural ligands or inhibitors may result in enzyme conformational changes which alter enzyme fluorescence. Using stopped-flow fluorescence equipment, this can be used to define the microscopic rate constants that describe binding. Alternatively, steady-state fluorescence titration methods can yield the overall dissociation constant for binding in the same way that these are accessed through enzyme inhibition experiments.

(2) Spectral Effects of Ligands. Where the ligands themselves are either fluorescent or possess chromophores that overlap with enzyme tryptophan fluorescence, binding can be detected either via changes in the ligand fluorescence properties (e.g. intensity, lifetime or polarization) or fluorescence resonance energy transfer with enzyme tryptophans. The ligands could either be inhibitors or variants of the natural ligands.

(3) Thermal Analysis of the Enzyme:Ligand Complex. Using calorimetric techniques (e.g. Isothermal Calorimetry, Differential Scanning Calorimetry) it is possible to detect thermal changes, or shifts in the stability of FabH which reports and therefore allows the characterization of ligand binding.

### EXAMPLE 3

### Crystallization of E. coli Wild-Type and Selenomethionine Mutant of FabH

A. Crystallization

All crystals were grown at room temperature using the sitting-drop vapor diffusion method. The drop solution was always a 1:1 mixture of the protein sample and the well solutions. For the crystal form 1 of the wild-type protein, the well solution contained 0.1 M HEPES buffer at pH 7.5 and 20% PEG8000. For the crystal form 2 of the selenomethionine mutant protein in complex with acetyl-CoA, the well solution contained 0.05 M Bis-Tris propane buffer at pH 7.0, 0.1 M MgCl₂ and 14% PEG6000. Crystals grew overnight and are approximately 0.1 to 0.2 mm in sizes.

B. X-ray Diffraction Characterization

All crystals were frozen in liquid nitrogen streams before their characterization using synchrotron X-ray radiation. Diffraction data for the apo form 1 crystal was collected to 2.0 Å resolution. The data is 97.1% complete and 6 fold redundant with a merging R- factor of 7.7%. The crystal belongs to the orthorhombic spacegroup P2₁2₁2₁, with cell dimensions a=63.1, b=65.1 and c=166.5 Å. For the Se-Met protein in complex with acetyl-CoA, data were collected at three different wavelengths: 0.9789, 0.9785 and 0.9414 Å. The three data set were of nearly identical quality, with about 80% completion, 6-fold redundancy, 8.5% merging R-factor, and 1.9 Å resolution. The form 2 crystal belongs to the tetragonal spacegroup P4₁2₁2, with a=b=72.4 and c=102.8 Å.

C. Structure Solution

The crystal structure of the Se-Met E. coli FabH mutant in complex with acetyl-CoA was solved to 1.9 Å resolution using the MAD phasing technique with the data sets collected at three different wavelengths and the program SOLVE (Terwilliger & Berendzen, 1999, Acta Cryst. D55, 849-861). All eight Se-Met were located by SOLVE. The overall MAD phasing figure of merit was 0.6 to 1.9 Å resolution, and the overall Z score was as high as 148. The resulting electron density map was of very high quality. The structure of the apo enzyme (crystal form 1) was solved with the molecular replacement method using the acetyl-CoA complex structure as the search model. This crystal form had a FabH dimer in the asymmetric unit, and the R-factor of the solution was only 33%. Two-fold averaged map was then calculated and used for model building.

D. Model Building and Refinement

The electron density for the acetyl-CoA complex was very clear and a structure model for the whole FabH protein, the bound acetyl group and CoA, as well as 98 solvent molecules were built in the first round. Standard structural refinement protocols and manual model building led to the current model, which has an R-factor of 27% to 1.9 Å resolution. The model for the apo FabH structure was also built readily, and refined to an R-factor of 18.9% (R_{free} of 24.4%) to 2.0 Å resolution. Both models have excellent geometry and do not have any outliers in the Ramanchandran plot, indicating high quality of the atomic coordinates, which contain an estimated error of less than 0.3 Å.

This invention is not to be limited in scope by the specific embodiments described herein. Indeed, various modifications of the invention in addition to those described herein will become apparent to those skilled in the art from the foregoing description. Such modifications are intended to fall within the scope of the appended claims. The disclosures of the patents, patent applications and publications cited herein are incorporated by reference in their entireties.

What is claimed is:

1. A composition comprising a E. coli FabH in crystalline form.

2. The composition according to claim 1 wherein said FabH is a dimer.

3. The composition according to claim 1 wherein said FabH comprises an active site cavity formed by amino acids comprising Cys112, His244 and Asn274

4. The composition of claim 1 wherein said FabH is a E. coli FabH.

5. The composition of claim 3 wherein said FabH is characterized by the coordinates selected from the group consisting of the coordinates of FIGS. 1-2 and Tables I, II, and III.

6. A E. coli FabH crystal.

7. A selenomethionine mutant crystal of a E. coli FabH.

8. An isolated, properly folded FabH molecule or fragment thereof having a conformation comprising the protein coordinates of FIGS. 1-2 and Tables I, II, and III.

9. The molecule according to claim 8 wherein said molecule is a dimer, wherein each monomer is characterized by two similar domains having core of five β-strands, each containing flanking helices, strands and loops, as illustrated in FIG. 3.

10. The molecule according to claim 8 wherein said molecule is a dimer characterized by the dimer interface of FIG. 3.

11. The molecule according to claim 10 which is E. coli FabH.

12. A peptide, peptidomimetic or synthetic molecule which interacts competitively or non-competitively with the active site of a FabH of claim 1.

13. A method of identifying an inhibitor compound capable of binding to, and inhibiting the enzymatic activity of, a E. coli FabH, said method comprising: introducing into a suitable computer program information defining an active site conformation of a E. coli FabH molecule comprising a conformation defined by the coordinates of FIGS. 1-2 and Tables I, II, and III, wherein said program displays the three-dimensional structure thereof; creating a three dimensional structure of a test compound in said computer program; displaying and superimposing the model of said test compound on the model of said active site; assessing whether said test compound model fits spatially into the active site; incorporating said test compound in a biological activity assay for a FabH characterized by said active site; and determining whether said test compound inhibits enzymatic activity in said assay.

14. The method according to claim 13 wherein said FabH molecule is a dimer, wherein each monomer is characterized by two similar domains having core of five β-strands, each containing flanking helices, strands and loops, as illustrated in FIG. 3.

15. A method of identifying an inhibitor compound capable of binding to, and inhibiting the enzymatic activity of, a E. coli FabH, said method comprising: introducing into a suitable computer program information defining an active site conformation of a FabH molecule comprising a conformation defined by the coordinates of FIGS. 1-2 and Tables I, II, and III, wherein said program displays the three-dimensional structure thereof; creating a three dimensional structure of a test compound in said computer program; displaying and superimposing the model of said test compound on the model of said active site; assessing whether said test compound model fits spatially into the active site; incorporating said test compound in a biological activity assay for a FabH characterized by said active site; and determining whether said test compound inhibits enzymatic activity in said assay.

16. The method according to claim 15 wherein said FabH molecule is a dimer, wherein each monomer is characterized by two similar domains having core of five -strands, each containing flanking helices, strands and loops, as illustrated in FIG. 3.

17. A peptide, peptidomimetic or synthetic molecule identified by the method of claim 13 or 15.

18. A method for solving a crystal form comprising using the structural coordinates of a E. coli FabH crystal or portions thereof, to solve a crystal form of a mutant, homologue or co-complex of said FabH by molecular rearrangement.

19. A method of drug design comprising the step of using the structural coordinates of a E. coli FabH crystal to computationally evaluate a chemical entity for associating with the active site and substrate binding sites of E. coli FabH.

20. The method of drug design according to claim 19 comprising the step of using the structure coordinates of E. coli FabH to identify an intermediate in a chemical reaction between said FabH and a compound with is a substrate or inhibitor of said enzyme.

21. The method according to claim 20, wherein said entity is a competitive or non-competitive inhibitor of a E. coli FabH.

22. The method of drug design according to claim 19, using the structure of a FabH homologue that has similar amino acid identities as well as spacial arrangements as those of E. coli FabH listed in Tables I-III.

23. The method of drug design according to claim 20 using the structure of a FabH homologue that has similar amino acid identities as well as spacial arrangements as those of E. coli FabH listed in Tables I-III.

24. The method of drug design according to claim 21 using the structure of a FabH homologue that has similar amino acid identities as well as spacial arrangements as those of E. coli FabH listed in Tables I-III.

25. The method according to claim 19 wherein said structure coordinates comprise the coordinates of FIGS. 1-2 and Tables I, II, and III.

26. The method according to claim 20 wherein said structure coordinates comprise the coordinates of FIGS. 1-2 and Tables I, II, and III.

27. The method according to claim 21 wherein said structure coordinates comprise the coordinates of FIGS. 1-2 and Tables I, II, and III.


SEQUENCE LISTING (3 sequences)

SEQ ID NO: 1 - protein, 317 aa, Escherichia coli
MYTKIIGTGSYLPEQVRTNADLEKMVDTSDEWIVTRTGIRERHIAAPNETVSTMGFEAATRAIEMAGIEKDQIGLIVVATTSATHAFPSAACQIQSMLGIKGCPAFDVAAACAGFTYALSVADQYVKSGAVKYALVVGSDVLARTCDPTDRGTIIIFGDGAGAAVLAASEEPGIISTHLHADGSYGELLTLPNADRVNPENSIHLTMAGNEVFKVAVTELAHIVDETLAANNLDRSQLDWLVPHQANLRIISATAKKLGMSMDNVVVTLDRHGNTSAASVPCALDEAVRDGRIKPGQLVLLEAFGGGFTWGSALVRF

SEQ ID NO: 2 - DNA, 28 bp, Escherichia coli
tatacatatgtatacgaagattattggt

SEQ ID NO: 3 - DNA, 30 bp, Escherichia coli
atatggatccctagaaacgaaccagcgcgg